(19) United States
(12) Patent Application Publication
(10) Pub. No.: US 2025/0340600 A1
(43) Pub. Date: Nov. 6, 2025

(54) LENTIVIRAL-BASED VECTORS AND RELATED SYSTEMS AND METHODS FOR EUKARYOTIC GENE EDITING

(71) Applicant: Wake Forest University Health Sciences, Winston-Salem, NC (US)

(72) Inventors: Baisong Lu, Winston-Salem, NC (US); Anthony Atala, Winston-Salem, NC (US)

(73) Assignee: Wake Forest University Health Sciences, Winston-Salem, NC (US)

(21) Appl. No.: 19/270,184

(22) Filed: Jul. 15, 2025

Related U.S. Application Data
(62) Division of application No. 17/051,296, filed on Oct. 28, 2020, now Pat. No. 12,398,182.
(60) Provisional application No. 62/665,080, filed on May 1, 2018.

(51) Int. Cl. C07K 14/155; C12N 9/22; C12N 15/113; C12N 15/86

(52) U.S. Cl. CPC C07K 14/155; C12N 9/22; C12N 15/113; C12N 15/86; C07K 2319/735; C12N 2310/16; C12N 2310/20; C12N 2740/15022

(57) ABSTRACT

Provided are compositions, systems, and methods useful for effecting gene editing in eukaryotic cells. Compositions include plasmids that encode one or more viral fusion proteins in which one or more viral proteins are fused with an aptamer-binding protein. Compositions also include plasmids that encode a non-viral nucleic acid sequence, wherein the non-viral nucleic acid sequence encodes a CRISPR system component. In some instances, the non-viral nucleic acid sequence also includes an aptamer sequence. The plasmids can be used to generate viral particles, including lentivirus-like particles that contain a viral fusion protein and a non-viral RNA sequence. Systems of producing such viral particles are provided. Also provided are methods of using the viral particles of the disclosure to effect gene editing in eukaryotic cells.

### PRIOR RELATED APPLICATION

This application is a divisional of U.S. patent application Ser. No. 17/051,296 filed Oct. 28, 2020, which is a 371 national phase entry of International Patent Application No. PCT/US2019/030198 filed on May 1, 2019, which claims the benefit of U.S. Provisional Application No. 62/665,080 filed on May 1, 2018, the contents of each of which are hereby incorporated by reference in their entirety for all purposes.

### REFERENCE TO A SEQUENCE LISTING SUBMITTED AS AN XML FILE

This application is accompanied by a sequence listing entitled 1511175-SL.xml, created Jul. 15, 2025, which is approximately 311,330 bytes in size. This sequence listing is incorporated herein by reference in its entirety. This sequence listing is submitted herewith and is in compliance with 37 C.F.R. §§ 1.831-1.835.

### BACKGROUND

The clustered regularly interspaced short palindromic repeats (CRISPR)/CRISPR-associated (Cas) system discovered in bacteria can be used as a tool to modify mammalian and human genomes, for gene therapy, gene expression regulation, DNA and RNA labeling. In CRISPR/Cas systems, a CRISPR-associated nuclease is targeted to a genomic site by complexing with a guide RNA (gRNA) that hybridizes to a target site in the genome. This results in a double stranded break that initiates either non-homologous end joining (NHEJ) or homology-directed repair (HDR) of genomic DNA via a double-stranded or single-stranded DNA template. Modified systems using a nickase (modified CRISPR-associated nuclease) that introduces a single stranded break have also been developed.

Currently, the common practice is to deliver CRISPR-associated nuclease expressing DNA by plasmid DNA, lentivirus, or adeno-associated virus. All of these delivery systems suffer from a risk of inducing mutagenesis due to the possibility of off-targets and prolonged nuclease expression. Thus, there is a need for efficient, transient delivery of the CRISPR/Cas9 components.

### SUMMARY

The present disclosure is directed to compositions, systems, and methods useful for effecting gene editing in eukaryotic cells. In some instances, the compositions, systems and methods can be used to package a CRISPR-associated endonuclease mRNA into a viral particle, for example, a lentiviral-particle. In some instances, the compositions, systems and methods can be used to package a CRISPR-associated endonuclease and a gRNA sequence, for example, as a ribonucleoprotein complex, into a viral particle. Compositions include plasmids that encode one or more viral fusion proteins in which one or more viral proteins are fused with an aptamer-binding protein. Compositions also include plasmids that encode a non-viral nucleic acid sequence, wherein the non-viral nucleic acid sequence encodes a CRISPR system component. In some instances, the non-viral nucleic acid sequence also includes an aptamer sequence. In some instances, a nucleic acid encoding a CRISPR-associated endonuclease comprises at least one aptamer sequence. In some cases, a gRNA coding sequence comprises at least one aptamer sequence. The plasmids can be used to generate viral particles, including lentivirus-like particles Systems of producing such viral particles are provided. Also provided are methods of using the viral particles described herein to effect gene editing in eukaryotic cells.

Also provided is a lentiviral packaging plasmid comprising a eukaryotic promoter operably linked to a Gag nucleotide sequence, wherein the Gag nucleotide sequence comprises a nucleocapsid (NC) coding sequence and a matrix protein (MA) coding sequence, wherein one or both of the NC coding sequence or the MA coding sequence comprises at least one non-viral aptamer-binding protein (ABP) nucleotide sequence, and wherein the packaging plasmid does not encode a functional integrase protein.

Further provided is a mammalian expression plasmid comprising an eukaryotic promoter operably linked to a Viral Protein R (VPR) coding sequence or a Negative Regulatory Factor (NEF) coding sequence, wherein the VPR coding sequence or the NEF coding sequence comprises at least one non-viral aptamer-binding protein (ABP) nucleotide sequence.

Also provided is a mammalian expression plasmid comprising an eukaryotic promoter operably linked to a non-viral nucleic acid sequence, wherein the non-viral nucleic acid sequence comprises at least one aptamer coding sequence, and wherein the non-viral nucleic acid sequence comprises (i) one or both of a CRISPR-associated endonuclease coding sequence or a guide RNA (gRNA) coding sequence, and (ii) at least one aptamer coding sequence.

Further provided is a lentiviral packaging system comprising: a) a packaging plasmid comprising a eukaryotic promoter operably linked to a Gag nucleotide sequence, wherein the Gag nucleotide sequence comprises a nucleocapsid (NC) coding sequence and a matrix protein (MA) coding sequence, wherein one or both of the NC coding sequence or the MA coding sequence comprises at least one non-viral aptamer-binding protein (ABP) nucleotide sequence, and wherein the packaging plasmid does not encode a functional integrase protein; b) at least one mammalian expression plasmid comprising a eukaryotic promoter operably linked to a non-viral nucleic acid sequence, wherein the non-viral nucleic acid sequence comprises a CRISPR-associated endonuclease coding sequence, a guide RNA (gRNA) coding sequence, or both a CRISPR-associated endonuclease coding sequence and a gRNA coding sequence; and c) an envelope plasmid comprising an envelope glycoprotein coding sequence.

Also provided is a lentiviral packaging system comprising: a) a packaging plasmid, and wherein the packaging plasmid does not encode a functional integrase protein; b) at least one mammalian expression plasmid comprising a eukaryotic promoter operably linked to a Viral Protein R (VPR) coding sequence or a Negative Regulatory Factor (NEF) coding sequence, wherein one or both of the VPR coding sequence or the NEF coding sequence comprises at least one non-viral aptamer-binding protein (ABP) nucleotide sequence; c) at least one mammalian expression plasmid comprising a eukaryotic promoter operably linked to a non-viral nucleic acid sequence, wherein the non-viral nucleic acid sequence comprises a CRISPR-associated endonuclease coding sequence, a guide RNA (gRNA) coding sequence, or both a CRISPR-associated endonuclease coding sequence and a gRNA coding sequence; and d) an envelope plasmid comprising an envelope glycoprotein coding sequence.

Further provided is a lentivirus-like particle comprising: a) a fusion protein comprising a nucleocapsid (NC) protein or a matrix (MA) protein, wherein the NC protein or MA protein comprises at least one non-viral aptamer binding protein (ABP); and b) at least one non-viral RNA molecule, wherein the non-viral RNA sequence comprises a CRISPR-associated endonuclease mRNA, a guide RNA (gRNA), or both a CRISPR-associated endonuclease mRNA and a gRNA; wherein the lentivirus-like particle does not comprise a functional integrase protein.

Also provided is a lentivirus-like particle comprising: a) a fusion protein comprising a Viral Protein R (VPR) protein or a Negative Regulatory Factor (NEF) protein, wherein VPR protein or the NEF protein comprises at least one non-viral aptamer binding protein (ABP); and b) at least one non-viral RNA molecule, wherein the non-viral RNA sequence comprises a CRISPR-associated endonuclease mRNA, a guide RNA (gRNA), or both a CRISPR-associated endonuclease mRNA and a gRNA; wherein the lentivirus-like particle does not comprise a functional integrase protein.

Further provided is a lentivirus-like particle comprising: a) a fusion protein comprising a Viral Protein R (VPR) protein or a Negative Regulatory Factor (NEF) protein, wherein VPR protein or the NEF protein comprises at least one non-viral aptamer binding protein (ABP); and b) a ribonucleotide protein (RNP) complex comprising a CRISPR-associated endonuclease and a guide RNA; wherein the lentivirus-like particle does not comprise a functional integrase protein.

Also provided is a method of producing a lentiviral particle, the method comprising: a) transfecting a plurality of eukaryotic cells with the packaging plasmid, the at least one mammalian expression plasmid, and the envelope plasmid of any one of the systems described herein; and b) culturing the transfected eukaryotic cells for sufficient time for lentiviral particles to be produced.

Further provided is a method of producing a lentiviral particle, the method comprising: a) transfecting a plurality of eukaryotic cells with the plasmids of any one of the systems described herein; and b) culturing the transfected eukaryotic cells for sufficient time for lentiviral particles to be produced.

Also provided is a method of modifying a genomic target sequence in a cell, the method comprising transducing a plurality of eukaryotic cells with a plurality of viral particles, wherein the plurality of viral particles comprise: i) any lentivirus-like particle described herein, wherein which the non-viral RNA sequence comprises a CRISPR-associated endonuclease mRNA and a gRNA, or ii) any lentivirus-like particle described herein, wherein the non-viral RNA sequence comprises a CRISPR-associated endonuclease mRNA and a second viral particle comprises a gRNA or a gRNA coding sequence, wherein a CRISPR-associated endonuclease is expressed from the CRISPR-associated endonuclease mRNA in cells transduced with the lentivirus-like particle, wherein, if the second viral particle comprises a gRNA coding sequence, a gRNA is expressed from the gRNA coding sequence in cells transduced with the second viral particle, and wherein the CRISPR-associated endonuclease and the gRNA form a complex that binds to the genomic target sequence in genomic DNA of the cell and the CRISPR-associated endonuclease cleaves the genomic DNA of the cell, thereby triggering cellular DNA repair mechanisms causing modification of the genomic target sequence.

Further provided is a method of modifying a genomic target sequence in a cell, the method comprising transducing a plurality of eukaryotic cells with a plurality of viral particles, wherein the plurality of viral particles comprise: i) a lentivirus-like particle comprising a ribonucleotide protein (RNP) complex (a CRISPR-associated endonuclease complexed with at least one gRNA), wherein the RNP binds to the genomic target sequence in genomic DNA of the cell and the CRISPR-associated endonuclease cleaves the genomic DNA of the cell, thereby triggering cellular DNA repair mechanisms causing modification of the genomic target sequence. Cells modified by any of the genomic modification described herein are also provided. Also provided are cells containing any of the lentivirus-like particles described herein.

Further provided is a method for treating a disease in a subject comprising: a) obtaining cells from the subject; b) modifying the cells of the subject using any of the methods of modifying a genomic target sequence described herein; and c) administering the modified cells to the subject. In some instances, the disease is cancer. In some instances the cells are T cells.

### BRIEF DESCRIPTION OF THE DRAWINGS

The present disclosure includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIGS. 1A-1B show schematics illustrating compositions, systems, and methods according to aspects of this disclosure. As shown in FIG. 1A, a plasmid is used to express a lentivirus protein fused with an aptamer-binding protein (ABP), referred to in the figures as a viral protein-ABP fusion protein. The plasmid encoding the viral protein-ABP fusion protein coding sequence may be a lentivirus packaging plasmid (e.g., for NC or MA fusion proteins) or a mammalian expression vector (e.g., for NEF or VPR fusion proteins). FIG. 1A also shows mammalian expression plasmids containing non-viral nucleic acid sequences encoding CRISPR system components (i.e. CRISPR-associated endonuclease and sgRNA) that are used together with a packaging plasmid and an envelope plasmid to transfect eukaryotic cells and generate lentivirus-like particles that contain the viral protein-ABP fusion protein and non-viral RNA molecules (i.e. CRISPR-associated endonuclease mRNA, sgRNA, or both). Optionally, the non-viral RNA molecules encoded by the mammalian expression plasmid(s) may comprise an aptamer sequence. If the viral protein-ABP fusion protein coding sequence is provided on a mammalian expression plasmid (e.g., for NEF and VPR fusion proteins), a lentiviral packaging plasmid is also used to transfect the eukaryotic cells. The mammalian expression plasmids may be lentiviral transfer plasmids encoding various viral genes. The packaging plasmids, whether they contain the viral protein-ABP fusion protein coding sequence or not, and the transfer plasmid, if used, may be modified to remove viral sequences so as to generate lentivirus-like particles. The lentivirus-like particles are collected from the eukaryotic cells. Optionally, sgRNA may be packaged in lentivirus particles or sgRNA coding sequence may packaged in DNA viruses instead of in lentivirus-like particles.

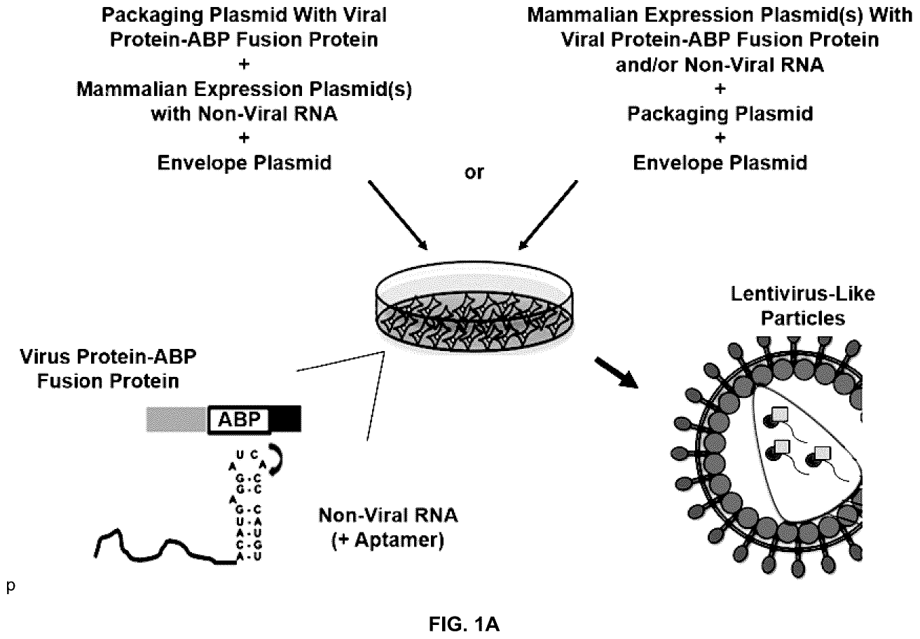

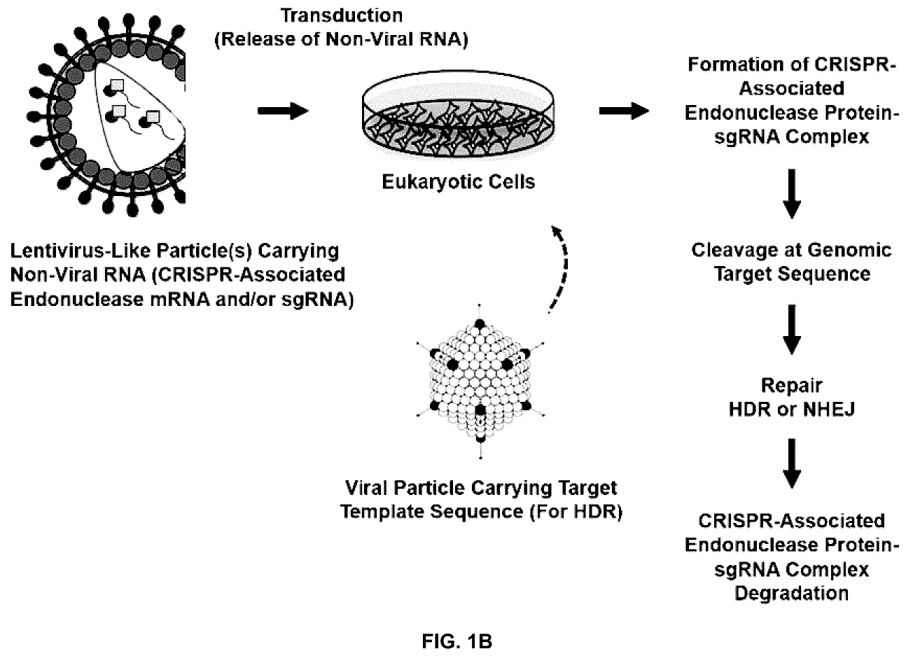

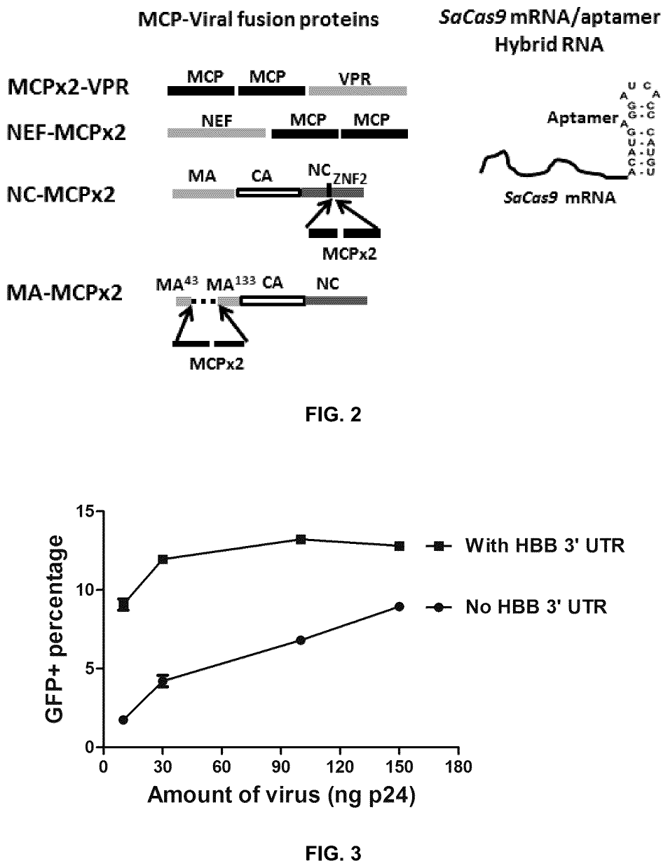

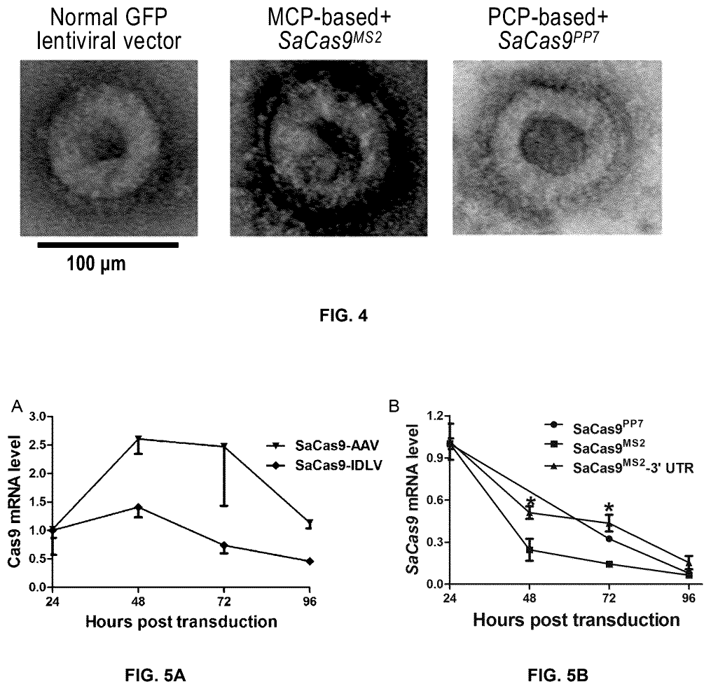

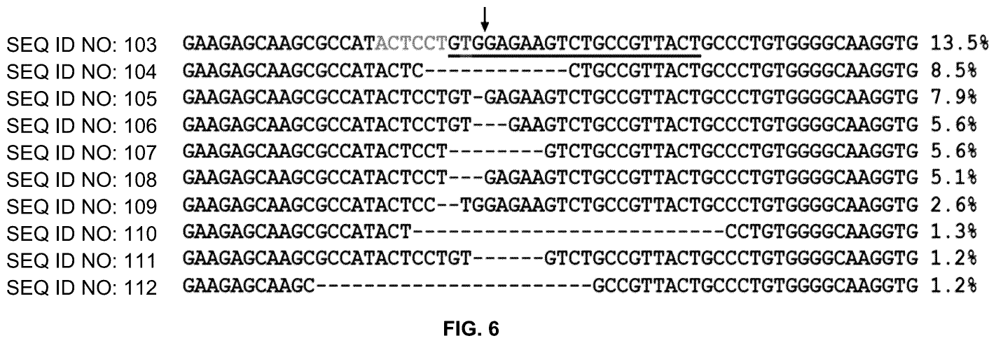

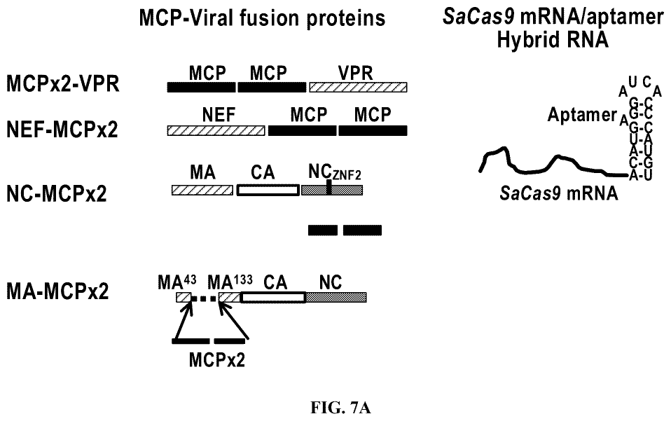

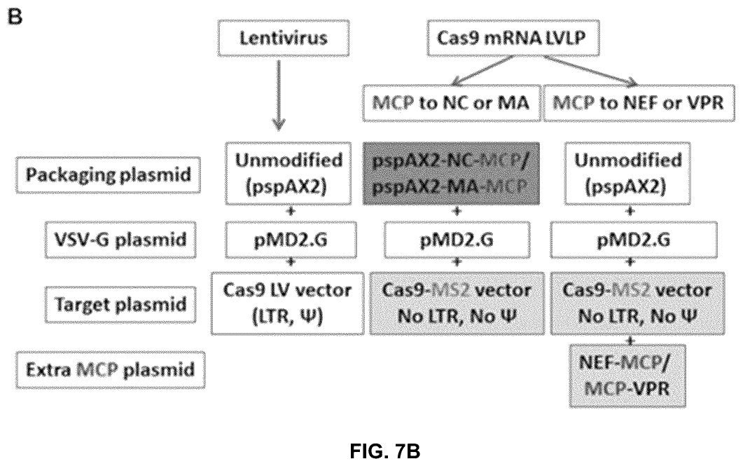

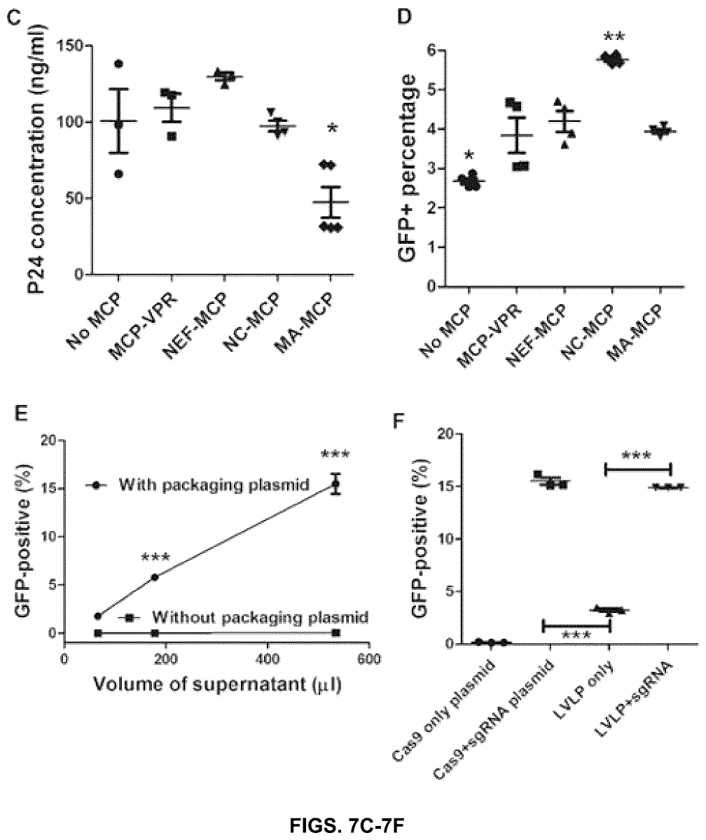

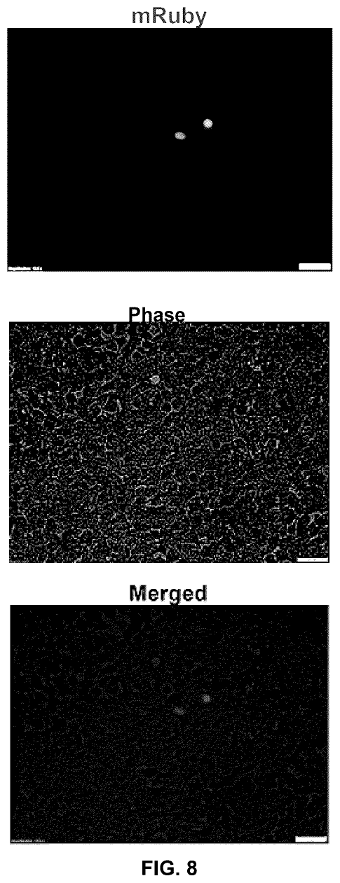

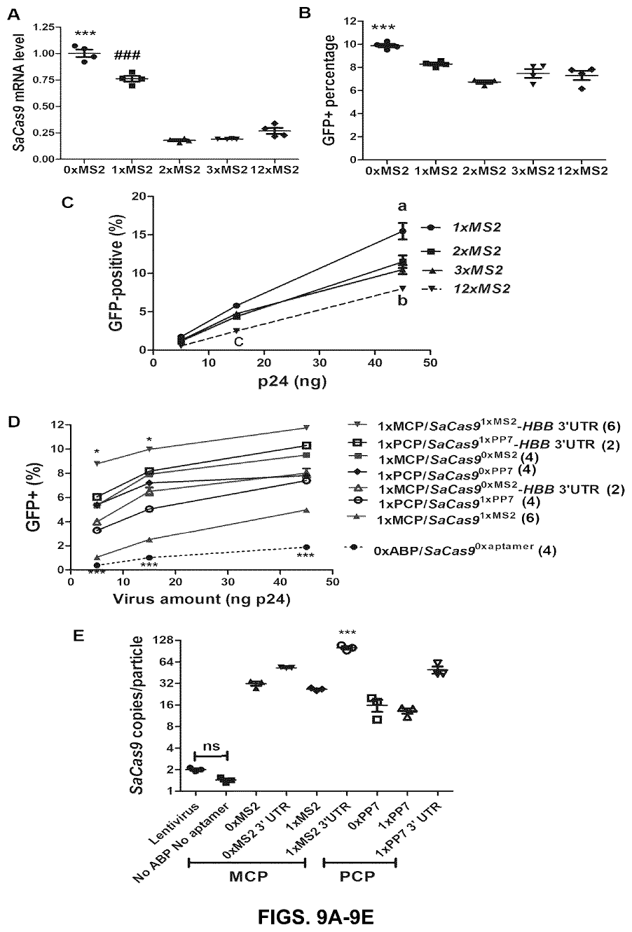

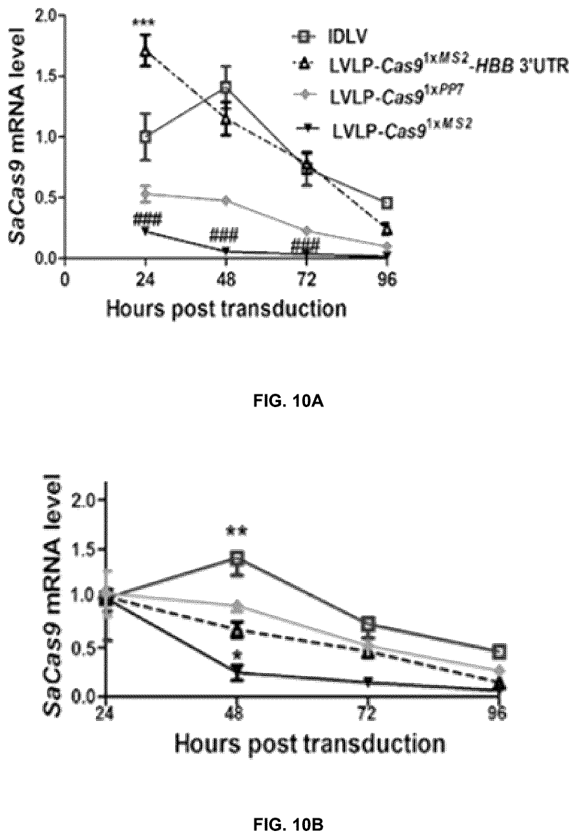

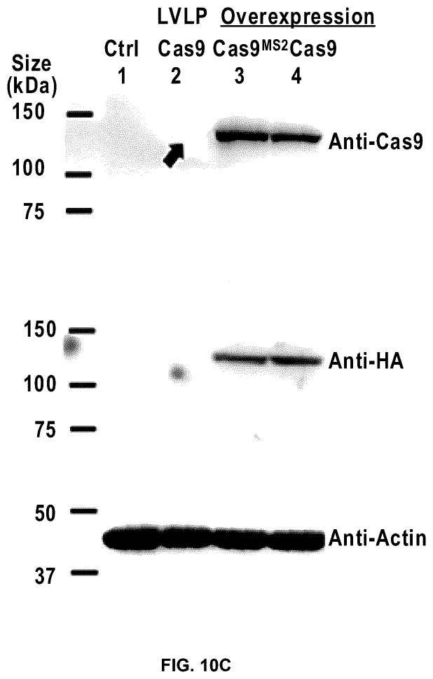

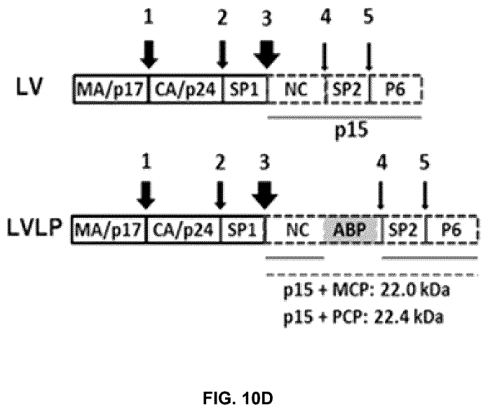

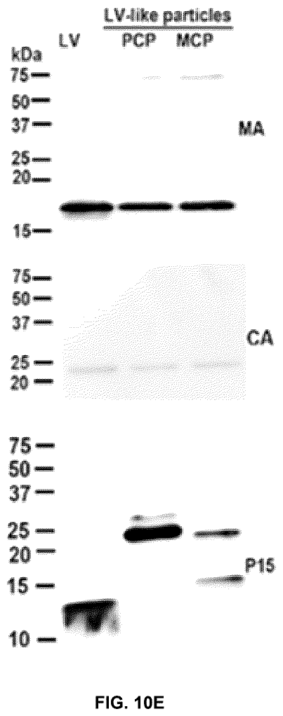

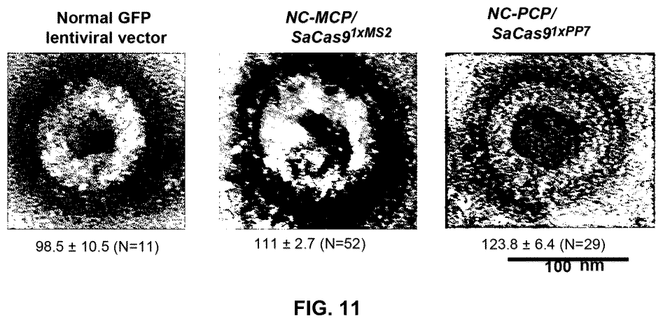

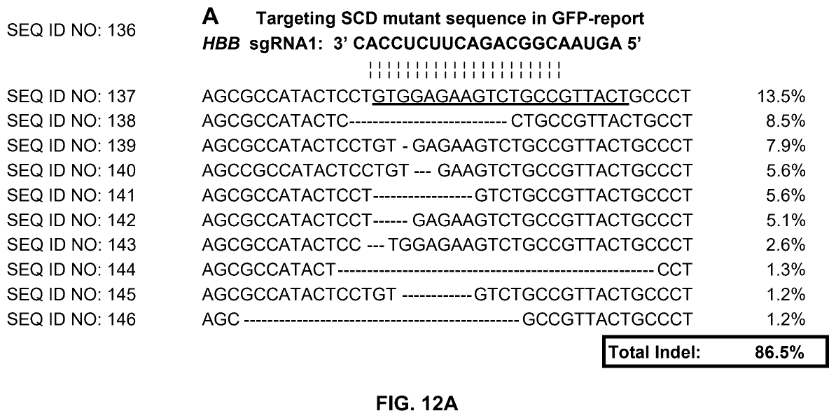

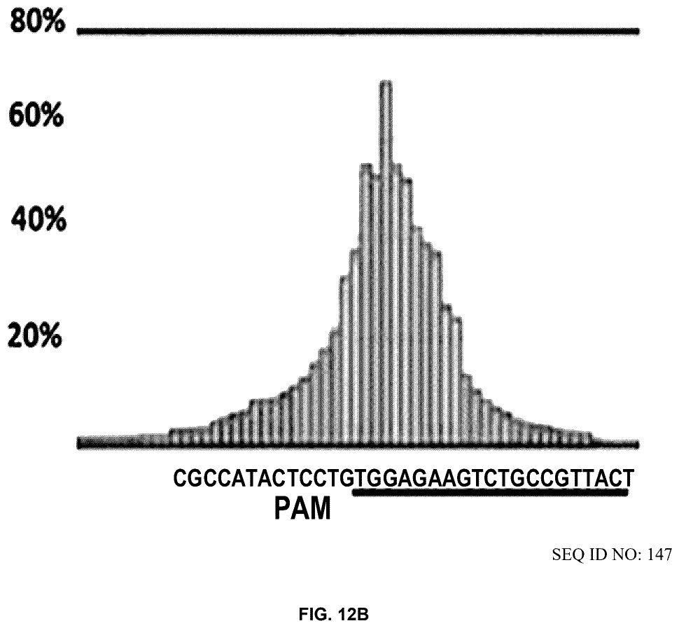

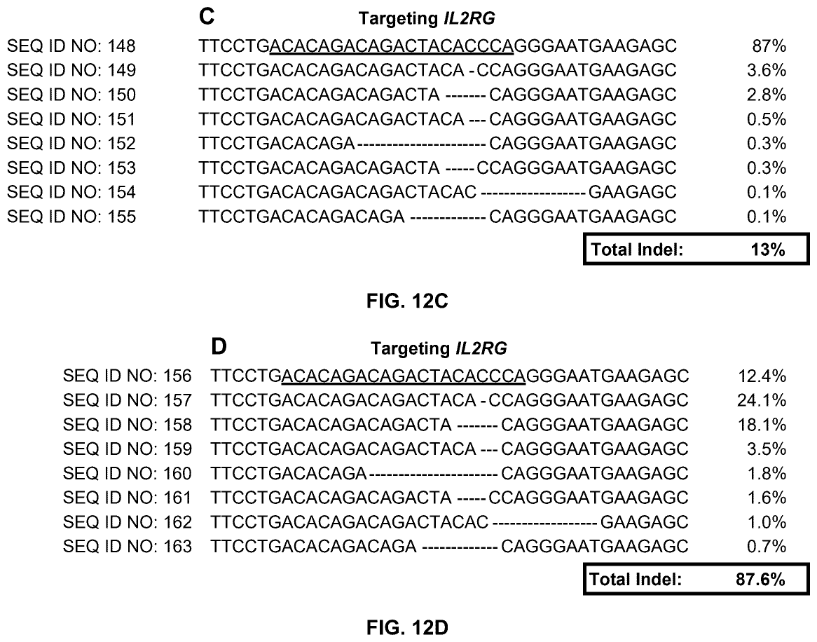

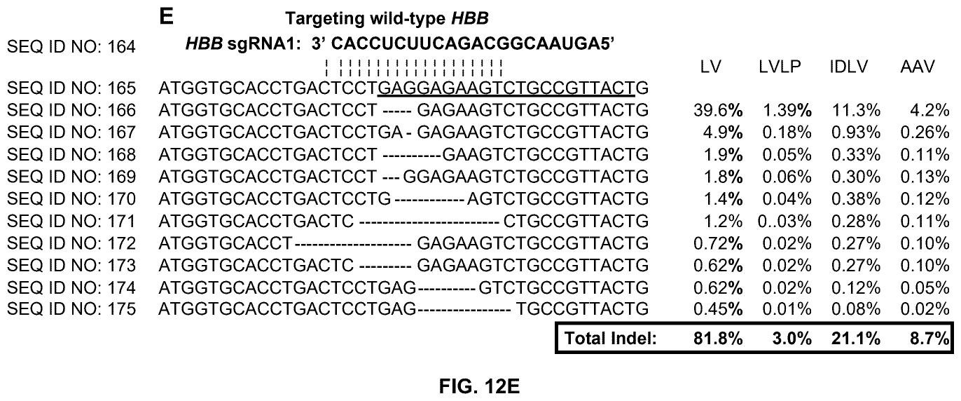

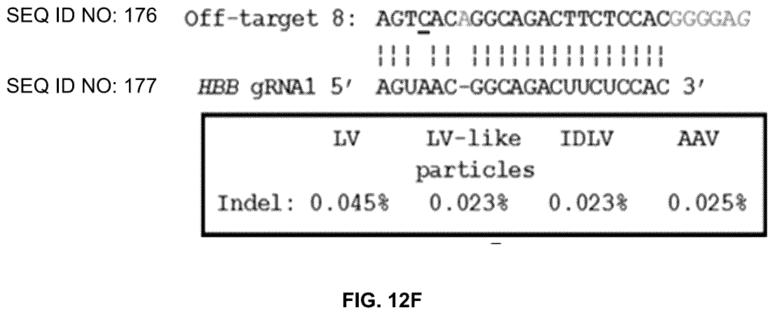

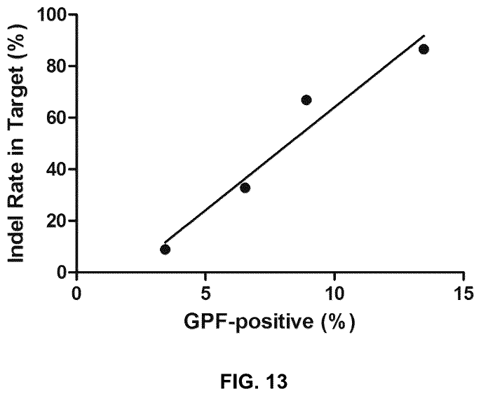

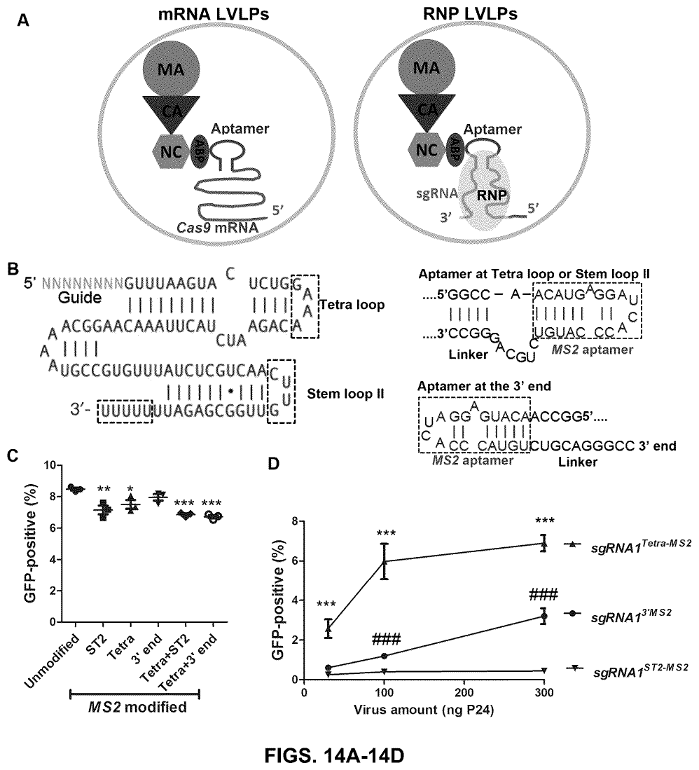

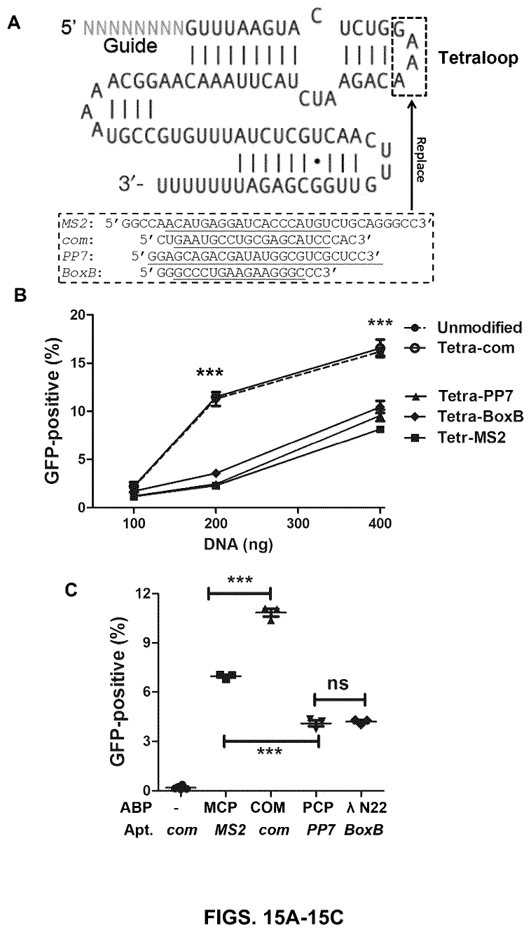

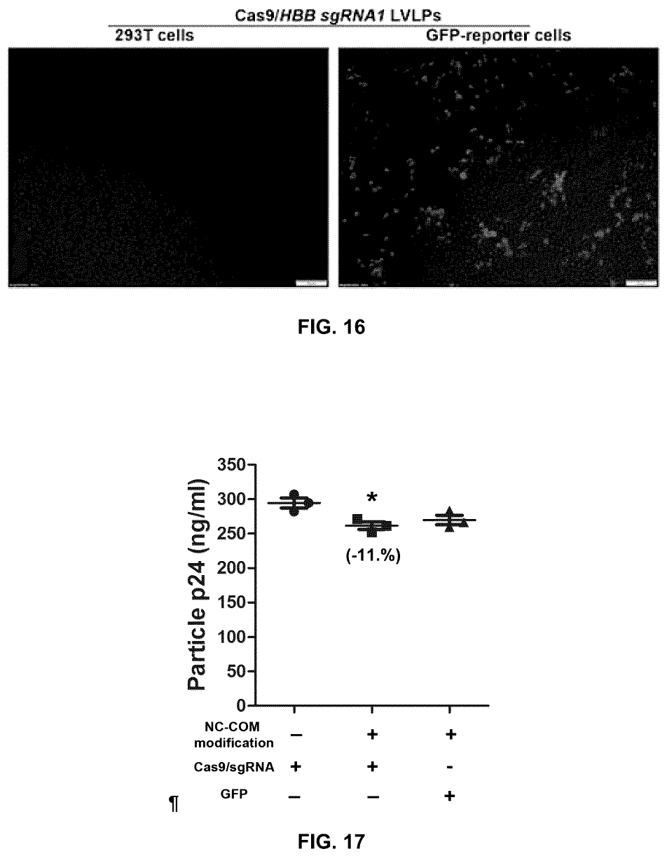

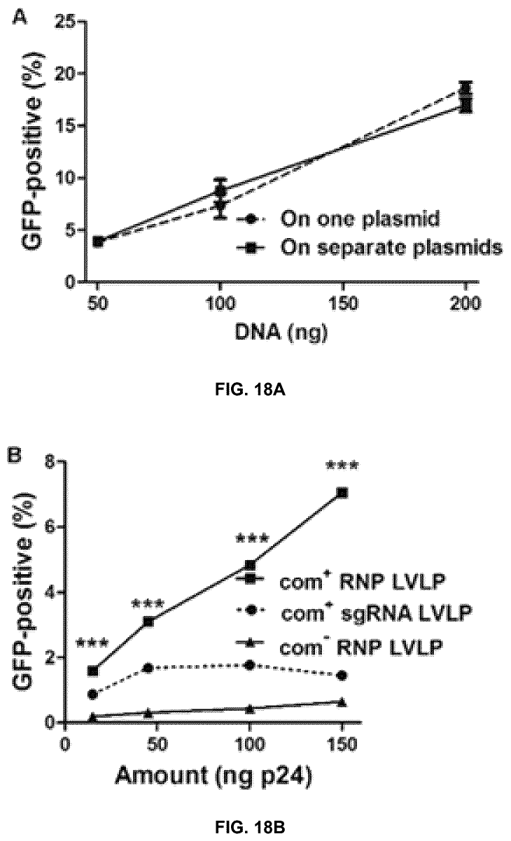

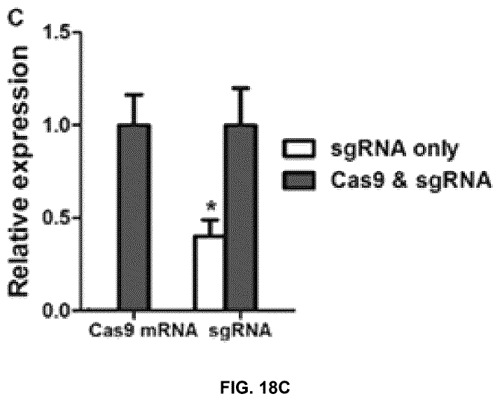

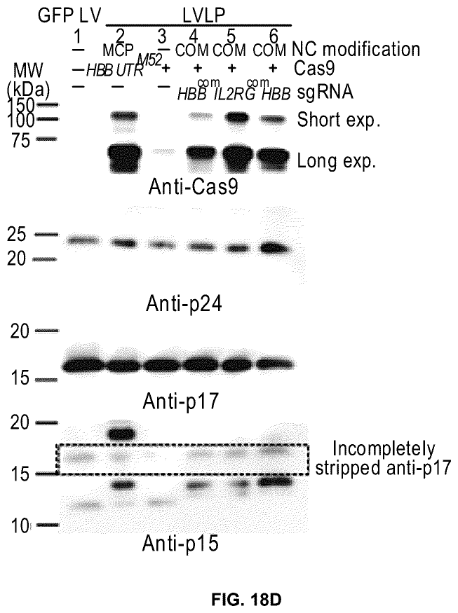

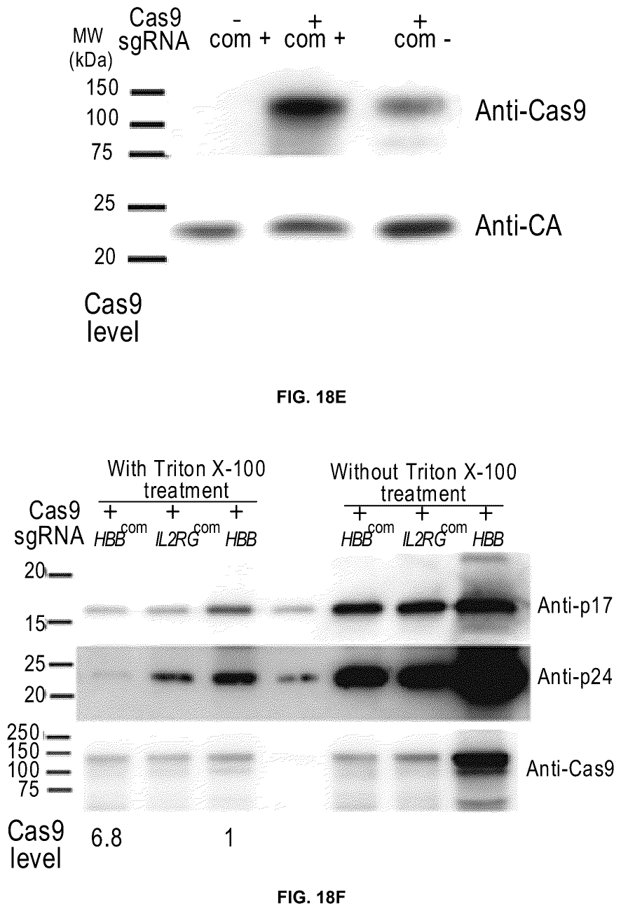

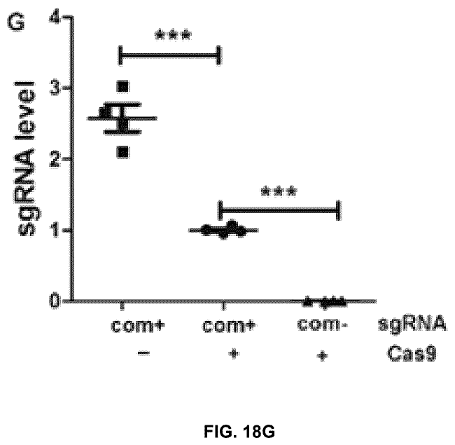

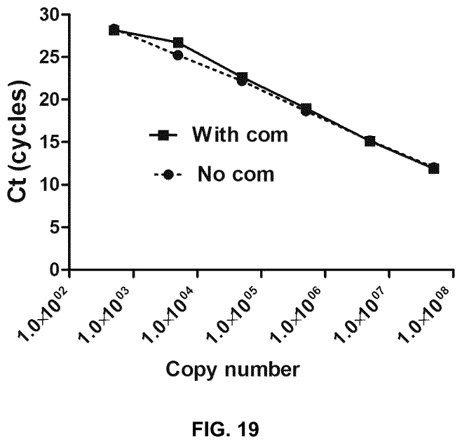

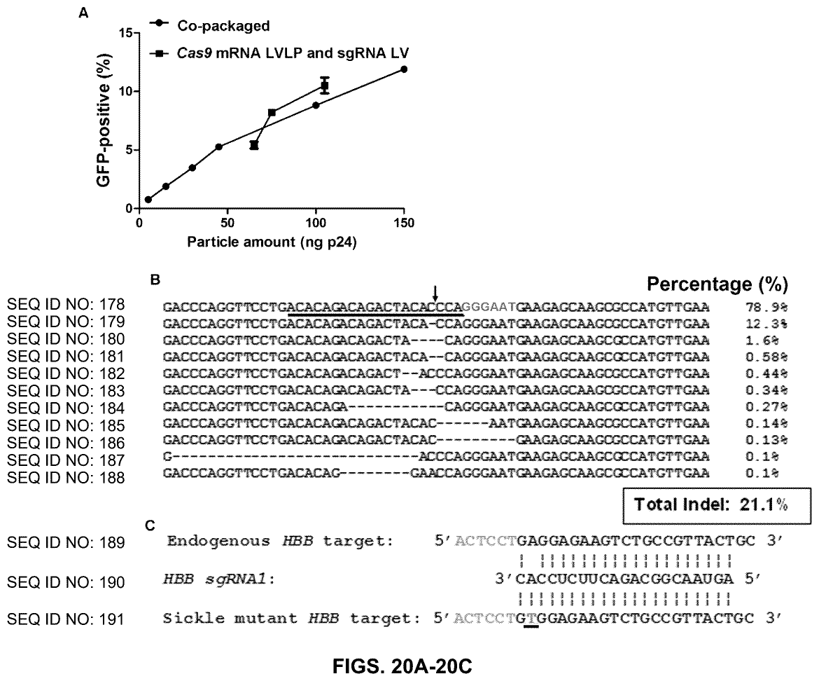

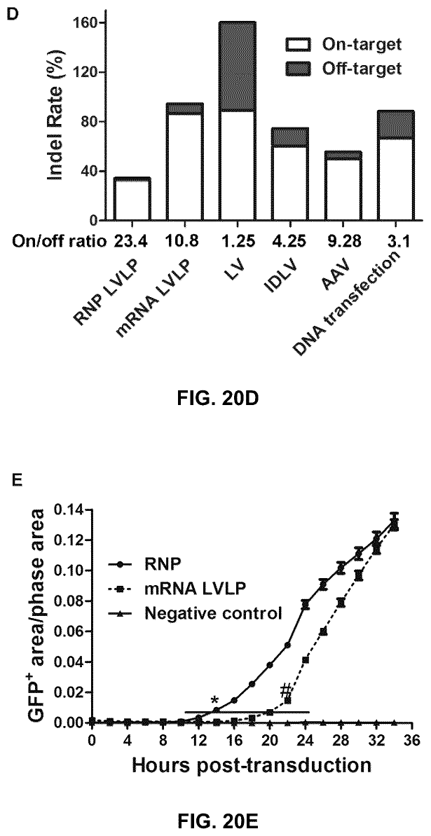

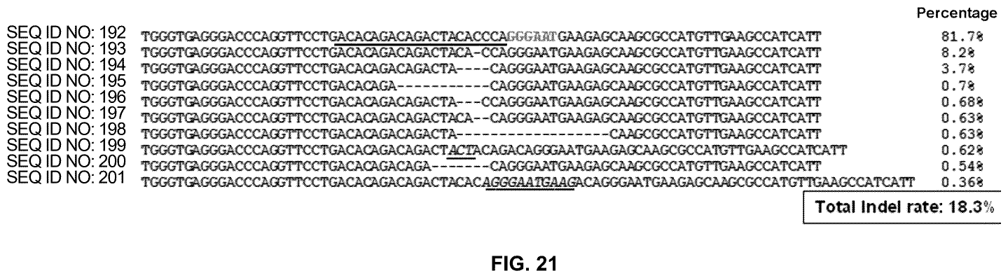

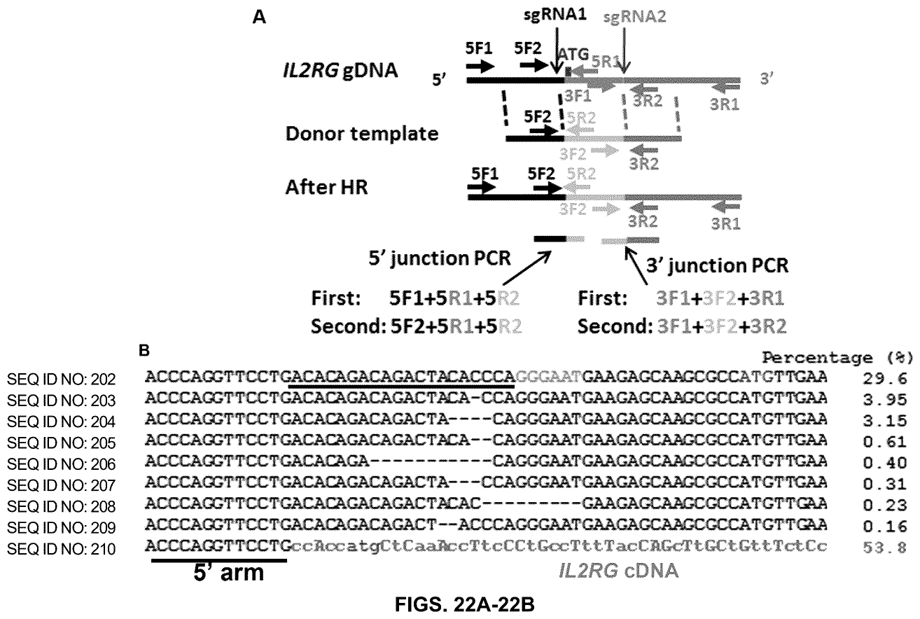

As shown in FIG. 1B, the lentivirus-like particles may be used to transduce eukaryotic cells thereby providing CRISPR-associated endonuclease mRNA, from which active protein is translated in the cells, and sgRNA molecules. These non-viral RNA molecules may be provided in the same lentivirus-like particle or in different viral particles. The CRISPR-associated endonuclease protein and the sgRNA interact to form a ribonucleoprotein complex that binds to a genomic target site (as directed by the sgRNA) and introduces breaks into the DNA. The breaks may be repaired by homology-directed repair (HDR) or non-homologous end-joining (NHEJ) mechanisms. If HDR is desired, the eukaryotic cells may also be transduced with a viral particle containing a target template sequence that can be used as the template for modification of the genomic target site. As the amount of CRISPR-associated endonuclease mRNA introduced into the cells is finite, eventually it will be degraded, as will proteins expressed from the mRNA. Optionally, the sgRNA may be provided in a lentiviral particle or DNA virus particle or, when the cells are in culture, as synthetic RNA molecules introduced into the cells via transfection. While FIG. 1B shows in vitro or ex vivo transduction of eukaryotic cells, the viral particles described may be administered to a subject to effect gene editing in vivo.

FIG. 2 shows schematic diagrams of MS2 coat protein (MCP) fusion proteins for packaging SaCas9 mRNA in lentivirus-like particles according to some aspects of this disclosure. VPR: Viral Protein R, NEF: Negative Regulatory Factor; NC, Nucleocapsid Protein; MA: Matrix Protein. Dashed line indicates the deleted region.

FIG. 3 shows a graph illustrating that addition of a HBB 3′ UTR sequence to the SaCas9 mRNA improved the gene editing activity of the viral like particles according to some aspects of this disclosure. Different amount of concentrated viral-like particles containing SaCas9 mRNA with a MS2 aptamer sequence followed by two copies of a HBB 3′ UTR sequence were co-transduced into 2.5×10⁴ GFP-reporter cells with equal amounts of lentivirus-like vectors expressing HBB sgRNA1. The GFP-positive cells were detected by flow cytometry. Each data point was the average of two repeats.

FIG. 4 shows images illustrating the production of lentivirus-like particles from MCP- and PCP-based lentiviral packaging plasmids according to aspects of this disclosure. The control lentiviral particle had the lentiviral genome expressing GFP. The MCP- and PCP-based lentivirus-like particles contained SaCas9^{1xMS2} mRNA and SaCas9^{1xPP7} mRNA, respectively. Means±sem of control, MCP- and PCP-based particles were 98.5±10.5, 105.7±3.1 and 123.9±5.8 nm respectively, n≥11.

FIG. 5A and FIG. 5B show graphs illustrating the transient expression of SaCas9 mRNA in HEK293T cells transduced with different viral vectors according to aspects of this disclosure. FIG. 5A shows expression of SaCas9 mRNA from adeno-associated virus (AAV) and integration defective lentivirus (IDLV) vectors. FIG. 5B shows expression of SaCas9 mRNA from lentivirus-like particles containing SaCas9^{1xPP7}, SaCas9^{1xMS2}, or SaCas9^{1xPP7-3′UTR}. RNA levels were measured by quantitative RT-PCR at 24, 48, 72 and 96 hours post transduction. * indicates p<0.05 between SaCas9^{1xMS2} and SaCas9^{1xMS2}-2×3′UTR by two-way ANOVA.

FIG. 6 shows Indels observed in the HBB sgRNA1 target sequence of GFP reporter cells transduced with lentivirus-like particles according to aspects of this disclosure. The GFP-reporter cells were co-transduced with lentivirus-like particles containing SaCas9^{1xMS2}-3′UTR mRNA and lentivirus expressing HBB-sgRNA1. The DNA was amplified by PCR and sequenced by Next Generation Sequencing. The top 10 most observed sequences are listed as SEQ ID NOs: 103-112 (top to bottom). 13.5% of the alleles were unmodified and the rest all had Indels. The target sequence is underlined and the PAM sequence is the 6 nucleotides preceding the target sequence. The arrow indicates the predicted cleavage position. The wild type HBB sequence has an “A” at the second position of the target sequence; the “T” at that position indicates the nucleotide difference relative to the wild type HBB sequence. The data were from 5774277 sequence readings.

FIGS. 7A-7F show schematics illustrating compositions, systems and results obtained using LVLPs for SaCas9 mRNA packaging. FIG. 7A shows MCP-viral protein fusion proteins and SaCas9-MS2 aptamer fusion RNA (SaCas9 fused to SEQ ID NO: 211) for packaging SaCas9 mRNA in LVLPs. MCP: MS2 coat protein; VPR: viral protein R, NEF: negative regulatory factor; NC, nucleocapsid protein; MA: matrix protein. Dashed line indicates the deleted region in MA. FIG. 7B shows plasmids for making lentivirus and LVLP. The aptamer-binging protein MCP and the MS2 aptamer are shown. LTR: long terminal repeats; Ψ: lentivirus packaging signal. FIG. 7C shows the effects of MCP-fusion proteins on lentivirus production. 5×10⁵ cells in 6-well plates were transfected with the indicated plasmids (5 μg total DNA). 24 hours after transfection the medium was replaced with 2 ml fresh medium and p24 was assayed after 24 hours. Individual data points and mean±SEM are shown. Only MA-MCP modification decreased virus production (p<0.05). FIG. 7D shows that the best gene editing activity was obtained when MCP was fused to NC. SaCas9^{1xMS2} was expressed during LVLP production. 250 μl LVLP-containing supernatant and 250 μl IDLV-expressing HBB sgRNA1 were co-transduced into 2.5×10⁴ GFP-reporter cells. ** indicates p<0.01 when LVLPs from NC-MCP fusion was compared with other particles; * indicates p<0.05 compared with any other particles. FIG. 7E shows flow cytometry analysis of GFP reporter cells transduced with particles made with or without NC-MCP modified packaging plasmid. Increasing volumes of supernatants were co-transduced with 50 ng of HBB sgRNA1-expressing IDLV into GFP reporter cells. When producing particles without packaging plasmid, the packaging plasmid was replaced with pKanCMV-mRuby3-10aa-H2B expressing mRuby. ***, p<0.001 when the GFP-positive rates of the two conditions were compared (Bonferroni posttests following ANOVA). FIG. 7F shows that plasmid DNA contributed little in generating GFP-positive reporter cells. pSaCas9^{1xMS2} (first column) or pSaCas9^{1xMS2}-HBB sgRNA1 plasmid DNA (second column) was transfected into GFP reporter cells to observe GFP-positive cells. pSaCas9^{1xMS2}-HBB sgRNA1 was also used to make LVLPs to transduce into GFP reporter cells alone (100 ng p24, the third column) or with 50 ng p24 of IDLV expressing HBB sgRNA1 (the fourth column). *** indicates p<0.001. For FIGS. 7C, 7D and 7F, Tukey's multiple comparison tests were performed following ANOVA analysis.

FIG. 8 shows that few mRuby-positive cells could be observed whed GFP-reporter cells were transduced with supernatant generated by replacing the packaging plasmid with the (nuclear located) mRuby expressing plasmid. Shown are images from one field of the cells treated with 534 μl of supernatant. Multiple fields were examined and 0-5 positive cells were observed per field. Three fields, with 2, 2 and 3 positive cells respectively, were estimated by imageJ to have 934 cells/field. The average positive rate was 0.0025%.

FIGS. 9A-9E show the Gene editing activities of various LVLPs. FIG. 9A shows the effects of MS2 aptamers on SaCas9 mRNA level. Plasmid DNA expressing SaCas9^{rxMS2} (250 ng) was co-transfected into GFP-reporter cells grown in 24-well plates with plasmid DNA expressing HBB sgRNA1 (250 ng). SaCas9 mRNA level was compared by RT-qPCR with HBB sgRNA1 as a control. *** and ### indicate p<0.001 when SaCas9^{0xMS2} was compared with SaCas9^{1xMS2}, and when SaCas9^{1xMS2} was compared with SaCas9 with more than one MS2 aptamer. FIG. 9B shows the effects of MS2 aptamers on SaCas9 gene editing activity. GFP-reporter cells were transfected as in FIG. 9A. Flow cytometry was performed 72 hours after transfection. *** indicates p<0.001 when SaCas9^{0xMS2} was compared with SaCas9 with at least one MS2 aptamer. FIG. 9C shows the effects of aptamer numbers on LVLP gene editing activity. LVLP-containing supernatants (titer determined by p24 ELISA) were co-transduced with 50 ng HBB sgRNA1-expressing IDLV into 2.5×10⁴ GFP-reporter cells. Flow cytometry was performed 72 hours after transduction. “a” indicates that 45 ng p24 of SaCas9^{1xMS2} LVLPs obtained significantly higher GFP-positive rate (p<0.001) than all other LVLPs at the same dosage; “b” indicates that 45 ng p24 of SaCas9^{12xMS2} LVLPs obtained significantly lower GFP-positive rate (p<0.001) than SaCas9^{2xMS2} or SaCas9^{3xMS2} LVLPs; “c” indicates that 15 ng p24 of SaCas9^{12xMS2} LVLPs obtained significantly lower GFP-positive rate (p<0.05) than all other LVLPs at the same dosage. Each data point is the mean of three replicates. FIG. 9D shows flow cytometry analysis of GFP-positive cells generated after transducing various SaCas9-containing LVLPs. Designated amounts of SaCas9-containing LVLPs were co-transduced with 60 ng p24 HBB sgRNA1-expressing IDLV into 2.5×10⁴ GFP-reporter cells. Each point was the average of indicated replicates (numbers in parentheses). * indicates p<0.05 when SaCas9^{1xMS2}-HBB 3′ UTR LVLP was compared with any other particles of the same dosage; *** indicates p<0.001 when LVLPs generated without ABP and aptamers were compared with any other particles of the same dosage. FIG. 9E shows RT-qPCR comparisons of SaCas9 mRNA copy numbers in different types of LVLPs. RNA was purified from lentivirus or LVLPs containing 30 ng p24. 30 ng p24 of GFP-lentivirus was added in each sample for experimental control. Copy numbers were compared to normal lentiviral vectors known to contain 2 RNA genomes/LV particle. Shown are individual data points and mean±SEM. ns, no significant difference; *** p<0.001 compared with any other groups. For FIGS. 9A, 9B and 9E, Tukey's multiple comparison tests were performed following ANOVA analysis. For FIG. 9C and FIG. 9D, Bonferroni posttests were performed following two-way ANOVA.

FIGS. 10A-10E show the Characterization of LVLPs. FIG. 10A shows transient expression of SaCas9 mRNA from LVLPs. SaCas9-expressing IDLV (35 ng p24) or LVLPs (35 ng p24) were transduced into 2.5×10⁴ HEK293T cells. SaCas9 mRNA levels were assayed at different time points. SaCas9 mRNA levels 24 h post transduction were normalized by housekeeping gene GAPDH and RPLP0 to obtain the relative SaCas9 mRNA expression level. No normalization was performed thereafter so that possible cell replication does not affect evaluation of mRNA degradation since no new mRNA was generated in LVLP transduced cells. *** indicates p<0.001 when SaCas9^{1xMS2}-HBB 3′UTR was compared with other particles at the same time. ### indicates p<0.001 when SaCas9^{1xMS2} was compared with other particles at the same time. FIG. 10B shows a time course of SaCas9 mRNA level from the same particle. mRNA levels of each particle 24 h post transduction were set as 1. Shown are mean±SEM of indicated replicates. * and ** indicate p<0.05 and p<0.01 when compared with other particles at the same time point. For FIGS. 10A and 10B, Bonferroni posttests were performed following two-way ANOVA. FIG. 10C shows Western blot analysis of SaCas9 protein. The four lanes were lysates from mock transfected HREK293T cells (lane 1), GFP-reporter cells co-transduced with 300 ng p24 of Cas9 LVLP and 50 ng p24 of HBB sgRNA1 IDLV (lane 2), HEK293T cells overexpressing Cas9^{1xMS2} (lane 3) or Cas9 mRNA (lane 4) by transfecting 0.25 μg DNA to 1.25×10⁵ cells. A very faint band in LVLP transduced cells was indicated by an arrow. FIG. 10D is a diagram showing the processing of Gag precursor by HIV protease. The wideness of the arrows is proportional to the processing speed at that site (1-5). The estimated sizes of the p15-ABP fusion proteins were listed. FIG. 10E shows Western blot analysis of lentiviral proteins. 200 ng p24 of GFP lentivirus, NC-MCP and NC-PCP modified LVLPs were analyzed.

FIG. 11 shows electron microscopy of NC-MCP and NC-PCP modified LVLPs. Shown below the images are the means±SEM with sample numbers in parentheses. No statistical differences were observed between groups.

FIGS. 12A-F shows genome editing by SaCas9 LVLP. FIG. 12A shows that SaCas9 LVLPs efficiently generated Indels in the perfectly matched HBB sgRNA1 target sequence. The most frequently observed sequences and their percentages from next-generation sequencing are listed. The “T” in red is the mutation in HBB causing Sickle cell disease. FIG. 12B shows the frequency of Indels at each position obtained from sequencing data in FIG. 12A. FIG. 12C shows Indels in the IL2RG gene of HEK293T cells generated by SaCas9 LVLPs. 2.5×10⁴ cells were co-transduced with 30 ng p24 of SaCas9^{1xMS2}-HBB 3′UTR LVLPs and 60 ng of IDLV expressing IL2RG sgRNA. FIG. 12D shows Indels in the IL2RG gene of lymphoblasts generated by 500 ng p24 of SaCas9^{1xMS2}-HBB 3′UTR LVLPs (500 ng p24 LVLPs on 2×10⁵ cells). FIG. 12E shows Indel rates in the wild-type HBB locus of the GFP reporter cells. There was one mismatch between the HBB sgRNA1 (highlighted in red) and the target sequence. FIG. 12F shows Indel rates in predicted off-target 8 of the GFP reporter cells. Off-target 8 has one mismatch with HBB sgRNA1 (underlined), one DNA bulge (in blue) and a non-typical PAM (a “G” instead of a “T” at the last position, in italics). For FIGS. 12A-12F: The protospacer adjacent motif (PAM) or its complementary sequence is in green. For FIGS. 12A-12E: The target sequences are underlined. Vertical lines indicate complementarity between HBB sgRNA1 and the target. 3-4 million readings were obtained for each sample.

FIG. 13 shows the relationship between target sequence Indel rates and GFP-positive rates in GFP reporter cells. The four data points were from GFP-reporter cells transduced with SaCas9^{1xMS2} LVLPs (about 750 ng p24 for 1.25×10⁶ cells), SaCas9 AAV6 (10⁴ vg/cell), SaCas9 IDLV (about 750 ng p24 for 1.25×10⁶ cells), and SaCas9^{1xMS2}-HBB 3′UTR (30 ng p24 for 2×5×10⁴ cells). The target sequences in the GFP-expression cassette were amplified and subjected to next generation sequencing. The Indel rates were plotted against the GFP-positive rates obtained by flow cytometry analysis.

FIG. 14A-14D show packaging of sgRNA in LVLPs. FIG. 14A is a diagram illustrating the different Cas9 mRNA LVLP (left) and Cas9/sgRNA RNP LVLP (right) as immature virions. Only one GAG precursor and one mRNA or RNP were shown for simplicity. ABP may bind to aptamer as dimers. FIG. 14B shows the locations for MS2 aptamer insertion in sgRNA scaffold. The original sgRNA was shown on the left (SEQ ID NO: 212). The “N”s indicate guide sequence. The three locations tested for MS2 aptamer insertion was indicated by the dashed black boxes. The inserted sequences were shown on the right (SEQ ID NO: 213 (aptamer at tetraloop) and SEQ ID O: 214 (aptamer at the 3′end). The blue letters in dashed blue boxes were the MS2 aptamers and the black letters were added linkers. Complementary ribonucleotides were indicated by vertical lines and atypical base-pairings were indicated by dots. FIG. 14C shows the effects of MS2 aptamer position on gene editing activity of the RNP with the modified sgRNA. Plasmid DNA co-expressing SaCas9 mRNA and various modified HBB sgRNA1 were transfected into the GFP-reporter cells and the GFP-positive percentage was determined by flow cytometry. Each data point indicates one independent experiment. *, ** and *** indicate p<0.05, p<0.01 and p<0.001 (Tukey's Multiple Comparison Test following ANOVA) when compared with unmodified HBB sgRNA1. FIG. 14D shows the effects of MS2 aptamer position on gene editing activity of modified sgRNA packaged in LVLPs. Indicated amounts of LVLPs containing MS2-modified HBB sgRNA1 and SaCas9 protein (or mRNA) were used to transduce 2.5×10⁴ GFP reporter cells and the GFP-positive percentages were determined by flow cytometry. Each data point is the average of three replicates. ***, p<0.001 when HBB sgRNA1^{Tetra MS2} was compared with HBB sgRNA1^{3′MS2}; ###, p<0.001 when HBB sgRNA1^{3′MS2} was compared with HBB sgRNA1^{ST2 MS2}.

FIGS. 15A-15C provide comparisons for aptamer/ABP pairs for packaging CRISPR/Cas9 RNA in LVLPs. FIG. 15A shows the replacement of the Tetraloop in SEq ID NO: 215 with different aptamers for sgRNA packaging. The boxed Tetra loop (GAAA) sequence was replaced with sequences containing various aptamers (underlined) ((MS2 (SEQ ID NO: 216), com (SEQ ID NO: 217), PP7 (SEQ ID NO: 218) and BoxB (SEQ ID NO: 219)) with or without linkers (not underlined). FIG. 15B shows a comparison of gene editing activities after the Tetraloop was replaced by different aptamers. Various amounts of plasmid DNA co-expressing SaCas9 and aptamer-modified HBB shRNA1 was transfected into 1.25×10⁵ GFP reporter cells. 48 hours after transfection the GFP-positive cells were analysed by flow cytometry. Each point was the average of three replicates. *** indicates p<0.001 when sgRNA without modification or with com modification was compared with PP7, BoxB or MS2 modified sgRNAs (Bonferroni posttests following ANOVA). FIG. 15C provides a comparison of gene editing activities of LVLPs made by different aptamer/ABP pairs. 400 μl of un-concentrated LVLPs were used to transduce 2.5×10⁴ GFP reporter cells, and the GFP-positive rate was determined by flow cytometry. Each point indicates one independent assay. *** indicates p<0.001 between the indicated pairs in Tukey's Multiple Comparison Test following ANOVA analysis. ns, not significant.

FIG. 16 shows that the LVLPs generated GFP-positive cells in GFP-reporter cells but not in HEK293T cells. 2.5×10⁴ cells were seeded in 24-well plates and 150 ng p24 of Cas9/HBB sgRNA1 RNP LVLPs were transduced into the cells. The cells were analyzed by fluorescent microscopy 48 hours after transduction.

FIG. 17 provides analysis of NC-COM modification on particle assembly efficiency. Packaging plasmid with or without COM modification was used to package Cas9/HBB sgRNA1, or GFP lentiviral vector. Transfection was done in 6-well plates; p24 was assayed in supernatants collected between 24-48 hours after transfection. * indicates p<0.05 by Tukey's Multiple Comparison Test following ANOVA.

FIG. 18A-18G show that RNP accounted for the gene editing activity. FIG. 18A shows that HBB sgRNA1 expressed from transfected plasmid DNA was functional. Indicated amounts of DNA were transfected into 1.25×10⁵ GFP reporter cells and the cells were analysed by flow cytometry 48 hour after transfection. In co-transfection experiments, the DNA amount indicated each of the Cas9 expressing- and the sgRNA expressing-plasmid DNA. Each point was the average of three replicates. FIG. 18B shows the importance of Cas9 co-packaging and com-aptamer modification of sgRNA on gene editing activity of the LVLPs. LVLPs packaged in the absence of Cas9 expression were inactive when co-transduced into GFP-reporter cells with functional Cas9-HBB-3′UTRMS2 mRNA LVLPs. 2.5×10⁴ GFP-reporter cells were transduced with Cas9/HBB sgRNA1 LVLPs (com-RNP LVLPs), Cas9/HBB sgRNA1^{tetra-com} LVLPs (com⁺ RNP LVLPs), or co-transduced with 45 ng p24 of Cas9-HBB-3′UTRMS2 mRNA LVLPs and various amounts of HBB sgRNA1tetra-com LVLPs (com⁺ sgRNA LVLPs). GFP-positive cells were determined by flow cytometry 48 hours after transduction. Each point is the average of 3 replicates. *** indicates p<0.001 when GFP-positive rates of cells treated with com⁺ RNP LVLPs were compared with cells treated with similar amounts of other particles (Bonferroni posttests following ANOVA).

FIG. 18C shows that co-expressing Cas9 increased HBB sgRNA1 level. Plasmids expressing only HBB sgRNA1^{Tetra-com} (200 ng) and only SaCas9 (200 ng) were transfected into HEK293T cells alone or together, and the sgRNA level was compared by RT-qPCR. A GFP-expressing plasmid (50 ng) was co-transfected so that GFP expression could be used to normalize transfection efficiency. Total plasmid DNA was brought to 450 ng by pCDNA3 plasmid DNA. * indicates p<0.05 when sgRNA level without Cas9 co-expression was compared with that of with Cas9 co-expression. FIG. 18D shows Western blot analysis of Cas9 protein in isolated lentiviral vectors and LVLPs. 200 ng p24 of GFP lentivirus (lane 1), NC-MCP modified Cas9-HBB-3′ UTR^{MS2} LVLPs (without sgRNA, lane 2), NC-unmodified Cas9^{MS2} LVLPs (without sgRNA, lane 3), NC-COM modified Cas9/HBB sgRNA1^{tetra-com} LVLPs (lane 4), NC-COM modified Cas9/IL2RG sgRNA1^{tetra-com} LVLPs (lane 5), and NC-COM modified Cas9/HBB sgRNA1 LVLPs (sgRNA without Tetra-com aptamer, lane 6) were loaded. FIG. 18E shows that a Tetra-com modification of sgRNA increased the Cas9 protein content in LVLPs. FIG. 18F show that Cas9 proteins in LVLPs with com-modified sgRNA are more detergent-resistant than Cas9 proteins in LVLPs with unmodified sgRNA. The same amount of starting LVLPs (200 ng of p24) was centrifuged through 1 ml of 10% sucrose with or without 0.5% Triton X-100. For (FIGS. 18E and 18F), Cas9 level was normalized by CA protein, based on dosimetry analysis (IMAGE J). FIG. 18G shows that packaging of sgRNA in LVLPs is com-aptamer but not Cas9 protein dependent. See FIG. 18E for evidence of similar particle input (CA) for RNA isolation. Each point indicates one repeat. *** indicates p<0.001 between the indicated pairs in Tukey's Multiple Comparison Test following ANOVA analysis.

FIG. 19 shows standard curves when the same primer pairs were used to amplify the sgRNA DNA sequence with and without com aptamer.

FIG. 20A-20E show that Cas9/sgRNA RNP LVLPs are efficient and specific in gene editing. FIG. 20A shows that Cas9/sgRNA RNP LVLPs has comparable gene editing activity as Cas9 mRNA LVLPs. For Cas9 mRNA LVLPs, the particle amount was the sum of Cas9 mRNA LVLPs and 60 ng p24 of HBB sgRNA1-expressing LVs. FIG. 20B shows that Indels generated by Cas9/IL2RG sgRNA1 RNP LVLPs on the endogenous IL2RG target sequence. The protospacer adjacent motif (PAM) is in grey and the target sequence is underlined. The predicted cleavage site is indicated by an arrow. The dashed lines indicate deletions. FIG. 20C is a diagram showing the sequence of HBB sgRNA1, the Sickle mutant sequence perfectly matching the gRNA, and the endogenous HBB sequence with one mismatch with the gRNA. The mutation causing Sickle cell disease is underlined. FIG. 20D is a comparison of on target and off-target Indel rate of Cas9/HBB sgRNA1 RNP LVLPs with those of other delivery vehicles. The on target to off-target Indel rate ratios (“on/off” ratios) are the results of on-target Indel rates divided by off-target Indel rates. Cells were harvested 72 hours after treatment. For cells grown in 24-well plates, 1.25×10⁵ and 2.5×10⁴ cells were used for transfection and transduction respectively. For RNP LVLPs, 150 ng p24 were used; for mRNA LVLPs, 45 ng p24 of Cas9 mRNA LVLPs and 60 ng p24 of HBB sgRNA1-expressing IDLV were used; for LV co-expressing Cas9 and HBB sgRNA1, 30 ng p24 were used; for IDLV co-expressing Cas9 and HBB sgRNA1, 30 ng p24 were used; for Cas9-expressing AAV6 and HBB sgRNA1-expressing AAV6, 10⁴ virus genome/cell were used for each virus. On-target Indel rates are all normalized to 100 for comparison. Original data are in FIG. 22. FIG. 20E shows that Cas9 RNP LVLPs showed faster actions than Cas9 mRNA LVLPs. 50 ng p24 of Cas9/IL2RG sgRNA1 RNP LVLPs, or 100 ng p24 of Cas9 mRNA LVLPs plus 100 ng p24 of IDLV expressing IL2RG sgRNA1 were transduced into 2.5×10⁴ GFP reporter cells and incubated in IncuCyte for scanning. * indicates the time point from which RNP-treated cells showed significantly higher GFP-positive area/phase area than negative control or mRNA LVLP-treated cells (p<0.05). # indicates the time point from which mRNA LVLP-treated cells showed significantly higher GFP-positive area/phase area than negative control cells (p<0.05). The line shows the time lag in Cas9 mRNA LVLP treated cells to reach the same level of GFP-positive area/phase area as RNP-treated cells.

FIG. 21 shows that Indels generated by Cas9/IL2RG RNP LVLPs in lymphocytes. The nucleotides in gray is the PAM. The nucleotides underlined are the target sequence. The underlined Italic nucleotides are insertions.

FIGS. 22A and 22B show that Cas9/sgRNA RNP LVLPs facilitated homologous recombination in the presence of donor template delivered by IDLV. FIG. 22A is a diagram showing the design of the donor template and the PCR strategies for detection of homologous recombination. FIG. 22B shows detection of homologous recombination events by NGS. The PAM is in green. The target sequence is underlined. The inserted IL2RG cDNA was in orange. The original start codon and the start codon for the inserted cDNA are in red. In the cDNA, the lower case letters are identical to the original cDNA sequence while the capital letters are different from the original sequence but the encoded protein is the same.

### DEFINITIONS

As used in this specification and the appended claims, the singular forms “a,” “an,” and “the” include plural reference unless the context clearly dictates otherwise.

The term “nucleic acid” or “nucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” can refer to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, guide RNA, or micro RNA

“Treating” refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present disclosure to prevent or delay, to alleviate, or to arrest or inhibit development of the symptoms or conditions associated with a disease, condition or disorder as described herein. The term “therapeutic effect” refers to the reduction, elimination, or prevention of the disease, symptoms of the disease, or side effects of the disease in the subject. “Treating” or “treatment” using the methods of the present disclosure includes preventing the onset of symptoms in a subject that can be at increased risk of a disease or disorder associated with a disease, condition or disorder as described herein, but does not yet experience or exhibit symptoms, inhibiting the symptoms of a disease or disorder (slowing or arresting its development), providing relief from the symptoms or side effects of a disease (including palliative treatment), and relieving the symptoms of a disease (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition. The term “treatment,” as used herein, includes preventative (e.g., prophylactic), curative, or palliative treatment.

A “promoter” is defined as one or more a nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass full-length proteins, truncated proteins, and fragments thereof, and amino acid chains, wherein the amino acid residues are linked by covalent peptide bonds.

As used herein, the term “complementary” or “complementarity” refers to specific base pairing between nucleotides or nucleic acids. Complementary nucleotides are, generally, A and T (or A and U), and G and C.

As used throughout, by subject is meant an individual. For example, the subject is a mammal, such as a primate, and, more specifically, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical uses and formulations are contemplated herein. The term does not denote a particular age or sex. Thus, adult and newborn subjects, whether male or female, are intended to be covered. As used herein, patient or subject may be used interchangeably and can refer to a subject afflicted with a disease or disorder.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter, followed by a transcription termination signal sequence. An expression cassette may or may not include specific regulatory sequences, such as 5′ or 3′ untranslated region from human globin genes.

A “reporter gene” encodes proteins that are readily detectable due to their biochemical characteristics, such as enzymatic activity or chemifluorescent features. These reporter proteins can be used as selectable markers. One specific example of such a reporter is green fluorescent protein. Fluorescence generated from this protein can be detected with various commercially-available fluorescent detection systems. Other reporters can be detected by staining. The reporter can also be an enzyme that generates a detectable signal when contacted with an appropriate substrate. The reporter can be an enzyme that catalyzes the formation of a detectable product. Suitable enzymes include, but are not limited to, proteases, nucleases, lipases, phosphatases and hydrolases. The reporter can encode an enzyme whose substrates are substantially impermeable to eukaryotic plasma membranes, thus making it possible to tightly control signal formation. Specific examples of suitable reporter genes that encode enzymes include, but are not limited to, CAT (chloramphenicol acetyl transferase; Alton and Vapnek (1979) Nature 282:864-869); luciferase (lux); β-galactosidase; LacZ; β.-glucuronidase; and alkaline phosphatase (Toh, et al. (1980) Eur. J. Biochem. 182: 231-238; and Hall et al. (1983) J. Mol. Appl. Gen. 2:101), each of which are incorporated by reference herein in its entirety. Other suitable reporters include those that encode for a particular epitope that can be detected with a labeled antibody that specifically recognizes the epitope.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types.

The CRISPR/Cas system classification as described in by Makarova, et al. (Nat Rev Microbiol. 2015 November; 13 (11): 722-36) defines five types and 16 subtypes based on shared characteristics and evolutionary similarity. These are grouped into two large classes based on the structure of the effector complex that cleaves genomic DNA. The Type II CRISPR/Cas system was the first used for genome engineering, with Type V following in 2015. Wild-type type II CRISPR/Cas systems utilize an RNA-mediated nuclease Cas protein or homolog (referred to herein as a “CRISPR-associated endonuclease”) in complex with guide RNA to recognize and cleave foreign nucleic acid. Cas9 proteins also use an activating RNA (also referred to as a transactivating or tracr RNA). Guide RNAs having the activity of either a guide RNA or both a guide RNA and an activating RNA, depending on the type of CRISPR-associated endonuclease used therewith, are also known in the art. In some cases, such dual activity guide RNAs are referred to as a single guide RNA (sgRNA). Synthetic guide RNAs that do not contain an activating RNA sequence may also be referred to as sgRNAs. In this disclosure, the terms sgRNA and gRNA are used interchangeably to refer to an RNA molecule that complexes with a CRISPR-associated endonuclease and localizes the ribonucleoprotein complex to a target DNA sequence. Methods and compositions for controlling inhibition and/or activation of transcription of target genes, populations of target genes (e.g., controlling a transcriptome or portion thereof) are described, e.g., in Cell. 2014 Oct. 23; 159 (3): 647-61, the contents of which are incorporated by reference in its entirety herein for all purposes.

As used herein, “activity” in the context of CRISPR/Cas activity, CRISPR-associated endonuclease activity, sgRNA activity, sgRNA: CRISPR-associated endonuclease nuclease activity and the like refers to the ability to bind to a target genetic element. Typically, activity also refers to the ability of the sgRNA: CRISPR-associated endonuclease nuclease complex to make double-strand breaks at a target genomic region. In some instances, the activity may refer to the ability to modulate transcription at or near a target genomic region. Such activity can be measured in a variety of ways as known in the art. For example, expression, activity, or level of a gene containing or adjacent to the target genomic region can be measured. In another example, the generation of insertions and deletions (Indels) in the genome of cells at a target genomic region can be measured.

As used herein, the phrase “editing” in the context of editing of a genome of a cell refers to inducing a structural change in the sequence of the genome at a target genomic region. For example, the editing can take the form of inserting a nucleotide sequence into or deleting a nucleotide sequence from the genome of the cell. The nucleotide sequence can encode a polypeptide or a fragment thereof. Such editing can be performed by inducing a double stranded break within a target genomic region, or a pair of single stranded nicks on opposite strands and flanking the target genomic region. Methods for inducing single or double stranded breaks at or within a target genomic region include the use of a CRISPR-associated endonuclease nuclease domain and a guide RNA, or pair of guide RNAs, directed to the target genomic region.

As used herein, non-homologous end joining (NHEJ) refers to a cellular process in which cut or nicked ends of a DNA strand can be directly ligated without the need for a homologous template nucleic acid. NHEJ can lead to the addition, the deletion, substitution, or a combination thereof, of one or more nucleotides at the repair site.

As used herein, the term homology directed repair (HDR) refers to a cellular process in which cut or nicked ends of a DNA strand are repaired by polymerization from a homologous template nucleic acid. Thus, the original sequence is replaced with the sequence of the template. The homologous template nucleic acid can be provided by homologous sequences elsewhere in the genome (sister chromatids, homologous chromosomes, or repeated regions on the same or different chromosomes). Alternatively, an exogenous template nucleic acid can be introduced to obtain a specific HDR-induced change of the sequence at the target site. In this way, specific mutations can be introduced at the cut site.

As used herein, a “target template sequence” refers to a DNA oligonucleotide that can be used by a cell as a template for HDR. A target template sequence may be a single-stranded DNA template or a double-stranded DNA template. Generally, the target template sequence has at least one region of homology to a target site in the genome of a cell (genomic target sequence or target genomic region). In some cases, the target template sequence has two homologous regions flanking a region that contains a heterologous sequence to be inserted at a target cut site in the genome of a cell (genomic target sequence or target genomic region).

As used herein, the term “ribonucleoprotein complex” “RNPs”, and the like refers to a complex between a CRISPR-associated endonuclease, for example, Cas9 protein, and a crRNA (e.g., guide RNA or single guide RNA), the Cas9 protein and a trans-activating crRNA (tracrRNA), the Cas9 protein and a guide RNA, or a combination thereof (e.g., a complex containing the Cas9 protein, a tracrRNA, and a crRNA guide.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

Provided herein are compositions, systems, methods of manufacture, and methods for efficient delivery of CRISPR/Cas system components to eukaryotic cells using viral particles. For example, components, systems, methods of manufacture, and methods for efficient delivery to cells of CRISPR-associated endonuclease mRNA or RNPs via lentivirus-like particles are provided. The CRISPR-associated endonuclease produced by the systems described herein is functional for gene editing in eukaryotic cells. CRISPR-associated endonuclease mRNA is delivered into eukaryotic cells and has a limited half-life, reducing the risk of off-target mediated mutagenesis. Delivery of RNPs into eukaryotic cells allows for efficient delivery, for example, in cells that are difficult to transfect, such as primary cells while reducing off-target effects.

The lentivirus-like particles contain a modified viral protein that is a fusion protein with an aptamer-binding protein. The modified viral protein may be structural or non-structural. In particular, the modified viral protein may be lentiviral regulatory proteins (VPR and NEF), nucleocapsid (NC) protein or matrix (MA) protein with the aptamer-binding protein be fused to the protein. The lentivirus-like particles also contain a non-viral nucleic acid sequence; specifically, one or both of a CRISPR-associated endonuclease coding sequence (a CRISPR-associated endonuclease mRNA) or a guide RNA (gRNA). These CRISPR/Cas system components are packaged within the lentivirus-like particles and delivered into the eukaryotic cells as shown in FIG. 1A and FIG. 1B. In some instances, the gRNA is delivered using lentivirus-like particles. For example, in some instances, the CRISPR-associated endonuclease coding sequence and gRNA may be packaged together into lentivirus-like particles. Alternatively, the CRISPR-associated endonuclease coding sequence and gRNA may be packaged into separate lentivirus-like particles. Additionally, other delivery mechanisms may be used to deliver the gRNA into the eukaryotic cells (such as, for example, lentivirus, adenovirus, adeno-associated virus, plasmids, gRNA transfection or electroporation).

The non-viral nucleic acid sequence may be modified with the addition of an aptamer sequence that is specifically bound by the aptamer-binding protein that is fused to the modified viral protein. In some embodiments, the aptamer sequence is attached to a nucleic acid encoding a CRISPR-associated endonuclease mRNA. The interaction between the aptamer sequence and the aptamer-binding protein that is fused to the modified viral protein facilitates packaging of the endonuclease mRNA into the lentiviral particle. FIG. 14A illustrates the packaging of a CRISPR-associated endonuclease mRNA in a lentiviral particle via the interaction between an aptamer attached to a Cas mRNA and an aptamer binding protein fused to a viral protein. In some embodiments, the aptamer sequence is attached to or inserted into a gRNA sequence. When the aptamer sequence attached to or inserted into the gRNA sequence interacts with the aptamer-binding protein, the gRNA sequence complexed with a CRISPR-associated endonuclease (RNP) is packaged in the lentiviral particle. FIG. 14B illustrates the packaging of an RNP (i.e., CRISPR-associated endonuclease complexed with gRNA) in a lentiviral particle via the association of an aptamer attached to the gRNA and an aptamer binding protein fused to a viral protein.

The presence of the viral fusion protein may increase packaging of non-viral nucleic acid sequence or RNPs within the lentivirus-like particles. In some instances, addition of an aptamer sequence to the non-viral nucleic acid sequence, for example, addition of an aptamer sequence to a nucleic acid sequence encoding a CRISPR-associated endonuclease mRNA, may further increase the amount of RNA packaged. In other instances, the non-viral nucleic acid sequence comprises a gRNA sequence and sequence encoding a CRISPR-associated endonuclease, wherein addition of an aptamer sequence to the gRNA sequence, may further increase the amount of RNPs packaged. This disclosure provides lentivirus-like particles as described above; other virus particles components; plasmids for generating such lentivirus-like particles; methods and systems using such plasmids to generate the lentivirus-like particles; and methods of using the lentivirus-like particles to modify a genomic target sequence in a cell.

### I. Introduction

Class 2 Clustered Regularly Interspaced Short Palindromic Repeat (CRISPR) systems, which form an adaptive immune system in bacteria, have been modified for genome engineering. Engineered CRISPR/Cas systems contain two components: a guide RNA (gRNA, also referred to as single guide RNA (sgRNA)) and a CRISPR-associated endonuclease. The gRNA is a short synthetic RNA composed of a scaffold sequence necessary for binding with the CRISPR-associated endonuclease and a user-defined ˜20 nucleotide spacer that defines the genomic target to be modified. Thus, one can change the genomic target of the CRISPR-associated endonuclease by simply changing the target sequence present in the gRNA. CRISPR was originally employed to knock out target genes in various cell types and organisms, but modifications to various CRISPR-associated endonucleases have extended CRISPR to selectively activate/repress target genes, purify specific regions of DNA, image DNA in live cells, and precisely edit DNA and RNA. Where homology-directed repair (HDR) of the genomic target is desired, such as when the aim is to correct a mutated genomic sequence, the system includes a target template sequence that provides the desired sequence to be introduced into the genome in place of the mutated genomic sequence. When non-homologous end joining (NHEJ) is the repair mechanism used to repair the break in the genomic DNA, no target template sequence is used and repair generally results in deletions or insertions at the site of repair. This mechanism is useful where the desired outcome from gene editing is to inactivate and/or impair the function of a genomic sequence, or restoring the reading frame of a gene disrupted by deletions or insertions.

Various mammalian expression systems have been used to deliver CRISPR/Cas systems into cells. These include lentiviral transduction, adeno-associated virus (AAV) transduction, mammalian expression vectors, direct delivery of CRISPR-associated endonuclease mRNA and gRNA, and direct delivery of CRISPR-associated endonuclease/gRNA ribonucleoprotein complexes.

In the case of lentiviral systems, the CRISPR-associated endonuclease and gRNA can be present in a single lentiviral vector or separate lentiviral vectors. The viral vectors may contain a reporter gene (such as GFP) to identify and enrich positive cells. Often the lentiviral vector will also contain a selection marker to generate stable cell lines. A safety feature of lentivirus systems is that the components necessary to produce an infectious viral particle (a virion) are generally divided among multiple plasmids. Packaging plasmids and envelope plasmids encode components of the viral capsid and envelope and are used in conjunction with a transfer plasmid that encodes the viral genome and one or both of the CRISPR-associated endonuclease or gRNA. These plasmids are simultaneously transfected into cells (such as 293 human embryonic kidney cells), the cells are allowed to incubate, and then the supernatant containing the viral particles is collected. These viral particles can then be used to transduce cells of interest. Portions of the lentiviral vector genome can then integrate into the genome of target cells, modulating target genes and their expression. However, use of lentiviral vector systems to deliver a CRISPR-associated endonuclease has the potential to cause oncogenic, infectious, and other transformative changes to infected cells.

Different types of lentiviral vector systems have been developed that seek to improve lentiviral vector system safety and efficacy. Second generation lentiviral systems contain a single packaging plasmid encoding the Gag, Pol, Rev, and Tat genes. Without an internal promotor, transgene expression is driven by the genomic 5′ LTR, which is a weak promotor and requires the presence of Tat to activate expression. Third generation systems improve on the safety of the second generation system in two ways. First, the packaging system is split into two packaging plasmids: one encoding Rev and one encoding Gag and Pol. Second, Tat is eliminated from the third generation system; expression of the transgene from this promoter is no longer dependent on Tat transactivation. A third generation transfer plasmid can be packaged by either a second or a third generation packaging system. While the second and third generation systems address concerns related to unintentional generation of replication-competent viruses, the systems are still vulnerable to causing mutagenesis and off target effects in transduced cells.

An AAV system can be used in which the CRISPR-associated endonuclease and/or gRNA are inserted into an AAV transfer vector and used to generate AAV particles. The packaging limit of the AAV particle is only approximately 4.5 kb, which limits which CRISPR-associated endonuclease can be used and the size of the gRNA.

When mammalian expression vectors are used, a heterologous promoter is used to drive CRISPR-associated endonuclease expression. The promoter can be constitutive or inducible. Often, U6 promoter is used for gRNA. The expression vectors may contain a reporter gene (such as green fluorescent protein; GFP) to identify and enrich positive cells or a selection marker to generate stable cell lines. Mammalian expression vectors can be used for transient or stable expression of the CRISPR-associated endonuclease and/or gRNA in a mammalian cell line that can be transfected at high efficiency.

CRISPR-associated endonuclease mRNA and gRNA (synthesized from plasmids using vitro transcription reactions) can be delivered to target cells using microinjection or electroporation. Similarly, purified CRISPR-associated endonuclease protein and in vitro transcribed gRNA can be combined in vitro to form a ribonucleoprotein complex that is delivered to cells using cationic lipids. Both direct delivery methods result in transient expression of CRISPR components as expression decreases as the CRISPR-associated endonuclease mRNA or protein and/or gRNA are degraded within the cell.

Aspects of this disclosure include modified lentiviral vector systems and components and, in some instances, may also include one or more of lentiviral components, AAV components, or mammalian expression vectors. In some instances, the modified lentiviral vector systems and components provided have been modified to eliminate all or a substantial portion of the lentiviral genome. Additionally, lentivirus-like particles produced from the modified system and components have a reduced risk of generating infection particles and causing mutagenesis and off target effects in transduced cells.

### II. Plasmid Components

Various plasmid compositions are provided in this disclosure. These include modified lentiviral packaging plasmids, modified lentiviral transfer plasmids, and mammalian expression plasmids.

### A. Modified Viral Protein Plasmids

An aspect of the disclosure are plasmids comprising a polynucleotide sequence that encodes a modified lentiviral protein that is a fusion protein of a lentiviral protein fused with an aptamer-binding protein. In the context of this disclosure, the modified viral protein may be structural or non-structural. In some instances, the modified viral protein is a structural protein and may be provided by a lentiviral packaging plasmid. In some instances, the modified viral protein is a non-structural protein and may be provided by a mammalian expression vector. Exemplary structural proteins are lentiviral nucleocapsid (NC) protein and matrix (MA) protein. Exemplary non-structural proteins are viral protein R (VPR) and negative regulatory factor (NEF).

In some embodiments, the modified viral protein is nucleocapsid (NC) protein. In other embodiments, the modified viral protein is matrix (MA) protein. Both the NC protein and the MA protein are encoded by the lentiviral Gag gene. In some instances, the coding sequence of the viral protein may be one of SEQ ID NO:1, SEQ ID NO:3, SEQ ID NO:5, or SEQ ID NO:7. In some instances, the amino acid sequence of the the viral protein may be one of SEQ ID NO:2, SEQ ID NO:4, SEQ ID NO:6, or SEQ ID NO:8. In some instances, the lentiviral packaging plasmid comprises a sequence encoding at least one of SEQ ID NO: 2, SEQ ID NO:4, SEQ ID NO:6, or SEQ ID NO:8 operably linked to a eukaryotic promoter. In some instances, if the viral protein is NEF, the polypeptide may comprise three mutations that enhances packaging in the viral capsid such as, for example, the following substitution mutations: G3C, V153L, and E177G.

The polynucleotide encoding the modified lentiviral protein comprises an aptamer-binding protein (ABP) coding sequence. In some instances, the ABP coding sequence is at the 5′ end or 3′ end of the the viral protein coding sequence. In some instances, the ABP coding sequence may be inserted into the viral protein coding sequence such that the encoded ABP is fused to the viral protein. The ABP coding sequence may be inserted in frame at an internal position within the viral protein coding sequence. When positioned in frame at an internal position near the 5′ or 3′ end of the viral protein coding sequence, the ABP coding sequence is positioned so as not to disrupt processing sequences such as those described in J. Virol. 65 (2): 922-30 (1991) and Biochimica et Biophysica Acta-Biomembranes 1614 (1): 62-72 (2003), which are incorporated herein by reference in their entirety. For example, the Gag nucleotide sequence encodes, inter alia, the NC coding sequence and the MA coding sequence, and the Gag precursor protein is processed by proteolytic cleavage into separate mature viral proteins. The in frame insertion of the ABP coding sequence would not disrupt the nucleotides encoding the processing sequences for proteolytic cleavage. In some instances, nucleotides in the viral protein coding sequence may be replaced with the ABP protein coding sequence. In some instances, a linker sequence encoding 3-6 amino acids may be positioned between the viral protein coding sequence and the ABP coding sequence, or flanking the ABP coding sequence, to help facilitate proper folding of the protein domains upon expression.

In one example, the modified viral protein is NC and the ABP coding sequence is inserted at the 5′ end or the 3′ end of the NC coding sequence. In another example, the modified viral protein is NC and the ABP coding sequence is inserted before or after one of the zinc finger (ZF) domains. For example, the ABP coding sequence may be inserted after the last codon of the second ZF (ZF2) domain. In another example, the ABP coding sequence may be inserted before the first codon of the ZF2 domain. In another example, the ABP coding sequence may be inserted before the first codon of the first ZF (ZF1) domain. In another example, the ABP coding sequence may be inserted after the last codon of the first ZF (ZF1) domain. In some instances, the ABP coding sequence is inserted into the NC coding sequence in a manner that does not disrupt the highly positive stretch of amino acids in the NC protein.

In another example, the modified viral protein is MA and the ABP coding sequence is inserted at the 5′ end or the 3′ end of the MA coding sequence. In another example, the ABP coding sequence is inserted in frame at an internal position within the MA coding sequence. In some instances, nucleotides in the MA coding sequence may be replaced with the ABP protein coding sequence. For example, the nucleotides encoding amino acids 44-132 of the MA protein may be replaced with the ABP coding sequence. In another example, the ABP coding sequence is inserted prior to the codon encoding amino acid 44 of the MA protein. In another example, the ABP coding sequence is inserted after the codon encoding amino acid 132 of the MA protein.

In another example, the modified viral protein is VPR and the ABP coding sequence is inserted at the 5′ end or the 3′ end of the VPR coding sequence. In one example, the ABP coding sequence is inserted at the 5′ end of the VPR coding sequence.

In another example, the modified viral protein is NEF and the ABP coding sequence is inserted at the 5′ end or the 3′ end of the NEF coding sequence. In one example, the ABP coding sequence is inserted at the 3′ end of the NEF coding sequence.

In one aspect, provided is a lentiviral packaging plasmid comprising a eukaryotic promoter operably linked to a Gag nucleotide sequence, wherein the Gag nucleotide sequence comprises a nucleocapsid (NC) coding sequence and a matrix protein (MA) coding sequence, and one or both of the NC coding sequence or the MA coding sequence comprises at least one non-viral aptamer-binding protein (ABP) nucleotide sequence. In some instances, the packaging plasmid does not encode a functional integrase protein. In some instances, lentiviral packaging plasmid comprises at least one non-viral ABP nucleotide sequence immediately downstream the second zinc finger domain of the NC coding sequence. In such instances, the NC coding sequence may comprises two functional zinc finger protein domains and functional native protease processing sequences. Retention of the zinc finger protein domains and native protease processing sequences ensures that the modified NC fusion protein expressed from the lentiviral packaging plasmid is processed correctly into a functional protein. In some instances, lentiviral packaging plasmid comprises at least one non-viral ABP nucleotide sequence in the MA coding sequence.

In some instances, the plasmids may encode one or more viral proteins that comprise two or more aptamer-binding proteins fused thereto. In certain instances, the Gag nucleotide sequence of the lentiviral packaging plasmid may comprise a NC coding sequence and a MA coding sequence and where one or both of the NC coding sequence or the MA coding sequence comprises a first non-viral ABP nucleotide sequence and a second non-viral ABP nucleotide sequence. The first non-viral ABP nucleotide sequence and the second non-viral ABP nucleotide sequence may both encode the same ABP. Alternatively, the first non-viral ABP nucleotide sequence and the second non-viral ABP nucleotide sequence encode different ABPs. In some instances, the Gag nucleotide sequence of the lentiviral packaging plasmid may comprise a NC coding sequence comprising at least one first non-viral ABP nucleotide sequence and a MA coding sequence comprising at least one second non-viral ABP nucleotide sequence. The at least one first non-viral ABP nucleotide sequence and the at least one second non-viral ABP nucleotide sequence may both encode the same ABP. Alternatively, the at least one first non-viral ABP nucleotide sequence and the at least one second non-viral ABP nucleotide sequence encode different ABPs.

In certain instances, the mammalian expression vector may encode a VPR coding sequence or a NEF coding sequence and where the VPR coding sequence or the NEF coding sequence comprises a first non-viral ABP nucleotide sequence and a second non-viral ABP nucleotide sequence. The first non-viral ABP nucleotide sequence and the second non-viral ABP nucleotide sequence may both encode the same ABP. Alternatively, the first non-viral ABP nucleotide sequence and the second non-viral ABP nucleotide sequence encode different ABPs.

A non-viral aptamer-binding protein (ABP) nucleotide sequence encodes a polypeptide sequence that binds to an RNA aptamer sequence. Several non-viral ABPs are suitable for use in this disclosure. In particular, suitable ABPs include bacteriophage RNA-binding proteins that bind specifically to RNA sequences that form stem-loop structures referred to as RNA aptamer sequences. Exemplary non-viral aptamer binding protein include MS2 coat protein, PP7 coat protein, lambda N peptide, and COM (Control of mom) protein. The lambda N peptide may be amino acids 1-22 of the lambda N protein, which are the RNA-binding domain of the protein. In some instances, the ABPs bind to their aptamers as dimers. Information about these ABP and the apatamer sequences to which they bind is provided below in Table 1. In some embodiments, the at least one non-viral ABP nucleotide sequence encodes a polypeptide having the sequence set forth in any of SEQ ID NO: 10, SEQ ID NO: 12, SEQ ID NO:14, or SEQ ID NO:16. In some embodiments, the at least one non-viral ABP nucleotide sequence comprises any of SEQ ID NO:9, SEQ ID NO:11, SEQ ID NO: 13, or SEQ ID NO:15.

TABLE 1
Aptamer-Binding Proteins and Corresponding Aptamer Sequences

| | Aptamer-Binding Proteins | | | |
|---|---|---|---|---|
| | | | lambda N peptide | |
| | MS2 coat protein | PP7 coat protein | (amino acids 1-22) | COM protein |
| Nucleic Acid | SEQ ID NO: 9 | SEQ ID NO: 11 | SEQ ID NO: 13 | SEQ ID NO: 15 |
| Sequence | | | | |
| Amino Acid | SEQ ID NO: 10 | SEQ ID NO: 12 | SEQ ID NO: 14 | SEQ ID NO: 16 |
| Sequence | | | | |
| Aptamer | SEQ ID NO: 17 | SEQ ID NO: 19 | SEQ ID NO: 21 | SEQ ID NO: 23 |
| (RNA) | | | (Box-B aptamer) | |
| Aptamer | SEQ ID NO: 18 | SEQ ID NO: 20 | SEQ ID NO: 22 | SEQ ID NO: 24 |
| (DNA) | | | | |

As discussed above, the lentiviral packaging plasmid may encode various lentiviral proteins. In some instances, the packaging plasmid contains only a Gag nucleotide sequence (gene), a Pol nucleotide sequence (gene), a Rev nucleotide sequence (gene), and a Tat nucleotide sequence (gene), which may be referred to as a second generation packaging plasmid. In some instances, the packaging plasmid contains only the Gag nucleotide sequence (gene) and Pol nucleotide sequence (gene), which may be referred to as a third generation packaging plasmid. In some instances, the lentiviral packaging plasmid may encode only those proteins needed for the viral shell (capsid). In some instances, coding sequences for one or more lentiviral proteins may be excluded in full or in part from the packaging plasmid. For example, in some instances, the packaging plasmid contains only the Gag nucleotide sequence (gene). In another example, the lentiviral packaging plasmid may comprise a deletion of all or a portion of a Pol nucleotide sequence (gene). In another example, the lentiviral packaging plasmid may comprise a deletion of all or a portion of an integrase (Int) coding sequence. In another example, the lentiviral packaging plasmid may comprise a deletion of all or a portion of a reverse transcriptase (RT) coding sequence.

A feature of the lentiviral packaging plasmids provided herein is that they may not encode a functional integrase protein. When the packaging plasmids do not encode a functional integrase protein and they are used in the systems and methods described herein, there is substantially reduced risk the nucleic acid molecules carried by the lentivirus-like particles produced using these packaging plasmids will integrate into the genome of the transduced eukaryotic cell. In some instances, the lentiviral packaging plasmid comprises an integrase coding sequence with an integrase-inactivating mutation therein. For example, the integrase-inactivating mutation may be an aspartic acid to valine mutation at amino acid position 64 (D64V) of the integrase protein encoded by the integrase coding sequence. In some instances, the lentiviral packaging plasmid comprises a deletion of all or a portion of an integrase coding sequence.

The lentiviral packaging plasmids comprise a eukaryotic promoter operably linked to the Gag nucleotide sequence. The mammalian expression plasmids comprise a eukaryotic promoter operably linked to the VPR coding sequence or the NEF coding sequence. In some instances, the eukaryotic promoter is a RNA polymerase II promoter. The RNA polymerase II promoter sequence is selected from a mammalian species. For example, the promoter sequence can be selected from a human, cow, sheep, buffalo, pig, or mouse, to name a few. In some examples, the RNA polymerase II promoter sequence is a CMV, FE1α, or SV40 sequence. In some examples, the RNA polymerase II sequence is a modified RNA polymerase II sequence. For example, the RNA polymerase II sequences having at least 80%, 85%, 90%, 95%, or 99% identity to a wild-type RNA polymerase II promoter sequence from any mammalian species can be used in the constructs provided herein. Those of skill in the art readily understand how to determine the identity of two polypeptides or nucleic acids. For example, the identity can be calculated after aligning the two sequences so that the identity is at its highest level. Another way of calculating identity can be performed by published algorithms. For example, optimal alignment of sequences for comparison can be conducted using the algorithm of Needleman and Wunsch, J. Mol. Biol. 48:443 (1970). In some instances, the eukaryotic promoter is an inducible promoter.

Coding sequences transcribed from a RNA pol II promoter include a poly (A) signal and a transcription terminator sequence downstream of the coding sequence. Commonly used mammalian terminators (SV40, hGH, BGH, and rbGlob) include the sequence motif AAUAAA which promotes both polyadenylation and termination. The role of the terminator, a sequence-based element, is to define the end of a transcriptional unit (such as a gene) and initiate the process of releasing the newly synthesized RNA from the transcription machinery. Terminators are found downstream of the gene to be transcribed, and typically occur directly after any 3′ regulatory elements, such as the polyadenylation or poly (A) signal.

In some instances, the lentiviral packaging plasmids may comprise an expression cassette. In some instances, the mammalian expression plasmids may comprise an expression cassette.

### B. CRISPR System Component Plasmids

Provided herein are mammalian expression plasmids that are used to deliver CRISPR component coding sequences into mammalian cells being used to generate the lentivirus-like particles of this disclosure. Once introduced into the mammalian cells together with the lentiviral packaging plasmids or the mammalian expression plasmids described in Section IIA, these mammalian expression plasmids act as template for generating non-viral CRISPR component RNA molecules that are packaged into the lentivirus-like particles. In some instances, the mammalian expression plasmids are lentiviral transfer vectors. Lentiviral transfer vectors generally encode the viral genome in addition to exogenous genes of interest. In some instances, any mammalian expression plasmid may be suitable for expression of CRISPR component coding sequences in the cells used to produce the lentivirus-like particles.

An aspect of this disclosure are mammalian expression plasmids that comprise the coding sequence for one or more CRISPR components. In some instances, the CRISPR component coding sequence comprises at least one aptamer sequence coding sequence. When the CRISPR component coding sequence is transcribed from the mammalian expression vector, the aptamer sequence is transcribed as well. The resulting transcribed RNA molecule comprises a CRISPR component RNA sequence and at least one aptamer sequence. As discussed in more detail below, the CRISPR component coding sequence may be a CRISPR-associated endonuclease coding sequence, a gRNA coding sequence, or both. In some instances, the CRISPR component coding sequence may be a CRISPR-associated endonuclease coding sequence. In some instances, the CRISPR-associated endonuclease coding sequence may comprise at least one aptamer coding sequence after the stop codon for CRISPR-associated endonuclease coding sequence but before any poly (A) signal and/or transcription terminator. In some instances, the CRISPR component coding sequence may be a gRNA coding sequence. In some instances, the gRNA coding sequence comprises at least one aptamer coding sequence. In some instances, the at least one aptamer coding sequence may be positioned at the 5′ end or the 3′ end of the gRNA. In some instances, the at least one aptamer coding sequence may be inserted at an internal position within the gRNA such as, for example, at one or more of the loops formed in the folded gRNA. For example, where the gRNA is for the SaCas9 protein, the at least one aptamer coding sequence may be positioned at the tetra loop, the stem loop 2, or the 3′ end of the gRNA. In some instances, the aptamer sequence is inserted as a tetraloop.

In some instances, the aptamer coding sequence attached to or inserted to a gRNA sequence is selected from the group consisting of an aptamer that binds an MS2 coat protein sequences, an aptamer that binds a PP7 coat protein, an aptamer that binds a lambda N peptide and an aptamer that binds a COM protein. In some instances, the aptamer coding sequence is an MS2 coat protein aptamer sequence, for example, SEQ ID NO: 17 (RNA) or SEQ ID NO: 18 (DNA). In some instances, the aptamer coding sequence is a PP7 coat protein aptamer sequence, for example, SEQ ID NO: 19 (RNA) or SEQ ID NO: 20 (DNA). In some instances, the aptamer coding sequence is a lambda N peptide aptamer sequence, for example, SEQ ID NO: 21 (RNA) or SEQ ID NO: 22 (DNA). In some instances, the aptamer coding sequence is a COM protein protein aptamer sequence, for example, SEQ ID NO: 17 (RNA) or SEQ ID NO: 18 (DNA).

In some instances, the mammalian expression plasmid comprises a CRISPR-associated endonuclease coding sequence and a gRNA coding sequence. In some instances, each of the CRISPR-associated endonuclease coding sequence and the gRNA coding sequence may comprise at least one aptamer coding sequence. In some instances, the CRISPR-associated endonuclease coding sequence comprises at least one aptamer coding sequence downstream thereof. In some instances, the gRNA coding sequence comprises at least one aptamer coding sequence. In some instances, a spacer of 1-30 nucleotides may be positioned between the CRISPR component coding sequence and the at least one aptamer coding sequence, or flanking the at least one aptamer coding sequence.

An aspect of this disclosure are mammalian expression plasmids that comprise the coding sequence for one or more CRISPR components. In some instances, the CRISPR component coding sequence comprises at least one aptamer sequence coding sequence

As used throughout, a sgRNA is a single guide RNA sequence that interacts with a CRISPR-associated endonuclease (a CRISPR site-directed nuclease) and specifically binds to or hybridizes to a target nucleic acid within the genome of a cell (genomic target sequence), such that the sgRNA and the CRISPR-associated endonuclease co-localize to the target nucleic acid in the genome of the cell. Each sgRNA includes a DNA targeting sequence or protospacer sequence of about 10 to 50 nucleotides in length that specifically binds to or hybridizes to a target DNA sequence in the genome. For example, the DNA targeting sequence may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. For example, the DNA targeting sequence may be about 15-30 nucleotides, about 15-25 nucleotides, about 10-25 nucleotides, or about 18-23 nucleotides. In one example, the DNA targeting sequence is about 20 nucleotides. In some embodiments, the sgRNA comprises a crRNA sequence and a transactivating crRNA (tracrRNA) sequence. In some embodiments, the sgRNA does not comprise a tracrRNA sequence.

Generally, the DNA targeting sequence is designed to complement (e.g., perfectly complement) or substantially complement (e.g., having 1-4 mismatches) to the target DNA sequence. In some cases, the DNA targeting sequence can incorporate wobble or degenerate bases to bind multiple genetic elements. In some cases, the 19 nucleotides at the 3′ or 5′ end of the binding region are perfectly complementary to the target genetic element or elements. In some cases, the binding region can be altered to increase stability. For example, non-natural nucleotides, can be incorporated to increase RNA resistance to degradation. In some cases, the binding region can be altered or designed to avoid or reduce secondary structure formation in the binding region. In some cases, the binding region can be designed to optimize G-C content. In some cases, G-C content is preferably between about 40% and about 60% (e.g., 40%, 45%, 50%, 55%, 60%). In some cases, the binding region, can be selected to begin with a sequence that facilitates efficient transcription of the sgRNA. For example, the binding region can begin at the 5′ end with a G nucleotide. In some cases, the binding region can contain modified nucleotides such as, without limitation, methylated or phosphorylated nucleotides.

As used herein, the term “complementary” or “complementarity” refers to base pairing between nucleotides or nucleic acids, for example, and not to be limiting, base pairing between a sgRNA and a target sequence. Complementary nucleotides are, generally, A and T (or A and U), and G and C. The guide RNAs described herein can comprise sequences, for example, DNA targeting sequence that are perfectly complementary or substantially complementary (e.g., having 1-4 mismatches) to a genomic sequence.

The sgRNA includes a sgRNA constant region that interacts with or binds to the CRISPR-associated endonuclease. In the constructs provided herein, the constant region of an sgRNA can be from about 75 to 250 nucleotides in length. In some examples, the constant region is a modified constant region comprising one, two, three, four, five, six, seven, eight, nine, ten or more nucleotide substitutions in the stem, the stem loop, a hairpin, a region in between hairpins, and/or the nexus of a constant region. In some instances, a modified constant region that has at least 80%, 85%, 90%, or 95% activity, as compared to the activity of the natural or wild-type sgRNA constant region from which the modified constant region is derived, may be used in the constructs described herein. In particular, modifications should not be made at nucleotides that interact directly with a CRISPR-associated endonuclease, for example, a Cas9 polypeptide, or at nucleotides that are important for the secondary structure of the constant region.

The CRISPR-associated endonuclease encoded on mammalian expression plasmids as described herein are RNA-guided site-directed nucleases. Examples include, but are not limited to, nucleases present in any bacterial species that encodes a Type II or a Type V CRISPR/Cas system. For example, and not to be limiting, the CRISPR-associated endonuclease can be a Cas9 polypeptide (Type II) or a Cpf1 polypeptide (Type V). See, for example, Abudayyeh et al., Science 2016 Aug. 5; 353 (6299):aaf5573; Fonfara et al. Nature 532:517-521 (2016), and Zetsche et al., Cell 163 (3): p. 759-771, 22 Oct. 2015. As used throughout, the term “Cas9 polypeptide” means a Cas9 protein, or a fragment or derivative thereof, identified in any bacterial species that encodes a Type II CRISPR/Cas system. See, for example, Makarova et al. Nature Reviews, Microbiology, 9:467-477 (2011), including supplemental information, hereby incorporated by reference in its entirety. CRISPR-associated endonucleases, such as Cas9 and Cas9 homologs, are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein (SpCas9). Another exemplary Cas9 protein is the Staphylococcus aureus Cas9 protein (SaCas9). Additional Cas9 proteins and homologs thereof are described in, e.g., Chylinksi, et al., RNA Biol. 2013 May 1; 10 (5): 726-737; Nat. Rev. Microbiol. 2011 June; 9 (6): 467-477; Hou, et al., Proc Natl Acad Sci USA. 2013 Sep. 24; 110 (39): 15644-9; Sampson et al., Nature. 2013 May 9; 497 (7448): 254-7; and Jinek, et al., Science. 2012 Aug. 17; 337 (6096): 816-21. The Cas9 nuclease domains can be optimized for efficient activity or enhanced stability in the host cell. Other CRISPR-associated endonucleases include Cpf1 (See, e.g., Zetsche et al., Cell, Volume 163, Issue 3, p759-771, 22 Oct. 2015) and homologs thereof.

Full-length Cas9 is an endonuclease comprising a recognition domain and two nuclease domains (HNH and RuvC, respectively) that creates double-stranded breaks in DNA sequences. In the amino acid sequence of Cas9, HNH is linearly continuous, whereas RuvC is separated into three regions, one left of the recognition domain, and the other two right of the recognition domain flanking the HNH domain. Cas9 is targeted to a genomic site in a cell by interacting with a guide RNA that hybridizes to a 20-nucleotide DNA sequence that immediately precedes an NGG motif recognized by Cas9. This results in a double-strand break in the genomic DNA of the cell. In some examples, a Cas9 nuclease that requires an NGG protospacer adjacent motif (PAM) immediately 3′ of the region targeted by the guide RNA can be utilized. As another example, Cas9 proteins with orthogonal PAM motif requirements can be utilized to target sequences that do not have an adjacent NGG PAM sequence. Exemplary Cas9 proteins with orthogonal PAM sequence specificities include, but are not limited to those described in Esvelt et al., Nature Methods 10:1116-1121 (2013).

In some embodiments, the Cas9 protein can be in an active endonuclease form, such that when bound to target nucleic acid as part of a complex with a guide RNA or part of a complex with a DNA template, a double strand break is introduced into the target nucleic acid. The double strand break can be repaired by NHEJ to introduce random mutations, or HDR to introduce specific mutations. Various Cas9 nucleases can be utilized in the methods described herein. For example, a Cas9 nuclease that requires an NGG protospacer adjacent motif (PAM) immediately 3′ of the region targeted by the guide RNA, such as SpCas9, can be utilized. Such Cas9 nucleases can be targeted to any region of a genome that contains an NGG sequence. In another example, a Cas9 nuclease that requires an NNGRRT or NNGRR(N) PAM immediately 3′ of the region targeted by the guide RNA, such as SaCas9, can be utilized. As another example, Cas9 proteins with orthogonal PAM motif requirements can be utilized to target sequences that do not have an adjacent NGG PAM sequence. Exemplary Cas9 proteins with orthogonal PAM sequence specificities include, but are not limited to those described in Nature Methods 10, 1116-1121 (2013), and those described in Zetsche et al., Cell, Volume 163, Issue 3, p759-771, 22 Oct. 2015.

In some cases, the Cas9 protein is a nickase, such that when bound to target nucleic acid as part of a complex with a guide RNA, a single strand break or nick is introduced into the target nucleic acid. A pair of Cas9 nickases, each bound to a structurally different guide RNA, can be targeted to two proximal sites of a target genomic region and thus introduce a pair of proximal single stranded breaks into the target genomic region. Nickase pairs can provide enhanced specificity because off-target effects are likely to result in single nicks, which are generally repaired without lesion by base-excision repair mechanisms. Exemplary Cas9 nickases include Cas9 nucleases having a D10A or H840A mutation.

In some instances, the CRISPR-associated endonuclease is a deactivated site-directed nuclease. For example, the CRISPR-associated endonuclease may be a dCas9 polypeptide. As used throughout this disclosure, a dCas9 polypeptide is a deactivated or nuclease-dead Cas9 (dCas9) that has been modified to inactivate Cas9 nuclease activity. Modifications include, but are not limited to, altering one or more amino acids to inactivate the nuclease activity or the nuclease domain. For example, and not to be limiting, D10A and H840A mutations can be made in Cas9 from Streptococcus pyogenes to inactivate Cas9 nuclease activity. Other modifications include removing all or a portion of the nuclease domain of Cas9, such that the sequences exhibiting nuclease activity are absent from Cas9. Accordingly, a dCas9 may include polypeptide sequences modified to inactivate nuclease activity or removal of a polypeptide sequence or sequences to inactivate nuclease activity. The dCas9 retains the ability to bind to DNA even though the nuclease activity has been inactivated. Accordingly, dCas9 includes the polypeptide sequence or sequences required for DNA binding but includes modified nuclease sequences or lacks nuclease sequences responsible for nuclease activity. It is understood that similar modifications can be made to inactivate nuclease activity in other site-directed nucleases, for example in Cpf1 or C2c2. In some examples, the dCas9 protein is a full-length Cas9 sequence from S. pyogenes lacking the polypeptide sequence of the RuvC nuclease domain and/or the HNH nuclease domain and retaining the DNA binding function. In other examples, the dCas9 protein sequences have at least 30%, 40%, 50%, 60%, 70%, 80%, 90%, 95%, 98% or 99% identity to Cas9 polypeptide sequences lacking the RuvC nuclease domain and/or the HNH nuclease domain and retains DNA binding function.

In some examples, the deactivated CRISPR-associated endonuclease is linked to an effector protein. Optionally, the site-directed nuclease is linked to the effector protein via a peptide linker. The linker can be between about 2 and about 25 amino acids in length. The effector protein can be a transcriptional regulatory protein or an active fragment thereof. The transcriptional regulatory protein can be a transcriptional activator or a transcriptional repressor protein or a protein domain of the activator protein or the inhibitor protein. Examples of transcriptional activators include, but are not limited to VP16, VP48, VP64, VP192, MyoD, E2A, CREB, KMT2A, NF-KB (p65AD), NFAT, TET1, p300Core and p53. Examples of transcriptional inhibitors include, but are not limited to KRAB, MXI1, SID4X, LSD1, and DNMT3A/B. The effector protein can also be an epigenome editor, such as, for example, histone acetyltransferase, histone demethylase, DNA methylase etc. The effector protein or an active fragment thereof can be operatively linked, in series, to the amino-terminus or the carboxy-terminus of the CRISPR-associated endonuclease. Optionally, two or more activating effector proteins or active domains thereof can be operatively linked to the amino-terminus or the carboxy-terminus of the CRISPR-associated endonuclease. Optionally, two or more repressor effector proteins or active domains thereof can be operatively linked, in series, to the amino-terminus or the carboxy-terminus of the CRISPR-associated endonuclease. Optionally, the effector protein can be associated, joined or otherwise connected with the nuclease, without necessarily being covalently linked to the CRISPR-associated endonuclease.

In some embodiment, the CRISPR-associated endonuclease is a Cpf1 polypeptide. Cpf1 protein is a Class II, Type V CRISPR/Cas system protein. Cpf1 is a smaller and simpler endonuclease than Cas9 (such as the spCas9). The Cpf1 protein has a RuvC-like endonuclease domain that is similar to the RuvC domain of Cas9 but does not have a HNH endonuclease domain. The N-terminal domain of Cpf1 also does not have the alpha-helical recognition lobe like the Cas9 protein. When cleaving DNA, Cpf1 introduces a sticky-end-like DNA double-stranded break with a 4 or 5 nucleotide overhang. The Cpf1 protein does not need a tracrRNA; rather, the Cpf1 protein functions with only a crRNA. In the context of this disclosure, where the CRISPR-associated endonuclease is a Cpf1 protein, the sgRNA does not comprise a tracr sequence. The sgRNA used with the Cpf1 protein may comprise only a crRNA sequence (constant region). In some examples, a Cpf1 protein that requires an TTTN or TTN PAM (depending on the species, where “N” is an nucleobase) immediately 5′ of the region targeted by the guide RNA can be utilized. Known Cpf1 proteins and derivatives thereof may be used in the context of this disclosure. For example, in some instances, the CRISPR-associated endonuclease is FnCpf1p and the PAM is 5′ TTN, where N is A/C/G or T. In some instances, the CRISPR-associated endonuclease is PaCpf1p and the PAM is 5′ TTTV, where Vis A/C or G In certain instances, the CRISPR-associated endonuclease is FnCpf1p and the PAM is 5′ TTN, where Nis A/C/G or T, and the PAM is located upstream of the 5′ end of the protospacer. In certain instances, the CRISPR-associated endonuclease is FnCpf1p and the PAM is 5′ CTA and is located upstream of the 5′ end of the protospacer or the target locus. In one example, the CRISPR-associated endonuclease is AsCpf1p and the PAM is 5′ TTTN.

The mammalian expression plasmids comprise a eukaryotic promoter operably linked to the non-viral nucleic acid sequence. In some instances, the eukaryotic promoter is a RNA polymerase II promoter and the non-viral nucleic acid sequence is a CRISPR-associated endonuclease coding sequence. In other instances, the eukaryotic promoter is a RNA polymerase III promoter and the non-viral nucleic acid sequence is a gRNA coding sequence. In some instances, wherein the non-viral nucleic acid sequence comprises both a CRISPR-associated endonuclease coding sequence and a gRNA coding sequence, and wherein a RNA polymerase II promoter is operably linked to the CRISPR-associated endonuclease coding sequence and a RNA polymerase III promoter operably linked to the gRNA coding sequence.

The RNA polymerase II promoter sequence is selected from a mammalian species. The RNA polymerase III promoter sequences is selected from a mammalian species. For example, these promoter sequences can be selected from a human, cow, sheep, buffalo, pig, or mouse, to name a few. In some examples, the RNA polymerase II promoter sequence is a CMV, FE1a, or SV40 sequence. In some examples, the RNA polymerase III promoter sequence is a U6 or an H1 sequence. In some examples, the RNA polymerase II sequence is a modified RNA polymerase II sequence. For example, the RNA polymerase II sequences having at least 80%, 85%, 90%, 95%, or 99% identity to a wild-type RNA polymerase II promoter sequence from any mammalian species can be used in the constructs provided herein. In some examples, the RNA polymerase III sequence is a modified RNA polymerase III sequence. For example, the RNA polymerase III sequences having at least 80%, 85%, 90%, 95%, or 99% identity to a wild-type RNA polymerase III promoter sequence from any mammalian species can be used in the constructs provided herein. Those of skill in the art readily understand how to determine the identity of two polypeptides or nucleic acids. For example, the identity can be calculated after aligning the two sequences so that the identity is at its highest level. Another way of calculating identity can be performed by published algorithms. For example, optimal alignment of sequences for comparison can be conducted using the algorithm of Needleman and Wunsch, J. Mol. Biol. 48:443 (1970). In some instances, the eukaryotic promoter is an inducible promoter.

Coding sequences transcribed from a RNA pol II promoter include a poly (A) signal and a transcription terminator sequence downstream of the coding sequence. Commonly used mammalian terminators (SV40, hGH, BGH, and rbGlob) include the sequence motif AAUAAA which promotes both polyadenylation and termination. Coding sequences transcribed from a RNA pol III promoter include a simple run of T residues downstream of the coding sequence as a terminator sequence. The role of the terminator, a sequence-based element, is to define the end of a transcriptional unit (such as a gene) and initiate the process of releasing the newly synthesized RNA from the transcription machinery. Terminators are found downstream of the gene to be transcribed, and typically occur directly after any 3′ regulatory elements, such as the polyadenylation or poly (A) signal.

In some instances, the mammalian expression vector comprises at least one aptamer coding sequence that encodes an aptamer sequence that is bound specifically by an aptamer-binding protein (ABP). In the context of this disclosure, an aptamer sequence is an RNA sequence that forms a tertiary loop structure that is specifically bound by an ABP. ABPs are RNA-binding proteins or RNA-binding protein domains. Suitable aptamer coding sequences include polynucleotide sequences that encode known bacteriophage aptamer sequences. Exemplary aptamer coding sequences include those encoding the aptamer sequences provided above in Table 1. In some instances, the aptamers are bound by a dimer of ABP. These aptamer sequences are RNA sequences known to be bound specifically by bacteriophage proteins. In some circumstances, the at least one aptamer coding sequence encodes an aptamer sequence bound specifically by an ABP selected from the group consisting of MS2 coat protein, PP7 coat protein, lambda N RNA-binding domain, or COM protein.

In some instances, the mammalian expression vector comprises a CRISPR component coding sequence that comprises one aptamer coding sequence downstream thereof. In other instances, the CRISPR component coding sequence may comprise 1, 2, 3, 4, 5, 6, 7, 8, 9, 10, 11, or 12 aptamer coding sequences. For example, in some instances, the CRISPR component coding sequence may comprise two aptamer coding sequences in tandem. Where the mammalian expression vector includes both a CRISPR-associated endonuclease coding sequence and a gRNA coding sequence, either or both may comprise one or more aptamer coding sequences. In one example, where the vector comprises both a CRISPR-associated endonuclease coding sequence and a gRNA coding sequence, the CRISPR-associated endonuclease coding sequence may comprise one or more aptamer coding sequences. In another example, where the vector comprises both a CRISPR-associated endonuclease coding sequence and a gRNA coding sequence, the gRNA coding sequence may comprise one or more aptamer coding sequences. In another example, where the vector comprises both a CRISPR-associated endonuclease coding sequence and a gRNA coding sequence, both the CRISPR-associated endonuclease coding sequence and the gRNA coding sequence may each comprise one or more aptamer coding sequences. Where the vector comprises both a CRISPR-associated endonuclease coding sequence and a gRNA coding sequence and both comprise one or more aptamer coding sequences, the one or more aptamer coding sequences of the CRISPR-associated endonuclease coding sequence and the one or more aptamer coding sequences of the gRNA coding sequence may be the same aptamer coding sequence or may be different aptamer coding sequences. In some instances, the mammalian expression plasmid comprises a CRISPR component coding sequence, and the CRISPR component coding sequence does not comprise an aptamer coding sequence.

In some instances, the mammalian expression plasmid comprises a CRISPR-associated endonuclease coding sequence comprising at least one first aptamer coding sequence and a gRNA coding sequence comprising at least one second aptamer coding sequence. In one example, the at least one first aptamer coding sequence and the at least one second coding aptamer sequence are the same aptamer coding sequence. In some instances, the at least one first aptamer coding sequence encodes an aptamer sequence bound specifically by a first ABP and the at least one second aptamer coding sequence encodes an aptamer sequence bound specifically by a second ABP, wherein the at least one first aptamer coding sequence and the at least one second aptamer coding sequence encode aptamer sequences bound by different first and second ABPs. For example, the at least one first aptamer coding sequence and the at least one second aptamer coding sequence may encode an aptamer sequence bound specifically by an ABP selected from the group consisting of MS2 coat protein, PP7 coat protein, lambda N protein RNA binding domain, and COM protein. In another example, the at least one first aptamer coding sequence and the at least one second aptamer coding sequence may encode aptamer sequence that are each bound specifically by a different ABP selected from the group consisting of MS2 coat protein, PP7 coat protein, lambda N protein RNA binding domain, and COM protein.

In some instances, the mammalian expression plasmid may also include at least one polynucleotide sequence encoding a RNA-stabilizing sequence positioned downstream of the CRISPR component coding sequence or the aptamer coding sequence if positioned downstream of the CRISPR component coding sequence. The polynucleotide sequence encoding the RNA-stabilizing sequence is transcribed downstream of the CRISPR/Cas system component coding sequence and stabilizes the longevity of the transcribed RNA sequence. In one example, the polynucleotide sequence encoding the RNA-stabilizing sequence is positioned downstream of the CRISPR-associated endonuclease coding sequence. In another example, the polynucleotide sequence encoding the RNA-stabilizing sequence is positioned downstream of the gRNA coding sequence. An exemplary RNA-stabilizing sequence is the sequence of the 3′ UTR of human beta globin gene as set forth in SEQ ID NO:25 (DNA) and SEQ ID NO:26 (RNA). Other RNA-stabilizing sequences are described in Hayashi, T. et al., Developmental Dynamics 239 (7): 2034-2040 (2010) and Newbury, S. et al., Cell 48 (2): 297-310 (1987). In some instances, a spacer of 1-30 nucleotides may be positioned between the CRISPR component coding sequence and the at least one polynucleotide sequence encoding RNA-stabilizing sequence.

In some instances, the mammalian expression plasmid may comprise an expression cassette. In some instances, the mammalian expression plasmid may also comprise a reporter gene.

### III. Systems

Another aspect of this disclosure are lentiviral packaging systems. Such systems include the lentiviral packaging plasmids and mammalian expression plasmids described in this disclosure. These systems are useful in providing components for introduction into mammalian cells to generate the lentivirus-like particles described in this disclosure.

In some instances, the system includes a lentiviral packaging plasmid comprising a eukaryotic promoter operably linked to a Gag nucleotide sequence, wherein the Gag nucleotide sequence comprises a nucleocapsid (NC) coding sequence and a matrix protein (MA) coding sequence, wherein one or both of the NC coding sequence or the MA coding sequence comprise at least one non-viral aptamer-binding protein (ABP) nucleotide sequence, and wherein the packaging plasmid does not encode a functional integrase protein. The lentiviral packaging plasmid may have any of the configurations described above in Section II.A. The system may include a second generation packaging plasmid or third generation packaging plasmids or modified versions thereof. In some instances, the packaging plasmid includes the Gag nucleotide sequence as described above and further comprises a Rev nucleotide sequence and a Tat nucleotide sequence. In other instances, the system includes a first packaging plasmid including a Gag nucleotide sequence as described above and a second packaging plasmid comprising a Rev nucleotide sequence. In each of the packaging plasmids, the viral protein coding sequences are operably linked to a eukaryotic promoter for example, each individually or one promoter for multiple protein coding sequences.

In other instances, the system includes mammalian expression plasmids comprising a eukaryotic promoter operably linked to a NEF coding sequence or a VPR coding sequence, wherein the NEF coding sequence or the VPR coding sequence comprises at least one non-viral ABP nucleotide sequence. The mammalian expression plasmid may have any of the configurations described above in Section II.A. The system may include a second generation packaging plasmid or third generation packaging plasmids or modified versions thereof. In some instances, the packaging plasmid includes a Gag nucleotide sequence, a Rev nucleotide sequence, and a Tat nucleotide sequence. In other instances, the system includes a first packaging plasmid including a Gag nucleotide sequence and a second packaging plasmid comprising a Rev nucleotide sequence.

The system also can include at least one mammalian expression plasmid comprising a eukaryotic promoter operably linked to a non-viral nucleic acid sequence, wherein the non-viral nucleic acid sequence comprises a CRISPR-associated endonuclease coding sequence, a guide RNA (gRNA) coding sequence, or both a CRISPR-associated endonuclease coding sequence and a gRNA coding sequence. The mammalian expression plasmid may have any of the configurations described above in Section II.B. In some instances, the system includes a mammalian expression plasmid that includes a eukaryotic promoter operably linked to a CRISPR-associated endonuclease coding sequence and a eukaryotic promoter operably linked to a gRNA coding sequence. In other instances, the system includes a first mammalian expression plasmid that includes a eukaryotic promoter operably linked to a CRISPR-associated endonuclease coding sequence and a second mammalian expression plasmid that includes a eukaryotic promoter operably linked to a gRNA coding sequence. In some instances, the non-viral nucleic acid in the at least one mammalian expression plasmid does not include an aptamer sequence. In other instances, the non-viral nucleic acid in the at least one mammalian expression plasmid comprises at least one aptamer sequence.

The system also can include an envelope plasmid having an envelope coding sequence that encodes a viral envelope glycoprotein. For example, the Env nucleotide sequence may encode VSV-G. The envelope coding sequence is operably linked to a eukaryotic promoter. In some instances, the eukaryotic promoter is a RNA pol II promoter. Appropriate eukaryotic promoters are described in Section II.

Also provided herein are kits the include the components of the systems described in this disclosure. In some embodiments, the kits include one or more of the plasmids described herein.

### IV. Production of Lentivirus-Like Particles

Provided herein are methods of producing lentivirus-like particles using the plasmids and systems described in this disclosure.

In one aspect, provided is a method of producing a lentiviral particle, the method including the steps of transfecting a plurality of eukaryotic cells with a packaging plasmid, at least one mammalian expression plasmid, and an envelope plasmid as described above in Sections II and III, and culturing the transfected eukaryotic cell for sufficient time for lentiviral particles to be produced. The method may employ a second generation packaging plasmid or third generation packaging plasmids. In some instances, a first and a second packaging plasmid as described in Section III are used. The plurality of eukaryotic cells may be mammalian cells. For example, the mammalian cells may be 293 human embryonic kidney cells or another suitable mammalian expression cell line.

In some instances, the fusion of one or more ABP nucleotide sequences to a viral protein coding sequence, such as the NC coding sequence, the MA coding sequence, or both, does not interfere with viral particle assembly when the plasmid encoding the viral fusion protein, the at least one mammalian expression plasmid encoding a non-viral nucleotide sequence, and the envelope plasmid are transfected into eukaryotic cells. In some instances, a lentiviral packaging plasmid encoding a NC-ABP fusion protein, a MA-ABP fusion protein, or both a NC-ABP fusion protein and a MA-ABP fusion protein does not interfere with viral particle assembly. In some instances, a mammalian expression plasmid encoding either a NEF-ABP fusion protein or a VPR-ABP fusion protein does not interfere with viral particle assembly.

Different lentivirus-like particles will be generated by the provided method depending on the plasmids used to transfect the eukaryotic cells. In some instances, the eukaryotic cells are transfected with a mammalian expression plasmid containing a CRISPR-associated endonuclease coding sequence. Cells transfected with this plasmid will produce lentivirus-like particles containing a CRISPR-associated endonuclease mRNA. In some instances, the eukaryotic cells are transfected with a mammalian expression plasmid containing a gRNA coding sequence. Cells transfected with this plasmid will produce lentivirus-like particles containing a gRNA. In some instances, the cells may be transfected with a first mammalian expression plasmid that contains a CRISPR-associated endonuclease coding sequence and a second mammalian expression plasmid that contains a gRNA coding sequence. Cells transfected with these plasmids will produce lentivirus-like particles containing both a CRISPR mRNA and a gRNA. In some instances, the cells may be transfected with a mammalian expression plasmid that contains a CRISPR-associated endonuclease coding sequence and a gRNA coding sequence. Cells transfected with this plasmid will produce lentivirus-like particles containing both a CRISPR mRNA and a gRNA.

Generally, the plasmids are simultaneously transfected into eukaryotic cells, the cells are allowed to incubate, and then the supernatant containing the virus-like particles is collected. The medium may be changed following transfection and prior to the incubation. The collected virus-like particles may be stored or centrifuged to concentrate particles. Routine cell transfection methods are used for generating lentiviral particles. Crude or concentrated virus-like particles can then be used to transduce the cells of interest. Viral titer may also be determined using known protocols.

A feature of the provided method is the amount of non-viral RNA molecules that can be packaged within the lentivirus-like particles. In a native lentivirus, the viral shell (capsid) contains two copies of the single stranded RNA genome. For example, wild-type HIV-1 viral particles contain two copies of the approximately 9.7 kb single stranded RNA genome. In the methods provided by this disclosure, many non-viral RNA molecules may be packaged within the viral shell. In some instances, the method may produce lentivirus-like particles containing up to 100 non-viral RNA molecules. For example, the lentivirus-like particles may contain about 5, 10, 15, 20, 25, 30, 35, 40, 45, 50, 55, 60, 65, 70, 75, 80, 85, 90, 95, or 100non-viral RNA molecules. For example, the method may produce particles containing 5-25 non-viral RNA molecules, 25-50 non-viral RNA molecules, 50-75 non-viral RNA molecules, or 75-100 non-viral RNA molecules. For example, the method may produce particles containing approximately 50-100 non-viral RNA molecules. In some instances, the method may produce particles containing up to 100 copies of CRISPR-associated endonuclease mRNA (such as, for example, SaCas9 mRNA). In some instances, the method may produce lentivirus-like particles containing several copies of a CRISPR-associated endonuclease mRNA, a gRNA, or a combination thereof. In some instances, the mammalian expression plasmid used in the method may comprise a non-viral nucleic acid sequence that comprises at least one aptamer coding sequence. In other instances, the mammalian expression plasmid used in the method may contain a non-viral nucleic acid sequence that does not comprise at least one aptamer sequence. In some instances, the mammalian expression plasmid does not require an aptamer coding sequence downstream of the non-viral nucleic acid sequence to generate lentiviral-particles containing several copies of non-viral RNA molecules.

In some instances, the mammalian expression plasmid contains a non-viral nucleic acid sequence encoding a CRISPR-associated endonuclease mRNA, wherein an aptamer coding sequence is attached to the nucleic acid encoding a CRISPR-associated endonuclease mRNA. The interaction between the aptamer sequence and an aptamer-binding protein in a viral fusion protein described herein facilitates packaging of the CRISPR-associated endonuclease mRNA into a lentiviral particle. In some instances, the mammalian expression plasmid contains a non-viral nucleic acid sequence encoding a CRISPR-associated endonuclease mRNA and a gRNA, wherein an aptamer coding sequence is attached to or inserted into the gRNA. The interaction between the aptamer sequence attached to or inserted into the gRNA with the aptamer-binding protein in a viral fusion protein described herein, facilitates packaging of the gRNA complexed with the CRISPR-associated endonuclease (RNP) into a lentiviral particle.

### V. Lentivirus-Like Particles

In another aspect, provided are lentivirus-like particles. The lentivirus-like particles contain a modified lentiviral protein that is a fusion protein in which at least one aptamer-binding protein is fused to one or more viral proteins. In the context of this disclosure, the modified viral protein may be structural or non-structural. Exemplary structural proteins are lentiviral nucleocapsid (NC) protein and matrix (MA) protein. Exemplary non-structural proteins are viral protein R (VPR) and negative regulatory factor (NEF). In some instances, the particles contain a fusion protein comprising a NC protein and a MA protein where one or both thereof are fused with at least one non-viral aptamer binding protein (ABP). The NC protein of the particles may have two functional zinc finger protein domains. In particular, retention of the second NC zinc finger domain may preserve the efficiency of viral assembly and budding. In some instances, the particles contain a fusion protein comprising a VPR protein or a NEF protein where the VPR protein or the NEF protein are fused with at least one non-viral ABP. The particles also contain at least one non-viral RNA sequence, wherein the non-viral RNA sequence comprises a CRISPR-associated endonuclease mRNA, a guide RNA (gRNA), or both a CRISPR-associated endonuclease mRNA and a gRNA. In some instances, the lentivirus-like particles do not contain a functional integrase protein. These virus-like particles are useful to transduce eukaryotic cells of interest.

The particles may comprise a viral fusion protein comprising one or more ABPs. In some instances, the particles contain a NC protein, a MA protein, or both, where one or both of the NC protein or MA protein are fused with one or more non-viral ABP. In some instances, lentivirus-like particles comprise a NC protein fused with at least one non-viral ABP. In some instances, lentivirus-like particles comprise a MA protein fused with at least one non-viral ABP. In some instances, the lentivirus-like particles may comprise a NC protein and a MA protein, where one or both of the NC protein or the MA protein may be fused with two non-viral ABP proteins, a first non-viral ABP and a second non-viral ABP fused to a C′ terminal end of the first non-viral ABP (i.e. in tandem). In certain instances, the particles may contain one or both of a NC protein or a MA protein fused with a first non-viral ABP and a second non-viral ABP. In some instances, the lentivirus-like particle contains a VPR protein or a NEF protein, where the VPR protein or the NEF protein is fused to one or more non-viral ABP. In some instances, the lentivirus-like particle contains a VPR protein or a NEF protein fused to two non-viral ABP, a first non-viral ABP and a second non-viral ABP fused to a C′ terminal end of the first non-viral ABP (i.e. in tandem). In some instances, the lentivirus-like particle contains a VPR protein or a NEF protein fused to a first non-viral ABP and a second non-viral ABP. The first non-viral ABP and the second non-viral ABP may both be the same ABP. Alternatively, the first non-viral ABP and the second non-viral ABP may be different ABPs. In some instances, the lentivirus-like particles may comprise a NC protein with at least one first non-viral ABP fused to MA protein with at least one second non-viral ABP fused to its C′ terminal end. The at least one first non-viral ABP and the at least one second non-viral ABP both be the same ABP. Alternatively, the at least one first non-viral ABP protein and the at least one second non-viral ABP may be different ABPs. The first non-viral ABP and the second non-viral ABP may both be the same ABP. Alternatively, the first non-viral ABP and the second non-viral ABP may be different ABPs.

A non-viral ABP is a polypeptide sequence that binds to an RNA aptamer sequence. Several non-viral ABPs are suitable for use in this disclosure. In particular, suitable ABPs include bacteriophage RNA-binding proteins that bind specifically to known RNA aptamer sequences, which are RNA sequences that form stem-loop structures. Exemplary non-viral aptamer binding protein include MS2 coat protein, PP7 coat protein, lambda N peptide, and COM (Control of mom) protein. The lambda N peptide may be amino acids 1-22 of the lambda N protein, which are the RNA-binding domain of the protein. Information about these ABP and the apatamer sequences to which they bind is provided above in Table 1. In some embodiments, the at least one non-viral ABP protein is a polypeptide having the sequence set forth in any of SEQ ID NO:10, SEQ ID NO:12, SEQ ID NO:14, or SEQ ID NO:16.

The lentivirus-like particles may comprise various lentiviral proteins. However, in some instances, the lentivirus-like particles do not comprise all of the types of proteins or nucleic acids found in native lentiviruses. In some instances, the particles may contain NC, MA, CA, SP1, SP2, P6, POL, ENV, TAT, REV, VIF, VPU, VPR, and/or NEF proteins, or a derivative, combination, or portion of any thereof. In some instances, the particles may contain NC, MA, CA, SP1, SP2, P6, and POL. In some instances, the lentivirus-like particles may comprise only those proteins that form the viral shell (capsid). In some instances, one or more lentiviral proteins may be excluded in full or in part from the lentivirus-like particles. For example, in some instances, the lentivirus-like particles may not contain a POL protein or may comprise a non-functional version of a POL protein such as, for example, a POL protein with an inactivating point mutation or an inactivating truncation. In another example, the lentivirus-like particles may not contain an integrase protein or may comprise a non-functional version of an integrase protein such as, for example, an integrase protein with an inactivating point mutation or an inactivating truncation. For example, the lentivirus-like particle may contain a non-functional integrase protein comprising an aspartic acid to valine mutation at amino acid position 64 (D64V). In another example, the lentivirus-like particles may not contain a reverse transcriptase protein or may comprise a non-functional version of a reverse transcriptase protein such as, for example, a reverse transcriptase protein with an inactivating point mutation or an inactivating truncation.

In some instances, the lentivirus-like particles contain at least one non-viral RNA molecule. The non-viral RNA molecule is at least one of a CRISPR/Cas system component or encodes a CRISPR/Cas system component. In some instances, the non-viral RNA molecule may be an mRNA that encode a CRISPR-associated endonuclease. Suitable CRISPR-associated endonucleases are discussed throughout this disclosure. In one example, the non-viral RNA molecule may be an mRNA that encodes a Cas9 protein or derivative thereof. In another example, the non-viral RNA molecule may an mRNA that encodes a Cpf1 protein or derivative thereof. In some instances, the non-viral RNA molecule may comprise a gRNA. Features of suitable gRNAs are discussed throughout this disclosure. The gRNA generally comprises a DNA targeting sequence and a constant region that interacts with the CRISPR-associated endonuclease. In some instances, the gRNA may comprise a transactivating crRNA (tracrRNA) sequence. For example, the gRNA may comprise a tracrRNA where it is to be used in conjunction with a Cas9 protein or derivative. In other instances, the gRNA does not comprise a tracrRNA sequence. For example, the gRNA may not comprise a tracrRNA sequence where it is to be used in conjunction with a Cpf1 protein or derivative. In some instances, the lentivirus-like particles contain both an mRNA encoding a CRISPR-associated endonuclease and a gRNA. In some instances, the particles may contain one of an mRNA encoding a CRISPR-associated endonuclease or a gRNA.

In some instances, the at least one non-viral RNA molecule may have at least one aptamer sequence positioned at the 3′ end thereof. The aptamer sequence is a sequence known to be bound specifically by the aptamer-binding protein (ABP) that is fused to the viral protein as described above. As discussed above, an aptamer sequence is an RNA sequence that forms a tertiary loop structure that is specifically bound by an ABP. Suitable aptamer sequences include known bacteriophage aptamer sequences. Exemplary aptamer sequences are provided above in Table 1. These aptamer sequences are RNA sequences known to be bound specifically by bacteriophage proteins. In some circumstances, the at least one aptamer sequence is bound specifically by an ABP selected from the group consisting of MS2 coat protein, PP7 coat protein, lambda N RNA-binding domain, or COM protein.

In some instances, the non-viral RNA molecule may be a CRISPR-associated endonuclease mRNA. In some instances, the CRISPR-associated endonuclease mRNA may comprise at least one aptamer sequence at its 3′ end. In some instances, the non-viral RNA molecule may be a gRNA coding sequence. In some instances, the gRNA comprises at least one aptamer sequence. In some instances, the at least one aptamer sequence may be positioned at the 5′ end or the 3′ end of the gRNA. In some instances, the at least one aptamer sequence may be inserted at an internal position within the gRNA such as, for example, at one or more of the loops formed in the folded gRNA. For example, where the gRNA is for the SaCas9 protein, the at least one aptamer sequence may be positioned at the tetra loop, the stem loop 2, or the 3′ end of the gRNA. In some instances, the at least one non-viral RNA molecule comprises a CRISPR-associated endonuclease mRNA and a gRNA. In some instances, each of the CRISPR-associated endonuclease mRNA and the gRNA may comprise at least one aptamer coding sequence. In some instances, the CRISPR-associated endonuclease mRNA comprises at least one aptamer sequence at its 3′ end. In some instances, the gRNA comprises at least one aptamer sequence. In some instances, a spacer of 1-30 ribonucleotides may be positioned between the CRISPR-associated endonuclease mRNA or the gRNA and the at least one aptamer sequence, or flanking the at least one aptamer sequence. In certain instances, at least one aptamer sequence does not interfere with lentivirus-like particle transduction of eukaryotic cells. For example, at least one non-viral ABP fused to one or more of the NC protein, the MA protein, the VPR protein, or the NEF protein may not interfere with lentivirus-like particle transduction of eukaryotic cells.

In some instances, the at least one non-viral RNA molecule comprises one aptamer sequence. In other instances, the at least one non-viral RNA molecule may comprise 1, 2, 3, 4, 5, 6, 7, 8, 9, 10, 11, or 12 aptamer sequences. For example, in some instances, lentivirus-like particles comprises at least one non-viral RNA molecule comprising two aptamer sequences. Where the lentivirus-like particle includes both a CRISPR-associated endonuclease mRNA and a gRNA, either or both may comprise one or more aptamer sequences. In one example, where the particles comprise both a CRISPR-associated endonuclease mRNA and a gRNA, the CRISPR-associated endonuclease mRNA may comprise one or more aptamer sequences positioned at the 3′ end. In another example, where the particles comprise both a CRISPR-associated endonuclease mRNA and a gRNA, the gRNA may comprise one or more aptamer sequences. In another example, where the particles comprise both a CRISPR-associated endonuclease mRNA and a gRNA, both the CRISPR-associated endonuclease mRNA and the gRNA may comprise one or more aptamer sequences. Where the particles comprise both a CRISPR-associated endonuclease mRNA and a gRNA and both comprise one or more aptamer sequences, the CRISPR-associated endonuclease mRNA and the gRNA may comprise the same aptamer sequence or may comprise different aptamer sequences.

In some instances, the lentivirus-like particles contain CRISPR-associated endonuclease mRNA with at least one first aptamer sequence positioned at the 3′ end thereof and a gRNA comprising at least one second aptamer sequence. In one example, the at least one first aptamer sequence and the at least one second aptamer sequence are the same aptamer sequence. In some instances, the at least one first aptamer sequence is an aptamer sequence bound specifically by a first ABP and the at least one second aptamer sequence os an aptamer sequence bound specifically by a second ABP, wherein the at least one first aptamer sequence and the at least one second aptamer sequence are bound by different first and second ABPs. For example, the at least one first aptamer sequence and the at least one second aptamer sequence may be bound specifically by an ABP selected from the group consisting of MS2 coat protein, PP7 coat protein, lambda N protein RNA binding domain, and COM protein. In another example, the at least one first aptamer sequence and the at least one second aptamer sequence may be each bound specifically by a different ABP selected from the group consisting of MS2 coat protein, PP7 coat protein, lambda N protein RNA binding domain, and COM protein.

In some instances, the non-viral RNA molecule may also include a RNA-stabilizing sequence positioned at the 3′ end of the CRISPR-associated endonuclease mRNA, at the 3′ end of the CRISPR-associated endonuclease mRNA, or after the at least one aptamer sequence if the at least one aptamer sequence is positioned at the 3′ end of the non-viral RNA molecule. The RNA-stabilizing sequence stabilizes the longevity of the non-viral RNA molecule. In one example, the non-viral RNA molecule comprises a CRISPR-associated endonuclease mRNA with one or more aptamer sequences and a RNA-stabilizing sequence after the one or more aptamer sequences. In another example, the non-viral RNA molecule comprises a gRNA and a RNA-stabilizing sequence downstream a the 3′ end of the gRNA (after any aptamer sequence positioned at the 3′ end of the gRNA). An exemplary RNA-stabilizing sequence is the sequence of the 3′ UTR of human beta globin gene as set forth in SEQ ID NO:25 (DNA) and SEQ ID NO: 26 (RNA). Other RNA-stabilizing sequences are described in Hayashi, T. et al., Developmental Dynamics 239 (7): 2034-2040 (2010) and Newbury, S. et al., Cell 48 (2): 297-310 (1987).

A feature of the lentivirus-like particles provided is the amount of non-viral RNA molecules that can be packaged within the particles. In a native lentivirus, the viral shell (capsid) contains two copies of the single stranded RNA genome. For example, wild-type HIV-1 viral particles contain two copies of the approximately 9.7 kb single stranded RNA genome. In the lentivirus-like particles of this disclosure, up to 100 copies of non-viral RNA molecules may be packaged within the viral shell. For example, the lentivirus-like particles may contain about 5, 10, 15, 20, 25, 30, 35, 40, 45, 50, 55, 60, 65, 70, 75, 80, 85, 90, 95, or 100 non-viral RNA molecules. For example, the particles may contain 5-25 non-viral RNA molecules, 25-50 non-viral RNA molecules, 50-75 non-viral RNA molecules, or 75-100 non-viral RNA molecules. For example, the particles may contain approximately 50-100 non-viral RNA molecules. In some instances, the lentivirus-like particles contain up to 100 copies of CRISPR-associated endonuclease mRNA (such as, for example, SaCas9 mRNA). In some instances, the lentivirus-like particles may contain several copies of a CRISPR-associated endonuclease mRNA, a gRNA, or a combination thereof. In some instances, the lentivirus-like particles comprise several copies of non-viral RNA molecules that comprise at least one aptamer sequence. In other instances, the lentivirus-like particles comprise several copies of non-viral RNA molecules that do not comprise at least one aptamer sequence. In some instances, an aptamer sequence is not required to generate lentiviral-particles containing several copies of non-viral RNA molecules.

In some instances, the lentivirus-like particles contain RNP(s), wherein the RNP is a complex between a CRISPR-associated endonuclease and a gRNA. In some instances, the lentivirus-like particle comprises a) a fusion protein comprising a nucleocapsid (NC) protein or a matrix (MA) protein, wherein the NC protein or MA protein comprises at least one non-viral aptamer binding protein (ABP); and b) a ribonucleotide protein (RNP) complex comprising a CRISPR-associated endonuclease and a guide RNA. In some instances, the lentivirus-like particle comprises a fusion protein comprising a Viral Protein R (VPR) protein or a Negative Regulatory Factor (NEF) protein, wherein VPR protein or the NEF protein comprises at least one non-viral aptamer binding protein (ABP); and b) a ribonucleotide protein (RNP) complex comprising a CRISPR-associated endonuclease and a guide RNA. In lentivirus-like particles that contain RNPs, the guide RNA comprises an apatamer that binds to the non-viral aptamer binding protein of the fusion protein.

Any of the mammalian expression plasmids described herein comprising a non-viral nucleic acid sequence, wherein the non-viral nucleic acid sequence encodes a CRISPR-associated endonuclease and a gRNA sequence, and wherein at least one aptamer is attached or inserted into the gRNA sequence, can be used to generate lentivirus-like particles containing RNPs. As described above, the interaction between the aptamer sequence attached to or inserted into the gRNA with the aptamer-binding protein in a viral fusion protein described herein, facilitates packaging of the gRNA complexed with the CRISPR-associated endonuclease (RNP) into a lentiviral particle.

### VI. Cells

Also provided are cells including the compositions described herein and cells modified by the compositions described herein. Cells or populations of cells comprising one or more nucleic acid constructs (plasmids) described herein are also provided. For example, a cell comprising a lentiviral packaging plasmid as described above are provided herein. For example, a cell comprising a mammalian expression plasmid as described above are provided herein. In another example, a cell comprising both a lentiviral packaging plasmid and a mammalian expression plasmid as described above are provided herein. In this way, lentivirus-like particles comprising CRISPR components of the systems described herein may be generated. In another example, a cell comprising a lentivirus-like particle as described above are provided herein. In this way, modification of target DNA sequences in a cell genome can be effected by the CRISPR components of the systems described herein. Cells or populations of cells comprising the lentiviral packaging systems described herein are also provided. Cells or populations of cells comprising the lentivirus-like particles described herein are also provided.

Cells include, but are not limited to, eukaryotic cells, prokaryotic cells, human cells, non-human animal cells, and fungal cells. Optionally, the cells are in a cell culture. Optionally, the cell is a mammalian cell, for example, a human cell. The cell can be in vitro, ex vivo or in vivo. The cell can also be a primary cell, a germ cell, a stem cell or a precursor cell. The precursor cell can be, for example, a pluripotent stem cell or a hematopoietic stem cell. Introduction of the composition into cells can be cell cycle dependent or cell cycle independent. Methods of synchronizing cells to increase a proportion of cells in a particular phase are known in the art. Depending on the type of cell to be modified, one of skill in the art can readily determine if cell cycle synchronization is necessary. In some cases, cells are removed from a subject, modified using any of the methods described herein and administered to the subject.

Optionally, the cells are T cells. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. T cells can be CD4⁺, CD8⁺, or CD4⁺ and CD8⁺. T cells can be helper cells, for example helper cells of type T_{H}1, T_{H}2, T_{H}3, T_{H}9, T_{H}17, or T_{FH}. T cells can be cytotoxic T cells. A T cell can be a recombinant T cell that has been genetically manipulated, for example a T cell expressing a chimeric antigen receptor or a recombinant T cell receptor

### VII. Gene Editing Methods

Described herein are methods of using the plasmids and systems provided in this disclosure in CRISPR/Cas systems for editing DNA targets or modulating transcription of one or more DNA targets. These methods can be used to repress, mutate, or activate a target genomic sequence, such as a gene, in the genome of a eukaryotic cell.

In the methods provided herein, eukaryotic cells comprising a target genomic sequence of interest to be modified are transduced with lentivirus-like particles that contain a viral fusion protein comprising a viral protein fused to at least one aptamer-binding protein (ABP) and a CRISPR-associated endonuclease mRNA. A feature of the described methods is that the eukaryotic cells do not stably express a CRISPR-associated endonuclease. The CRISPR-associated endonuclease is not constitutively expressed; rather, a finite amount of CRISPR-associated endonuclease mRNA is provided to the transduced cells via the lentivirus-like particles. An advantage of the provided methods is reduced off-target gene editing events because the CRISPR-associated endonuclease is not present and, as such, not active for a long period of time in the cells. Also, when lentivirus-like particles lacking integrase activity are used in the method, there is reduced risk of integration into the cell genome of any of the nucleic acids carried by the particles, particularly the CRISPR-associated endonuclease coding sequence. In some instances, the lentiviral-particles used lack portions of the lentiviral genomic sequences that are essential for viral replication and, as such, reduce the risk of continued particle production. Another advantage of the provided components is that the viral fusion protein may increase packaging of non-viral RNA molecules, such as CRISPR-associated endonuclease mRNA, into the lentivirus-like particles, which in turn increase genome editing efficiency. In some instances, the non-viral RNA molecules packaged in the lentivirus-like particles include at least one aptamer sequence positioned at the 3′ end.

In some instances, the transduced eukaryotic cells are mammalian cells. In some instances, the eukaryotic cells may be in vitro cultured cells. In some instances, the eukaryotic cells may ex vivo cells obtained from a subject. In other instances, the eukaryotic cells are present in a subject. As used throughout, by subject is meant an individual. For example, the subject is a mammal, such as a primate, and, more specifically, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical uses and formulations are contemplated herein. The term does not denote a particular age or sex. Thus, adult and newborn subjects, whether male or female, are intended to be covered. As used herein, patient or subject may be used interchangeably and can refer to a subject afflicted with a disease or disorder. The viral particles of the system provided by this disclosure may be injected into a subject according to known, routine methods. In some instances, the viral particles of the system are injected intravenously (IV), intraperitoneally (IP), intramuscularly, or into a specific organ.

In some instances, the provided methods are for modifying a target locus of interest, the method comprising comprising transducing a plurality of eukaryotic cells with a plurality of viral particles, wherein the plurality of viral particles comprise a fusion protein comprising a Viral Protein R (VPR) protein or a Negative Regulatory Factor (NEF) protein, wherein VPR protein or the NEF protein comprises at least one non-viral aptamer binding protein (ABP); and a ribonucleotide protein (RNP) complex comprising a CRISPR-associated endonuclease and a guide RNA, wherein the RNP binds to the genomic target sequence in genomic DNA of the cell and the CRISPR-associated endonuclease cleaves the genomic DNA of the cell, thereby triggering cellular DNA repair mechanisms causing modification of the genomic target sequence. In some instances, the plurality of viral particles comprise a) a fusion protein comprising a nucleocapsid (NC) protein or a matrix (MA) protein, wherein the NC protein or MA protein comprises at least one non-viral aptamer binding protein (ABP); and b) a ribonucleotide protein (RNP) complex comprising a CRISPR-associated endonuclease and a guide RNA, wherein the RNP binds to the genomic target sequence in genomic DNA of the cell and the CRISPR-associated endonuclease cleaves the genomic DNA of the cell, thereby triggering cellular DNA repair mechanisms causing modification of the genomic target sequence.

As described above, the RNPs are packaged into the viral particles via the interaction of an aptamer sequence attached to or inserted into a gRNA sequence that binds to at least one non-viral aptamer binding protein in a fusion protein described herein. The gRNA sequence binds to at least one non-viral aptamer binding protein in a fusion protein described herein and interacts with the CRISPR-associated endonuclease to form a complex (RNP).

In some instances, the provided methods are for modifying a target locus of interest, the method comprising delivering to said locus a CRISPR-associated endonuclease and one or more nucleic acid components, wherein the CRISPR-associated endonuclease forms a complex with the one or more nucleic acid components and upon binding of the said complex to the locus of interest the CRISPR-associated endonuclease induces the modification of the target locus of interest. In a preferred embodiment, the modification is the introduction of a strand break. In the context of this disclosure, a lentivirus-like particle containing a CRISPR-associated endonuclease mRNA is used to transduce cells containing a target locus of interest to be modified, and a CRISPR-associated endonuclease is expressed in the cells from the CRISPR-associated endonuclease mRNA.

In the provided methods, lentivirus-like particles containing CRISPR-associated endonuclease mRNA can be used to effect genome editing in conjunction with other viral particles that provide the gRNA component of the editing system. In some instances, the plurality of eukaryotic cells are transduced with a lentivirus-like particle that contains both CRISPR-associated endonuclease mRNA and gRNA. In some instances, the plurality of eukaryotic cells are transduced with a lentivirus-like particle containing CRISPR-associated endonuclease mRNA and a second viral particle that provides a gRNA or a gRNA coding sequence. Where the second viral particle is a type of virus that comprises an RNA genome, the viral particle may contain a gRNA or a gRNA coding sequence. For example, the second viral particle may be a lentivirus-like particle that contains a gRNA. In another example, the second viral particle may be a lentiviral particle that contains a gRNA coding sequence. Where the second viral particle is a type of virus that comprises a DNA genome, the viral particle contains a gRNA coding sequence in which the gRNA coding sequence is operably linked to a eukaryotic promoter. In another example, the second viral particle may be an adenovirus or an adeno-associated virus that contains a gRNA coding sequence that is operably linked to a eukaryotic promoter.

In some instances, the provided methods of genome editing or modifying sequences associated with or at a target locus of interest comprises introducing CRISPR/Cas system components including a CRISPR-associated endonuclease mRNA into a eukaryotic cell, whereby the CRISPR/Cas system components effectively function to integrate a DNA insert into the genome of the eukaryotic cell. In certain embodiments, the genome is a mammalian genome. In some instances, the integration of the DNA insert is facilitated by non-homologous end joining (NHEJ) or homology-directed recombination (HDR). In some instances, the DNA insert is an exogenously introduced target template sequence, such as a DNA template or a repair template. The target template sequence comprises a nucleic acid sequence with a desired insertion or modification to the genomic target sequence present in the eukaryotic cells. The target template sequence also comprises nucleic acid sequences homologous to genomic DNA flanking the genomic target sequence.

The target template sequence may be introduced into the eukaryotic cells by transducing the cells with an adenovirus particle, adeno-associated virus, or integration defective lentivirus particle containing the target template sequence. In some instances, the adenovirus particle, adeno-associated virus, or integration defective lentivirus particle may also comprise a gRNA coding sequence operably linked to a eukaryotic promoter.

In homology-directed repair, the target template sequence acts as a donor template, and the natural DNA-repair mechanisms of the cell result in insertion of the nucleic acid sequence with the desired insertion or modification to the genomic target sequence. Genome modification carried out in this way can be used to insert novel genes or knock out existing genes. NHEJ-mediated repair of CRISPR/Cas system breaks can be useful if the intent is to make a null allele (“knockout”) in the genomic target of interest, as it is prone to generating indel errors. Indel errors generated in the course of repair by NHEJ are typically small (1-10 bp) but extremely heterogeneous. There is consequently about a two-thirds chance of the NHEJ-mediated repair causing a frameshift mutation. NHEJ does not obligatorily introduce indels. Given the end structure of the double stranded break (blunt or near-blunt ends without nucleotide damage), indels are rare; in some instances, accounting for less than 5% of repair events. However, the products of accurate repair are easily re-cleaved while indel products are not (no longer pair with the gRNA), so extended exposure of the genomic DNA to the CRISPR system components will favor accumulation of the latter products. In some instances, a pair of gRNAs that flank regions of hundreds of base pairs or more can simultaneously introduce a pair of chromosome breaks to result in deletion of the intervening DNA (“pop-out” deletions) if NHEJ joins the distal ends together. Similarly, it may be possible to direct insertion of an exogenous DNA fragment at a CRISPR-associated endonuclease targeted break (or pair of breaks) by NHEJ-dependent repair (“pop-in” insertion) provided a target template sequence containing compatible overhangs is used.

The methods described can be used with any CRISPR-associated endonuclease that requires a constant region of an sgRNA for function. These include, but are not limited to RNA-guided site-directed nucleases. Examples include nucleases present in any bacterial species that encodes a Type II or V CRISPR/Cas system. Suitable CRISPR-associated endonucleases are described throughout this disclosure. For example, and not to be limiting, the site-directed nuclease can be a Cas9 polypeptide or a Cpf1 polypeptide, or derivatives thereof.

In some instances, CRISPR-associated endonuclease can be in an active Cas9 polypeptide, such that when bound to target nucleic acid as part of a complex with a gRNA or part of a complex with a DNA template, a double strand break is introduced into the target nucleic acid. The double strand break can be repaired by NHEJ to introduce random mutations or HDR to introduce specific mutations. Various Cas9 nucleases can be utilized in the methods described herein. For example, a Cas9 nuclease that requires an NGG protospacer adjacent motif (PAM) immediately 3′ of the region targeted by the guide RNA can be utilized. Such Cas9 nucleases can be targeted to any region of a genome that contains an NGG sequence. As another example, Cas9 proteins with orthogonal PAM motif requirements can be utilized to target sequences that do not have an adjacent NGG PAM sequence. Exemplary Cas9 proteins with orthogonal PAM sequence specificities include, but are not limited to, Cpf1 protein and fragments and derivatives thereof, those described in Esvelt et al., Nature Methods 10 (11): 1116-1121 (2013), and those described in Zetsche et al., Cell 163 (3): 759-771 (2015).

In some cases, the CRISPR-associated endonuclease is a modified Cas9 protein that is a nickase, such that when bound to target nucleic acid as part of a complex with a gRNA, a single strand break or nick is introduced into the target nucleic acid. A pair of Cas9 nickases, each bound to a structurally different guide RNA, can be targeted to two proximal sites of a target genomic region and thus introduce a pair of proximal single stranded breaks into the target genomic region. Nickase pairs can provide enhanced specificity because off-target effects are likely to result in single nicks, which are generally repaired without lesion by base-excision repair mechanisms. Exemplary Cas9 nickases include Cas9 nucleases having a D10A or H840A mutation.

In one example, where the lentivirus-like particles encode a Cas9 polypeptide, which generates blunt ended double-stranded cuts, the method may be used to disable genes, as repair may readily proceed by NHEJ. In another example, where the lentivirus-like particles encode a Cpf1 polypeptide, which generates sticky ends, the method may aid in the incorporation of new sequences of DNA, such as to insert a gene or generate a knock-in. In some instances, a Cpf1 protein may be used to produce gene introductions. However, either CRISPR-associated endonuclease may be used to either introduce genetic sequences or remove (and disable) genes. In some instances, modified Cas9 nickase can be used to introduced single strand breaks near each other in opposite strands to result in a DSB with long overhangs.

Generally, the sgRNA is targeted to specific regions at or near a gene. In some instances, the sgRNA is targeted to regions to affect transcription of a gene. For example, a sgRNA can be targeted to a region at or near the 0-750 bp region 5′ (upstream) of the transcription start site of a gene. In some cases, the 0-750 bp targeting of the region can provide or provide increased, transcriptional activation by an sgRNA: deactivated CRISPR-associated endonuclease. For example, the sgRNA can form a complex with a deactivated CRISPR-associated endonuclease, such as dCas9 polypeptide, to a transcriptional activator to provide, or provide increased transcriptional activation of a gene by the complex. As another example, a sgRNA can be targeted to a region at or near the 0-1000 bp region 3′ (downstream) of the transcription start site of a gene. In some cases, targeting this region may be done to provide increased transcriptional repression by an sgRNA: deactivated CRISPR-associated endonuclease complex. For example, the sgRNA can form a complex with a CRISPR-associated endonuclease, such as dCas9 polypeptide, linked to a transcriptional inhibitor to provide, or provide increased transcriptional repression of a gene by the complex.

In some examples, the sgRNA is targeted to a genomic region that is predicted to be relatively free of nucleosomes. The locations and occupancies of nucleosomes can be assayed through use of enzymatic digestion with micrococcal nuclease (MNase), such as by MNase-seq analysis. Thus, in some examples, the sgRNA is targeted to a genomic region that has a low MNase-Seq signal. In some cases, the sgRNA is targeted to a region predicted to be highly transcriptionally active. For example, the sgRNA can be targeted to a region predicted to have a relatively high occupancy for RNA polymerase II (Pol II). Such regions can be identified by Pol II chromatin immunoprecipitation sequencing (ChIP-seq). Thus, in some cases, the sgRNA is targeted to regions having a high Pol II ChIP-seq signal as disclosed in the ENCODE-published Pol II ChIP-seq database (Landt, et al., Genome Research 22 (9): 1813-1831 (2012)). As another example, the sgRNA can be targeted to a region predicted to be highly transcriptionally active as identified by run-on sequencing or global run-on sequencing (GRO-seq). Thus, in some cases, sgRNAs are targeted to regions having a high GRO-seq signal as disclosed in a published GRO-seq data (e.g., Core et al., Science. 2008 Dec. 19; 322 (5909): 1845-8; and Hah et al., Genome Res. 2013 August; 23 (8): 1210-23).

In some instances, the modifications to the system components as described in this disclosure do not impair how the system components function following transduction into eukaryotic cells. Rather, the components may function similarly or better than unmodified components upon transduction into eukaryotic cells. For example, the viral fusion proteins in the lentivirus-like particles may not interfere with the lentivirus-like particle transduction of eukaryotic cells. Similarly, if the non-viral RNA molecule packaged in the lentivirus-like particles comprises at least one aptamer sequence, the at least one aptamer sequence may not interfere with the lentivirus-like particle transduction of eukaryotic cells. For example, where the non-viral RNA molecule is a CRISPR-associated endonuclease mRNA, the presence of at least one aptamer sequence may not impair expression of the CRISPR-associated endonuclease from the mRNA molecule. In another example, the CRISPR-associated endonuclease expressed from the CRISPR-associated endonuclease mRNA with the at least one aptamer sequence immediately downstream thereof may be as functional as if expressed from an mRNA molecule lacking the at least one aptamer sequence. In another example, the gRNA comprising at least one aptamer sequence may be as functional as a gRNA lacking any aptamer sequence. In some instances, the lentivirus-like proteins containing viral fusion protein may result in greater gene editing upon transduction into eukaryotic cells relative to lentivirus-like particles that do not comprise a viral fusion protein. In one example the viral fusion protein may be a NC-ABP fusion protein, such as a NC-MS2 fusion protein or NC-PP7 fusion protein. In one example, the NC fusion protein is fused to one or two ABPs, such as one or two MS2 proteins, one or two PP7 proteins, or one MS2 protein and one PP7 protein.

The eukaryotic cells can be in vitro, ex vivo or in vivo. In some embodiments, the cell is a primary cell (isolated from a subject). As used herein, a primary cell is a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. In some embodiments, the cells are cultured under conditions effective for expanding the population of modified cells. In some embodiments, cells modified by any of the methods provided herein are purified. In some cases, cells are removed from a subject, modified using any of the methods described herein and re-administered to the patient.

In some instances, once the cells have been transduced with the viral particles described above, the cells are cultured for a sufficient amount of time to allow for gene editing to occur, such that a pool of cells expressing a detectable phenotype can be selected from the plurality of transduced cells. The phenotype can be, for example, cell growth, survival, or proliferation. In some examples, the phenotype is cell growth, survival, or proliferation in the presence of an agent, such as a cytotoxic agent, an oncogene, a tumor suppressor, a transcription factor, a kinase (e.g., a receptor tyrosine kinase), a gene (e.g., an exogenous gene) under the control of a promoter (e.g., a heterologous promoter), a checkpoint gene or cell cycle regulator, a growth factor, a hormone, a DNA damaging agent, a drug, or a chemotherapeutic. The phenotype can also be protein expression, RNA expression, protein activity, or cell motility, migration, or invasiveness. In some examples, the selecting the cells on the basis of the phenotype comprises fluorescence activated cell sorting, affinity purification of cells, or selection based on cell motility.

In some examples, the selecting the cells comprises analysis of the genomic DNA of the cells such as by amplification, sequencing, SNP analysis, etc. Sequencing methods include, but are not limited to, shotgun sequencing, bridge PCR, Sanger sequencing (including microfluidic Sanger sequencing), pyrosequencing, massively parallel signature sequencing, nanopore DNA sequencing, single molecule real-time sequencing (SMRT) (Pacific Biosciences, Menlo Park, CA), ion semiconductor sequencing, ligation sequencing, sequencing by synthesis (Illumina, San Diego, Ca), Polony sequencing, 454 sequencing, solid phase sequencing, DNA nanoball sequencing, heliscope single molecule sequencing, mass spectroscopy sequencing, pyrosequencing, Supported Oligo Ligation Detection (SOLID) sequencing, DNA microarray sequencing, RNAP sequencing, tunneling currents DNA sequencing, and any other DNA sequencing method identified in the future. One or more of the sequencing methods described herein can be used in high throughput sequencing methods. As used herein, the term “high throughput sequencing” refers to all methods related to sequencing nucleic acids where more than one nucleic acid sequence is sequenced at a given time.

Others have assessed the utility of using lentiviral particles to deliver CRISPR/Cas components to eukaryotic cells. Choi et al., Gene Therapy 23 (7): 627-633 (2016) described a system in which Cas9 coding sequence was inserted into the lentiviral genome, resulting in lentiviral particles containing Cas9 protein. That system has low virus titer and low gene editing activity. Compared to Choi et al.'s system, the advantages of the systems and components described herein include: 1) production of lentivirus-like particles at titers similar to conventional lentiviral system; 2) the packaging of multiple CRISPR-associated endonuclease mRNA molecules into each lentivirus-like particle, each mRNA useful for translation of multiple CRISPR-associated endonuclease proteins; and 3) the CRISPR-associated endonuclease expressed from the mRNA molecules packaged in the lentivirus-like particles are fully active and do not require post translational processing. For instance, Choi et al. describe obtaining an Indel rate of 30% (using particles reflecting 150 ng of P24). In contrast, an Indel rate of over 80% was observed by the inventors of this disclosure when using lentivirus-like particles described in this disclosure (particles reflecting 45 ng of P24; 2×10⁴ HEK293T cells; sickle cell mutation correction).

Others have assessed the utility of using lentiviral particles to deliver mRNA to eukaryotic cells (Mock et al., Scientific Reports 4:6409 (2014) and Prel et al., Molecular Therapy Methods & Clinical Development 2:15039 (2015)). Neither group assessed the effectiveness of using lentivirus-like particles to introduce CRISPR/Cas system components into eukaryotic cells. Mock et al. used the lentiviral genome to deliver TALEN mRNA molecules. In this system, only two copies of TALEN mRNA molecules were packaged per viral particle. The extent of translation from the TALEN mRNA was relatively low, leading to inefficient gene editing activity. The inventors of this disclosure tested Mock et al.'s system to deliver SaCas9 mRNA molecules to GFP reporter eukaryotic cells useful for detecting gene editing events. Lentivirus with a SaCas9 coding sequence inserted in the genome was packaged using a packaging plasmid encoding an inactivated reverse transcriptase. The resulting lentivirus particles were co-transduced into the GFP reporter cells described in the Examples with a lentivirus expressing HBB sgRNA1. No GFP+ reporter cells could be detected, indicating that gene editing was not occurring. As such, the system described by Mock et al. was not useful for CRISPR/Cas system gene editing. Prel et al. used lentiviral particles to deliver Cre mRNA molecules into eukaryotic cells in vivo and in vitro. The lentivirus-like particles contained a NC fusion protein in which the second zinc finger domain of the NC protein was replaced with an MS2 protein. The Cre mRNA also had 12 copies of the MS2 aptamer sequence added to the 3′ UTR. That system had inefficient viral particle production and low copy number packaging of Cre mRNA molecules into the viral particles. Compared to Prel et al.'s system, the system of this disclosure generates viral particles efficiently and with high copy number of non-viral RNA molecules per viral particle. Prel et al. reported observing packaging of 5 to 6 copies of Cre mRNA per particle. In contrast, using lentivirus-like particles described in this disclosure made to express NC-MS2 fusion protein (Plasmid No. 11 as packaging plasmid), the inventors of this disclosure observed 100 copies of SaCas9 mRNA per particle when SaCas9 mRNA was modified with human HBB 3′ UTR (Plasmid No. 36) or 30 copies of SaCas9 mRNA per particle when the human HBB 3′ UTR was not added (Plasmid No. 16).

### VIII. Methods of Treatment

Any of the methods and compositions described herein can be used to treat a disease (e.g., cancer, a blood disorder (for example, sickle cell anemia or beta thalassemia), an infectious disease, an autoimmune disease, transplantation rejection, graft vs. host disease or other inflammatory disorder) in a subject.

In some methods, the cancer to be treated is selected from a cancer of B-cell origin, breast cancer, gastric cancer, neuroblastoma, osteosarcoma, lung cancer, colon cancer, chronic myeloid cancer, leukemia (e.g., acute myeloid leukemia, chronic lymphocytic leukemia (CLL) or acute lymphocytic leukemia (ALL)), prostate cancer, colon cancer, renal cell carcinoma, liver cancer, kidney cancer, ovarian cancer, stomach cancer, testicular cancer, rhabdomyosarcoma, and Hodgkin's lymphoma. In some embodiments, the cancer of B-cell origin is selected from the group consisting of B-lineage acute lymphoblastic leukemia, B-cell chronic lymphocytic leukemia, and B-cell non-Hodgkin's lymphoma

In some methods, the cells of the subject are modified in vivo. In some methods, the method of treating a disease in a subject comprises: a) obtaining cells from the subject; b) modifying the cells using any of the methods provided herein; and c) administering the modified cells to the subject. Optionally, the disease is selected from the group consisting of cancer, a blood disorder (for example, sickle cell anemia or beta thalassemia), an infectious disease, an autoimmune disease, transplantation rejection, graft vs. host disease or other inflammatory disorder in a subject. In some methods for treating cancer, the cells obtained form the subject are modified to express a tumor specific antigen. As used throughout, the phrase “tumor-specific antigen” means an antigen that is unique to cancer cells or is expressed more abundantly in cancer cells than in in non-cancerous cells. Optionally, the cells obtained from the subject are T cells. Optionally, the modified cells are expanded prior to administration to the subject.

All patents, patent publications, patent applications, journal articles, books, technical references, and the like discussed in the instant disclosure are incorporated herein by reference in their entirety for all purposes.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure.

The examples presented herein are intended to illustrate potential and specific implementations of the disclosure. It can be appreciated that the examples are intended primarily for purposes of illustration of the disclosure for those skilled in the art. There may be variations to these diagrams or the operations described herein without departing from the spirit of the disclosure. For instance, in certain cases, method steps or operations may be performed or executed in differing order, or operations may be added, deleted or modified.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

Different arrangements of the components depicted in the drawings or described above, as well as components and steps not shown or described are possible. Similarly, some features and sub-combinations are useful and may be employed without reference to other features and sub-combinations. Embodiments of the disclosure have been described for illustrative and not restrictive purposes, and alternative embodiments will become apparent to readers of this patent. Accordingly, the present disclosure is not limited to the embodiments described above or depicted in the drawings, and various embodiments and modifications can be made without departing from the scope of the claims below.

### EXAMPLES

### Example 1. Materials and Methods

Next-Generation Sequencing and Data Analysis. Genomic DNA was isolated from cells with the QIAamp DNA Mini Kit (Qiagen, Germantown, MD) according to the manufacturer's instructions. Two DNA regions were amplified for Next Generation Sequencing analysis. To amplify the endogenous HBB target sequence for sequencing, a nested PCR strategy was used to avoid amplifying the sequence from the viral vector template. First, primers HBB-1849F and HBB-5277R (SEQ ID NOs: 96 and 97) were used to amplify the 3.4 kb region from the HBB gene locus. These two primers are unable to amplify sequence from the templates in the viral vectors. Then HBB-MUT-F and the HBB-MUT-R primers were used to amplify the target DNA for sequencing (Table 3; SEQ ID NOs: 34-40). To amplify the HBB target sites from the integrated EGFP reporter for sequencing, Reporter-mut-F and Reporter-mut-R primers were used (Table 3; SEQ ID NOs: 98-102). The proofreading HotStart® ReadyMix from KAPA Biosystems (Wilmington, MA) was used for PCR. The PCR products were analyzed by next-generation sequencing using the Illumina NextSeq 500 as described before by Javidi-Parsijani, P. et al., PLOS One 2017; 12 (5): e0177444. The Cas-Analyzer online software described by Park, J. et al., Bioinformatics 33 (2): 286-288 (2017) was used for mutation analysis of the sequencing data.

Plasmids. pRSV-Rev (Addgene Cat. No. 12253), pMD2.G (Addgene Cat. No. 12259), pMDLg/pRRE (Addgene Cat. No. 12251), psPAX2 (Addgene Cat. No. 12260), psPAX2-D64V (Addgene Cat. No. 63586), pSL-MS2×12 (Addgene Cat. No. 27119), and pX601-AAV-CMV::NLS-SaCas9-NLS-3xHA-bGHpA;U6::BsaI-sgRNA (Addgene Cat. No. 61591). pCDH-GFP was purchased from Systems Biosciences Inc. (Cat. No. CD513B-1). The plasmids described in Table 2 were produced by the inventors of this disclosure and used in the following examples. Synthetic DNA sequences as identified below were synthesized by GenScript based on custom orders. The primers listed in Table 3 below were synthesized by Eurofins Genomics based on custom orders.

TABLE 2
Plasmids Used in Examples

| No. | Name | Purpose | Generation strategy |
|---|---|---|---|
| 1 | pCDNA3.1/ | Expressing MS2x2-VPR | Synthesized DNA coding for MS2x2-VPR (Genscript, |
| | MS2x2-VPR | fusion protein | Piscataway, NJ) was inserted between the NheI and NotI |
| | | | sites of pCDNA3.1(+) (Genscript). The inserted DNA |
| | | | sequence is SEQ ID NO: 61 and the corresponding amino |
| | | | acid sequence is SEQ ID NO: 62. |
| 2 | pCDNA3.1/ | Expressing NEF-MS2x2 | Synthesized DNA coding for NEF-MS2x2 was inserted |
| | NEF-MS2x2 | fusion protein. Three | between the NheI and NotI sites of pCDNA3.1(+) |
| | | AA of NEF were | (Genscript). The inserted DNA sequence is SEQ ID |
| | | mutated: Gly³, Val¹⁵³ | NO: 63 and the corresponding amino acid sequence is |
| | | and Gly¹⁷⁷ were changed | SEQ ID NO: 64. |
| | | to Cyc, Leu and Glu, | |
| | | respectively. | |

Packaging Plasmids

| | | | |
|---|---|---|---|
| 3 | pMDLg/pR | Third generation | AflII-AgeI fragment of pMDLg/pRRE was replaced by |
| | RE-D64V | lentivirus packaging | the AflII-AgeI fragment of psPAX2-D64V. |
| | | plasmid for generating | |
| | | integration defective | |
| | | lentivirus. | |
| 4 | pMDLg/pR | Third generation | AvrII-SbfI fragment of pMDLg/pRRE-D64V was |
| | RE-D64V- | lentivirus packaging | replaced by a synthetic AvrII-SbfI DNA fragment |
| | NC-MS2x1 | plasmid with MCP | encoding a modified NC sequence in which one copy of |
| | | inserted after NC ZF2. | MCP coding DNA (stop codons removed) was inserted |
| | | | in frame after the last codon of the NC zinc finger 2 |
| | | | domain. The inserted DNA sequence is SEQ ID NO: 65 |
| | | | and the corresponding amino acid sequence is SEQ ID |
| | | | NO: 66. |
| 5 | pMDLg/pR | Third generation | AvrII-SbfI fragment of pMDLg/pRRE-D64V was |
| | RE-D64V- | lentivirus packaging | replaced by a synthetic AvrII-SbfI DNA fragment |
| | NC-MS2x2 | plasmid with two copies | encoding a modified NC sequence in which two copies |
| | | of MCP inserted after | of MCP coding DNA (stop codons removed) were |
| | | NC ZF2. | inserted in frame after the last codon of NC zinc finger 2 |
| | | | domain. The inserted DNA sequence is SEQ ID NO: 67 |
| | | | and the corresponding amino acid sequence is SEQ ID |
| | | | NO: 68. |
| 6 | pMDLg/pR | Third generation | AvrII-SbfI fragment of pMDLg/pRRE-D64V was |
| | RE-D64V- | lentivirus packaging | replaced by a synthetic AvrII-SbfI DNA fragment |
| | NC-PP7x1 | plasmid with one copy | encoding a modified NC sequence in which one copy of |
| | | of PP7 coat protein | PCP coding DNA (stop codons removed) was inserted in |
| | | (PCP) inserted after NC | frame after the last codon of NC zinc finger 2 domain. |
| | | ZF2. | The inserted DNA sequence is SEQ ID NO: 69 and the |
| | | | corresponding amino acid sequence is SEQ ID NO: 70. |
| 8 | pMDLg/pR | Third generation | The PmlI-SphI fragment of pMDLg/pRRE-D64V was |
| | RE-D64V- | lentivirus packaging | replaced by a synthetic PmlI-SphI DNA fragment |
| | MA-MS2x2 | plasmid with two copies | encoding a modified MA in which two copies of MCP |
| | | of MCP replacing AA | coding DNA (stop codons removed) replaced DNA |
| | | 44-132 of MA. | coding for AA 44-132 of MA. The inserted DNA |
| | | | sequence is SEQ ID NO: 71 and the corresponding amino |
| | | | acid sequence is SEQ ID NO: 72. |
| 9 | pMDLg/pR | Third generation | The PmlI-SphI fragment of pMDLg/pRRE-D64V was |
| | RE-D64V- | lentivirus packaging | replaced by a synthetic PmlI-SphI DNA fragment |
| | MA-PP7x1 | plasmid with one copy | encoding a modified MA in which one copy of PCP |
| | | of PCP replacing AA | coding DNA (stop codons removed) replaced DNA |
| | | 44-132 of MA. | coding for AA 44-132 of MA. The inserted DNA |
| | | | sequence is SEQ ID NO: 73 and the corresponding amino |
| | | | acid sequence is SEQ ID NO: 74. |
| 10 | pMDLg/pR | Third generation | The PmlI-SphI fragment of pMDLg/pRRE-D64V was |
| | RE-D64V- | lentivirus packaging | replaced by a synthetic PmlI-SphI DNA fragment |
| | MA-PP7x2 | plasmid with two copies | encoding a modified MA in which two copies of PCP |
| | | of PCP replacing AA | coding DNA (stop codons removed) replaced DNA |
| | | 44-132 of MA. | coding for AA 44-132 of MA. The inserted DNA |
| | | | sequence is SEQ ID NO: 75 and the corresponding amino |
| | | | acid sequence is SEQ ID NO: 76. |
| 11 | psPAX2- | Second generation | The SphI-SbfI fragment of psPAX2-D64V (without NC- |
| | D64V-NC- | lentivirus packaging | MS2) was replaced by the SphI-SbfI fragment of |
| | MS2 | plasmid with MCP | pMDLg/pRRE-D64V-NC-MS2x1 (with one copy of |
| | | inserted after NC ZF2. | MS2) to make psPAX2-D64V-NC-MS2. This is the |
| | | | second generation packaging plasmid with NC-MS2 |
| | | | insertion, corresponding to pMDLg/pRRE-D64V-NC- |
| | | | MS2x1 (Plasmid No. 4), which is the third generation |
| | | | packaging plasmid. |
| 12 | psPAX2- | Second generation | The SphI-SbfI fragment of psPAX2-D64V (without NC- |
| | D64V-NC- | lentivirus packaging | PP7) was replaced by the SphI-SbfI fragment of |
| | PP7 | plasmid with one copy | pMDLg/pRRE-D64V-NC-PP7x1 (with one copy of |
| | | of PCP inserted after NC | PP7). |
| | | ZF2. | |
| 13 | psPAX2- | Second generation | The SphI-SbfI fragment of psPAX2-D64V (without NC- |
| | D64V-NC- | lentivirus packaging | MS2x2) was replaced by the SphI-SbfI fragment of |
| | MS2X2 | plasmid with 2 copies of | pMDLg/pRRE-D64V-NC-MS2X2 (with NC-MS2x2), so |
| | | MS2 inserted after NC | that now we added NC-MS2 to pspAX2-D64V. This is to |
| | | ZF2 | make the second generation packaging plasmid with NC- |
| | | | MS2x2. |
| 14 | psPAX2- | Second generation | The PvuI-SphI fragment of psPAX2-D64V was replaced |
| | D64V-MA- | lentivirus packaging | by the PvuI-SphI fragment of pMDLg/pRRE-D64V-MA- |
| | MS2X2 | plasmid with two copies | MS2X2. |
| | | of MS2 replacing AA | |
| | | 44-132 of MA | |

Mammalian Expression Plasmids

| | | | |
|---|---|---|---|
| 15 | pSaCas9 | Adeno associated viral | pX601-AAV-CMV::NLS-SaCas9-NLS-3xHA- |
| | | (AAV) plasmid | bGHpA; U6::BsaI-sgRNA (Addgene Cat. No. 61591) |
| | | expressing SaCas9 | was cut with NotI and Acc65I (to remove the Sa sgRNA |
| | | | expression cassette) treated with DNA Pol I Klenow |
| | | | polymerase, and re-ligated by T4 DNA ligase. |
| 16 | pSaCas9^{1xms2} | Plasmid expressing | A synthetic dsDNA oligo was generated by annealing |
| | | SaCas9 mRNA with a | oligo 1xloop-F (SEQ ID NO: 41) and oligo 1xloop-R |
| | | MS2 stem loop at the 3′ | (SEQ ID NO: 42), which was then inserted by Infusion ™ |
| | | untranslated region | reaction (Clontech) into the EcoRI site (after the stop |
| | | (UTR) | codon) of pSaCas9. |
| 17 | pSaCas9^{2xms2} | Plasmid expressing | A synthetic dsDNA oligo was generated by annealing |
| | | SaCas9 mRNA with two | oligo 2xloop-F (SEQ ID NO: 43) and oligo 2xloop-R |
| | | MS2 stem loops at the 3′ | (SEQ ID NO: 44), which was then inserted by Infusion ™ |
| | | UTR | reaction (Clontech) into the EcoRI site of pSaCas9. |
| 18 | pSaCas9^{3xms2} | Plasmid expressing | A synthetic dsDNA oligo was generated by annealing |
| | | SaCas9 mRNA with | oligo 3xloop-F (SEQ ID NO: 45) and oligo 3xloop-R |
| | | three MS2 stem loops at | (SEQ ID NO: 46), which was then inserted by Infusion ™ |
| | | the 3′ UTR | reaction (Clontech)into the EcoRI site of pSaCas9- |
| | | | 2xMS2. |
| 19 | pSaCas9^{12xms2} | Plasmid expressing | The EcoR1-Xho1 fragment from pSL-MS2-12X |
| | | SaCas9 mRNA with | (Addgene Cat. No. 27119), encoding the 12 MS2 stem |
| | | twelve MS2 stem loops | loops, was inserted between the EcoR1-SalI sites of |
| | | at the 3′ UTR | pSaCas9-1xMS2, which is after the stop codon of |
| | | | SaCas9 but before the polyA signal sequence. The |
| | | | original fragment coding for 1 MS2 stem loop was |
| | | | replaced with the 12 MS2 step loop fragment. |
| 20 | pSaCas9^{1xPP7} | Plasmid expressing | pSaCas9-^{1xPP7}-HBB-sgRNA1^{3′MS2} was cut with Acc65I |
| | | SaCas9 mRNA with one | and NotI to excise the HBB sgRNA cassette. The |
| | | PP7 stem loop after the | plasmid was treated with DNA Pol I Klenow |
| | | stop codon of saCas9. | polymerase, and re-ligated with T4 DNA ligase. The |
| | | | starting plasmid had a PP7 aptamer coding sequence |
| | | | after the stop codon of the SaCas9 coding sequence and a |
| | | | MS2 aptamer coding sequence after the sgRNA coding |
| | | | sequence. The sgRNA cassette was removed to make a |
| | | | plasmid with a SaCas9 coding sequence followed by a |
| | | | PP7 aptamer coding sequence. |
| 21 | pSaCas9^{12xpp7} | Plasmid expressing | The BglII-BamHI fragment of pDZ617 pKAN 12xPP7 |
| | | SaCas9 mRNA with 12 | V4 (Addgene Cat. No. 72237), encoding the 12 PP7 |
| | | PP7 stem loops at the 3′ | loops, was inserted into the BamHI site of pSaCas9 |
| | | UTR. | (after the coding sequence of Sacas9 but before the HA |
| | | | tag, so that the HA tag is removed but the Sacas9 is |
| | | | complete). |
| 22 | pSaCas9- | Plasmid expressing | A dsDNA oligo was generated by annealing oligo Sickle- |
| | HBB- | SaCas9 mRNA and | g1F (SEQ ID NO: 49) and oligo Sickle-g1R (SEQ ID |
| | sgRNA1 | HBB sgRNA1 targeting | NO: 50), which was then inserted into the BsaI site of |
| | | the region causing sickle | pX601-AAV-CMV::NLS-SaCas9-NLS-3xHA- |
| | | cell disease. | bGHpA; U6::BsaI-sgRNA by T4 DNA ligase. |
| 23 | pSaCas9- | Plasmid expressing | A synthetic DNA fragment was generated by annealing |
| | HBB- | SaCas9 mRNA and | oligo Sickle-g2F (SEQ ID NO: 47) and oligo Sickle-g2R |
| | sgRNA2 | HBB sgRNA2 targeting | (SEQ ID NO: 48), which was then inserted into the BsaI |
| | | the region causing sickle | site of pX601-AAV-CMV::NLS-SaCas9-NLS-3xHA- |
| | | cell disease. | bGHpA; U6::BsaI-sgRNA by T4 DNA ligase. |
| 24 | pSaCas9- | Plasmid expressing | A synthetic DNA fragment (by Genscript) (SEQ ID |
| | HBB- | SaCas9 mRNA and the | NO: 77) was cut with BsaI and NotI and inserted between |
| | sgRNA1^{3′ms2} | guide RNA for HBB; | the BsaI-NotI sites of pX601-AAV-CMV::NLS-SaCas9- |
| | | the 3′ of the sgRNA has | NLS-3xHA-bGHpA; U6::BsaI-sgRNA. This DNA |
| | | a MS2 stem loop. | fragment encodes a HBB sgRNA1 with a MS2 aptamer |
| | | | at the 3′ end. |
| 25 | pSaCas9- | Plasmid expressing | A synthetic DNA fragment (by Genscript Inc) (SEQ ID |
| | HBB- | SaCas9 mRNA and the | NO: 78) was cut with BsaI and NotI and inserted between |
| | sgRNA1^{Tetrams2} | guide RNA for HBB; a | the BsaI-NotI sites of pX601-AAV-CMV::NLS-SaCas9- |
| | | MS2 stem loop was | NLS-3xHA-bGHpA; U6::BsaI-sgRNA. This DNA |
| | | inserted into the tetra | fragment encodes a HBB sgRNA1 with a MS2 aptamer |
| | | loop of the sgRNA. | at the Tetra loop position. |
| 26 | pSaCas9- | Plasmid expressing | A synthetic DNA fragment (by Genscript Inc) (SEQ ID |
| | HBB- | SaCas9 mRNA and the | NO: 79) was cut with BsaI and NotI and inserted between |
| | sgRNA1^{ST2ms2} | guide RNA for HBB; a | the BsaI-NotI sites of pX601-AAV-CMV::NLS-SaCas9- |
| | | MS2 stem loop was | NLS-3xHA-bGHpA; U6::BsaI-sgRNA. This DNA |
| | | inserted into the stem | fragment encodes a HBB sgRNA1 with a MS2 aptamer |
| | | loop 2 of the sgRNA. | at the ST2 loop position. |
| 27 | pSaCas9- | Plasmid expressing | A synthetic DNA fragment (SEQ ID NO: 80) was cut |
| | HBB- | SaCas9 mRNA and the | with BsaI and NotI and inserted between the BsaI-NotI |
| | sgRNA1^{Tetra-ST2ms2} | guide RNA for HBB; a | sites of pX601-AAV-CMV::NLS-SaCas9-NLS-3xHA- |
| | | MS2 stem loop was | bGHpA; U6::BsaI-sgRNA. This DNA fragment includes |
| | | inserted into the stem | a HBB sgRNA1 with one MS2 loop at the Tetra loop |
| | | loop 2 of the sgRNA. | position and one at the ST2 loop position. |
| | | and a MS2 stem loop | |
| | | was inserted into the | |
| | | tetra loop of the sgRNA. | |
| 28 | pSaCas9^{1ms2}- | Plasmid expressing | A single MS2 stem loop dsDNA oligo was generated by |
| | HBB- | SaCas9-1MS2 mRNA | annealing synthetic DNA oligo 1xloop-F (SEQ ID |
| | sgRNA1^{3′ms2} | and the guide RNA for | NO: 41) and oligo 1xloop-R (SEQ ID NO: 42) and |
| | | HBB sgRNA-3′MS2. | inserted into the EcoRI site of pSaCas9-HBB-sgRNA1- |
| | | | 3′MS2 (after the stop codon of SaCas9 and before the |
| | | | PolyA signal sequence). |
| 29 | pSaCas9^{1PP7}- | Plasmid expressing | A single PP7 stem loop dsDNA oligo was generated by |
| | HBB- | SaCas9^{1PP7} mRNA and | annealing synthetic DNA oligo PP7-F (SEQ ID NO: 51) |
| | sgRNA1^{3′ms2} | the guide RNA for HBB | and oligo PP7-R (SEQ ID NO: 52) and inserted into the |
| | | sgRNA^{3′MS2}. | EcoRI site of pSaCas9-HBB-sgRNA1-3′MS2 (after the |
| | | | stop codon of SaCas9 and before the PolyA signal |
| | | | sequence). |
| 30 | pSaCas9^{1PP7}- | Plasmid expressing | A synthetic DNA oligo (SEQ ID NO: 81) was cut with |
| | HBB- | SaCas9^{1PP7} mRNA and | KpnI and NotI and inserted between the KpnI-NotI sites |
| | sgRNA1^{3′PP7} | the guide RNA for HBB, | of pSaCas9^{1PP7}-HBB-sgRNA1^{3′MS2}. This results in |
| | | the 3′ region of the | excision of the U6-HBB sgRNA1-MS2 aptamer and |
| | | sgRNA has a PP7 stem | replacing it with the U6-HBB sgRNA1-PP7 aptamer |
| | | loop. | coding sequence. |
| 31 | pCK002- | Lentiviral vector | A dsDNA oligo was generated by annealing oligo sickle- |
| | HBB- | expressing SaCas9 and | g1-LV-F (SEQ ID NO: 53) and oligo sickle-g1-LV-R |
| | sgRNA1 | the sgRNA1 for HBB to | (SEQ ID NO: 54), which was then inserted into the |
| | | treat sickle cell disease. | BsmBI site of pCK002_U6-Sa-sgRNA(mod)_EFS- |
| | | | SaCas9-2A-Puro_WPRE (Addgene Cat. No. 85452). |
| | | | The dsDNA oligo is the guide sequence for HBB |
| | | | sgRNA1. |
| 32 | pAAV- | Adeno associated viral | A synthetic DNA encoding the human HBB target |
| | HBB- | (AAV) vector | template sequence and the U6 driven HBB sgRNA2 |
| | sgRNA2 | containing the HBB | expression cassette (SEQ ID NO: 82) was inserted into |
| | | template for | the NotI site of pAAV-MCS (Agilent Genomics, Cat. |
| | | homologous | No. 240071). |
| | | recombination to correct | |
| | | the Sickle Cell Disease | |
| | | mutation causing sickle | |
| | | cell disease. The SaCas9 | |
| | | target sites were | |
| | | removed but the | |
| | | encoding amino acids | |
| | | were identical to the | |
| | | wild type. The vector | |
| | | also contains the cassette | |
| | | for U6 driven expression | |
| | | of sgRNA2, targeting | |
| | | the HBB gene close to | |
| | | the mutation causing | |
| | | sickle cell disease. | |
| 33 | pAAV- | Adeno associated viral | Primer Sickle-g1HD-F (SEQ ID NO: 55) and Primer |
| | HBB- | vector containing the | Sickle-g1HD-R (SEQ ID NO: 56) were used to amplify |
| | sgRNA1 | HBB template for | the sickle-sgRNA1 expression cassette from pSaCas9- |
| | | homologous | HBB-sgRNA1 using high fidelity DNA polymerase. The |
| | | recombination to correct | amplified DNA was inserted into the XhoI and XbaI sites |
| | | the mutation causing | of pAAV-HBB-sickle-sgRNA2 by Infusion (Clontech). |
| | | sickle cell disease. The | |
| | | vector also contains the | |
| | | cassette for U6 driven | |
| | | expression of sgRNA1, | |
| | | targeting the HBB gene | |
| | | close to the mutation | |
| | | causing sickle cell | |
| | | disease. Since we found | |
| | | that sickle-sgRNA1 | |
| | | outperformed sickle- | |
| | | sgRNA2. | |
| 34 | pAAV- | Adeno associated viral | pAAV-HBB-sgRNA2 was cut with XhoI and XbaI, |
| | HBB(n)- | vector containing the | treated with Klenow DNA polymerase and the DNA was |
| | sgRNA1 | HBB template for | ligated to remove the cassette expressing sickle- |
| | | homologous | sgRNA2. Then the NcoI-PasI fragment of the resulted |
| | | recombination to change | plasmid was replaced by the annealed DNA from oligo |
| | | the wild type HBB gene | HBB-tem-F (SEQ ID NO: 57) and oligo HBB-tem-R |
| | | into the version causing | (SEQ ID NO: 58) to modify the template. Finally, the |
| | | sickle cell disease. This | NcoI-BstxI of the plasmid was replaced by the NcoI- |
| | | will facilitate the | BstxI fragment from pAAV-HBB-sgRNA1 to add the |
| | | detection of gene editing | HBB sgRNA1 expression cassette. |
| | | events in normal cells. | |
| | | The Sacas9 target sites | |
| | | were removed but the | |
| | | encoded amino acids | |
| | | were still wild type | |
| | | except for the disease | |
| | | causing mutation. The | |
| | | vector also contains the | |
| | | cassette for U6 driven | |
| | | expression of sgRNA1 | |
| | | for HBB, targeting the | |
| | | sequences close to the | |
| | | mutation causing sickle | |
| | | cell disease. | |
| 35 | pFCK- | Lentiviral vector | The sequences for human HBB template and the U6 |
| | HBB- | containing the human | driven HBB sgRNA1 expression cassette were amplified |
| | sgRNA1 | HBB template | from pAAV-HBB(n)-sgRNA1 with primer HBB-LT-F |
| | | (containing the mutation | (SEQ ID NO: 57) and primer HBB-LT-R (SEQ ID |
| | | causing sickle cell | NO: 58) using high fidelity DNA polymerase |
| | | disease) and the U6 | (proofreading HotStart Ready Mix from KAPA |
| | | driven HBB sgRNA1 | Biosystems (Wilmington, MA)). The DNA was cut |
| | | expression cassette. | with XbaI and EcoRV and inserted into XbaI and EcoRV |
| | | | sites of FCK-ChR2-GFP (Addgene Cat. No. 15814). |
| 36 | pSaCas9^{1xms2}- | Plasmid having two | A synthetic DNA of two copies of the human HBB 3′ |
| | 2x3′UTR | copies 3′ untranslated | UTR sequences (SEQ ID NO: 83) was inserted between |
| | | region (UTR) from | the BamHI-EcoRI sites of pSaCas9^{1xms2}. The synthetic |
| | | human HBB gene were | DNA was designed with a NheI site between the two |
| | | inserted after the Sacas9 | human HBB 3′ UTR sequences. |
| | | coding sequences and | |
| | | before the SM2 stem | |
| | | loop of pSaCas9^{1xms2}. | |
| 37 | pSaCas9^{1xms2}- | Plasmid having one | pSaCas9^{1xms2}-2x3′UTR was cut with NheI and EcoRI to |
| | 1x3′UTR | copy of HBB gene 3′ | remove one copy of HBB 3′ UTR. The backbone was |
| | | untranslated region | treated with Klenow DNA polymerase and re-ligated |
| | | (UTR) was after the | with T4 DNA ligase. |
| | | Sacas9 coding | |
| | | sequences and before the | |
| | | SM2 stem loop of | |
| | | pSaCas9^{1xms2}. | |
| 38 | pspCas9^{1xms2} | Plasmid having one | A dsDNA oligo was generated by annealing oligo sp- |
| | | copy of MS2 stem loop | loop1F (SEQ ID NO: 88) and oligo sp-loop1R (SEQ ID |
| | | was added after the stop | NO: 89) was inserted between the HindIII and EcoRI |
| | | codon of sp. Cas9 | sites of pU6-sgRosa26-1_CBh-Cas9-T2A-BFP (Addgene |
| | | mRNA. | Cat. No. 64216) by Infusion ™ reaction (Takara, In- |
| | | | Fusion ® HD Cloning Plus, Cat. 638909). |
| 39 | pLH-IL2RG-sp- | A lentiviral expression | A dsDNA oligo was generated by annealing oligo Il2RG- |
| | sgRNA | plasmid expressing a | sp-g1F1 (SEQ ID NO: 90) and oligo Il2RG-sp-g1R (SEQ |
| | | spCas9 sgRNA targeting | ID NO: 91) was inserted into the BbsI site of pLH- |
| | | the start codon region of | sgRNA1 (Addgene Cat. No. 75388) by T4 DNA ligase. |
| | | human IL2RG gene. | |
| 40 | pSaCas9^{1xMS2}- | A plasmid expressing | A synthetic dsDNA oligo was generated by annealing |
| | HBB-sgRNA1 | SaCas9^{1xMS2} and HBB | oligo 1xloop-F (SEQ ID NO: 41) and oligo 1xloop-R |
| | | sgRNA1. | (SEQ ID NO: 42), which was then inserted by infusion |
| | | | into the EcoRI site of pSaCas9-HBB-sgRNA1. |
| 41 | pSaCas9^{1xMS2}- | SaCas9 has a MS2 | A dsDNA oligo synthesized by Genscript (SEQ ID |
| | 2x3′UTR- | aptamer and two copies | NO: 84) was inserted into the Eag1 site of |
| | HBB | of HBB 3′ UTR, the | pSaCas91xMS2-2x3′UTR by T4 DNA ligase. The oligo |
| | sgRNA^{3′PP7} | HBB sgRNA1 has one | contains in 5′ to 3′ order: a U6 promoter, a HBB sgRNA |
| | 1x3′UTR | copy of PP7 aptamer | coding sequence (for Sickle mutation (g1)), a PP7 |
| | | and one copy of HBB 3′ | aptamer coding sequence, and a HBB 3′ UTR coding |
| | | UTR. | sequence. The U6 promoter direction is the same as the |
| | | | CMV promoter in the construct. |
| 42 | pSaCas9^{1xMS2}- | SaCas9 has a MS2 | A dsDNA oligo synthesized by Genscript (SEQ ID |
| | 2x3′UTR- | aptamer and two copies | NO: 85) was inserted between the EcoRV and Not1 |
| | HBB | of HBB 3′ UTR, the | sites of pSaCas9^{1xMS2}-2x3′UTR-HBB sgRNA^{3′PP7} |
| | sgRNA^{3′PP7} | HBB sgRNA1 has one | 1x3′UTR (Plasmid No. 41). The sequence |
| | 2x3′UTR | copy of PP7 aptamer | encoding one PP7 aptamer followed by one HBB |
| | | and two copy of HBB 3′ | 3′ UTR was replaced with a sequence encoding |
| | | UTR. | one PP7 aptamer followed by two HBB 3′ UTR |
| | | | sequences. |
| 43 | pSaCas9^{1xms2}- | One aptamer and 2 | Synthetic oligo MS2-F1 (SEQ ID NO: 92) and oligo |
| | 2x3′UTR- | copies of HBB 3′ UTR | MS2-R1 (SEQ ID NO: 93) were annealed and the dsDNA |
| | HBB- | sequences were added to | oligo was inserted between the Afe1 and EvoRV sites of |
| | sgRNA1^{3′MS2}- | both SaCas9 and HBB | pSaCas9^{1xMS2}-2x3′UTR-HBB sgRNA^{3′PP7} |
| | 2x3′UTR | sgRNA1. | 2x3′UTR (Plasmid No. 42) by Infusion ™ reaction |
| | | | (Takara, In-Fusion ® HD Cloning Plus. Cat. 638909). |
| 44 | pLVX-ad- | Lentivirus vector to | pLVX-EF1α-IRES-zsGreen1 was digested with MluI and |
| | IL2RG-rep | express the sgRNA2 | Clal to remove the zsGreen1-expression cassette and |
| | | targeting IL2RG gene. It | ligated with an adaptor to introduce the NotI site. Then |
| | | also contains the | the synthetic NotI DNA fragment containing the IL2RG |
| | | homologous | sgRNA2 expression cassette and the homologous |
| | | recombination arms for | recombination template were inserted into the NotI site |
| | | the insertion of IL2RG | of the modified vector (see SEQ ID 103). |
| | | cDNA into the target | |
| | | site. | |
| 45 | pLVX- | Lentivirus vector to | pLVX-EF1α-IRES-zsGreen1 was digested with MluI and |
| | HBB- | express the HBB | ClaI to remove the zsGreen1-expression cassette and |
| | correct | sgRNA1. It also contains | ligated with an adaptor to introduce the NotI site. Then |
| | | the homologous | the synthetic NotI DNA fragment (see SEQ ID 104) |
| | | recombination arms for | containing the HBB sgRNA1 expression cassette and the |
| | | the correction of the | homologous recombination template (to change the |
| | | Sickle mutation to the | Sickle mutation to the wild type HBB) were inserted into |
| | | wild type HBB gene. | the NotI site of the modified vector. |
| 46 | pSpCas9- | Plasmid for the | pspCas9-1loop was cut with FseI + NotI to |
| | 1loop- | expression of sp. Cas9 | remove the sequence containing the 1x MS2 |
| | 3′UTR | mRNA, with 2 copies of | aptamer and the bGH polyA signal, and the |
| | | HBB 3′UTR and one | vector backbone was recovered. Then the 600 |
| | | copy of MS2 aptamer | bp FseI + Eag1 fragment from pX601-1loop- |
| | | after the Cas9 stop | 2x3′UTR (containing 2xHBB 3′UTR, 1xMS2 |
| | | codon. | aptamer, and the bGH polyA signal) was |
| | | | ligated into the linearized the vector. |

TABLE 3
Primers Used in Examples

| Primer name | SEQ ID NO | Use |
|---|---|---|
| SaCas9-2576F | SEQ ID NO: 28 | SaCas9 QPCR |
| SaCas9-2713R | SEQ ID NO: 29 | SaCas9 QPCR |
| SgRNA-F1 | SEQ ID NO: 30 | sgRNA QPCR |
| sgRNA-R1 | SEQ ID NO: 31 | sgRNA QPCR |
| EGFP-RT-F | SEQ ID NO: 32 | EGFP QPCR |
| EGFP-RT-R | SEQ ID NO: 33 | EGFP QPCR |
| HHB-mut-F1 | SEQ ID NO: 34 | Next generation sequencing for the HBB target site. |
| HHB-mut-F2 | SEQ ID NO: 35 | Next generation sequencing for the HBB target site |
| HHB-mut-F3 | SEQ ID NO: 36 | Next generation sequencing for the HBB target site |
| HHB-mut-F4 | SEQ ID NO: 37 | Next generation sequencing for the HBB target site |
| HHB-mut-F5 | SEQ ID NO: 38 | Next generation sequencing for the HBB target site |
| HHB-mut-F6 | SEQ ID NO: 39 | Next generation sequencing for the HBB target site |
| HHB-mut-R1 | SEQ ID NO: 40 | Next generation sequencing for the HBB target site |
| HBB-1849F | SEQ ID NO: 96 | PCR amplification of the endogenous HBB locus |
| HBB-5277R | SEQ ID NO: 97 | PCR amplification of the endogenous HBB locus |
| Reporter-mut- | SEQ ID NO: 98 | PCR amplification of the HBB target sequence in |
| F1 | | the GFP expression cassette together with |
| | | Reporter-mut-R1 |
| Reporter-mut- | SEQ ID NO: 99 | PCR amplification of the HBB target sequence in |
| F2 | | the GFP expression cassette together with |
| | | Reporter-mut-R1 |
| Reporter-mut- | SEQ ID NO: 100 | PCR amplification of the HBB target sequence in |
| F3 | | the GFP expression cassette together with |
| | | Reporter-mut-R1 |
| Reporter-mut- | SEQ ID NO: 101 | PCR amplification of the HBB target sequence in |
| F4 | | the GFP expression cassette together with |
| | | Reporter-mut-R1 |
| Reporter-mut- | SEQ ID NO: 102 | PCR amplification of the HBB target sequence in |
| R1 | | the GFP expression cassette together with |
| | | Reporter-mut-F1, Reporter-mut-F2, Reporter- |
| | | mut-F3 or Reporter-mut-F4. |

Assays for Assessing Gene Editing Activity. To assess gene editing, the mutated sequence causing sickle cell disease was chosen as the genomic target sequence for editing. The gene defect that cause sickle cell disease is a point mutation of A nucleotide to T nucleotide of the human beta hemoglobin gene (converts a GAG codon into GUG), which results in glutamic acid (E/Glu) being substituted by valine (V/Val) at amino acid position 6 of the β-globin protein. The sequence of the targeted strand is ACTCCTGTGGAGAAGTCTGCCGTTACT (SEQ ID NO: 94); the first 6 nucleotides of this sequence (underlined) correspond to the protospacer adjacent motif. Two assays were used to detect gene editing activities: (1) a GFP-reporter assay and (2) the Surveyor® Mutation Detection Kit (Integrated DNA Technologies, Cat. No. 706020).

For the GFP-reporter assay, enhanced green fluorescent protein (EGFP) reporter cells described in Javidi-Parsijani, P. et al., PLOS One 2017; 12 (5): e0177444 (referred to throughout the Examples as “GFP reporter cells” or “reporter cells”) were used for the detection of genome editing activities by microscopic observation or flow cytometry analysis of GFP-positive cells. This cell line does not express EGFP due to disruption of the EGFP reading frame by the insertion of a 119-base pairs sequence between the start codon ATG and the second codon of EGFP cDNA (insertion is missing 1 bp to make 40 in-frame codons). The 119 bp insertion is

(SEQ ID NO: 95)
gaacccaggttcctgacacagacagactacacccagggaatgaagagcaa
gcgccatACTCCTGTGGAGAAGTCTGCCGTTACTGCCCTGTGGGGCAAGG
TGAACGTGGATTGGCTAGC,

and contains a 57 bp IL2RG target sequence (underlined) and a 62 bp HBB target sequence (capitalized). Without genome editing, EGFP will not be expressed due to the presence of the insertion, which disrupts the EGFP reading frame. This was confirmed by the absence of EGFP-positive cells in the non-transfected reporter cells and in the reporter cells transfected with plasmid DNA expressing SaCas9 without any sgRNA. Upon genome editing in the inserted sequence, the double strand breaks are repaired by non-homologous end joining. Due to the possibility of introducing insertion or deletion (INDELs), deletions of 3N+2 or insertions of 3N+1 base pairs will restore the EGFP reading frame and, thus, EGFP protein expression.

To test whether the reporter cells were functional, the cells were transduced with adeno-associated virus particles containing SaCas9 mRNA and sgRNA for HBB (made from pSaCas9-HBB-sgRNA1; Plasmid No. 22). Flow cytometry analysis showed that 4.5% to 15% of the cells were EGFP-positive 24 hours post-transfection. The genomic DNA from the reporter cells was purified three days after the transduction, amplified the reporter region by PCR with high-fidelity DNA polymerase, and sequenced the target region by next-generation sequencing. The percentage of sequences with INDELs was analyzed. 68% of the reads were found to have INDELs. As only some INDELs will restore EGFP expression, and analysis was performed 72 hours after transfection, it is not surprising that a lower EGFP-positive rate than INDEL rate was observed. These data indicate that the reporter cells are functional and useful for analysis of gene editing events in the context of this disclosure.

The Surveyor® Mutation Detection Kit was used according to the kit's manual to detect gene editing activities. The key component of the kit, Surveyor Nuclease, a member of the CEL family of mismatch-specific nucleases, recognizes and cleaves mismatches due to the presence of single nucleotide polymorphisms (SNPs) or small insertions or deletions. Briefly, target sequences were amplified using the proofreading HotStart ReadyMix from KAPA Biosystems (Wilmington, MA), a high fidelity, thermostable DNA polymerase, to minimize the incorporation of errors that would result in background mismatches. The amplified DNA was denatured for 5 mins at 95° C. and renatured by slowly lowing the temperature to room temperature. The DNA was then treated with the Surveyor Nuclease to cleave mismatches. The cleaved DNA fragments were separated by agarose gel electrophoresis, stained with ethidium bromide and observed under UV light.

Packaging Cas9 mRNA in Lentivirus-like Particles. Lentivirus-like particles with genomes were produced with Addgene second or the third generation packaging systems as described in Javidi-Parsijani, P. et al., PLOS One 2017; 12 (5): e0177444. To package SaCas9 mRNA into the lentivirus-like particles, HEK293T cells were transfected with the desired combination of plasmids. Transfection was mediated by Polyethylenimine (PEI, Polysciences Inc.) using a DNA:PEI ratio of 1:2 (mass:mass). Table 4 provides exemplary conditions used to generate certain Cas9 mRNA packaged lentivirus-like particles. Cell culture and DNA transfection were performed as described in Javidi-Parsijani, P. et al., PLOS One 2017; 12 (5): e0177444. The viral particles were either used without concentration or after concentration by one of the three different methods: 1) by laying the supernatant containing the viral particles on 10 ml 20% sucrose cushion, and then centrifuge at 20000 g 4° C. for 4 hours, 2) by using the Lenti-X™ Concentrator using the manufacturers protocol (Clontech, Cat. No. 631232), 3) by using the KR2i TFF System [KrosFlo® Research 2i Tangential Flow Filtration System] (Spectrum Lab, Cat. No. SYR2-U20). Transduction experiments found that virus produced by the third method worked the best (judged by the percentage of GFP positive cells generated in GFP reporter assays), so most data were generated with viral particles concentrated by this method.

TABLE 4
Plasmid DNA used to transfect HEK293T cells to make
lentiviral particles loaded with SaCas9 mRNA^{a}

| | MCP-fused | MCP-fused | PCP-fused | PCP-fused | VPR or NEF |
|---|---|---|---|---|---|
| | second | third | second | third | mediated |
| | generation | generation | generation | generation | SaCas9 |
| | packaging | packaging | packaging | packaging | mRNA |
| | system | system | system | system | packaging |
| Gag-pol | pspAX2-D64V- | pMDLg/pRRE- | pspAX2-D64V- | pMDLg/pRRE- | pspAX2-D64V |
| packaging | NC-MS2 | D64V-NC-MS2 | NC-PP7 | D64V-NC-PP7 | |
| plasmid | | | | | |
| (16.5 μg) | | | | | |
| SaCas9 | pSaCas9^{1xMS2} | pSaCas9^{1xMS2} | pSaCas9^{1xPP7} | pSaCas9^{1xPP7} | pSaCas9^{1xMS2} |
| plasmid | | | | | |
| (12 μg) | | | | | |
| pMD2G | + | + | + | + | + |
| (8 μg) | | | | | |
| pRSV-REV | − | + | − | + | − |
| (4.5 μg) | | | | | |
| VPR or NEF | − | − | − | − | + |
| MCP fusion | | | | | |
| plasmid | | | | | |
| (12 μg) | | | | | |

^{a}13 × 10⁶ cells were seeded in 15-cm dishes 24 hours before transfection.

Viral titer detection. Viral titer was determined by measuring p24 by enzyme-linked immunosorbent assay (ELISA) using the QuickTiter™ Lentivirus Titer Kit (Cell Biolabs, Cat. No. VPK-107). As per manufacturer's instructions, the viral particles were precipitated prior to the ELISA analysis so that the soluble p24 protein was not detected.

Viral RNA Isolation and Quantification. Two methods were used to isolate viral RNA. In one method, the viral particles were centrifuged at 120,000 g for 90 mins, and then the viral RNA was purified with the miRNeasy Mini Kit (QIAGEN®, Cat No. 217004). Alternatively, viral RNA was isolated directly from 140 μl viral supernatant with the QIAamp Viral RNA Mini Kit (QIAGEN®, Cat. No. 52904). Reverse transcription of viral RNA was done with the QuantiTect® Reverse Transcription Kit (QIAGEN). Custom designed TaqMan™ probes were used for quantitative PCR (QPCR). Quantitation was performed by QPCR or by digital PCR. Absolute QPCR using the standard curve method was performed using SYBR™ Green qPCR master mixture (ThermoFisher). A standard curve was prepared with 50, 500, 5000, 50000 and 500000 copies of pSacas9-HBB-sgRNA1 (for SaCas9 and sgRNA) or pCDH-GFP (for EGFP) plasmid DNA. The concentration of plasmid DNA used for preparing standards was determined using a NanoDrop™ 2000 (Cat. No. ND-2000) spectrophotometer (ThermoFisher). PCR was run on an ABI 7500 instrument (Applied Biosystems, ThermoFisher). Dissociation analysis was performed after the amplification to determine the specificity of the PCR product (if only one peak was observed in the dissociation curve, the amplification was specific). Alternatively, digital PCR was performed for quantification with the QuantStudio™ 3D Digital PCR System (ThermoFisher Scientific).

Lentivirus-like Particle and Lentiviral Particle Transduction. Various amount of concentrated viral particles (equivalent to 10˜200 ng p24 protein) were added to GFP-reporter cells (2×10⁴) as described above and grown in 24-well plates, with 8-12 μg/ml polybrene in DMEM/10% FBS. Supernatant containing non-concentrated virus was used to transduce GFP-reporter cells with half fresh medium and half virus containing supernatant. The cells were incubated with the virus-containing medium for 12 hours, after which the medium was replaced with fresh DMEM medium with 10% FBS.

Genomic DNA Extraction From Human cells. Genomic DNA was isolated from cells with the QIAamp DNA Mini Kit (Qiagen, Germantown, MD) according to the manufacturer's instructions.

Western blotting. Lentivirus or lentivirus-like particles (200 ng) were directly lysed in 50 μl of 1xSDS loading buffer, heated at 95° C. for 5 min. The proteins were separated by SDS-PAGE, transferred to PVDF membranes and immunoblotted for MA (p17) with anti-p17 antibody (Fisher Scientific, Cat #PA14954, 1:1000), for CA (p24) with anti-p24 antibody (Cell Biolabs, Cat. No. 310810, 1:1000), and for NC with anti-p15 antibody (Abcam, Cat. No. ab66951, 1:1000). Horseradish peroxidase (HRP)-conjugated secondary antibodies and chemiluminescent reagents were purchased from ThermoFisher Scientific. The LAS-3000 system (Fujifilm) was used to capture Western blotting images. Densitometry of protein bands was analyzed with Image J software.

### Example 2. Fusing MS2 Coat Protein (MCP) to Lentiviral Proteins for SaCas9 mRNA Packaging

The aim was to package SaCas9 mRNA in lentivirus-like particles using the high affinity interaction between bacteriophage coat proteins and their respective RNA aptamers. Aptamer-binding proteins were fused with various lentiviral proteins. The corresponding aptamer sequence was added to the 3′ untranslated region (UTR) of SaCas9 (see FIG. 2). Four different lentiviral proteins were tested as the fusion recipient for MS2 coat protein (MCP), a commonly used aptamer-binding protein. These fusion proteins were HIV accessory proteins Viral Protein R (VPR) (Wu, Liu et al., J. Virol. 69 (6): 3389-3398 (1995)) and Negative Regulatory Factor and HIV structure proteins Nucleocapsid Protein (NC) and Matrix Protein (MA) (referred to as MCPx2-VPR, NEF-MCPx2, NC-MCPx2, and MA-MCPx2, respectively). The NEF protein included three mutations (G3C, V153L and G177E) to increase incorporation into lentivirus-like particles as described in Muratori, C., et al., Methods Mol Biol 2010; 614:111-124. Two tandem copies of MCPs were fused to the N-terminus of VPR and the C-terminus of NEF following published studies Wu, X., et al., J Virol 1995; 69 (6): 3389-3398 and Muratori, C., et al., Methods Mol Biol 2010; 614:111-124. For the NC fusion protein, two copies of MCPs were inserted after NC zinc finger 2 of the Gag precursor protein in packaging plasmids pMDLg/pRRE^{D64V} (third generation packaging plasmid) and psPAX2^{D64V} (second generation packaging plasmid), preserving the NC/P1 protease cleavage site. For the MA fusion protein, amino acids 44-132 of MA in these packaging plasmids were replaced with two copies of MCP. Deleting this region in MA is known not interfere with viral particle assembly or budding (Jalaguier, P., et al., PLOS One 2011; 6 (11): e28314). Fusing MCP to MA or NC did not disrupt the reading frame of the Gag precursor protein or the protease processing sites.

Neither expressing MCPx2-VPR or NEF-MCPx2 during lentiviral vector production, nor fusing MCP with NC or MA impaired the efficiency of lentiviral particle production. P24 ELISA revealed that in these situations the lentiviral production was equally efficient as virus production by the packaging plasmids without MCP fusion (Table 5). The control used was the unmodified packaging plasmid without MCP fusion. The plasmid name is pspAX2-D64V.

TABLE 5
Viral production efficiency and gene editing activities
of packaging plasmids with various fusion proteins.

| | No MCP | | | | |
|---|---|---|---|---|---|
| Constructs | fusion | VPR-MCP | NEF-MCP | MA-MCP | NC-MCP |
| Viral production | 100.0 ± 3.2 | 104 ± 14.2 | 120.3 ± 2.7 | 142.0 ± 14.8 | 100.1 ± 26.7 |
| efficiency^{a} (ng/ml) | (8) | (3) | (3) | (6) | (7) |
| GFP⁺ percentage^{b} | 2.46 ± 0.06 | 3.17 ± 0.35 | 3.74 ± 0.33 | 2.87 ± 0.68 | 5.22 ± 0.11 |
| | (4) | (3) | (3) | (3) | (3)*** |

aThe GFP-expressing lentivirus vector pCDH-GFP was packaged. The viral production efficiency of the control packaging plasmid without fusion proteins was set as 100%. Mean ± Sem was shown. Numbers in parentheses mean number of repeats for each group. No difference was noted between groups by ANOVA analysis.
^{b}SaCas9^{1xMS2} was expressed during lentivirus-like particle production. 250 μl of un-concentrated lentivirus-like particles were co-transduced into 4 × 10⁴ GFP-reporter cells with a lentiviral vector expressing HBB sgRNA1.
***indicates p < 0.0001 when viral particles from NC-MCP fusion was compared with viral particles by packaging plasmid without MCP fusion.

The impact of these fusion proteins on gene editing was then assessed. One copy of MS2 aptamer was added after the stop codon of SaCas9 mRNA (SaCas9^{1xMS2}) as described in Table 2. Lentivirus-like particles were made and packaged with SaCas9^{1xMS2} as described in Example 1. SaCas9^{1xMS2} mRNA was transcribed in HEK293T cells during lentivirus-like particle production in the presence of any one of the four different fusion proteins (see Table 4 for how the viral particles were made). The unconcentrated viral-like particles (collected medium) were co-transduced with a lentiviral vector expressing human beta hemoglobin (HBB) sgRNA1 (Plasmid No. 35, pFCK-HBB-sgRNA1) into the GFP-reporter cells as described in Example 1. Gene editing in these reporter cells results insertions or deletions (Indels) in the HBB target sequence inserted after the EGFP start codon, which restore the EGFP reading frame. Viral-like particles produced from unmodified packaging plasmid produced about 2.5% GFP⁺ reporter cells. Viral-like particles expressing VPR-MCP, NEF-MCP and MA-MCP fusion proteins did not significantly increase the GFP⁺ reporter cells, while NC-MCP fusion generated over 5% GFP⁺ reporter cells (see Table 5). As only one third of the Indels in the reporter cells could restore the EGFP reading frame, only a third of gene editing events are detectable using these reporter cells (see Javidi-Parsijani, P., et al., PLOS One 2017; 12 (5): e0177444). As such, un-concentrated lentivirus-like particles expressing NC-MCP fusion protein were able to generate Indels in over 15% of the reporter cells (3× the detected rate of 5%). In addition, GFP⁺ cells could only be observed when the lentivirus-like particles were co-transduced with the lentiviral vector expressing HBB sgRNA1. Thus, gene editing events were limited only to cells transduced both by the fusion protein lentivirus-like particles and the virus particles carrying the HBB sgRNA1. As such, the rate of gene editing occurring when the NC-MCP fusion protein particles is used in the system is likely greater than 15%. In view of these results, further experiments were performed using NC as the recipient protein for fusion with RNA-binding aptamer proteins.

The performance of packaging plasmids with one or two copies of MCP fused to NC was compared (Plasmid Nos. 11 and 13). SaCas9^{1xMS2} containing viral-like particles generated by NC-MCPx1 packaging plasmid produced more GFP+ cells in GFP reporter assays than particles generated by NC-MCPx2 packaging plasmid (NC-MCPx1: 6.2%±0.1%, N=4; NC-MCPx2: 4.8%±0.4%, N=4; p<0.05). In view of these results, the NC-MCPx1 packaging plasmid was used in subsequent experiments unless otherwise specified.

As the lentiviral Env protein VSV-G promotes extracellular vesicle production (Rolls, M. M., et al., Cell 1994; 79 (3): 497-506), it was possible that SaCas9^{1xMS2} mRNA could be being introduced into the reporter cells from extracellular vesicles rather than increased packaging into the lentivirus-like particles. To test this, an experiment was conducted comparing the gene editing rate induced by lentivirus-like particles expressing NC-MCPx1 (using psPAX2-D64V-NC-MS2, Plasmid No. 11) or a control generated using an mRuby-expressing plasmid (pKanCMV-mRuby3-10aa-H2B; Addgene Cat. No. 74258) that did not express any of the lentiviral packaging proteins (mRuby is a red fluorescent protein). The mRuby expressing plasmid does not express any lentivirus packaging proteins so no lentivirus will be produced from it alone. If extracellular vesicles but not lentivirus-like particles are the structures mediating Cas9 mRNA transfer, then mRuby mRNA or plasmid should be similarly incorporated into the extracellular vesicles and the mRuby protein should be expressed with similar efficiency as Cas9 protein. The particles were produced as described in Example 1 and were concentrated by ultracentrifugation, which was expected to precipitate both lentivirus-like particles and extracellular vesicles. To measure gene editing, the precipitate was used to transduce GFP reporter cells together with lentivirus particles containing HBB sgRNA1. Particles generated with the NC-MCP modified packaging plasmid generated over 10% GFP⁺ reporter cells, while particles generated with the mRuby plasmid instead generated less than 1% GFP+ reporter cells as well as less than 1% mRuby⁺ reporter cells. The precipitate containing lentivirus-like particles containing NC-MCPx1 also containing Cas9 mRNA, which led to the high GFP+ reporter cell rate observed. Without the NC-MS2 packaging plasmid (replaced by mRuby plasmid), the precipitate only contained extracellular vesicles, which led to the low GFP+ reporter cell rate observed. This showed that extracellular vesicles did not have much Cas9 mRNA or mRuby mRNA (mRuby also low percentage). As VSV-G was expressed in both conditions, the key difference was whether the NC-MCP modified packaging plasmid was used. The results suggest that extracellular vesicles do not play a major role in generating GFP⁺ reporter cells in this system.

To test whether GFP+ reporter cells were simply generated by the plasmid DNA left over from the lentivirus-like particle production, a plasmid expressing both SaCas9^{1xMS2} mRNA and HBB sgRNA1 (Plasmid No. 40, pSaCas9^{1xMS2}-HBB-sgRNA1) was transfected into the packaging cells during lentivirus-like particle production with the NC-MCP modified packaging plasmid (Plasmid No. 11). The sgRNA1 does not include an aptamer sequence. As such, although both SaCas9^{1xMS2} mRNA and HBB sgRNA1 are transcribed from the plasmid, only SaCas9^{1xMS2} mRNA could be packaged into lentivirus-like particles with detectable efficiency. The resulting lentivirus-like particles were used to transduce the GFP reporter cells alone (condition 1) or in combination with lentiviral particles containing HBB sgRNA1 (made using Plasmid No. 35) (condition 2). In condition 2, 10% GFP+ reporter cells were observed, while less than 3% GFP+ cells were observed in condition 1. Simply transfecting the GFP-reporter cells with pSaCas9^{1xMS2}-HBB-sgRNA1 DNA generated over 10% GFP reporter cells. The data suggest that plasmid DNA left over during lentivirus-like particle production is not a major contribution to the SaCas9^{1xMS2} mRNA and support a conclusion that SaCas9^{1xMS2} mRNA is packaged into lentivirus-like particles.

### Example 3. Effects of MS2 Aptamer on SaCas9 mRNA Stability

Two possible mechanisms might contribute to the packaging of SaCas9^{0xMS2} mRNA in lentivirus-like particles. First, cellular mRNA (including the SaCas9 mRNA) could be packaged into the lentivirus-like particles non-specifically, as described by Rulli, S. J., et al., J Virol 2007; 81(12): 6623-6631. Second, SaCas9 mRNA might have some RNA structures that could be bound by the NC-MCP fusion protein or other lentiviral proteins. When the genome editing activity of un-concentrated lentivirus-like particles containing either SaCas9^{1xMS2} or SaCas9^{0xMS2} by GFP cell reporter assay was compared, the percentages of GFP+ cells generated by the two showed hardly any difference (9.4%±0.1%, N=4; 9.1%±0.2%, N=4; p=0.17). Although the aptamer is not essential for SaCa9 mRNA to be packaged in the lentivitus-like particles, NC-MCP fusion greatly enhanced the packaging of SaCas9 mRNA by 10 times compared with the amount of SaCas9 mRNA packaged by unmodified packaging plasmid.

To examine the effects of MS2 aptamers on SaCas9 mRNA level in the cells, an experiment was conducted to assess the effects of 0, 1, 2, 3 and 12 copies of MS2 aptamers added to the 3′ end of the SaCas9 mRNA (Plasmids Nos. 15-19) on mRNA level. Equal amounts of plasmid DNA encoding SaCas9^{rxMS2} (n indicates 0, 1, 2, 3 or 12) was transfected into HEK293T cells, and the steady-state level of SaCas9 mRNA was compared by real time RT-PCR. It was found that addition of 1 aptamer after the SaCas9 coding sequence slightly decreased the steady-state level of SaCas9 mRNA, while the addition of 2 or more aptamers significantly decreased the steady-state level of SaCas9 mRNA (Table 6). Consistent with the mRNA level decrease, when the SaCas9^{mxMS2}-encoding plasmids (n indicates 0, 1, 2, 3 or 12) (250 ng) were co-transfected with a plasmid expressing the HBB sgRNA1 (250 ng) into the GFP reporter cells (24-well plates), one aptamer slightly decreased the percentage of GFP⁺ reporter cells observed, while more aptamers further decreased the number observed (Table 6). Flow cytometry and qRT-PCR were performed 72 hours after transfection. HBB sgRNA1 was used as the transfection efficiency control for qRT-PCR. The data suggest that addition of the MS2 aptamer decreased SaCas9 stability. This mRNA stability decrease could have a measurable effect on genome editing activity because SaCas9 mRNA cannot be replaced (regenerated) once they are released from the lentivirus-like particles.

TABLE 6
Effects of MS2 aptamers on SaCas9 expression

| | No | 1 | 2 | 3 | 12 |
|---|---|---|---|---|---|
| | aptamer | aptamer | aptamers | aptamers | aptamers |
| SaCas9 | 1.0 ± 0.03 | 0.76 ± 0.03^{#} | 0.18 ± 0.01^{##} | 0.19 ± 0.01^{##} | 0.27 ± 0.03^{##} |
| mRNA | | | | | |
| level | | | | | |
| GFP⁺ | 9.9 ± 0.15** | 8.3 ± 0.12* | 6.7 ± 0.15 | 7.5 ± 0.38 | 7.3 ± 0.39 |
| percentage | | | | | |

Mean ± s.e.m of 4 replicates were presented.
^{#}p < 0.0001 compared with no aptamer;
^{##}p < 0.0001 compared with 1 aptamer;
*p < 0.05 when 1 aptamer was compared to 2 aptamer;
**p < 0.01 when No aptamer was compared with 1, 2, 3, or 12 aptamers.

### Example 4. Human HBB Gene 3′ UTR Sequence Increased SaCas9 mRNA Stability

An experiment was conducted to assess whether addition of one or two copies of human HBB gene 3′ UTR sequences after the MS2 aptamer of SaCas9^{1xMS2} could enhance SaCas9 mRNA stability and translatability in the presence of aptamer. Plasmid pSaCas9^{1xms2} and pSaCas9^{1xms2}-2×3′UTR was transfected into HEK293T cells (Plasmid Nos. 16 and 36). The steady-state level of SaCas9 mRNA in the cells was assessed by real time RT-PCR. The addition of two human HBB gene 3′ UTR sequences was found to increase the steady-state level of SaCas9 mRNA level by 30% (1.0±0.03 versus 1.3±0.08, n=4, p<0.05). GFP reporter cells were co-transduced with lentivirus-like particles containing SaCas9^{1xMS2}-2×3′UTR mRNA or lentivirus-like particles containing SaCas9^{1xMS2} mRNA (no stabilizing sequences), and lentivirus particles containing HBB sgRNA1 (generated using Plasmid No. 35). Transduction with the lentivirus-like particles containing SaCas9^{1xMS2}-2×3′UTR mRNA resulted in a significantly increased percentage of GFP⁺ reporter cells than lentivirus-like particles containing SaCas9^{1xMS2} mRNA (see FIG. 3). Thus, addition of 2 copies of HBB 3′ UTR sequences to SaCas9^{1xMS2} improved the performance of the system.

### Example 5. SaCas9 Lentivirus-like Particles Based on PP7 Coat Protein and Its Aptamer Showed High Genome Editing Activity

PP7 coat protein (PCP) is another RNA aptamer-binding protein (see Lim, F., et al., J Biol Chem 2001; 276 (25): 22507-22513 and Wu, B., et al., Biophysical Journal 2012; 102 (12): 2936-2944). An experiment was conducted to compare the utility of the PP7 protein and its cogent aptamer sequence in this system in place of the MCP and its aptamer sequence. A packaging plasmid was produced in which MCP was replaced with PCP thereby creating a NC-PCP fusion. Also constructed was a SaCas9 mRNA expression plasmid in which the MS2 aptamer coding sequence was replaced by the PP7 aptamer coding sequence (Plasmid No. 20, pSaCas9^{1xPP7}). These plasmids were used to make SaCas9^{1xPP7} mRNA packaged lentivirus-like particles. Upon co-transducing GFP reporter cells with lentiviral vectors expressing the HBB targeting sgRNA1, using SaCas9^{1xPP7}-containing lentivirus-like particles resulted more GFP+ reporter cells than SaCas9^{1xMS2}-containing lentivirus-like particles (9.4%±0.14%, N=4; 8.1%±0.04%, N=4, p<0.01).

The amount of SaCas9 mRNA packaged into the lentivirus-like particles was assessed by real time RT-PCR, as summarized in Table 7. The types of particles included lentivirus (column 2), lentivirus-like particles produced by packaging plasmid with no ABP fusion (column 3), by NC-MCP fusion packaging plasmid (columns 4-6), and by NC-PCP fusion packaging plasmid (columns 7-9). The SaCas9 mRNA contained no aptamer (columns 2, 3, 4 and 7), one aptamer (columns 5 and 8), or one aptamer and 2 copies of HBB 3′ UTR (columns 6 and 9). RNA was purified from various lentivius or lentivirus-like particles containing 200 ng of p24. Equal amount of GFP-lentivirus was included in each sample to normalize the RNA purification, RT, and PCR. Copy number estimation is based on the assumption that each lentivirus particle contains 2 copies of the lentivirus genome. By comparing the SaCas9 mRNA levels in the same amount of SaCas9-expressing lentivirus (known to have 2 copies of genome per particle) or lentivirus-like particles, we calculated the average copy numbers of SaCas9 mRNA per particle in each condition. In Table 7, means±SEM are shown, and the numbers in the parentheses are the numbers of replicates.

TABLE 7
QRT-PCR comparison of the copy number of SaCas9 in various lentivirus-like particles

| | | No ABP | NC-MCP packaged | | | NC-PCP packaged | | |
|---|---|---|---|---|---|---|---|---|
| | | No | | | 1 MS2 | | | 1 PP7 |
| | Lentivirus | aptamer | 0 MS2 | 1 MS2 | 3′UTR | 0 PP7 | 1 PP7 | 3′ UTR |
| Relative | 1 (3) | 0.71 ± 0.06 | 15.8 ± 1.1 | 13.3 ± 0.3 | 50.1 ± 2.6 | 7.9 ± 1.5 | 6.6 ± 0.6 | 21.8 ± 0.1 |
| SaCas9 | | (2) | (3) | (3) | (3) | (3) | (3) | (2) |
| mRNA level | | | | | | | | |
| SaCas9 | 2 | 1.4 | 31.6 | 26.6 | 100.2 | 15.8 | 13.2 | 43.6 |
| mRNA copy | | | | | | | | |
| number | | | | | | | | |

Without ABP fused to NC and without aptamer after the stop codon of SaCas9, lentivirus-like particles were found to have less than two copies of SaCas9 mRNA per particle. This could be the result of randomly packaging highly expressed cellular RNA by the virus-like particles. With ABP (MCP or PCP) fused to NC, the SaCas9 mRNA copy number per particle increased 10 to 20 times regardless whether an aptamer sequence is appended to SaCas9. Addition of HBB 3′ UTR increased the copy numbers 3 fold with both ABPs, which is most likely the result of increasing the RNA stability before and after packaging. Adding one aptamer to SaCas9 mRNA did not increase SaCas9 mRNA copy number per particle compared with SaCas9 mRNA without aptamers, which is most likely the combined result of aptamer increasing the binding of SaCas9 mRNA to the NC-ABP fusion proteins but decreasing the stability of SaCas9 mRNA. These effects of aptamers on SaCas9 mRNA are also consistent with the observation of HBB 3′ UTR addition increasing SaCas9 mRNA copy number per particle, as HBB 3′ UTR increases mRNA stability although unable to increase the binding to ABPs.

The expression of viral proteins MA, CA, and p15 (where NC is processed from) was also assessed in normal GFP lentiviral vectors and in MCP and PCP-based lentivirus-like particles by Western blotting. The NC-PCP fusion protein was found to migrate as a single band with the expected size of about 22 kDa, while an additional small band of about 14 kDa was observed for the NC-MCP fusion protein, indicating partial degradation. The MA and CA proteins were observed to migrate at the expected sizes from all of the particles, suggesting that the insertion of MCP or PCP into NC protein did not affect the processing of other lentiviral proteins. Electron microscopy analyses of the MCP- and PCP-based lentivirus-like particles revealed similar particles sizes (see FIG. 4).

### Example 6. Transient Expression of SaCas9 mRNA from Lentivirus-Like Particles

To determine the duration of the SaCas9 mRNA released from lentivirus-like particles, lentivirus-like particles containing SaCas9^{1xPP7} mRNA (Plasmid No. 20), SaCas9^{1xMS2} mRNA (Plasmid No. 16), or SaCas9^{1xMS2}-2×3′UTR mRNA (Plasmid No. 36) were transduced into HEK293T cells and the level of SaCas9 mRNA at 24, 48, 72 and 96 hours post transduction was measured by quantitative RT-PCR. As controls, the SaCas9 mRNA levels expressed from adeno associated virus (AAV, made using Plasmid No. 15) and integration defective lentivirus (IDLV, made using Plasmid No. 31) were also assessed. All DNA was eliminated before reverse transcription, thus all SaCas9 nucleic acid detected was mRNA. For both AAV and IDLV, SaCas9 mRNA was increased 48 hours after transduction, consistent with the start of transcription from the DNA templates as shown in FIG. 5A. The level of SaCas9 mRNA in AAV-transduced cells was still higher at 96 hour post-transduction than at 24 hours post-transduction. The SaCas9 mRNA level in IDLV-transduced cells started to decrease after 72 hours but expression at 96 hours after transduction was still about half of that at 24 hour post transduction. In cells transduced with lentivirus-like particles packaged with modified SaCas9, the level of SaCas9 mRNA decreased after 24 hours post-transduction as shown in FIG. 5B. At 96 hours post-transduction, the level of SaCas9 mRNA was less than 25% of that of 24 hours post-transduction. The addition of the HBB 3′ UTR to the SaCas9 mRNA slowed its degradation.

### Example 7. Efficient Genome Editing Activity from Transient Expression of SaCas9 mRNA

Lentivirus-like particles containing SaCas9^{1xMS2}-2×3′ UTR mRNA (made using Plasmid No. 36) and lentivirus particles expressing HBB sgRNA1 (made using Plasmid No. 35) were made and concentrated by Tangential Flow Filtration as described in Example 1. The virus concentration was determined by p24 ELISA. The lentivirus-like particles and lentivirus particles (30 ng p24 protein of each) were co-transduced into 2.5×10⁴ GFP reporter cells as described in Example 1. The HBB sgRNA1 was designed against a mutated HBB sequence known to cause Sickle Cell Disease that has a one nucleotide mismatch with the endogenous HBB sequence in the reporter cells as described in Example 1. The target sequence of HBB sgRNA1 inserted between the first and second codon of the GFP coding sequence in the transduced GFP reporter cells was amplified and Next Generation Sequencing was performed to detect Indels. Sequence analysis found that 86.5% of the alleles had Indels. Thus, the described system was very efficient in editing the Sickle Cell Disease mutation. A close examination of the positions of the Indels found that most of the Indels were around the expected cleavage site (3 nt from the PAM) (see FIG. 6, listing SEQ ID NOs: 103-112 from top to bottom), suggesting that the Indels observed were from gene editing guided by HBB sgRNA1.

### Example 8. Packaging sgRNA in Lentivirus-Like Particles

An experiment was conducted to determine whether sgRNA could also be packaged into lentivirus-like particles via the interactions between coat proteins and their respective aptamers. Other studies have found that aptamer sequences can be added at the stem loop II, tetra loop, or after the 3′ end of SpCas9 sgRNA (Shechner, D. M., et al., Nat Methods 2015; 12 (7): 664-670 and Konermann, S., et al., Nature 2015; 517 (7536): 583-588). However, no information was available about the locations that can tolerate aptamer addition in SaCas9 sgRNA. Modified plasmids were generated by adding one or two MS2 aptamers in the SaCas9 sgRNA at stem loop II (ST2), tetra loop (Tetra), or after the 3′ end in plasmid pSaCas9-HBB-sgRNA1 (Plasmid No. 22), which expresses both SaCas9 mRNA and the HBB sgRNA1. The modified plasmid DNA was transfected into GFP reporter cells. The addition of one aptamer at the 3′ end of sgRNA showed the least decrease in percentage of GFP+ reporter cells (Table 8). In view of these results, sgRNA with a 3′ addition of aptamer was used in subsequent experiments.

TABLE 8
Effects of Aptamer addition on sgRNA performance

| | No | | | Tetra + | | Tetra + |
|---|---|---|---|---|---|---|
| | aptamer | ST2 | Tetra | ST2 | 3′ end | 3′ end |
| GFP⁺ | 8.5 ± 0.1 | 7.2 ± 0.3 | 7.5 ± 0.3 | 6.9 ± 0.1* | 8.4 ± 0.6 | 6.7 ± 0.1* |
| (%) | | | | | | |

Shown are mean ± sem (N = 3).
*indicates significantly lower than control (No aptamer) by ANOVA and Tukey's Multiple Comparison Test (p < 0.05).

Another experiment was performed to assess whether the 3′ MS2 aptamer-modified HBB sgRNA1 (HBB sgRNA13′MS2) could be packaged in lentivirus-like particles expressing NC-MCP fusion protein using packaging plasmid. Plasmid DNA expressing modified and unmodified HBB sgRNA1 (Plasmid No. 22 for sgRNA without aptamer, Plasmid No. 24 for sgRNA with a 3′ MS2 aptamer) were co-transfected into HEK293T cells during lentivirus-like particle production using MCP-based packaging plasmids (pspAX2-D64V-NC-MCP; Plasmid No. 11). Real-time PCR detected 30 times more HBB sgRNA3′MS2 than unmodified HBB sgRNA after normalized by lentivirus-like particle input. In agreement with previous data, NC-MCPx1 packaged 5 times more HBB sgRNA13′MS2 than did NC-MCPx2.

To tested whether SaCas9 mRNA and HBB sgRNA could be simultaneously packaged into lentiviral particles, plasmid DNA expressing both SaCas9^{1xMS2} and HBB sgRNA3′MS2 (Plasmid No. 28) was co-transfected into HEK293T cells during lentivirus-like particle production with MCP-based packaging plasmid (psPAX2-D64V-NC-MS2, Plasmid No. 11). Transducing the resulted lentivirus-like particles (70 ng p24 for 2.5×10⁴ cells) into GFP reporter cells as described in Example 1 produced up to 3.6% GFP-positive cells. However, co-transducing the GFP reporter cells with HBB sgRNA1 containing lentivirus (made using Plasmid No. 35) increased the percentage of GFP⁺ cells to over 10%. These experiments indicated that the sgRNA could be packaged into lentivirus-like particles but that the amount of sgRNA packaged resulted in low editing efficiency. Another experiment was performed using lentivirus-like particles packaged with sgRNA having two copies of the HBB 3′ UTR sequences at the 3′ end (made using Plasmid No. 43). This modification was found not to improve the packaging/functionality of the sgRNA. Thus, for high efficiency gene editing, the sgRNA may need to be expressed from lentivirus or AAV.

### Example 9. Lentivirus-Like Particles Delivery of SaCas9 mRNA Reduced Risks of Off-Target Gene Editing Events

The HBB sgRNA1 was designed to target the Sickle mutation and has one nucleotide mismatch with the wild type HBB sequence. As such, the corresponding wild type HBB gene sequence in the GFP reporter cells can be regarded as the “off target” for SaCas9/HBB sgRNA1. An experiment was conducted to determine if the provided lentivirus-like delivery system offers lower off target gene editing rates. The GFP reporter cells were transduced with four different combinations of virus/virus-like particles and the Indel rates in the wild type HBB locus of the GFP-reporter cells was determined and compared. The virus or lentivirus-like particles used were as follows: Condition 1: the lentivirus-like particles containing SaCas9^{1xMS2} mRNA (made using Plasmid No. 16); Condition 2: AAV containing SaCas9 mRNA (made using Plasmid No. 15, pSaCas9); Condition 3: integration defective lentivirus (made using Plasmid No. 31, pCK002-HBB-sgRNA, and packaged using pspAX2-D64V (Addgene Cat No. 63586), which contains the inactive integrase), the IDLV expressing both SaCas9 mRNA and HBB sgRNA1; and Condition 4: integration competent lentivirus (Plasmid No. 31, pCK002-HBB-sgRNA1, and packaged with plasmid pspAX2 (Addgene Cat No. 12260), which contains an active integrase. For Conditions 1 and 2, The GFP reporter cells were co-transduced with lentivirus (pFCK-HBB-sgRNA1, Plasmid No. 35) in Condition 1 or AAV (pAAV-HBB (n)-sgRNA1, Plasmid No. 34) in Condition 2 to express HBB sgRNA1. Results are shown in Table 9. Due to the generation of Indels in the Sickle mutation in the GFP cassette of the GFP reporter cells, variant percentages of the GFP reporter cells in all four treatments were observed. The transduced cells were sorted by GFP-activated sorting. The GFP-positive cell percentage was 89-95%, indicating the presence of functional SaCas9/HBB sgRNA1 in majority of the cells in each of the four conditions. The DNA from the endogenous HBB locus was amplified and sequenced by Next Generation Sequencing. The lentivirus-like particle system described in Condition 1 had the lowest Indel rate, demonstrating that delivering SaCas9 mRNA by lentivirus-like particles for gene editing is safer than other virus delivery systems.

TABLE 9
Indel rates in the wild type HBB locus of the GFP reporter cells

| Group name | Condition 1 | Condition 2 | Condition 3 | Condition 4 |
|---|---|---|---|---|
| Vector | Lentivirus- | AAV | IDLV | Lentivirus |
| delivering | Like Particles | Particles | Particles | Particles |
| SaCas9 | | | | |
| GFP+ (%) | 88.9 | 90.8 | 93.3 | 95.4 |
| Total sequence | 3432717 | 3865920 | 3444935 | 4023043 |
| readings | | | | |
| Indel in | 3.0 | 8.7 | 21.1 | 81.8 |
| wild type | | | | |
| HBB (%) | | | | |

TABLE 10
SEQUENCE LISTING

| SEQ ID | | |
|---|---|---|
| NO | Sequence Type | Sequence |
| SEQ ID | HIV-1 | ATACAGAAAGGCAATTTTAGGAACCAAAGAAAGACTGTTA |
| NO: 1 | Nucleocapsid | AGTGTTTCAATTGTGGCAAAGAAGGGCACATAGCCAAAAA |
| | (NC) DNA | TTGCAGGGCCCCTAGGAAAAAGGGCTGTTGGAAATGTGGA |
| | Sequence | AAGGAAGGACACCAAATGAAAGATTGTACTGAGAGACAG |
| | | GCTAAT |
| SEQ ID | HIV-1 | IQKGNFRNQRKTVKCFNCGKEGHIAKNCRAPRKKGCWKCG |
| NO: 2 | Nucleocapsid | KEGHQMKDCTERQAN |
| | (NC) Amino Acid | |
| | Sequence | |
| SEQ ID | HIV-1 Matrix | atgggtgcgagagcgtcagtattaagcgggggagaattagatcgatgggaaaaaattcggt |
| NO: 3 | protein (MA) | taaggccagggggaaagaaaaaatataaattaaaacatatagtatgggcaagcagggagct |
| | DNA Sequence | agaacgattcgcagttaatcctggcctgttagaaacatcagaaggctgtagacaaatactg |
| | | ggacagctacaaccatcccttcagacaggatcagaagaacttagatcattatataatacag |
| | | tagcaaccctctattgtgtgcatcaaaggatagagataaaagacaccaaggaagctttaga |
| | | caagatagaggaagagcaaaacaaaagtaagaaaaaagcacagcaagcagcagctgacaca |
| | | ggacacagcaatcaggtcagccaaaattac |
| SEQ ID | HIV-1 Matrix | GARASVLSGGELDRWEKIRLRPGGKKKYKLKHIVWASRELE |
| NO: 4 | protein (MA) | RFAVNPGLLETSEGCRQILGQLQPSLQTGSEELRSLYNTVATL |
| | Amino Acid | YCVHQRIEIKDTKEALDKIEEEQNKSKKKAQQAAADTGHSN |
| | Sequence | QVSQNY |
| SEQ ID | HIV-1 Viral | ATGGAACAAGCCCCAGAAGACCAGGGACCGCAGAGGGAA |
| NO: 5 | Protein (VPR) | CCATACAATGAATGGACACTAGAACTTTTAGAGGAACTCA |
| | DNA Sequence | AGCGGGAAGCAGTCAGACACTTTCCTAGACCATGGCTTCA |
| | | TGGCTTAGGACAACATATCTATGAAACCTATGGAGATACT |
| | | TGGACGGGGGTGGAAGCTATAATAAGAATTCTGCAACGAC |
| | | TACTGTTTGTCCATTTCAGAATTGGGTGCCAGCATAGCCGA |
| | | ATAGGCATTCTAAGACAGAGAAGAGCAAGAAATGGAGCC |
| | | AGTAGATCCTAA |
| SEQ ID | HIV-1 Viral | MEQAPEDQGPQREPYNEWTLELLEELKREAVRHFPRPWLHG |
| NO: 6 | Protein (VPR) | LGQHIYETYGDTWTGVEAIIRILQRLLFVHFRIGCQHSRIGILR |
| | Amino Acid | QRRARNGASRS |
| | Sequence | |
| SEQ ID | HIV-1 Negative | atgggtTgcaagtggtcaaaaagtagtgtgattggatggcctgctgtaagggaaagaatga |
| NO: 7 | Regulatory Factor | gacgagctgagccagcagcagatggggtgggagcagtatctcgagacctagaaaaacatgg |
| | (NEF) DNA | agcaatcacaagtagcaatacagcagctaacaatgctgcttgtgcctggctagaagcacaa |
| | Sequence with | gaggaggaagaggtgggttttccagtcacacctcaggtacctttaagaccaatgacttaca |
| | codon changes to | aggcagctgtagatcttagccactttttaaaagaaaaggggggactggaagggctaattca |
| | enhance | gctcccaaagaagacaaatatccttgatctgtggatctaccacacacaaggctacttccct |
| | packaging in the | gattggcagaactacacaccagggccaggggtcagatatccactgacctttggatggtgct |
| | virus core (G3C, | acaagctagtaccagttgagccagataagCtGgaagaggccaataaaggagagaacaccag |
| | V153L, and | cttgttacaccctgtgagcctgcatggaatggatgaccctgGAagagaagtgttagagtgg |
| | E177G mutations: | aggtttgacagccgcctagcatttcatcacgtggcccgagagctgcatccggagtacttca |
| | underlined) | agaactgc(The yellow positions are changed to code for the |
| | | changes explained in seq ID. 8. |
| SEQ ID | HIV-1 Negative | MGCKWSKSSVIGWPAVRERMRRAEPAADGVGAVSRDLEKH |
| NO: 8 | Regulatory Factor | GAITSSNTAANNAACAWLEAQEEEEVGFPVTPQVPLRPMTY |
| | (NEF) Amino | KAAVDLSHFLKEKGGLEGLIHSQRRQDILDLWIYHTQGYFPD |
| | Acid Sequence | WQNYTPGPGVRYPLTFGWCYKLVPVEPDKLEEANKGENTSLL |
| | with mutation to | HPVSLHGMDDPGREVLEWRFDSRLAFHHVARELHPEYFKNC |
| | enhance | |
| | packaging in the | |
| | virus core (G3C, | |
| | V153L, and | |
| | E177G mutations: | |
| | underlined) | |
| SEQ ID | MS2 coat protein | ATGGCTTCTAACTTTACTCAGTTCGTTCTCGTCGACAATGG |
| NO: 9 | (MCP) DNA | CGGAACTGGCGACGTGACTGTCGCCCCAAGCAACTTCGCT |
| | Sequence | AACGGGATCGCTGAATGGATCAGCTCTAACTCGCGTTCAC |
| | | AGGCTTACAAAGTAACCTGTAGCGTTCGTCAGAGCTCTGC |
| | | GCAGAATCGCAAATACACCATCAAAGTCGAGGTGCCTAAA |
| | | GGCGCCTGGCGTTCGTACTTAAATATGGAACTAACCATTC |
| | | CAATTTTCGCCACGAATTCCGACTGCGAGCTTATTGTTAAG |
| | | GCAATGCAAGGTCTCCTAAAAGATGGAAACCCGATTCCCT |
| | | CAGCAATCGCAGCAAACTCCGGCATCTAC |
| SEQ ID | MS2 coat protein | MASNFTQFVLVDNGGTGDVTVAPSNFANGIAEWISSNSRSQA |
| NO: 10 | (MCP) Amino | YKVTCSVRQSSAQNRKYTIKVEVPKGAWRSYLNMELTIPIFA |
| | Acid Sequence | TNSDCELIVKAMQGLLKDGNPIPSAIAANSGIY |
| SEQ ID | PP7 coat protein | tccaaaacaatagtcctctccgtaggggaggcaacacggactttgaccgaaatccagtcaa |
| NO: 11 | (PCP) DNA | ccgctgaccgacaaatctttgaagagaaagtagggcctcttgtgggccgactgcgcttgac |
| | Sequence | tgcaagcttgcgacaaaacggcgcaaagactgcctatagggtcaaccttaaactcgaccaa |
| | | gccgacgtggtcgatagcggtctccctaaggttcggtatacgcaggtctggagtcatgacg |
| | | taacaatcgtagcaaacagcacagaagcctcccgaaaaagcctctacgatctgacgaaatc |
| | | cttggtggctacgtcacaggtggaagacctcgttgtcaaccttgtacctctgggtcga |
| SEQ ID | PP7 coat protein | SKTIVLSVGEATRTLTEIQSTADRQIFEEKVGPLVGRLRLTASL |
| NO: 12 | (PCP) Amino | RQNGAKTAYRVNLKLDQADVVDSGLPKVRYTQVWSHDVTI |
| | Acid Sequence | VANSTEASRKSLYDLTKSLVATSQVEDLVVNLVPLGR |
| SEQ ID | lambda N RNA- | ATGGATGCACAAACACGCCGCCGCGAACGTCGCGCAGAG |
| NO: 13 | binding domain | AAACAGGCTCAATGGAAAGCAGCAAAT |
| | (positions | |
| | (1-22) | |
| | DNA Sequence | |
| SEQ ID | lambda N RNA- | MDAQTRRRERRAEKQAQWKAAN |
| NO: 14 | binding domain | |
| | (positions | |
| | (1-22) | |
| | Amino Acid | |
| | Sequence | |
| SEQ ID | COM Protein | atgaaatcaattcgctgtaaaaactgcaacaaactgttatttaaggcggattcctttgatc |
| NO: 15 | DNA Sequence | acattgaaatcaggtgtccgcgttgcaaacgtcacatcataatgctgaatgcctgcgagca |
| | | tcccacggagaaacattgtgggaaaagagaaaaaatcacgcattctgacgaaaccgtgcgt |
| | | tattgagtat |
| SEQ ID | COM Protein | MKSIRCKNCNKLLFKADSFDH1EIRCPRCKRHIIMLNACEHPT |
| NO: 16 | Amino Acid | EKHCGKREKITHSDETVRY |
| | Sequence | |
| | (GenBank | |
| | AAF01130.1) | |
| SEQ ID | MS2 aptamer | ACAUGAGGAUCACCCAUGU |
| NO: 17 | sequence (RNA) | |
| SEQ ID | MS2 aptamer | ACATGAGGATCACCCATGT |
| NO: 18 | sequence (DNA) | |
| SEQ ID | PP7 aptamer | GGAGCAGACGAUAUGGCGUCGCUCC |
| NO: 19 | sequence (RNA) | |
| SEQ ID | PP7 aptamer | GGAGCAGACGATATGGCGTCGCTCC |
| NO: 20 | sequence (DNA) | |
| SEQ ID | Box-B: lambda N | GGGCCCUGAAGAAGGGCCC |
| NO: 21 | RNA-binding | |
| | domain aptamer | |
| | sequence (RNA) | |
| SEQ ID | Box-B: lambda N | GGGCCCTGAAGAAGGGCCC |
| NO: 22 | RNA-binding | |
| | domain aptamer | |
| | sequence (DNA) | |
| SEQ ID | COM aptamer | CUGAAUGCCUGCGAGCAUC |
| NO: 23 | RNA sequence | |
| SEQ ID | COM aptamer | CTGAATGCCTGCGAGCAT |
| NO: 24 | DNA sequence | |
| SEQ ID | human beta | gctcgctttcttgctgtccaatttctattaaaggttcctttgttccctaagtccaactact |
| NO: 25 | hemoglobin | aaactgggggatattatgaagggccttgagcatctggattctgcctaataaaaaacattta |
| | (HBB) 3′ UTR | ttttcattgc |
| | (DNA) | |
| SEQ ID | human beta | gcucgcuuucuugcuguccaauuucuauuaaagguuccuuuguucccuaaguccaacu |
| NO: 26 | hemoglobin | acuaaacugggggauauuaugaagggccuugagcaucuggauucugccuaauaaaaaa |
| | (HBB) 3′ UTR | cauuuauuuucauugc |
| | (RNA) | |
| SEQ ID | human | Ctcttctggtccccacagactcagagagaac |
| NO: 27 | hemoglobin alpha | |
| | (HBA)5′ UTR | |
| | (DNA) | |
| SEQ ID | SaCas9-2576F | AAACCGGGAACTACCTGACC |
| NO: 28 | | |
| SEQ ID | SaCas9-2713R | TCACGACCTTGTTTCTGCTG |
| NO: 29 | | |
| SEQ ID | SgRNA-F1 | GAGTAACGGCAGACTTCTCCA |
| NO: 30 | | |
| SEQ ID | sgRNA-R1 | CGGCATTTTGCCTTGTTTAAG |
| NO: 31 | | |
| SEQ ID | EGFP-RT-F | CAGTGCTTCAGCCGCTACCC |
| NO: 32 | | |
| SEQ ID | EGFP-RT-R | AGCTCGATGCGGTTCACCAG |
| NO: 33 | | |
| SEQ ID | HHB-mut-F1 | TCGTCGGCAGCGTCAGATGTGTATAAGAGACAGTGTTCAC |
| NO: 34 | | TAGCAACCTCAAACAG |
| SEQ ID | HHB-mut-F2 | TCGTCGGCAGCGTCAGATGTGTATAAGAGACAGATGTTCA |
| NO: 35 | | CTAGCAACCTCAAACAG |
| SEQ ID | HHB-mut-F3 | TCGTCGGCAGCGTCAGATGTGTATAAGAGACAGGATGTTC |
| NO: 36 | | ACTAGCAACCTCAAACAG |
| SEQ ID | HHB-mut-F4 | TCGTCGGCAGCGTCAGATGTGTATAAGAGACAGCGATGTT |
| NO: 37 | | CACTAGCAACCTCAAACAG |
| SEQ ID | HHB-mut-F5 | TCGTCGGCAGCGTCAGATGTGTATAAGAGACAGTCGATGT |
| NO: 38 | | TCACTAGCAACCTCAAACAG |
| SEQ ID | HHB-mut-F6 | TCGTCGGCAGCGTCAGATGTGTATAAGAGACAGATCGATG |
| NO: 39 | | TTCACTAGCAACCTCAAACAG |
| SEQ ID | HHB-mut-R1 | GTCTCGTGGGCTCGGAGATGTGTATAAGAGACAGTCCAAT |
| NO: 40 | | AGGCAGAGAGAGTCAGTG |
| SEQ ID | Oligo 1xloop-F | TTACGCTTAAGAATTCTAGAAAACATGAGGATCACCCATG |
| NO: 41 | | TCTGCAGGTCGACTCTAGAAAATTCCTAGAGCTCG |
| SEQ ID | Oligo 1xloop-R | CGAGCTCTAGGAATTTTCTAGAGTCGACCTGCAGACATGG |
| NO: 42 | | GTGATCCTCATGTTTTCTAGAATTCTTAAGCGTAA |
| SEQ ID | Oligo 2xloop-F | TTACGCTTAAGAATTCTAGAAAACATGAGGATCACCCATG |
| NO: 43 | | TCTGCAGGTCGACTCTAGAAAACATGAGGATCACCCATGT |
| | | CTGCAAAATTCCTAGAGCTCG |
| SEQ ID | Oligo 2xloop-R | CGAGCTCTAGGAATTTTGCAGACATGGGTGATCCTCATGTT |
| NO: 44 | | TTCTAGAGTCGACCTGCAGACATGGGTGATCCTCATGTTTT |
| | | CTAGAATTCTTAAGCGTAA |
| SEQ ID | Oligo 3xloop-F | TTACGCTTAAGAATTCTAGAAAACATGAGGATCACCCATG |
| NO: 45 | | TCTGCAGGTCGACTCTAGAAAACATGAGGATCACCCATGT |
| | | CTGCAAAATTCTAGAAAACAT |
| SEQ ID | Oligo 3xloop-R | ATGTTTTCTAGAATTTTGCAGACATGGGTGATCCTCATGTT |
| NO: 46 | | TTCTAGAGTCGACCTGCAGACATGGGTGATCCTCATGTTTT |
| | | CTAGAATTCTTAAGCGTAA |
| SEQ ID | Oligo Sickle-g2F | CACCGCCCTGTGGGGCAAGGTGAAC |
| NO: 47 | | |
| SEQ ID | Oligo Sickle-g2R | AAACGTTCACCTTGCCCCACAGGGC |
| NO: 48 | | |
| SEQ ID | Oligo Sickle-g1F | CACCGAGTAACGGCAGACTTCTCCAC |
| NO: 49 | | |
| SEQ ID | Sickle-g1R | AAACGTGGAGAAGTCTGCCGTTACTC |
| NO: 50 | | |
| SEQ ID | Oligo PP7-F | TTACGCTTAAGAATTCGGAGCAGACGATATGGCGTCGCTC |
| NO: 51 | | CGAATTCCTAGAGCTCG |
| SEQ ID | Oligo PP7-R | CGAGCTCTAGGAATTCGGAGCGACGCCATATCGTCTGCTC |
| NO: 52 | | CGAATTCTTAAGCGTAA |
| SEQ ID | Oligo Sickle-g1- | caccGAGTAACGGCAGACTTCCCAC |
| NO: 53 | LV-F | |
| SEQ ID | Oligo sickle-g1- | gaacGTGGAGAAGTCTGCCGTTACTC |
| NO: 54 | LV-R | |
| SEQ ID | Primer Sickle- | TTGCAATGATCTCGAGGGCCTATTTCCCATGATTC |
| NO: 55 | g1HD-F | |
| SEQ ID | Primer Sickle- | CTGCGGCCGCTCTAGAAAAATCTCGCCAACAAGTTG |
| NO: 56 | g1HD-R | |
| SEQ ID | oligo HBB-tcm-F | CATGGTGCATCTGACACCTGTGGAGAAGTCTGCCGTTACT |
| NO: 57 | | GCCCTGTGGGGCAAGGTGAACGTGGATGAAGTTGGTGGTG |
| | | AGGCC |
| SEQ ID | oligo FIBB-tem-R | CAGGGCCTCACCACCAACTTCATCCACGTTCACCTTGCCCC |
| NO: 58 | | ACAGGGCAGTAACGGCAGACTTCTCCACAGGTGTCAGATG |
| | | CAC |
| SEQ ID | HHB-LT-F | CTTCTGATTTTCTAGATTGTGTAATCGTAGTTTCAGAG |
| NO: 59 | | |
| SEQ ID | HHB-LT-R | GCTTGATATCGAATTAAAAAATCTCGCCAACAAGTTGAC |
| NO: 60 | | |
| SEQ ID | pCDNA3.1/MS2x | GCTAGCcaccATGGCTTCTAACTTTACTCAGTTCGTTCTCGTCGA |
| NO: 61 | 2-VPR DNA | CAATGGCGGAACTGGCGACGTGACTGTCGCCCCAAGCAACTT |
| | Oligo Insert | CGCTAACGGGATCGCTGAATGGATCAGCTCTAACTCGCGTTCA |
| | | CAGGCTTACAAAGTAACCTGTAGCGTTCGTCAGAGCTCTGCGC |
| | | AGAATCGCAAATACACCATCAAAGTCGAGGTGCCTAAAGGCGC |
| | | CTGGCGTTCGTACTTAAATATGGAACTAACCATTCCAATTTTCG |
| | | CCACGAATTCCGACTGCGAGCTTATTGTTAAGGCAATGCAAGG |
| | | TCTCCTAAAAGATGGAAACCCGATTCCCTCAGCAATCGCAGCA |
| | | AACTCCGGCATCTACGGTGGTGGAGGAGGAATGGCGTCCAAT |
| | | TTCACGCAGTTCGTCCTGGTTGACAACGGGGGGACTGGGGAC |
| | | GTTACGGTCGCTCCGAGCAACTTTGCCAATGGTATTGCGGAGT |
| | | GGATTTCTTCTAATTCACGGTCCCAAGCTTACAAAGTGACCTGT |
| | | TCCGTGCGGGAAAGTTCTGCTCAGAATAGAAAGTACACTATAA |
| | | AGGTCGAAGTCCCTAAGGGGGCCTGGCGATCATATCTCAATAT |
| | | GGAGCTTACCATCCCAATATTTGCCACTAATTCTGATTGTGAAT |
| | | TGATTGTCAAAGCAATGCAAGGACTCTTGAAAGACGGAAACCC |
| | | AATCCCCAGCGCAATCGCAGCCAACTCCGGTATATACGGAGG |
| | | TGGTGGAGGAATGGAACAAGCCCCAGAAGACCAGGGACCGCA |
| | | GAGGGAACCATACAATGAATGGACACTAGAACTTTTAGAGGAA |
| | | CTCAAGCGGGAAGCAGTCAGACACTTTCCTAGACCATGGCTTC |
| | | ATGGCTTAGGACAACATATCTATGAAACCTATGGAGATACTTGG |
| | | ACGGGGGTGGAAGCTATAA1AAGAAITCTGCAACGACTACTGT |
| | | TTGTCCATTTCAGAATTGGGTGCCAGCATAGCCGAATAGGCAT |
| | | TCTAAGACAGAGAAGAGCAAGAAATGGAGCCAGTAGATCCTAA |
| | | gcggccgc |
| SEQ ID | pCDNA3.1/MS2x | MASNFTQFVLVDNGGTGDVTVAPSNFANGIAEWISSNSRSQAYKV |
| NO: 62 | 2-VPR Oligo | TCSVRQSSAQNRKYTIKVEVPKGAWRSYLNMELTIPIFATNSDCEL |
| | Insert Amino Acid | IVKAMQGLLKDGNPIPSAIAANSGIYGGGGGMASNFTQFVLVDN |
| | Sequence | GGTGDVTVAPSNFANGIAEWISSNSRSQAYKVTCSVRQSSAQNRK |
| | | YTIKVEVPKGAWRSYLNMELTIPIFATNSDCELIVKAMQGLLXDG |
| | | NPIPSAIAANSGIYGGGGGMEQAPEDQGPQREPYNEWTLELLEE |
| | | LKREAVRHFPRPWLHGLGQHIYETYGDTWIGVEAIIRILQRLLFV |
| | | HFRIGCQHSRIGILRQRRARNGASRS |
| SEQ ID | pCDNA3.1/NEF- | GCTAGCcaccatgggtTgcaagtggtcaaaaagtagtgtgattggcttggcctgctgtaag |
| NO: 63 | MS2x2 DNA | ggaaagaatgagctcgagctgagccagcagcagatggggtgggagcagtatctcgagacct |
| | Oligo Insert | agaaaaacatggagcaatcacaagtagcaatacagcagctactcaatgctgcttgtgcctg |
| | | gctagaagcacaagaggaggaagaggtgggttttccagtcacacctcaggtacctttaaga |
| | | ccaatgacttacaaggcagctgtagatcttagccactttttaaaagaaaaggggggactgg |
| | | aagggctaattcactcccaaagaagacaagatatccttgatctgtggatctaccacacaca |
| | | aggctacttccctgattggcagaactacacaccagggccaggggtcagatatccactgacc |
| | | tttggatggtgctacaagctagtaccagttgagccagataagCtGgaagaggccaataaag |
| | | gagagaacaccagcttgttctcaccctgtgctgcctgcatggaatggatgaccctgGAaga |
| | | gaagtgttagagtggaggtttgacagccgcctagcatttcatcacgtggcccgagagctgc |
| | | atccggagtacttcaagaactgcGGAGGTGGTGGAGGAATGGCTTCTAACTTTACTC |
| | | AGTTCGTTCTCGTCGACAATGGCGGAACTGGCGACGTGACTG |
| | | TCGCCCCAAGCAACTTCGCTAACGGGATCGCTGAATGGATCA |
| | | GCTCTAACTCGCGTTCACAGGCTIACAAAGTAACCTGTAGCGT |
| | | TCGTCAGAGCTCTGCGCAGAATCGCAAATACACCATCAAAGTC |
| | | GAGGTGCCTAAAGGCGCCTGGCGTTCGTACTTAAATATGGAAC |
| | | TAACCATTCCAATTTTCGCCACGAATTCCGACTGCGAGCTTATT |
| | | GTTAAGGCAATGCAAGGTCTCCTAAAAGATGGAAACCCGATTC |
| | | CCTCAGCAATCGCAGCAAACTCCGGCATCTACGGTGGTGGAG |
| | | GAGGAATGGCGTCCAATTTCACGCAGTTCGTCCTGGTTGACAA |
| | | CGGGGGGACTGGGGACGTTACGGTCGCTCCGAGCAACTTTGC |
| | | CAATGGTATTGCGGAGTGGATTTCTTCTAATTCACGGTCCCAA |
| | | GCTTACAAAGTGACCTGTTCCGTGCGGCAAAGTTCTGCTCAGA |
| | | ATAGAAAGTACACTATAAAGGTCGAAGTCCCTAAGGGGGCCTG |
| | | GCGATCATATCTCAATATGGAGCTTACCATCCCAATATTTGCCA |
| | | CTAATTCTGATTGTGAATTGATTGTCAAAGCAATGCAAGGACTC |
| | | TTGAAAGACGGAAACCCAATCCCCAGCGCAATCGCAGCCAACT |
| | | CCGGTATATACTGAgcggccgc |
| SEQ ID | pCDNA3.1/NEF- | TMGCKWSKSSVIGWPAVRERMRRAEPAADGVGAVSRDLEK |
| NO: 64 | MS2x2 Oligo | HGAITSSNTAANNAACAWLEAQEEEEVGFPVTPQVPLRPMT |
| | Insert Amino Acid | YKAAVDLSHFLKEKGGLEGLIHSQRRQDILDLWIYHTQGYFP |
| | Sequence | DWQNYTPGPGVRYPLTFGWCYKLVPVEPDKLEEANKGENTS |
| | | LLHPVSLHGMDDPGREVLEVVRFDSRLAFHHVARELHPEYFK |
| | | NCGGGGGMASNFTQFVLVDNGGTGDVTVAPSNFANGIAEWI |
| | | SSNSRSQAYKVTCSVRQSSAQNRKYTIKVEVPKGAWRSYLN |
| | | MELTIPIFATNSDCELIVKAMQGLLKDGNPIPSAIAANSGI |
| | | YGGGGGMASNFTQFVLVDNGGTGDVTVAPSNFANGIAEWIS |
| | | SNSRSQAYKVTCSVRQSSAQNRKYTIKVEVPKGAWRSYLNM |
| | | ELTIPIFATNSDCELIVKAMQGLLKDGNPIPSAIAANSGIY |
| SEQ ID | pMDLg/pRRE- | cctaggaaaaagggctgttggaaatgtggaaaggaaggacaccaaatgaaagattgttccg |
| NO: 65 | D64V-NC-MS2x1 | gtggaggtggatccggtggaggttccatggcgtccaatttcacgcagttcgtcctggttga |
| | DNA Oligo Insert | caacggggggactggggacgttacggtcgctccgagcaactttgccaatggtattgcggag |
| | | tggatttcttctaattcacggtcccaagcttacaaagtgacctgttccgtgcggcaaagtt |
| | | ctgctcagaatagaaagtacactataaaggtcgaagtccctaagggggcctggcgatcata |
| | | tctcaatatggagcttaccatcccaatatttgccactaattctgattgtgaattgattgtc |
| | | aaagcaatgcaaggactcttgaaagacggaaacccaatccccagcgcaatcgcagccaact |
| | | ccggtatatactccggaggtggaggtggaactgagagacaggctaattttttagggaagat |
| | | ctggccttcccacaagggaaggccagggaattttcttcagagcagaccagagccaacagcc |
| | | ccaccagaagagagcttcaggtttggggaagagacaacaactccctctcagaagcaggagc |
| | | cgatagacaaggaactgtatcctttagcttccctcagatcactctttggcagcgacccctc |
| | | gtcacaataaagataggggggcaattaaaggaagctctattagatacaggagcagatgata |
| | | cagtattagaagaaatgaatttgccaggaagatggaaaccaaaaatgatagggggaattgg |
| | | aggttttatcaaagtaagacagtatgatcagatactcatagaaatctgcggacataaagct |
| | | ataggtacagtattagtaggacctacacctgtcaacataattggaagaaatctgttgactc |
| | | agattggctgcactttaaattttcccattagtcctattgagactgtaccagtaaaattaaa |
| | | gccaggaatggatggcccaaaagttaaacaatggccattgacagaagaaaaaataaaagca |
| | | ttagtagaaatttgtacagaaatggaaaaggaaggaaaaatttcaaaaattgggcctgaaa |
| | | atccatacaatactccagtatttgccataaagaaaaaagacagtactaaatggagaaaatt |
| | | agtagatttcagagaacttaataagagaactcaagatttctgggaagttcaattaggaata |
| | | ccacatcctgcagg |
| SEQ ID | pMDLg/pRRE- | PRKKGCWKCGKEGHQMKDCSGGGGSGGGSMASNFTQFVLV |
| NO: 66 | D64V-NC-MS2x1 | DNGGTGDVTVAPSNFANGIAEWISSNSRSQAYKVTCSVRQSS |
| | Oligo Insert | AQNRKYTIKVEVPKGAWRSYLNMELTIPIFATNSDCELIVKA |
| | Amino Acid | MQGLLKDGNPIPSAIAANSGIYSGGGGGTERQANFLGKIWPS |
| | Sequence | HKGRPGNFLQSRPEPTAPPEESFRFGEETTTPSQKQEPIDKELY |
| | | PLASLRSLFGSDPSSQ |
| SEQ ID | pMDLg/pRRE- | CCTAGGAAAAAGGGCTGTTGGAAATGTGGAAAGGAAGGA |
| NO: 67 | D64V-NC-MS2x2 | CACCAAATGAAAGATTGTTCCGGTGGAGGTGGATCCATGG |
| | DNA Oligo Insert | CTTCTAACTTTACTCAGTTCGTTCTCGTCGACAATGGCGGA |
| | | ACTGGCGACGTGACTGTCGCCCCAAGCAACTTCGCTAACG |
| | | GGATCGCTGAATGGATCAGCTCTAACTCGCGTTCACAGGC |
| | | TTACAAAGTAACCTGTAGCGTTCGTCAGAGCTCTGCGCAG |
| | | AATCGCAAATACACCATCAAAGTCGAGGTGCCTAAAGGCG |
| | | CCTGGCGTTCGTACTTAAATATGGAACTAACCATTCCAATT |
| | | TTCGCCACGAATTCCGACTGCGAGCTTATTGTTAAGGCAAT |
| | | GCAAGGTCTCCTAAAAGATGGAAACCCGATTCCCTCAGCA |
| | | ATCGCAGCAAACTCCGGCATCTACGGATCCGGTGGAGGTT |
| | | CCATGGCGTCCAATTTCACGCAGTTCGTCCTGGTTGACAAC |
| | | GGGGGGACTGGGGACGTTACGGTCGCTCCGAGCAACTTTG |
| | | CCAATGGTATTGCGGAGTGGATTTCTTCTAATTCACGGTCC |
| | | CAAGCTTACAAAGTGACCTGTTCCGTGCGGCAAAGTTCTG |
| | | CTCAGAATAGAAAGTACACTATAAAGGTCGAAGTCCCTAA |
| | | GGGGGCCTGGCGATCATATCTCAATATGGAGCTTACCATC |
| | | CCAATATTTGCCACTAATTCTGATTGTGAATTGATTGTCAA |
| | | AGCAATGCAAGGACTCTTGAAAGACGGAAACCCAATCCCC |
| | | AGCGCAATCGCAGCCAACTCCGGTATATACTCCGGAGGTG |
| | | GAGGTGGAACTGAGAGACAGGCTAATTTTTTAGGGAAGAT |
| | | CTGGCCTTCCCACAAGGGAAGGCCAGGGAATTTTCTTCAG |
| | | AGCAGACCAGAGCCAACAGCCCCACCAGAAGAGAGCTTC |
| | | AGGTTTGGGGAAGAGACAACAACTCCCTCTCAGAAGCAGG |
| | | AGCCGATAGACAAGGAACTGTATCCTTTAGCTTCCCTCAG |
| | | ATCACTCTTTGGCAGCGACCCCTCGTCACAATAAAGATAG |
| | | GGGGGCAATTAAAGGAAGCTCTATTAGATACAGGAGCAG |
| | | ATGATACAGTATTAGAAGAAATGAATTTGCCAGGAAGATG |
| | | GAAACCAAAAATGATAGGGGGAATTGGAGGTTTTATCAAA |
| | | GTAAGACAGTATGATCAGATACTCATAGAAATCTGCGGAC |
| | | ATAAAGCTATAGGTACAGTATTAGTAGGACCTACACCTGT |
| | | CAACATAATTGGAAGAAATCTGTTGACTCAGATTGGCTGC |
| | | ACTTTAAATTTTCCCATTAGTCCTATTGAGACTGTACCAGT |
| | | AAAATTAAAGCCAGGAATGGATGGCCCAAAAGTTAAACA |
| | | ATGGCCATTGACAGAAGAAAAAATAAAAGCATTAGTAGA |
| | | AATTTGTACAGAAATGGAAAAGGAAGGAAAAATTTCAAA |
| | | AATTGGGCCTGAAAATCCATACAATACTCCAGTATTTGCC |
| | | ATAAAGAAAAAAGACAGTACTAAATGGAGAAAATTAGTA |
| | | GATTTCAGAGAACTTAATAAGAGAACTCAAGATTTCTGGG |
| | | AAGTTCAATTAGGAATACCACATCCTGCAGG |
| SEQ ID | pMDLg/pRRE- | PRKKGCWKCGKEGHQMKDCSGGGGSMASNFTQFVLVDNG |
| NO: 68 | D64V-NC-MS2x2 | GTGDVTVAPSNFANGIAEWISSNSRSQAYKVTCSVRQSSAQN |
| | Oligo Insert | RKYTIKVEVPKGAWRSYLNMELTIPIFATNSDCELIVKAMQG |
| | Amino Acid | LLKDGNPIPSAIAANSGIYGSGGGSMASNFTQFVLVDNGGTG |
| | Sequence | DVTVAPSNFANGIAEWISSNSRSQAYKVTCSVRQSSAQNRKY |
| | | TIKVEVPKGAWRSYLNMELTIPIFATNSDCELIVKAMQGLLK |
| | | DGNPIPSAIAANSGIYSGGGGGTERQANFLGKIWPSHKGRPG |
| | | NFLQSRPEPTAPPEESFRFGEETTTPSQKQEPIDKELYPLASLR |
| | | SLFGSDPSSQ |
| SEQ ID | pMDLg/pRRE- | cctaggaaaaagggctgttggaaatgtggaaaggaaggacaccaaatgaaagattgttccg |
| NO: 69 | D64V-NC-PP7x1 | gtggaggtggatcctccaaaacaatagtcctctccgtaggggaggcaacacggactttgac |
| | DNA Oligo Insert | cgaaatccagtcaaccgctgaccgacaaatctttgaagagaaagtagggcctcttgtgggc |
| | | cgactgcgcttgactgcaagcttgcgacaaaacggcgcaaagactgcctatagggtcaacc |
| | | ttaaactcgaccaagccgacgtggtcgatagcggtctccctaaggttcggtatacgcaggt |
| | | ctggagtcatgacgtaacaatcgtagcaaacagcacagaagcctcccgaaaaagcctctac |
| | | gatctgacgaaatccttggtggctacgtcacaggtggaagacctcgttgtcaaccttgtac |
| | | ctctgggtcggtccggaggtggaggtggaactgagagacaggctaattttttagggaagat |
| | | ctggccttcccacaagggaaggccagggaattttcttcagagcagaccagagccaacagcc |
| | | ccaccagaagagagcttcaggtttggggaagagacaacaactccctctcagaagcaggagc |
| | | cgatagacaaggaactgtatcctttagcttccctcagatcactctttggcagcgacccctc |
| | | agtcacaataaagataggggggcaattaaaggaagctctttagatacaggagcagatgata |
| | | cagtattagaagaaatgaatttgccaggaagatggaaaccaaaaatgatagggggaattgg |
| | | aggttttatcaaagtaagacagtatgatcagatactcatagaaatctgcggacataaagct |
| | | ataggtacagtattagtaggacctacacctgtcaacataattggaagaaatctgttgactc |
| | | agattggctgcactttaaattttcccattagtcctattgagactgtaccagtaaaattaaa |
| | | gccaggaatggatggcccaaaagttaaacaatggccattgacagaagaaaaaataaaagca |
| | | ttagtagaaatttgtacagaaatggaaaaggaaggaaaaatttcaaaaattgggcctgaaa |
| | | atccatacaatactccagtatttgccataaagaaaaaagacagtactaaatggagaaaatt |
| | | agtagatttcagagaacttaataagagaactcaagatttctgggaagttcaattaggaata |
| | | ccacatcctgcaga |
| SEQ ID | pMDLg/pRRE- | PRKKGCWKCGKEGHQMKDCSGGGGSSKTIVLSVGEATRTLT |
| NO: 70 | D64V-NC-PP7x1 | EIQSTADRQIFEEKVGPLVGRLRLTASLRQNGAKTAYRVNL |
| | Oligo Insert | KLDQADVVDSGLPKVRYTQVWSHDVTIVANSTEASRKSLYD |
| | Amino Acid | LTKSLVATSQVEDLVVNLVPLGRSGGGGGTERQANFLGKIW |
| | Sequence | PSHKGRPGNFLQSRPEPTAPPEESFRFGEETTTPSQKQEPI |
| | | DKELYPLASLRSLFGSDPSSQ |
| SEQ ID | pMDLg/pRRE- | CACGTGAGATCTGAATTCGAGATCTGCCGCCGCCATGGGT |
| NO: 71 | D64V-MA- | GCGAGAGCGTCAGTATTAAGCGGGGGAGAATTAGATCGAT |
| | MS2x2 DNA | GGGAAAAAATTCGGTTAAGGCCAGGGGGAAAGAAAAAAT |
| | Oligo Insert | ATAAATTAAAACATATAGTATGGGCAAGCAGGGAGCTAG |
| | | AACGAGGAGGTGGTGGAGGAATGGCTTCTAACTTTACTCA |
| | | GTTCGTTCTCGTCGACAATGGCGGAACTGGCGACGTGACT |
| | | GTCGCCCCAAGCAACTTCGCTAACGGGATCGCTGAATGGA |
| | | TCAGCTCTAACTCGCGTTCACAGGCTTACAAAGTAACCTGT |
| | | AGCGTTCGTCAGAGCTCTGCGCAGAATCGCAAATACACCA |
| | | TCAAAGTCGAGGTGCCTAAAGGCGCCTGGCGTTCGTACTT |
| | | AAATATGGAACTAACCATTCCAATTTTCGCCACGAATTCC |
| | | GACTGCGAGCTTATTGTTAAGGCAATGCAAGGTCTCCTAA |
| | | AAGATGGAAACCCGATTCCCTCAGCAATCGCAGCAAACTC |
| | | CGGCATCTACGGTGGTGGAGGAGGAATGGCGTCCAATTTC |
| | | ACGCAGTTCGTCCTGGTTGACAACGGGGGGACTGGGGACG |
| | | TTACGGTCGCTCCGAGCAACTTTGCCAATGGTATTGCGGA |
| | | GTGGATTTCTTCTAATTCACGGTCCCAAGCTTACAAAGTGA |
| | | CCTGTTCCGTGCGGCAAAGTTCTGCTCAGAATAGAAAGTA |
| | | CACTATAAAGGTCGAAGTCCCTAAGGGGGCCTGGCGATCA |
| | | TATCTCAATATGGAGCTTACCATCCCAATATTTGCCACTAA |
| | | TTCTGArrGTGAATrGATTGTCAAAGCAATGCAAGGACTCT |
| | | TGAAAGACGGAAACCCAATCCCCAGCGCAATCGCAGCCA |
| | | ACTCCGGTATATACCAGGTCAGCCAAAATTACCCTATAGT |
| | | GCAGAACATCCAGGGGCAAATGGTACATCAGGCCATATCA |
| | | CCTAGAACTTTAAATGCATGGGTAAAAGTAGTAGAAGAGA |
| | | AGGCTTTCAGCCCAGAAGTGATACCCATGTTTTCAGCATTA |
| | | TCAGAAGGAGCCACCCCACAAGATTTAAACACCATGCTAA |
| | | ACACAGTGGGGGGACATCAAGCAGCCATGCAAATGTTAA |
| | | AAGAGACCATCAATGAGGAAGCTGCAGAATGGGATAGAG |
| | | TGCATCCAGTGCATGC |
| SEQ ID | pMDLg/pRRE- | MGARASVLSGGELDRWEKIRLRPGGKKKYKLKHIVWASREL |
| NO: 72 | D64V-MA- | ERGGGGGMASNFTQFVLVDNGGTGDVTVAPSNFANGIAEWI |
| | MS2x2 Oligo | SSNSRSQAYKVTCSVRQSSAQNRKYTIKVEVPKGAWRSYLN |
| | Insert Amino Acid | MELTIPIFATNSDCELIVKAMQGLLKDGNPIPSAIAANSGI |
| | Sequence | YGGGGGMASNFTQFVLVDNGGTGDVTVAPSNFANGIAEWIS |
| | | SNSRSQAYKVTCSVRQSSAQNRKYTIKVEVPKGAWRSYLNM |
| | | ELTIPIFATNSDCELIVKAMQGLLKDGNPIPSAIAANSGIY |
| | | QVSQNYPIVQNIQGQMVHQAISPRTLNAWVKVVEEKAFSPE |
| | | VIPMFSALSEGATPQDLNTMLNTVGGHQAAMQMLKETINEE |
| | | AAEWDRVHPVH |
| SEQ ID | pMDLg/pRRE- | Cacgtgagatctgaattcgagatctgccgccgccatgggtgcgagagcgtcagtattaagc |
| NO: 73 | D64V-MA-PP7x1 | gggggagaattagatcgatgggaaaaaattcggttaaggccagggggaaagaaaaaatata |
| | DNA | aattaaaacatatagtatgggcaagcagggagctagaacgaggaggtggtggaggaatggc |
| | Oligo Insert | ttctaactttactcagttcgttctcgtcgactccaaaacaatagtcctctccgtaggggag |
| | | gcaacacggactttgaccgaaaiccagtcaaccgctgaccgacaaatctttgaagagaaag |
| | | tagggcctcttgtgggccgactgcgcttgactgcaagcttgcgacaaaacggcgcaaagac |
| | | tgcctatagggtcaaccttaaactcgaccaagccgacgtggtcgatagcggtctccctaag |
| | | gttcggtatacgcaggtctggagtcatgacgtaacaatcgtagcaaacagcacagaagcct |
| | | cccgaaaaagcctctacgatctgacgaaatccttggtggctacgtcacaggtggaagacct |
| | | cgttgtcaaccttgtacctctgggtcgaaaccaggtcagccaaaattaccctatagtgcag |
| | | aacatccaggggcaaatggtacatcaggccatatcacctagaactttaaatgcatgggtaa |
| | | aagtagtagaagagaaggctttcagcccagaagtgatacccatgttttcagcattatcaga |
| | | aggagccaccccacaagatttaaacaccatgctaaacacagtggggggacatcaagcagcc |
| | | atgcaaatgttaaaagagaccatcaatgaggaagctgcagaatgggatagagtgcatccag |
| | | tgcatgc |
| SEQ ID | pMDLg/pRRE- | MGARASVLSGGELDRWEKIRLRPGGKKKYKLKHIVWASREL |
| NO: 74 | D64V-MA-PP7x1 | ERGGGGGMASNFTQFVLVDSKTIVLSVGEATRTLTEIQSTAD |
| | Oligo Insert | RQIFEEKVGPLVGRLRLTASLRQNGAKTAYRVNLKLDQADV |
| | Amino Acid | VDSGLPKVRYTQVWSHDVT1VANSTEASRKSLYDLTKSLVA |
| | Sequence | TSQVEDLVVNLVPLGRNQVSQNYPIVQNIQGQMVHQAISPRT |
| | | LNAWVKVVEEKAFSPEVIPMFSALSEGATPQDLNTMLNTVG |
| | | GHQAAMQMLKETINEEAAEWDRVHPVH |
| SEQ ID | pMDLg/pRRE- | cacgtgagatctgaattcgagatctgccgccgccatgggtgcgagagcgtcagtattaagc |
| NO: 75 | D64V-MA-PP7x2 | gggggagaattagatcgatgggaaaaaattcggttaaggccagggggaaagaaaaaatata |
| | DNA | aattaaaacatatagtatgggcaagcagggagctagaacgaggaggtggtggaggaatggc |
| | Oligo Insert | ttctaactttactcagttcgttctcgtcgactccaaaacaatagtcctctccgtaggggag |
| | | gcaacacggactttgaccgaaatccagtcaaccgctgaccgacaaatctttgaagagaaag |
| | | tagggcctcttgtgggccgactgcgcttgactgcaagcttgcgacaaaacggcgcaaagac |
| | | tgcctatagggtcaaccttaaactcgaccaagccgacgtggtcgatagcggtctccctaag |
| | | gttcggtatacgcaggtctggagtcatgacgtaacaatcgtagcaaacagcacagaagcct |
| | | cccgaaaaagcctctacgatctgacgaaatccttggtggctacgtcacaggtggaagacct |
| | | cgttgtcaaccttgtacctctgggtcgagcggatccgctcgcatcaaaaactattgtgctc |
| | | tccgtgggagaagccacccgcacgcttaccgaaattcaatcaacggcagacagacaaatct |
| | | ttgaggagaaagtaggtccgttggtgggtcggttgcgcttgaccgcaagcctccgccaaaa |
| | | cggagcgaaaaccgcataccgcgtaaacttgaagctggaccaagccgatgttgttgactcc |
| | | ggcttgcccaaagtgcgatatactcaggtctggtctcatgatgtcacaatcgtcgctaatt |
| | | ccactgaggctagtcgcaaaagtctgtatgacttgacaaagtccttggtagccacgtcaca |
| | | ggtggaagatttggtggtgaacctcgttccactgggaagaaaccaggtcagccaaaattac |
| | | cctatagtgcagaacatccaggggcaaatggtacatcaggccatatcacctagaactttaa |
| | | atgcatgggtaaaagtagtagaagagaaggctttcagcccagaagtgatacccatgttttc |
| | | agcattatcagaaggagccaccccacaagatttaaacaccatgciaaacacagtgggggga |
| | | catcaagcagccatgcaaatgttaaaagagaccatcaatgaggaagctgcagaatgggata |
| | | gagtgcatccagtgcatgc |
| SEQ ID | pMDLg/pRRE- | MGARASVLSGGELDRWEKIRLRPGGKKKYKLKHIVWASREL |
| NO: 76 | D64V-MA-PP7x2 | ERGGGGGMASNFTQFVLVDSKTIVLSVGEATRTLTEIQSTAD |
| | Oligo Insert | RQIFEEKVGPLVGRLRLTASLRQNGAKTAYRVNLKLDQADV |
| | Amino Acid | VDSGLPKVRYTQVWSHDVTIVANSTEASRKSLYDLTKSLVA |
| | Sequence | TSQVEDLVVNLVPLGRADPLASKTIVLSVGEATRTLTEIQSTA |
| | | DRQIFEEKVGPLVGRLRLTASLRQNGAKTAYRVNLKLDQAD |
| | | VVDSGLPKVRYTQVVVSHDVTIVANSTEASRKSLYDLTKSLV |
| | | ATSQVEDLVVNLVPLGRNQVSQNYPIVQNIQGQMVHQAISPR |
| | | TLNAWVKVVEEKAFSPEVIPMFSALSEGATPQDLNTMLNTV |
| | | GGHQAAMQMLKETINEEAAEWDRVHPVH |
| SEQ ID | DNA oligo cloned | GGTCTCGCACCGAGTAACGGCAGACTTCTCCACGTTTAAG |
| NO: 77 | into Plasmid No. | TACTCTGGAAACAGAATCTACTTAAACAAGGCAAAATGCC |
| | 24 (pSaCas9- | GTGTTTATCTCGTCAACTTGTTGGCGAGATGGCCAACATGA |
| | HBB-sgRNA1^{3′ms2}) | |
| | encoding a | |
| | HBB-sgRNA1 | |
| | with a MS2 | |
| | aptamer at the 3′ | |
| | end of the sgRNA | |
| SEQ ID | DNA oligo cloned | GGTCTCGCACCGAGTAACGGCAGACTTCTCCACGTTTAAG |
| NO: 78 | into Plasmid No. | TACTCTGGGCCAACATGAGGATCACCCATGTCTGCAGGGC |
| | 25 (pSaCas9- | CCAGAATCTACTTAAACAAGGCAAAATGCCGTGTTTATCT |
| | HBB-sgRNA1^{Tetrams2}) | CGTCAACTTGTTGGCGAGATTTTTTTGCGGCCGC |
| | encoding a | |
| | HBB-sgRNA1 | |
| | with a MS2 | |
| | aptamer inserted | |
| | into the Tetra loop | |
| | of the sgRNA | |
| SEQ ID | DNA oligo cloned | GGTCTCGCACCGAGTAACGGCAGACTTCTCCACGTTTAAG |
| NO: 79 | into Plasmid No. | TACTCTGGAAACAGAATCTACTTAAACAAGGCAAAATGCC |
| | 26 (pSaCas9- | GTGTTTATCTCGTCAAGGCCAACATGAGGATCACCCATGT |
| | HBB-sgRNA1^{ST2ms2}) | CTGCAGGGCCTTGGCGAGATTTTTTTGCGGCCGC |
| | encoding a | |
| | HBB-sgRNA1 | |
| | with a MS2 | |
| | aptamer inserted | |
| | into the stem loop | |
| | 2 of the sgRNA | |
| SEQ ID | DNA oligo cloned | GGTCTCGCACCGAGTAACGGCAGACTTCTCCACGTTTAAG |
| NO: 80 | into Plasmid No. | TACTCTGGGCCAACATGAGGATCACCCATGTCTGCAGGGC |
| | 27 (pSaCas9- | CCAGAATCTACTTAAACAAGGCAAAATGCCGTGTTTATCT |
| | HBB-sgRNA1^{ST2ms2}) | CGTCAAGGCCAACATGAGGATCACCCATGTCTGCAGGGCC |
| | encoding a | TTGGCGAGATTTTTTTGCGGCCGC |
| | HBB-sgRNA1 | |
| | with one MS2 | |
| | aptamer inserted | |
| | into the Tetra loop | |
| | position and one at | |
| | the ST2 loop | |
| | position | |
| SEQ ID | DNA oligo cloned | GGTACCGAGGGCCTATTTCCCATGATTCCTTCATATTTGCA |
| NO: 81 | into Plasmid No. | TATACGATACAAGGCTGTTAGAGAGATAATTGGAATTAAT |
| | 30 (pSaCas9^{1PP7}- | TTGACTGTAAACACAAAGATATTAGTACAAAATACGTGAC |
| | HBB-sgRNA1^{3′PP7}) | GTAGAAAGTAATAATTTCTTGGGTAGTTTGCAGTTTTAAAA |
| | encoding the | TTATGTTTTAAAATGGACTATCATATGCTTACCGTAACTTG |
| | U6 promoter, | AAAGTATTTCGATTTCTTGGCTTTATATATCTTGTGGAAAG |
| | HBB sgRNA1 and | GACGAAACACCGAGTAACGGCAGACTTCTCCACGTTCTAG |
| | PP7 aptamer | TACTCTGGAAACAGAATCTACTAGAACAAGGCAAAATGCC |
| | | GTGTTTATCTCGTCAACTTGTTGGCGAGATGGAGCAGACG |
| | | ATATGGCGTCGCTCCTTTTTTTGCGGCCGC |
| SEQ ID | DNA oligo cloned | gcggccgcttgtgtaatcgtagtttcagagtgttagagctgaaaggaagaagtaggagaaa |
| NO: 82 | into Plasmid No. | catgcaaagtaaaagtataacactttccttactaaaccgacatgggtttccaggtaggggc |
| | 32 (pAAV-HBB- | aggattcaggatgactgacagggcccttagggaacactgagaccctacgctgacctcataa |
| | sgRNA2) | atgcttgctacctttgctgttttaattacatcttttaatagcaggaagcagaactctgcac |
| | encoding the | ttcaaaagtttttcctcacctgaggagttaatttagtacaaggggaaaaagtacaggggga |
| | human HBB target | tgggagaaaggcgatcacgttgggaagctatagagaaagaagagtaaattttagtaaagga |
| | template sequence | ggtttaaacaaacaaaatataaagagaaataggaacttgaatcaaggaaatgattttaaaa |
| | and the U6 driven | cgcagtattcttagtggactagaggaaaaaaataatctgagccaagtagaagaccttttcc |
| | HBB sgRNA2 | cctcctacccctactttctaagtcacagaggctttttgttcccccagacactcttgcagat |
| | expression | tagtccaggcagaaacagttagatgtccccagttaacctcctatttgacaccactgattac |
| | cassette | cccattgatagtcacactttgggttgtaagtgactttttatttatttgtatttttgactgc |
| | | attaagaggtctctagttttttatctcttgtttcccaaaacctaataagtaactaatgcac |
| | | agagcacattgatttgtatttattctatttttagacataatttattagcatgcatgagcaa |
| | | attaagaaaaacaacaacaaatgaatgcatatatatgtatatgtatgtgtgtatatataca |
| | | ccacatatatatatatattttttcttttcttaccagaaggttttaatccaaataaggagaa |
| | | gatatgcttagaaccgaggtagagttttcatccattctgtctgtaagtattttgcatattc |
| | | tggagacgcaggaagagatccatctacatatcccaaagctgaattatggtagacaaaactc |
| | | ttccacttttagtgcatcaacttcttatttgtgtaataagaaaattgggaaaacgatcttc |
| | | aatatgcttaccaagctgtgattccaaatattacgtaaatacacttgcaaaggaggatgtt |
| | | tttagtagcaatttgtactgatggtatggggccaagagatatatcttagagggagggctga |
| | | gggtttgaagtccaactcctaagccagtgccagaagagccaaggacaggtacggctgtcat |
| | | cacttagacctcaccctgtggagccacaccctagggttggccaatctactcccaggagcag |
| | | ggagggcaggagccagggctgggcataaaagtcagggcagagccatctattgcttacattt |
| | | gcttctgacacaactgtgttcactagcaacctcaaacagacaccatggtgcatctgactcc |
| | | tgaggagaaaagcgctgtgacagctctctggggaaaagtcaatgtcgacgaagttggtggt |
| | | gaggccctgggcaggttggtatcaaggttacaagacaggtttaaggagaccaatagaaact |
| | | gggcatgtggagacagagaagactcttgggtttctgataggcactgactctctctgcctat |
| | | tggtctattttcccacccttaggctgctggtggtctacccttggacccagaggttctttga |
| | | gtcctttggggatctgtccactcctgatgctgttatgggcaaccctaaggtgaaggctcat |
| | | ggcaagaaagtgctcggtgcctttagtgatggcctggctcacctggacaacctcaagggca |
| | | cctttgccacactgagtgagctgcactgtgacaagctgcacgtggatcctgagaacttcag |
| | | ggtgagtctatgggacgcttgatgttttctttccccttcttttctatggttaagttcatgt |
| | | cataggaaggggataagtaacagggtacagtttagaatgggaaacagacgaatgattgcat |
| | | cagtgtggaagtctcaggatcgttttagtttcttttatttgctgttcataacaattgtttt |
| | | cttttgtttaattcttgctttctttttttttcttctccgcaatttttactattatacttaa |
| | | tgccttaacattgtgtataacaaaaggaaatatctctgagatacattaagtaacttaaaaa |
| | | aaaactttacacagtctgcctagtacattactatttggaatatatgtgtgcttatttgcat |
| | | attcataatctccctactttattttcttttatttttaattgatacataatcattatacata |
| | | tttatgggttaaagtgtaatgttttaatatgtgtacacatattgaccaaatcagggtaatt |
| | | ttgcatttgtaattttaaaaaatgctttcttcttttaatatacttttttgtttatcttatt |
| | | tctaatactttccctaatctctttctttcagggcaataatgatacaatgtatcatgcctct |
| | | attgcaccattctaaagaataacagtgtaatttctgggttaaggcaatagcaatatctctg |
| | | catataaatatttctgcatataaattgtaactgatgtaagaggtttcatattgctaatagc |
| | | agctacaatccagctaccattctgcttttattttatggttgggataaggctggattattct |
| | | gagtccaagctaggcccttttgctaatcatgttcatacctcttatcttcctcccacagctc |
| | | ctgggcaacgtgctggtctgtgtgctggcccatcactttggcaaagaattcaccccaccag |
| | | tgcaggctgcctatcagaaagtggtggctggtgtggctaatgccctggcccacaagtatca |
| | | ctaagctcgctttcttgctgtccaatttctattaaaggttcctttgttccctaagtccaac |
| | | tactaaactgggggatattatgaagggccttgagcatctggattctgcctaataaaaaaca |
| | | tttattttcattgcaatgatctcgagggcctatttcccatgattccttcatatttgcatat |
| | | acgatacaaggctgttagagagataattggaattaatttgactgtaaacacaaagatatta |
| | | gtacaaaatacgtgacgtagaaagtaataatttcttgggtagtttgcagttttaaaattat |
| | | gttttaaaatggactatcatatgcttaccgtaacttgaaagtatttcgatttcttggcttt |
| | | atatatcttgtggaaaggacgaaacaccgccctgtggggcaaggtgaacgttttagtactc |
| | | tggaaacagaatctactaaaacaaggcaaaatgccgtgtttatctcgtcaacttgttggcg |
| | | agatttttctagagcggccgc |
| SEQ ID | DNA oligo cloned | ggatcctaagctcgctttcttgctgtccaatttctattaaaggttcctttgttccctaagt |
| NO: 83 | into Plasmid No. | ccaactactaaactgggggatattatgaagggccttgagcatctggattctgcctaataaa |
| | 36 (pSaCas9^{1xms2}- | aaacatttattttcattgctagctcgctttcttgctgtccaatttctattaaaggttcctt |
| | 2x3′UTR) | tgttccctaagtccaactactaaactgggggatattatgaagggccttgagcatctggatt |
| | encoding two | ctgcctaataaaaaacatttattttcattgc |
| | copies of the | |
| | human HBB 3′ | |
| | UTR | |
| SEQ ID | DNA oligo cloned | CGGCCGAGGGCCTATTTCCCATGATTCCTTCATATTTGCAT |
| NO: 84 | into Plasmid No. | ATACGATACAAGGCTGTTAGAGAGATAATTGGAATTAATT |
| | 41 (pSaCas9^{1xMS2}- | TGACTGTAAACACAAAGATATTAGTACAAAATACGTGACG |
| | 2x3′UTR-HBB | TAGAAAGTAATAATTTCTTGGGTAGTTTGCAGTTTTAAAAT |
| | sgRNA^{3′PP7} | TATGTTTTAAAATGGACTATCATATGCTTACCGTAACTTGA |
| | 1x3′UTR) | AAGTATTTCGATTTCTTGGCTTTATATATCTTGTGGAAAGG |
| | encoding HBB | ACGAAACACCGAGTAACGGCAGACTTCTCCACGTTCTAGT |
| | sgRNA1 with one | ACTCTGGAAACAGAATCTACTAGAACAAGGCAAAATGCCG |
| | PP7 aptamer and | TGTTTATCTCGTCAACTTGTTGGCGAGATATCGGAGCAGAC |
| | one copy of HBB | GATATGGCGTCGCTCCAGCGCTCGCTTTCTTGCTGTCCAAT |
| | 3′ UTR | TTCTATTAAAGGTTCCTTTGTTCCCTAAGTCCAACTACTAA |
| | downstream of U6 | ACTGGGGGATATTATGAAGGGCCTTGAGCATCTGGATTCT |
| | promoter | CG |
| SEQ ID | DNA oligo cloned | GATATCGGAGCAGACGATATGGCGTCGCTCCAGCGCTCGC |
| NO: 85 | into Plasmid No. | TTTCTTGCTGTCCAATTTCTATTAAAGGTTCCTTTGTTCCCT |
| | 42 (pSaCas9^{1xMS2}- | AAGTCCAACTACTAAACTGGGGGATATTATGAAGGGCCTT |
| | 2x3′UTR-HBB | GAGCATCTGGATTCTGCCTAATAAAAAACATTTATTTTCAT |
| | sgRNA^{3′PP7} | TGCGCTCGCTTTCTTGCTGTCCAATTTCTATTAAAGGTTCCT |
| | 2x3′UTR) | TTGTTCCCTAAGTCCAACTACTAAACTGGGGGATATTATGA |
| | encoding one PP7 | AGGGCCTTGAGCATCTGGATTCTGCCTAATAAAAAACATT |
| | aptamer sequence | TATTTTCATTGCTTTTTTTGCGGCCGC |
| | followed by two | |
| | HBB 3′ UTR | |
| | sequences | |
| SEQ ID | Oligo HBA-5′-F | Ctggctaactaccggctcttctggtccccacagactcagagagaaccggtgccaccatgg |
| NO: 86 | | |
| SEQ ID | Oligo HBA-5′-R | ccatggtggcaccggttctctctgagtctgtggggaccagaagagccggtagttagccaG |
| NO: 87 | | |
| SEQ ID | Oligo sp-loop1F | AAAAAAGAAAAAGCTTTAGAAAACATGAGGATCACCCAT |
| NO: 88 | | GTCTGCAGGTCGACTCTAGAATTCCTAGAGCTCG |
| SEQ ID | Oligo sp-loop1R | CGAGCTCTAGGAATTCTAGAGTCGACCTGCAGACATGGGT |
| NO: 89 | | |
| SEQ ID | Il2RG-sp-g1F1 | ACCGGCGCTTGCTCTTCATTCCCT |
| NO: 90 | | |
| SEQ ID | Il2RG-sp-g1R | AAACAGGGAATGAAGAGCAAGCGC |
| NO: 91 | | |
| SEQ ID | MS2-F1 oligo | ACTTGTTGGCGAGATATCTAGAAAACATGAGGATCACCCA |
| NO: 92 | | TGTCTGCAGAGCGCTCGCTTTCTTGCT |
| SEQ ID | MS2-R1 oligo | AGCAAGAAAGCGAGCGCTCTGCAGACATGGGTGATCCTCA |
| NO: 93 | | TGTTTTCTAGATATCTCGCCAACAAGT |
| SEQ ID | Human beta | ACTCCTGTGGAGAAGTCTGCCGTTACT |
| NO: 94 | hemoglobin DNA | |
| | sequence (The “T” | |
| | in the underlined | |
| | codon is the | |
| | mutated | |
| | nucleotide. The | |
| | underlined codon | |
| | encodes the sixth | |
| | amino acid of the | |
| | β globin protein.) | |
| SEQ ID | 119 bp DNA | GAACCCAGGTTCCTGACACAGACAGACTACACCCAGGGAA |
| NO: 95 | Insertion in human | TGAAGAGCAAGCGCCATACTCCTGTGGAGAAGTCTGCCGT |
| | beta hemoglobin | TACTGCCCTGTGGGGCAAGGTGAACGTGGATTGGCTAGC |
| | (HBB) gene in | |
| | EGFP reporter | |
| | cells as described | |
| | in Javidi- | |
| | Parsijani, | |
| | P. et al., PLoS | |
| | One 2017; 12(5): | |
| | e0177444 | |
| SEQ ID | HBB-1849F | CGATCACGTTGGGAAGCTATAGAG |
| NO: 96 | | |
| SEQ ID | HBB-5277R | AACATCCTGAGGAAGAATGGGAC |
| NO: 97 | | |
| SEQ ID | Reporter-mut-F1 | TCGTCGGCAGCGTCAGATGTGTATAAGAGACAGTtccatttcagg |
| NO: 98 | | tgtcgtgag |
| SEQ ID | Reporter-mut-F2 | TCGTCGGCAGCGTCAGATGTGTATAAGAGACAGATtccatttca |
| NO: 99 | | ggtgtcgtgag |
| SEQ ID | Reporter-mut-F3 | TCGTCGGCAGCGTCAGATGTGTATAAGAGACAGGATtccatttc |
| NO: 100 | | aggtgtcgtgag |
| SEQ ID | Reporter-mut-F4 | TCGTCGGCAGCGTCAGATGTGTATAAGAGACAGCGATtccatt |
| NO: 101 | | tcaggtgtcgtgag |
| SEQ ID | Reporter-mut-R1 | GTCTCGTGGGCTCGGAGATGTGTATAAGAGACAGTGAACT |
| NO: 102 | | TCAGGGTCAGCTTGC |
| SEQ ID | Synthetic DNA | GAAGAGCAAGCGCCATACTCCTGTGGAGAAGTCTGCCGTT |
| NO: 103 | sequence (Indel) | ACTGCCCTGTGGGGCAAGGTG |
| SEQ ID | Synthetic DNA | GAAGAGCAAGCGCCATACTCCTGCCGTTACTGCCCTGTGG |
| NO: 104 | sequence (Indel) | GGCAAGGTG |
| SEQ ID | Synthetic DNA | GAAGAGCAAGCGCCATACTCCTGTGAGAAGTCTGCCGTTA |
| NO: 105 | sequence (Indel) | CTGCCCTGTGGGGCAAGGTG |
| SEQ ID | Synthetic DNA | GAAGAGCAAGCGCCATACTCCTGTGAAGTCTGCCGTTACT |
| NO: 106 | sequence (Indel) | GCCCTGTGGGGCAAGGTG |
| SEQ ID | Synthetic DNA | GAAGAGCAAGCGCCATACTCCTGTCTGCCGTTACTGCCCT |
| NO: 107 | sequence (Indel) | GTGGGGCAAGGTG |
| SEQ ID | Synthetic DNA | GAAGAGCAAGCGCCATACTCCTGAGAAGTCTGCCGTTACT |
| NO: 108 | sequence (Indel) | GCCCTGTGGGGCAAGGTG |
| SEQ ID | Synthetic DNA | GAAGAGCAAGCGCCATACTCCTGGAGAAGTCTGCCGTTAC |
| NO: 109 | sequence (Indel) | TGCCCTGTGGGGCAAGGTG |
| SEQ ID | Synthetic DNA | GAAGAGCAAGCGCCATACTCCTGTGGGGCAAGGTG |
| NO: 110 | sequence (Indel) | |
| SEQ ID | Synthetic DNA | GAAGAGCAAGCGCCATACTCCTGTGTCTGCCGTTACTGCC |
| NO: 111 | sequence (Indel) | CTGTGGGGCAAGGTG |
| SEQ ID | Synthetic DNA | GAAGAGCAAGCGCCGTTACTGCCCTGTGGGGCAAGGTG |
| NO: 112 | sequence (Indel) | |
| SEQ ID | Synthetic | GCGGCCgcTCTCGAACTCCTCAAGCAATCCACCTGCCTTGG |
| NO: 113 | fragment | CCTCCCAAAGTGCTGGGATTGCAGGCGTGAGTCACTGCAC |
| | containing IL2RG | CCAGCCGAGAGAATAAATTTCTGTTGGTTTAAGCCACTCA |
| | sgRNA2- | GTTTGGGGATAACTTATGGCAGCCCTAGCAAACTAATACA |
| | expression | TACTAAAGATACATACTAAATACTAAGCTGGGCCATATAG |
| | cassette and | TCCAGTTTTCCTGAGACTCCCAGGCAAGTGCTGTTTTTCTT |
| | IL2RG template | TGCTTAATATCCTACACCACTTTCTGTCTGGTAAAATTACA |
| | for homologous | CTCATTCTTTAAGATGCCACTGAAATAGCACCTCTTCAGCA |
| | recombination. | CAGCCTTCACTAAACTATCCCCCTCTCCATCTTGGTAAATT |
| | 5′ arm and 3′ arm | TAGTTACTTCCTCTTCTGTGCTCACATACTTTGTAGTATCTC |
| | flank underlined: | TACATTTATGCTATAGGACTTGTTACACTATGTTGTATTAC |
| | IL2RG cDNA; | TTGTTTATGTCTTCCCCACTTTTCTGTGAGTGTCTAGAAAT |
| | Followed by | ATGAGGATGTCTTGTTGGTCTATTTCCAGAACATAAGCAC |
| | IL2RG sgRNA2 | AGTGCCTGGCACATATTAAAAACGTAATAAATGTTTGCTG |
| | and sgRNA4 | AATAAATAGTTTCTGTAAGTGGCTTCTCCAATCACCTCTGT |
| | expression | GTTTTCGGGGAAGGTAAAACTGGCAACAGGATGAAGAAT |
| | cassettes | GGATTAGAGAGCAGAGGGCCTTTAGAAAGGGAGGCCAGT |
| | (italicized). | TGATGGAGTCTAGATAGAATCATGACTAGAGCTAATGAAA |
| | | GACTGATTTAGCAGAGTGGCTGTGGTAATGGAAAGGAGGA |
| | | AACCGTTGGGAGAAACACCACAGAAGCAGAGTGGGTTAT |
| | | ATTCTCTGGGTGAGAGAGGGGGAGAAATTGAAGCTGATTC |
| | | TGAGGTTTCAAGTCTGGGTGACTGAGAGGGTGACGATACC |
| | | ATTGACTGAGGTGGGGAAGGCAGGAAGAGAAGCAGAGTT |
| | | GGGGGAAGATGGGAAGCTTGAAGCTAGTATTGTTGTTCCT |
| | | CCATTTCTAGAATATTTTTGTATTATAAGTCACACTTCCTC |
| | | GCCAGTCTCAACAGGGACCCAGCTCAGGCAGCAGCTAAGG |
| | | GTGGGTATTCTGGTTTGGATTAGATCAGAGGAAAGACAGC |
| | | TGTATATGTGCCCACAGGAGCCAAGACGGTATTTTCCATC |
| | | CTCCCAAAACAGTAGAGCTTTGACAGAGATTTAAGGGTGA |
| | | CCAAGTCAAGGAAGAGGCATGGCATAGAACGGTGATGTC |
| | | GGGGGTGGGGGTTCAGAACTTCCATTATAGAAGGTAATGA |
| | | TTTAGAGGAGAAGGTGGTTGAGAATGGTGCTAGTGGTAGT |
| | | GAACAGATCCTTCCCAGGATCTAGGTGGGCTGAGGATTTT |
| | | TGAGTCTGTGACACTATTGTATATCCAGCTTTAGTTTCTGT |
| | | TTACCACCTTACAGCAGCACCTAATCTCCTAGAGGACTTA |
| | | GCCCGTGTCACACAGCACATATTTGCCACACCCTCTGTAA |
| | | AGCCCTGGTTTATAAGGTTCTTTCCACCGGAAGCTATGACA |
| | | GAGGAAACGTGTGGGTGGGGAGGGGTAGTGGGTGAGGGA |
| | | CCCAGGTTCCTGccAccatgCtCaaAccTtcCCtGccTttTacCAGcTtG |
| | | CtGttTctCcagctCccTctCctCggCgtCggActCaaTacAacTatCctGacAcc |
| | | TaaCggAaaCgaGgaTacAacCgcCgaCttTttTctCacAacCatgccTacAga |
| | | TAGcTtGTCCgtGAGcacCctCccTctGccTgaAgtGcagtgCttCgtCttTaa |
| | | CgtGgaAtaTatgaaCtgTacCtggaaTTCcTCcAGCgaAccTcagccAacAa |
| | | aTctGacActCcaCtaCtggtaTaaAaaTAGCgaCaaCgaCaaGgtGcagaaAt |
| | | gTTCccaTtaCTTGttTAGCgaGgaGatTacCAGCggAtgCcagCtCcaGa |
| | | aGaaAgaAatTcaTctGtaTcaGacCttCgtGgtGcagctGcaggaTccTAgAga |
| | | GccTagAagGcaggcTacCcagatgTTGaaGctCcagaaCctCgtCatTccTtgg |
| | | gcCccTgaAaaTctGacCTtGcaTaaGctCTCCgaGAGccagctGgaGctCaa |
| | | TtggaaTaacagGttTCtgaaTcaTtgCCtggaAcaTCTCgtCcaAtaTAGAac |
| | | CgaTtgggaTcaTTCctggacCgaGcaGAGCgtCgaCtaCCgGcaCaaAttT |
| | | AGcCtgccAagCgtCgaCggAcagaaGAgAtaTacCttCAgAgtGAgATCc |
| | | AgAttCaaTccTctGtgCggCTCCgcAcagcaCtggTCCgaGtggTCccaTcc |
| | | TatTcaTtggggATCcaaCacCAGCaaGgaAaaCccAttTctgttCgcTCTgga |
| | | GgcTgtCgtGatTAGCgtGggAAGcatgggCCtgatCatTTCcTTGctGtgC |
| | | gtTtaCttTtggctggaGAgAacCatgccTAgGatCccTacActCaaAaaTctGga |
| | | AgaCTTGgtGacAgaGtaTcaTggAaaTttCAGCgcTtggTCCggAgtCA |
| | | GCaaAggCctCgcCgaAagCctCcagccTgaTtaTagCgaGAgGctGtgTctG |
| | | gtGagCgaAatCccTccTaaGggCggAgcTctGggCgaAggAccAggCgCT |
| | | AGCCCTTGTAATCAGCACTCCCCTTATTGGGCTCCTCCTTG |
| | | CTATACATTGAAACCAGAGACATAAGGGAACCCAGGAGA |
| | | CAGGCCACACAGATGCTAAAACTGCAGAATCTGGGTAATT |
| | | TGGAAAGAAAGGGTCAAGAGACCAGGGATACTGTGGGAC |
| | | ATTGGAGTCTACAGAGTAGTGTTCTTTTATCATAAGGGTAC |
| | | ATGGGCAGAAAAGAGGAGGTAGGGGATCATGATGGGAAG |
| | | GGAGGAGGTATTAGGGGCACTACCTTCAGGATCCTGACTT |
| | | GTCTAGGCCAGGGGAATGACCACATATGCACACATATCTC |
| | | CAGTGATCCCCTGGGCTCCAGAGAACCTAACACTTCACAA |
| | | ACTGAGTGAATCCCAGCTAGAACTGAACTGGAACAACAGA |
| | | TTCTTGAACCACTGTTTGGAGCACTTGGTGCAGTACCGGAC |
| | | TGACTGGGACCACAGCTGGACTGTGAGTGACTAGGGACGT |
| | | GAATGTAGCAGCTAAGGCCAAGAAAGTAGGGCTAAAGGA |
| | | TTCAACCAGACAGATAGAAGGACCTAATATCAAGCTCCTG |
| | | TTCTCTGCCTCCCAGCTTCTCTGCTCACCCCCTACCCTCCCT |
| | | CCTCCAACTCCTTTCCTCGAGGGCCTATTTCCCATGATTCCTT |
| | | CATATTTGCATATACGATACAAGGCTGTTAGAGAGATAATTGGA |
| | | ATTAATTTGACTGTAAACACAAAGATATTAGTACAAAATACGTGA |
| | | CGTAGAAAGTAATAATTTCTTGGGTAGTTTGCAGTTTTAAAATTA |
| | | TGTTTTAAAATGGACTATCATATGCTTACCGTAACTTGAAAGTAT |
| | | TTCGATTTCTTGGCTTTATATATCTTGTGGAAAGGACGAAACAC |
| | | CGTGGCCTGTCTCCTGGGTTCCCGTTTTAGTACTCTGGAAACA |
| | | GAATCTACTAAAACAAGGCAAAATGCCGTGTTTATCTCGTCAAC |
| | | TTGTTGGCGAGATTTTTTGTTTTAGAGCTAGAAATAGCAAGT |
| | | TAAAATAAGGCTAGTCCGTTTTTAGCGCGTGCGCCAATTCT |
| | | GCAGACAAATGAGGGCCTATTTCCCATGATTCCTTCATATTTG |
| | | CATATACGATACAAGGCTGTTAGAGAGATAATTGGAATTAATTT |
| | | GACTGTAAACACAAAGATATTAGTACAAAATACGTGACGTAGAA |
| | | AGTAATAATTTCTTGGGTAGTTTGCAGTTTTAAAATTATGTTTTA |
| | | AAATGGACTATCATATGCTTACCGTAACTTGAAAGTATTTCGAT |
| | | TTCTTGGCTTTATATATCTTGTGGAAAGGACGAAACACCGGACA |
| | | CAGACAGACTACACCCAGTTTTAGTACTCTGGAAACAGAATCTA |
| | | CTAAAACAAGGCAAAATGCCGTGTTTATCTCGTCAACTTGTTGG |
| | | CGAGATTTTTggtAccggtGCGGCCGC |
| SEQ ID | HBB sgRNA1 | GCggccgcttgtgtaatcgtagtttcagagtgttagagctgaaaggaagaagtaggagaaa |
| NO: 114 | expression | catgcaaagtaaaagtataacactttccttactaaaccgacatgggtttccaggtaggggc |
| | cassette and the | aggattcaggatgactgacagggcccttagggaacactgagaccctacgctgacctcataa |
| | wild type | atgcttgctacattgctottaattacatcuttaatagcaggaagcagaactctgcacttca |
| | template. | aaagtttttcctcacctgaggagttaatttagtacaaggggaaaaagtacagggggatggg |
| | italicized: | agaaaggcgatcacgttgggaagctatagagaaagaagagtaaattttagtaaaggaggtt |
| | HBB 5′ arm. | taaacaaacaaaatataaagagaaataggaacttgaatcaaggaaatgattttaaaacgca |
| | underlined: HBB | gtattcttagtggactagaggaaaaaaataatctgagccaagtagaagaccttttcccctc |
| | 3′ arm | ctacccctactttctaagtcacagaggctttttgttcccccagacactcttgcagattagt |
| | Bold: HBB | ccaggcagaaacagttagatgtccccagttaacctcctatttgacaccactgattacccca |
| | sgRNA1 | ttgatagtcacactttgggttgtaagtgactttttatttatttgtatttttgactgcatta |
| | expression | agaggtctctagttttttatctcttgtttcccaaaacctaataagtaactaatgcacagag |
| | cassette. | cacattgatttgtatttattctatttttagacataatttattagcatgcatgagcaaatta |
| | | agaaaaacaacaacaaatgaatgcatatatatgtatatgtatgtgtgtatatatacacaca |
| | | tatatatatatattttttcttttcttaccagaaggttttaatccaaataaggagaagatat |
| | | gcttagaaccgaggtagagttttcatccattctgtcctgtaagtattttgcatattctgga |
| | | gacgcaggaagagatccatctacatatcccaaagctgaattatggtagacaaaactcttcc |
| | | acttttagtgcatcaacttcttatttgtgtaataagaaaattgggaaaacgatcttcaata |
| | | tgcttaccaagctgtgattccaaatattacgtaaatacacttgcaaaggaggatgttttta |
| | | gtagcaatttgtactgatggtatggggccaagagatatatcttagagggagggctgagggt |
| | | ttgaagtccaactcctaagccagtgccagaagagccaaggacaggtacggctgtcatcact |
| | | tagacctcaccctgtggagccacaccctagggttggccaatctactcccaggagcagggag |
| | | ggcaggagccagggctgggcataaaagtcagggcagagccatctattgcttacatttgctt |
| | | ctgacacaactgtgttcactagcaacctcaaacagacaccatggtgcatctgactcctgag |
| | | gagaaaagcgctgtgacagctctctggggaaaagtcaatgtcgacgaagttggtggtgagg |
| | | ccctgggcaggttggtatcaaggttacaagacaggtttaaggagaccaatagaaactgggc |
| | | atgtggagacagagaagactcttggatttctgataggcactgactctctctgcctattggt |
| | | ctattttcccacccttaggctgctggtggtctacccttggacccagaggttctttgagtcc |
| | | tttggggatctgtccactcctgatgctgttatgggcaaccctaaggtgaaggctcatggca |
| | | agaaagtgctcggtgcctttagtgatggcctggctcacctggacaacctcaagggcacctt |
| | | tgccacactgagtgagctgcactgtgacaagctgcacgtggatcctgagaacttcagggtg |
| | | agtctatgggacgcttgatgttttctttccccttcttttctatggttaagttcatgtcata |
| | | ggaaggggataagtaacagggtacagtttagaatgggaaacagacgaatgattgcatcagt |
| | | gtggaagtctcaggatcgttttagtttcttttatttgctgttcataacaattgttttcttt |
| | | tgtttaattcttgctttctttttttttcttctccgcaatttttactattatacttaatgcc |
| | | ttaacattgtgtataacaaaaggaaatatctctgagatacattaagtaacttaaaaaaaaa |
| | | ctttacacagtctgcctagtacattactatttggaatatatgtgtgcttatttgcatattc |
| | | ataatctccctactttattttcttttatttttaattgatacataatcattatacatattta |
| | | tgggttaaagtgtaatgttttaatatgtgtacacatattgaccaaatcagggtaattttgc |
| | | atttgtaattttaaaaaatgctttcttcttttaatatacttttttgtttatcttatttcta |
| | | atactttccctaatctctttctttcagggcaataatgatacaatgtatcatgcctattgca |
| | | ccattctaaagaataacagtgataatttctgggttaaggcaatagcaatatctctgcatat |
| | | aaatatttctgcatataaattgtaactgatgtaagaggtttcatattgctaatagcagcta |
| | | caatccagctaccattctgcttttattttatggttgggataaggctggattattctgagtc |
| | | caagctaggcccttttgctaatcatgttcatacctcttatcttcctcccacagctcctggg |
| | | gcaacgtgctggtctgtgtgctggcccatcactttggcaaagaattcaccccaccatgcag |
| | | gctgcctatcagaaagtggtggctggtgtggctaatgccctggcccacaagtatcactaag |
| | | ctcgctttcttgctgtccaatttctattaaaggttcctttgttccctaagtccaactacta |
| | | aactgggggatattatgaagggccttgagcatctggattctgcctaataaaaaacatttat |
| | | tttcattgcaatgatctcgagggcctatttcccatgattccttcatatttgcatatacgat |
| | | acaaggctgttagagagataattggaattaatttgactgtaaacacaaagatattagtaca |
| | | aaatacgtgacgtagaaagtaataatttcttgggtagtttgcagttttaaaattatgfftt |
| | | aaaatggactatcatatgcttaccgtaacttgaaagtatttcgatttcttggctttatata |
| | | tcttgtggaaaggacgaaacaccGAGTAACGGCAGACTTCTCCACgttttagtactctgga |
| | | aacagaatctactaaaacaaggcaaaatgccgtgtttatctcgtcaacttgttggcgagat |
| | | ttttgcGGCCGC |
| SEQ ID | HBB-F2 | GGGCAGAGCCATCTATTGCTTA |
| NO: 115 | | |
| SEQ ID | HBB-R3 | TGGGAAAATAGACCAATAGGCAGAG |
| NO: 116 | | |
| SEQ ID | sgRNA-R2 | CGCCAACAAGTTGACGAGAT |
| NO: 117 | | |
| SEQ ID | sgRNA-R3 | GATAAACACGGCATTTTGCCTTG |
| NO: 118 | | |
| SEQ ID | HBB_off-R1 | GTCTCGTGGGCTCGGAGATGTGTATAAGAGACAGTggctgagg |
| NO: 119 | | tgggagaatcac |
| SEQ ID | HBB_off-F2 | TCGTCGGCAGCGTCAGATGTGTATAAGAGACAGTatttctcctctg |
| NO: 120 | | cactgccc |
| SEQ ID | HBB_off-R2 | GTCTCGTGGGCTCGGAGATGTGTATAAGAGACAGTggagcctc |
| NO: 121 | | ggcctagatttc |
| SEQ ID | HBB_off-F3 | TCGTCGGCAGCGTCAGATGTGTATAAGAGACAGTTGATCC |
| NO: 122 | | CTTCCACCAATGTC |
| SEQ ID | HBB_off-R3 | GTCTCGTGGGCTCGGAGATGTGTATAAGAGACAGTCACGG |
| NO: 123 | | CTAGGAATAGCAAGG |
| SEQ ID | HBB_off-F4 | TCGTCGGCAGCGTCAGATGTGTATAAGAGACAGTAAAGGA |
| NO: 124 | | AATTCCATCAGACTAACG |
| SEQ ID | HBB_off-R4 | GTCTCGTGGGCTCGGAGATGTGTATAAGAGACAGTCTTTG |
| NO: 125 | | AAAATGCCGTCCATC |
| SEQ ID | HBB_off-F5 | TCGTCGGCAGCGTCAGATGTGTATAAGAGACAGTAAGACA |
| NO: 126 | | AAAGCAGAAGGTAAGC |
| SEQ ID | HBB_off-R5 | GTCTCGTGGGCTCGGAGATGTGTATAAGAGACAGTAAGTT |
| NO: 127 | | GAAGATAGGACCCCACTC |
| SEQ ID | HBB_off-R6 | GTCTCGTGGGCTCGGAGATGTGTATAAGAGACAGTGGCAA |
| NO: 128 | | CAAGAGCAAAACTCC |
| SEQ ID | HBB_off-F7 | TCGTCGGCAGCGTCAGATGTGTATAAGAGACAGTCCAGAA |
| NO: 129 | | AGGGAAAACTTGCAC |
| SEQ ID | HBB_off-R7 | GTCTCGTGGGCTCGGAGATGTGTATAAGAGACAGTCCACT |
| NO: 130 | | GACCCAATCTTTTCC |
| SEQ ID | HBB_off-F8 | TCGTCGGCAGCGTCAGATGTGTATAAGAGACAGTCTGCTC |
| NO: 131 | | TTTGCCTGTTGGAG |
| SEQ ID | HBB_off-R8 | GTCTCGTGGGCTCGGAGATGTGTATAAGAGACAGTGCTAA |
| NO: 132 | | AGCTGGAAGGCTGTG |
| SEQ ID | IL2RG-3301R | ATGCCCTCTGTAGTGGGTTG |
| NO: 133 | | |
| SEQ ID | IL2RG-mut-F1 | GGCAGCTGCAGGAATAAGAG |
| NO: 134 | | |
| SEQ ID | IL2RG-mut-R4 | TCGTCGGCAGCGTCAGATGTGTATAAGAGACAGTGAAGCT |
| NO: 135 | | ATGACAGAGGAAACG |

### Example 10. System for Packaging SaCas9 mRNA into LVLPs

Described herein is a system to efficiently package SaCas9 mRNA into LVLPs. The LVLPs enabled transient SaCas9 expression and highly efficient genome editing. They generated lower off-target rates compared with AAV and lentiviral delivery. The SaCas9 LVLPs described here have the transient expression feature of RNP-, mRNA- and nanoparticle-delivery strategies, but retain the transduction efficiency of lentiviral vectors. This system can be used for packaging various editor protein-encoding mRNA for genome editing.

Plasmids. pRSV-Rev (Addgene #12253), pMD2.G (Addgene #12259), pMDLg/pRRE (Addgene #12251), psPAX2-D64V (Addgene #63586), pSL-MS2×12 (Addgene #27119), pKanCMV-mRuby3-10aa-H2B (addgene #74258) and pX601-AAV-CMV::NLS-SaCas9-NLS-3xHA-bGHpA;U6::BsaI-sgRNA (Addgene #61591) were purchased from Addgene. pCDH-GFP was purchased from SBI (CD513B-1). The remaining plasmids were generated (see Table 2). Gene synthesis was done by GenScript Inc. All constructs generated were sequence confirmed. Sequence information for primers, oligos, and synthesized DNA fragments is in Table 10.

GFP reporter assay for gene editing activities The EGFP reporter cell line described in Javidi-Parisjani et al. was used to detect gene editing activity of SaCas9/human beta hemoglobin (HBB) sgRNA1 on the target sequences inserted in the GFP-reporter cassette. The GFP-reporter cells (derived from HEK293T cells) expressed no EGFP due to disruption of the EGFP reading frame by the insertion of the HBB sickle mutation and IL2RG target sequences between the start codon and the second codon of EGFP coding sequence. Indels formed after gene editing may restore the reading frame of the EGFP, resulting in EGFP expression. GFP-positive cells were analyzed by fluorescence microscopy or flow cytometry (BD Biosciences, Accuri C6). A single cell suspension was made in PBS/0.5% FBS for analysis. The cells without fluorescent protein expression were used as negative controls and a marker was placed at the position so that 99.9% of the cells were on the left side of the marker. In treated samples, cells on the right side of the marker were considered positive.

AAV6 virus production and transduction Adeno-associated virus expressing SaCas9 and HBB sgRNA1 were made from the AAV vector pSaCas9 (expresses SaCas9) and pSaCas9-HBB-sgRNA1 (expresses HBB sgRNA1, and contains donor template for homologous recombination to change the wild-type HBB gene to the Sickle mutation) respectively. AAV serotype 6 (AAV6) production and concentration were performed by Virovek, Inc. (Hayward, CA). For AAV6 transduction, the cells were changed to serum-free medium or OPTI-MEM, and AAV6 was added to the cells at a titer of 10³-10⁴ virus genome/cell. 24 hours later the medium was changed to serum-containing growth medium.

Lentivirus and LVLP production Lentivirus was produced with the second- or the third-generation packaging systems, as described previously in Javidi-Parisjani et al. Lentiviral vector pCK002-HBB-sgRNA1 expressing both SaCas9 and HBB sgRNA1 was used to produce integration-competent lentivirus (packaged by packaging plasmid pspAX2 or pMDLg/pRRE), and integration-defective lentivirus (IDLV) packaged by packaging plasmid pspAX2-D64V or pMDLg/pRRE-D64V). To produce LVLPs packaged with SaCas9 mRNA, HEK293T cells were transfected with mixed DNA of the packaging plasmid (NC-MCP or NC-PCP modified), envelope plasmid (pMD2.G), and the SaCas9 expression plasmid with DNA ratio as shown in Table 11. When VPR or NEF fusion proteins were used for packaging, SaCas9 mRNA, plasmid DNA for their expression was also included. Transfection was mediated by polyethylenimine (PEI, Polysciences Inc.) with a DNA:PEI ratio of 1:2. Cell culture and DNA transfection were performed as described in in Javidi-Parisjani et al. 24 h after transfection, the medium was changed to Opti-MEM and the lentivirus or LVLPs were collected twice with one collection each 24 h. The supernatant was spun for 10 min at 500 g to remove cell debris before further processing described below.

TABLE 11
Plasmid DNA used to transfect HEK293T cells to make
lentiviral particles loaded with SaCas9 mRNA^{a}

| | MCP-fused | MCP-fused | PCP-fused | PCP-fused | VPR or NEF |
|---|---|---|---|---|---|
| | second | third | second | third | mediated |
| | generation | generation | generation | generation | SaCas9 |
| | packaging | packaging | packaging | packaging | mRNA |
| | system | system | system | system | packaging |
| Gag-pol | pspAX2- | pMDLg/pR | pspAX2- | pMDLg/pR | pspAX2- |
| packaging | D64V-NC- | RE-D64V- | D64V-NC- | RE-D64V- | D64V |
| plasmid | MS2 | NC-MS2 | PP7 | NC-PP7 | |
| (16.5 μg) | | | | | |
| SaCas9 | pSaCas9^{1xMS2} | pSaCas9^{1xMS2} | pSaCas9^{1xPP7} | pSaCas9^{1xPP7} | pSaCas9^{1xMS2} |
| plasmid | | | | | |
| (12 μg) | | | | | |
| pMD2G | 8 | 8 | 8 | 8 | 8 |
| (μg) | | | | | |
| pRSV-REV | 0 | 4.5 | 0 | 4.5 | 0 |
| (μg) | | | | | |
| VPR or NEF | 0 | 0 | 0 | 0 | 12 |
| MCP fusion | | | | | |
| plasmid (μg) | | | | | |

^{a}13 × 10⁶ cells were seeded in 15-cm dishes 24 hours before transfection.

Concentrating lentivirus and LVLPs Three methods were used to concentrate lentivirus and LVLPs: 1) The supernatant containing the virus or LVLPs was laid on a 10 ml 20% sucrose cushion, and then centrifuged at 20,000 g 4° C. for 4 hours; 2) The supernatant containing the virus or LVLPs was mixed with the Lenti-X™ Concentrator (Takara, Cat. No. 631232) at a ratio of 3:1 (volume/volume), incubated at 4° C. for 30 mins and then centrifuged at 4° C. 1,500×g for 45 minutes; 3) The supernatant was concentrated with the KR2i TFF System (KrosFlo® Research 2i Tangential Flow Filtration System) (Spectrum Lab, Cat. No. SYR2-U20) using the concentration-diafiltration-concentration mode. Typically, 150-300 ml supernatant was first concentrated to about 50 ml, diafiltrated with 500 ml to 1000 ml PBS, and finally concentrated to about 8 ml. The hollow fiber filter modules were made from modified polyethersulfone, with a molecular weight cut-off of 500 kDa. The flow rate and the pressure limit were 80 ml/min and 8 psi for filter module D02-E500-05-N, and 10 ml/min and 5 psi for the filter module C02-E500-05-N. Since the TFF method produced lentivirus and LVLPs with the best activities, data were generated with virus or LVLPs concentrated by the TFF system, unless otherwise stated.

Lentivirus and LVLP quantification Viral titer was determined by p24 based ELISA (Cell Biolabs, QuickTiter™ Lentivirus Titer Kit Catalog Number VPK-107). When unpurified samples were assayed, the viral particles were precipitated according to the manufacturer's instructions so that the soluble p24 peptide was not detected.

Transmission electron microscopy Transmission electron microscopy was performed at the Cellular Imaging Shared Resource of Wake Forest Baptist Health Center (Winston-Salem, NC). For negative staining, 30 ml virus containing supernatant was concentrated to about 1 ml using an ultracentrifuge. Then, plain carbon grids were soaked in 20 μl of virus samples and the particles were stained with phosphotungstic acid. The samples were dried and observed under a FEI Tecnai G2 30 electron microscope (FEI, Hillsboro, OR).

Western blotting analyses To analyze viral proteins from lentivirus and LVLPs, purified lentivirus or LVLPs (200 ng p24 by ELISA) were lysed in 20 μl of 1× Laemmli sample buffer. The proteins in each sample were separated on SDS-PAGE gels and analyzed by Western blotting. The antibodies used include HIV1 p17 antibody for MA (ThermoFisher Scientific, Cat No. PA1-4954, 1:1000), HIV1 p15 polyclonal antibody for NC (Abcam, Cat No. ab66951, 1:1000) and p24 monoclonal antibody for CA (Cell Biolabs, Cat No. 310810, 1:1000).

To detect SaCas9 protein, HEK293T cells or GFP reporter cells were transfected with SaCas9 expression plasmid DNA or were co-transduced with 100 ng SaCas9 mRNA packaged LVLPs and 100 ng p24 of IDLV expressing HBB sgRNA1. 48 hours after transfection or transduction, the cells were lysed with 100 μl of 1× Laemmli sample buffer and equal volumes of each sample was loaded for SDS-PAGE separation and Western blotting analysis by anti-HA antibody (ProteinTech, 51064-2-AP, 1:1000) and anti-Cas9 antibody (Millipore Sigma, MAB131872, clone 6F7, 1:1000) to detect the HA-tagged SaCas9. Anti-beta actin (Sigma, A5441, 1:5000) was used to compare the amount of input.

HRP conjugated anti-Mouse IgG (H+L) (ThermoFisher Scientific, Cat No. 31430, 1:5000) and anti-Rabbit IgG (H+L) (Cat No. 31460, 1:5000) secondary antibodies were used in Western blotting. Chemiluminescent reagents (Pierce) were used to visualize the protein signals under the LAS-3000 system (Fujifilm).

RNA isolation from lentivirus or LVLPs An miRNeasy Mini Kit (QIAGEN Cat No./ID: 217004) was used to isolate RNA from concentrated lentivirus or LVLPs. Alternately, RNA was isolated directly from 140 μl particle containing supernatant with the QIAamp Viral RNA Mini Kit (QIAGEN).

RT-PCR analysis of RNA The QuantiTect Reverse Transcription Kit (QIAGEN) was used to reverse-transcribe the RNA to cDNA. Custom designed Hydrolysis probes specific for SaCas9, HBB sgRNA1 and EGFP (ThermoFisher Scientific) were used in qPCR, together with TaqMan Universal PCR Master Mix (ThermoFisher Scientific). PCR was run on an ABI 7500 instrument.

Lentivirus and LVLP transduction Various amounts of lentivirus or LVLPs (ng p24 protein) were added to 2.5×10⁴ cells grown in 24-well plates, with 8 μg/ml polybrene. The cells were incubated with the particle-containing medium for 12-24 hours, after which normal medium was replaced.

Gene editing in human cells For gene editing with Cas9 expressed from AAV6, the SaCas9 expressing AAV6 and the HBB sgRNA1 expressing AAV6 were co-transduced into GFP reporter cells. For gene editing with LV or IDLV (packaged with packaging plasmids containing a D64V mutation in the integrase) expressing both SaCas9 and HBB sgRNA1, virus equivalent to 10-300 ng p24 was used to transduce 2.5×10⁴ cells in 24 well plates. For gene editing with LVLPs, various amounts of SaCas9 mRNA containing LVLPs were co-transduced into HEK293T cells or the GFP reporter cells with an IDLV expressing HBB sgRNA1. 48-72 hours after transduction, gene editing activity was analyzed by GFP-reporter assay or next-generation sequencing.

To examine gene editing in human lymphoblastoid cells immortalized by Epstein-Barr virus transformation, human lymphoblastoid cell line GM16265 was purchased from the Corrie Institute. The lymphoblasts were cultured in RPMI 1640 with 2 mmol/L L-glutamine and 15% fetal bovine serum at 37° C. under 5% carbon dioxide. For LVLP and IDLV transduction, 2×10⁵ cells were added to 0.5 ml RPMI growth medium. Then, 100 p24 or 500 p24 of SaCas9 LVLP and IL2RG sgRNA IDLV were added to the cells. Polybrene was added in the medium to a final concentration of 8 μg/ml. Fresh medium was replaced twenty hour hours after transduction. The cells were collected 72 hours after transduction for DNA analysis by next-generation sequencing.

Next-generation sequencing and data analyses The following DNA regions were amplified for next-generation sequencing analysis: 1) the endogenous HBB target sequence, 2) the endogenous IL2RG target sequence, and 3) the HBB target sequence in the integrated GFP-expression cassette. To amplify the endogenous HBB target sequence for sequencing, a nested PCR strategy was used to avoid amplifying the sequence from the viral vector template. First, primers HBB-1849F and HBB-5277R were used to amplify the 3.4 kb region from the HBB gene locus. These two primers cannot amplify sequences from the templates in the viral vectors. Then two inside primers HBB-MUT-F and the HBB-MUT-R primers were used to amplify the target DNA for sequencing (Table 10; SEQ ID NOs: 34-40). To amplify the endogenous IL2RG target sequence, primers IL2RG-1029F and IL2RG-3301R were used to amplify the target region from the treated cells (unable to amplify sequences from the templates in the viral vectors), then two inside primers IL2RG-mut-F1 and IL2RG-mut-R4 were used to amplify the target DNA from the first PCR product for sequencing. To amplify the HBB target sequence from the integrated EGFP reporter for sequencing, Reporter-mut-F and Reporter-mut-R primers were used (Table 10; SEQ ID NOs: 98-102). The proofreading HotStart® ReadyMix from KAPA Biosystems (Wilmington, MA) was used for PCR. In order to increase sequence diversity during next generation sequencing, variant numbers of stagger nucleotide were added after the barcodes in the 5′ primers to amplify the same target from different samples. Single-end reads from the 5′ of the PCR products were obtained by next-generation sequencing using the Illumina NextSeq 500 as described in Javidi-Parisjani et al. Low quality reads were removed. All sequences were sorted based on the barcodes.

After removing the 3′ linker and 5′ barcode sequences, the resulting reads were submitted to the online Cas-Analyzer software for mutation analysis. When submitting the reference sequence, the primer sequences were excluded if they were at least 12 nt away from the sgRNA target. Otherwise, primer sequences were included in the reference. Only those readings containing both the 5′ 12 nt and 3′ 12 nt of the reference sequence were included in Indel analysis. The total Indel rate was the difference between 1 and the percentage of readings without mutation. The top 8-10 most frequently observed readings were presented.

SaCas9 mRNA decay analysis To compare the SaCas9 RNA levels expressed from IDLV and LVLPs, 2.5×10⁴ HEK293 cells were transduced with 35 ng p24 of IDLV or LVLPs. IDLV particles could express SaCas9, and LVLPs were packaged with SaCas9 mRNA. 24 hours after transduction, the cells were maintained in medium containing 0.5% FBS to limit cell division. Fresh medium was changed every 48 hours. The cells were collected 24, 48, 72, and 96 hours post transduction, followed by RNA extraction and RT-qPCR analysis. Three hydrolysis probes, SaCas9, GAPDH and RPLP0 were used for expression analysis. The relative Cas9 mRNA levels of different particles 24 hours after transduction were determined by normalization to RPLP0. Since no new mRNA was generated with LVLPs after transduction, no normalization was performed when evaluating mRNA decay of Cas9 mRNA from the same particle, in order to avoid interference of possible cell proliferation on analysing RNA decay. However, RPLP0 and GAPDH expression were examined for all samples to make sure sample preparation was successful and consistent.

Statistical analysis GraphPad Prism software (version 5.0, GraphPad Software Inc) was used for statistical analyses. T-tests were used to compare the averages of two groups. Analysis of Variance (ANOVA) was performed followed by Tukey post hoc tests to analyze data from more than two groups. Bonferroni post hoc tests were performed following ANOVA in cases of two factors. p<0.05 was regarded as statistically significant.

Fusing Nucleocapsid (NC) protein with RNA-binding proteins enabled efficient packaging of SaCas9 mRNA in lentivirus-like particles In order to package SaCas9 mRNA into LVLPs via specific MS2/MCP interaction MS2-binding protein MCP was fused with lentiviral proteins and and MCP interacting MS2 aptamer was added after the stop codon of SaCas9 mRNA. To find the MCP fusion partner with the most efficient mRNA packaging, four different lentiviral proteins, viral protein R (VPR), negative regulatory factor (NEF), nucleocapsid protein (NC) and matrix protein (MA), were explored as the recipient proteins for MCP based on the following observation: 1) A mutant NEF (with three mutations: G3C, V153L and G177E) can be incorporated into lentiviral particles for up to 1100 molecules/capsid and has been used as a vehicle for foreign protein delivery; 2) VPR is incorporated into the viral core for up to 550 copies/particle and has also been used as the protein delivery vehicle; 3) NC, which is processed from each one of the 2500-5000 Gag precursors forming a lentiviral particle, is the major RNA binding protein in the viral core; and 4) MA, which is also processed from Gag protein, is shown to bind tRNA.

MCP was fused with NEF and VPR in the configuration shown in FIG. 2 and FIG. 7A, which are duplicate figures. MCP-VPR and NEF-MCP were expressed from co-transfected plasmid DNA during LVLP production with the unmodified packaging plasmid (FIG. 7B). To fuse MCP with NC, the packaging plasmid was modified by inserting MCP coding sequence after that of the second zinc finger of NC. Because the second zinc finger of NC is necessary for virion production and deleting this domain reduced virion production 10 fold this domain was preserved rather than replacing it with MCP. To fuse MCP with MA, the coding sequence for MA amino acid 44-132 was replaced in the packaging plasmid with that of MCP, since this region of MA is not necessary for virus production. An MCP coding sequence was inserted into NC or MA of the packaging plasmid in a way that the Gag precursor protein reading frame and the protease processing sites were preserved.

To examine whether the incorporation of MCP affected virion production, GFP-lentivirus production efficiency using these constructs was compared. Expressing MCP-VPR or NEF-MCP in packaging cells did not affect GFP lentivirus production with the original packaging plasmid. NC-MCP modified packaging plasmid generated GFP lentivirus with similar efficiency as the original packaging plasmid, but MA-MCP modified packaging plasmid showed reduced virus production efficiency (FIG. 7C).

In order to have a simple assay to compare genome editing activities, the GFP-reporter cells (derived from HEK293T cells transduced with GFP-reporter lentiviral vectors) were used. These GFP-reporter cells were integrated with a GFP expression cassette, wherein the Sickle mutation sequence of human beta hemoglobin (HBB) targeted by HBB sgRNA1 was inserted between the EGFP start and the second codons, disrupting the reading frame. Insertions or deletions (Indels) in the target sequence resulting from gene editing may restore the EGFP reading frame and GFP expression. To test whether fusing MCP to virus proteins could enhance the packaging of SaCas9 mRNA into LVLPs, SaCas9^{1xMS2} (SaCas9 mRNA with 1 copy of MS2 aptamer after the stop codon) was expressed during LVLP generation by the original packaging plasmid (with or without MCP-VPR or NEF-MCP expression), or by the MCP modified packaging plasmid (FIG. 7B). These LVLPs were then co-transduced into the GFP-reporter cells with an integration-defective lentivirus (IDLV) vector expressing HBB sgRNA1 targeting the Sickle mutation of human beta hemoglobin (HBB) (32). LVLPs made from NC-MCP modified packaging plasmid generated significantly more GFP reporter cells than LVLPs generated by all other strategies of MCP incorporation (p<0.01) (FIG. 7D) NC was thus used as the MCP recipient protein for further experiments.

MCP dimers, but not the monomers or oligomers, had RNA aptamer-binding activities. When the performance of packaging plasmids wherein NC was fused with one or two copies of MCP was compared, LVLPs generated by packaging plasmid fused with one copy of MCP produced more GFP+ reporter cells than that fused with two copies of MCP (5.7%±0.1%, N=4, versus 4.4%±0.3%, N=4; p<0.01. Thus, the modified packaging plasmid fused with one copy of MCP was used in subsequent experiments.

To test whether the gene editing activity observed was caused by nucleic acid in extracellular vesicles promoted by VSV-G, a control where the NC-MCP modified packaging plasmid was replaced with the plasmid expressing mRuby fluorescent protein, but none of the lentivirus packaging proteins, was included. Equal volumes of supernatants were co-transduced onto GFP-reporter cells with HBB sgRNA1 expressing IDLV, supernatant produced with the packaging plasmid generated up 15% GFP⁺ reporter cells. Whereas supernatant produced without packaging plasmid generated lower than 0.5% GFP+ reporter cells under all conditions (FIG. 7E) and the mRuby positive rate was lower than 0.5% in cells treated with the highest volume of supernatant (FIG. 8). Since VSV-G was present in both conditions, the data suggested that extracellular vesicles did not having a major role in generating GFP⁺ reporter cells. The data also suggested that residual plasmid DNA contributed little in the total gene editing activities observed.

To further test the contribution of residual plasmid DNA from LVLP production in generating GFP+ reporter cells, constructs expressing only SaCas9^{1xMS2} mRNA (pSaCas9^{1xMS2}) and expressing both SaCas9^{1xMS2} mRNA and HBB sgRNA1 (pSaCas9^{1xMS2}-HBB sgRNA1) were made. As expected, transfecting pSaCas9^{1xMS2}-HBB sgRNA1 plasmid DNA into the GFP reporter cells generated significantly more GFP+ reporter cells than transfecting pSaCas9^{1xMS2} plasmid DNA (FIG. 7F). Since HBB sgRNA1 without the MS2 aptamer could not be packaged, LVLPs made from pSaCas9^{1xMS2}-HBB sgRNA1 plasmid DNA were expected to contain insufficient copies of HBB sgRNA1. Indeed, these LVLPs produced significantly less GFP+ reporter cells when transduced alone than when co-transduced with IDLV expressing HBB sgRNA1 (p<0.0001, FIG. 7F). Note that in this experiment, nonspecifically packaged sgRNA in LVLPs also contributed to the background activity, which explains why higher background activity was observed in FIG. 7F than in FIG. 7E. These observations argue against a major contribution from plasmid DNA left over during LVLP production, and support more effective packaging of SaCas9^{1xMS2} mRNA when using NC-MCP LVLPs.

Human beta hemoglobin (HBB) 3′ untranslated region greatly improved the genome editing activities of SaCas9 mRNA packaged in LVLPs To determine the best copy number of MS2 aptamer for packaging of SaCas9 mRNA, the effects of 0, 1, 2, 3 and 12 copies of MS2 aptamers on SaCas9 mRNA expression were examined. Equal amounts of plasmid DNA expressing SaCas9^{nxMS2} (n indicates 0, 1, 2, 3 or 12) was transfected into HEK293T cells and the steady-state level of SaCas9 mRNA was compared by RT-qPCR. Addition of 1 aptamer after the stop codon of SaCas9 slightly decreased the steady-state level of SaCas9 mRNA, while adding more than 1 aptamer significantly decreased SaCas9 mRNA (p<0.001, FIG. 9A). Consistent with the decrease in mRNA, when SaCas9^{nxMS2}-expressing DNA was co-transfected into GFP reporter cells with the plasmid expressing the HBB sgRNA1, one aptamer slightly decreased the percentage of GFP⁺ reporter cells, while more aptamers caused a further decrease (FIG. 9B). Consistently, when LVLPs containing SaCas9^{1xMS2}, SaCas9^{2xMS2}, SaCas9^{3xMS2}, or SaCas9^{12xMS2} were co-transduced into GFP-reporter cells with HBB sgRNA1-expressing lentivirus, SaCas9^{1xMS2} LVLPs generated the most GFP-positive cells, and SaCas9^{12xMS2} LVLPs generated the least at all concentrations tested (FIG. 9C).

To enhance SaCas9 mRNA stability and translatability with or without aptamer, two copies of 3′ untranslated region (UTR) sequences from the human HBB gene were added after the SaCas9 stop codon but before the MS2 aptamer. Plasmid DNA transfection in HEK293T cells showed that the steady-state level of SaCas9^{1xMS2}-HBB 3′ UTR was 1.3 fold of that of SaCas9^{0xMS2} (1.3±0.08 versus 1.0±0.03, n=4, p<0.05).

Since more than one MS2 aptamer significantly decreased the gene editing activities of the LVLPs, one copy of MS2 aptamer was further studied. LVLPs containing SaCas9 mRNA with or without MS2 aptamer and HBB 3′UTR (SaCas9^{0xMS2}, SaCas9^{1xMS2}, SaCas9^{0xMS2}-HBB 3′UTR, SaCas9^{1xMS2}-HBB 3′UTR) were made, and each type of particle was transduced into the GFP-reporter cells with HBB sgRNA1-expressing IDLV. Flow cytometry analysis revealed that without MCP, the LVLP produced GFP-positive cells inefficiently (dashed lines in FIG. 9D). Among LVLPs generated with MCP, the presence of both MS2 and HBB 3′UTR resulted in the highest genome editing activity. Surprisingly, SaCas9 mRNA without MS2 aptamer can also generate appreciable GFP-positive cells. SaCas9^{1xMS2} LVLPs showed lower activities than SaCas9^{0xMS2} LVLPs, consistent with a previous observation of MS2 decreasing SaCas9 mRNA stability. HBB 3′UTR significantly improved the performance of SaCas9^{1xMS2} LVLPs but not that of SaCas9^{0xMS2} LVLPs, another observation consistent with previous data. These data suggest that 1) MCP is necessary for efficient packaging of SaCas9 mRNA; and 2) MS2 aptamer decreases SaCas9 mRNA stability, although they may increase SaCas9 mRNA/NC-MCP association; and 3) HBB 3′UTR increased SaCa9 mRNA stability, and the presence of MS2 and HBB 3′UTR improved SaCas9 mRNA/NC-MCP association and SaCa9 mRNA stability.

Considering the possible packaging of dCas9 mRNA for CRISPR-mediated gene regulation, where sgRNA may need to be modified with aptamers to enhance gene regulation, more than one aptamer may be used in the same experiment. A PP7/PP7 coat protein (PCP) based packaging system was also developed to enable aptamer combinations. PP7/PCP is another RNA aptamer/ABP pair used in RNA studies. MCP was replaced with PCP in the packaging plasmid and the MS2 aptamer was replaced by the PP7 aptamer in the SaCas9 mRNA expression plasmid (expressing SaCas9^{1xPP7}). PP7/PCP packaged SaCas9 LVLPs could also efficiently generate GFP+ reporter cells when co-transduced into the GFP-reporter cells with the HBB sgRNA1-expressing IDLV (FIG. 9D). Surprisingly, a PCP-modified packaging system can also package PP7-free SaCas9 mRNA, and in this case, the packaging is PCP-dependent.

The GFP-reporter assay data were corroborated by RT-qPCR analysis of SaCas9 mRNA copy numbers per LVLP (FIG. 9E). Consistent with the observation that LVLPs containing SaCas9^{1xMS2}-HBB 3′UTR resulted in the highest percentage of GFP-positive cells, they had the highest SaCas9 mRNA copy number (˜50 fold RNA molecules contained in lentiviral vectors with similar p24 protein). Although SaCas9^{0xMS2} and SaCas9^{1xMS2} LVLPs had similar copies of Cas9 mRNA/particle, the gene editing activity of Cas9^{1xMS2} LVLPs was consistently lower than that of Cas9^{0xMS2} LVLPs (FIG. 9D). This could be the result of the MS2 aptamer decreasing SaCas9 mRNA stability.

Comparing the LVLPs packaged by MCP/MS2 and PCP/PP7 based systems, PCP/PP7 packaged SaCas9 LVLPs had better gene editing activities than those of MCP/MS2 packaged LVLPs when HBB 3′UTR was not added to SaCas9 mRNA. The opposite was true when HBB 3′UTR was added to SaCas9 mRNA (FIG. 9D). SaCas9^{1xMS2}-HBB 3′UTR LVLPs consistently showed the best gene editing activity in multiple repeats.

The expression duration of the mRNAs delivered by the LVLPs were examined and compared with that of IDLV. 24 hours after transduction, SaCas9^{1xMS2}-HBB 3′UTR LVLPs showed the highest Cas9 mRNA level, consistent with their high mRNA copy number/particle and mRNA stability (FIG. 10A). SaCas9^{1xMS2} LVLPs had the lowest mRNA expression, consistent with their low gene editing activity. In contrast to IDLV's increased Cas9 mRNA expression at 48 hours post transduction, suggesting transcription of new Cas9 mRNA, all mRNAs delivered by LVLPs decreased steadily with different speed. Compared with the respective mRNA levels of 24 hours post transduction, SaCas9^{1xPP7} showed similar decay rates as SaCas9^{1xMS2}-HBB 3′UTR (FIG. 10B), while SaCas9^{1xMS2} showed much faster decay rates. This could also explain why SaCas9^{1xMS2}-HBB 3′UTR and SaCas9^{1xPP7} performed better than SaCas9^{1xMS2} in GFP-reporter assays. Thus, SaCas9^{1xMS2}-HBB 3′UTR LVLPs were able to achieve transient and high expression compared to IDLV virus vectors.

Protein expression from the LVLPs was examined. Cas9 protein expression was readily detected in overexpressed HEK293T cells or GFP reporter cells with either the SaCas9 or HA (the Tag at the C-terminus of Cas9) antibodies, but could hardly detect Cas9 expression in GFP-reporter cells 48 hours after transducing even 300 ng LVLPs (FIG. 10C). These cells were co-transduced with HBB sgRNA1 IDLV and 15% of them were GFP-positive in flow cytometry analysis, suggesting successful gene editing in about 45% of the cells, even though Cas9 protein were difficult to detect in these cells by Western blotting. The data showed that the LVLPs were able to express sufficient Cas9 protein for efficient gene editing, although hardly detectable by the method we used. Since the rate of off-targets increases with increasing Cas9 protein, the relatively low Cas9 protein from LVLPs will also ensure low off-targets.

The Gag precursor protein was processed into mature proteins by sequential protease cleavage with different cleavage speed at different site (FIG. 10D). Antibodies specific for MA (p17), CA (p24) and p15 (from which NC is processed) were used to analyze the sizes of the processed proteins in normal GFP lentiviral vectors, MCP-based SaCas9^{1xMS2} LVLPs, and PCP-based SaCas9^{1xPP7} LVLPs. MA and CA protein from all particles showed the same expected size, indicating proper cleavage at the time of analysis (FIG. 10E). Anti-MA antibody detected weak 75 kDa bands in MCP and PCP-based LVLPs. However, similar bands were not detected by anti-CA or anti-p15 antibodies, arguing against the presence of unprocessed Gag precursor proteins. The anti-p15 antibody detected a band slightly smaller than 15 kDa in GFP lentivirus. This size indicates that at the time of analysis, cleavage at sites 4 and 5 did not occur at a detectable level, consistent with their low speed. p15 antibody detected bands between 20-25 kDa in PCP- and MCP-based LVLPs, matching the expected sizes of NC-PCP and NC-MCP fusion proteins in LVLPs (FIG. 10D). In MCP-based LVLPs, an additional small band was detected by the p15 antibody, indicating partial degradation of the p15-MCP fusion protein. These data suggest that the insertion of MCP or PCP in NC had little effect on Gag precursor cleavage at sites 1 (MA/CA), 2 (CA/SP1) and 3 (SP1/NC). The effects on cleavage at sites 4 (NC/SP2) and 5 (SP2/P6) were unknown since at the time of analysis these sites were not cleaved even in normal LV, judged from the size of peptide detected by p15 antibody. Electron microscopy analyses of the MCP- and PCP-based lentiviral-like particles revealed similar particle sizes as GFP lentivirus (FIG. 11).

LVLP packaged SaCas9 mRNA enables highly efficient genome editing To further determine the gene editing activity of the SaCas9 mRNA packaged LVLPs, we co-transduced LVLPs (30 ng p24 protein) packaged with SaCas9^{1xMS2}-HBB 3′UTR mRNA into 2.5×10⁴ GFP reporter cells with 60 ng p24 of HBB sgRNA1-expressing IDLV. 48 hours after transduction, 13% of the reporter cells became GFP-positive. The target DNA for HBB sgRNA1 in the GFP expression cassette was amplified and sequenced by next-generation sequencing. Overall, 86.5% of the alleles had Indels (FIG. 12A), mostly around the predicted cleavage site, which is 3 nt away from the PAM (FIG. 12B). The data demonstrated that delivering SaCas9 mRNA by the LVLPs is highly efficient in generating Indels.

To test the activities of SaCas9 LVLPs on endogenous targets, IDVL expressing sgRNA targeting human IL2RG, a mutation of which causes X-linked severe combined immunodeficiency (SCID-X1), were prepared. In HEK293T cells, 30 ng p24 of SaCas9 LVLPs and 60 ng of IL2RG sgRNA IDLV generated 13% Indels in the target sequence (FIG. 12C). In human lymphoblasts (2×10⁵ cells), co-transduction of 100 or 500 ng p24 of both types of particles generated 11% or 87.6% Indels in the target sequence (FIG. 12D). Although the Indel rates were different under different conditions, the top most frequently observed mutations were the same. The data showed that the SaCas9 LVLPs could be used to target different target genes in multiple cells.

LVLP packaged SaCas9 mRNA showed lower off-target rates than those of viral vectors. The HBB sgRNA1 was designed to target the HBB Sickle cell mutation and has one nucleotide mismatch with the wild-type HBB sequence (FIG. 12E). As such, the corresponding wild-type HBB gene sequence in the GFP reporter cells can be regarded as the “off-target” for SaCas9/HBB sgRNA1. Indel rates in the wild-type HBB locus were compared when SaCas9 was delivered by LVLP, AAV6, IDLV, or LV expressing SaCas9. To control the percentage of cells having functional SaCas9/HBB sgRNA1, GFP-positive cells were sorted by GFP-activated sorting 48 hours after transduction. At the time of sorting (48 hours after transduction), the percentage of GFP-positive rate was 11.1%, 2.1%, 8.5% and 6.5% for cells treated with LV, LVLP, IDLV, and AAV6 (10⁴ vg/cell), respectively. Although the cells were treated with the same number of LV, LVLP and IDLV particles based on p24 amount (about 750 ng p24 for 1.25×10⁶ cells), integration-competent LV treated cells already showed higher GFP-positive rates than IDLV due to continuous CRISPR/Cas9 expression. After sorting, the GFP-positive rates were 95.4%, 88.9%, 93.3%, and 90.8% respectively, suggesting the presence of functional SaCas9/HBB sgRNA1 in most cells under each condition. The DNA from the endogenous HBB locus was then amplified from the sorted cells 1 week after sorting and subjected for next-generation sequencing. The LVLP system had the lowest Indel rate in the endogenous HBB site and LV had the highest (FIG. 12E). These data demonstrate that delivering SaCas9 mRNA by LVLPs for gene editing is safer than other virus delivery systems.

In the cells described above, Indels in 9 possible off-targets predicted by Cas-Offinder) and CRISPOR were searched for. No Indels were detected in 8 of the 9 potential HBB sgRNA1 off-targets even in LV treated cells. However, in off-target 8 (chromosome 20, 33982230 bp-33982337 bp), slightly more Indels were detected in LV-transduced cells than in LVLP transduced cells (FIG. 12F).

Since there were GFP-reporter cells transduced with different particles that showed different percentages of GFP-positive cells (cells used in FIG. 12A and unsorted cells for FIG. 12E), the relationship between GFP-positive percentages and target sequence (in the GFP-expression cassette) Indel rates in these cells was analyzed. It was found that Indel rates increased with the GFP-positive percentage linearly (FIG. 13, r²=0.9497 by linear regression). The data validated the GFP-reporter assay described herein for comparing the genome editing activities of different particle types.

### Example 11. Packaging Cas9/sgRNA RNP into Lentivirus-Like Particles

Plasmids pMD2.G (Addgene #12259), psPAX2-D64V (Addgene #63586), pCSII-EF-miRFP709-hCdt (1/100) (Addgene #80007), and pX601-AAV-CMV::NLS-SaCas9-NLS-3xHA-bGHpA;U6::BsaI-sgRNA (Addgene #61591) were purchased from Addgene and have been described previously. Plasmids are described in Example 10 or Table 2. All constructs generated were sequence confirmed. Sequence information for primers, oligoes, and synthesized DNA fragments is in Table 10.

GFP reporter assays for gene editing activities The EGFP reporter cells described above were used to detect gene editing activity of SaCas9/human beta hemoglobin (HBB) sgRNA1 or SaCas9/IL2RG-sgRNA1 on the inserted target sequences in the GFP-reporter cassette. The GFP-reporter cells expressed no EGFP due to the disruption of the EGFP reading frame by the insertion of the HBB sickle mutation and IL2RG target sequences between the start codon and the second codon of EGFP coding sequence. Indels formed after gene editing may restore the EGFP reading frame, resulting in EGFP expression. GFP-positive cells were analyzed by fluorescence microscopy or flow cytometry (BD Biosciences, Accuri C6) as described above and in Lu et al., Nucleic Acids Res. 2019 doi: 10.1093/nar/gkz093.

AAV6 virus production and transduction Adeno-associated virus expressing SaCas9 and HBB sgRNA1 was made from the AAV vector pSaCas9 (expresses SaCas9) and pSaCas9-HBB-sgRNA1 (expresses HBB sgRNA1, and contains donor template for homologous recombination to change the wild-type HBB gene to the Sickle mutation) respectively. AAV serotype 6 (AAV6) production and quantification were performed by Virovek, Inc. (Hayward, CA). AAV6 transduction was performed in serum-free medium or OPTI-MEM at a titer of 10³-10⁴ virus genome/cell. 24 hours after transduction the cells were returned to serum-containing growth medium.

Lentivirus and LVLP production Lentiviral vector plasmid pCK002-HBB-sgRNA1 expressing both SaCas9 and HBB sgRNA1 was used to produce integration-competent lentiviral vector (packaged by packaging plasmid pspAX2) and integration-defective lentiviral (IDLV) vector (packaged by packaging plasmid pspAX2-D64V) as described above. SaCas9 mRNA LVLP production is also described above. To produce Cas9/sgRNA RNP LVLPs, 13 million actively dividing HEK293T cells, grown in a 15-cm dish were changed to 10 ml Opti-MEM. 16 μg of ABP-modified packaging plasmid pspAX2-D64V-NC-ABP (ABP could be MCP, PCP, 2 N22 or COM), 6 μg envelope plasmid (pMD2.G), and 16 μg plasmid DNA co-expressing SaCas9 and the aptamer-modified sgRNA were mixed in 1 ml Opti-MEM. 76 μl of 1 mg/ml polyethylenimine (PEI, Polysciences Inc.) was mixed in 1 ml Opti-MEM. The DNA mixture and the PEI mixture were then mixed and incubated at room temperature for 15 mins. The DNA/PEI mixture was then added to the cells in Opti-MEM. 24 h after transfection, the medium was changed to 15 ml Opti-MEM and the Cas9/sgRNA RNP LVLPs were collected twice with one collection each 24 h. The supernatant was spun for 10 min at 500 g to remove cell debris before further processing described below.

Concentrating lentivirus and LVLPs The collected and cleared supernatant was concentrated with the KR2i TFF System (KrosFlo® Research 2i Tangential Flow Filtration System) (Spectrum Lab, Cat. No. SYR2-U20) using the concentration-diafiltration-concentration mode. Typically, 150-300 ml supernatant was first concentrated to about 50 ml, diafiltrated with 500 ml to 1000 ml PBS, and finally concentrated to about 8 ml. The hollow fiber filter modules were made from modified polyethersulfone, with a molecular weight cut-off of 500 kDa. The flow rate and the pressure limit were 80 ml/min and 8 psi for filter module D02-E500-05-N, and 10 ml/min and 5 psi for the filter module C02-E500-05-N.

Lentiviral vector and LVLP quantification Viral titer was determined by p24 based ELISA (Cell Biolabs, QuickTiter™ Lentivirus Titer Kit Catalog Number VPK-107). When unpurified samples were assayed, the viral particles were precipitated according to the manufacturer's instructions so that the soluble p24 protein was not detected.

Western blotting analysis of viral proteins from lentivirus and LVLPs Purified lentivirus or LVLPs (200 ng p24 by ELISA) were lysed in 20 μl of 1× Laemmli sample buffer. The proteins in each sample were separated on SDS-PAGE gels and analysed by Western blotting. The antibodies used include mouse monoclonal anti-SaCas9 antibody (Millipore Sigma, MAB131872, clone 6F7, 1:1000), rabbit polyclonal HIV1 p17 antibody for MA (ThermoFisher Scientific, Cat No. PA1-4954, 1:1000), rabbit polyclonal HIV1 p15 antibody for NC (Abcam, Cat No. ab66951, 1:1000) and p24 mouse monoclonal antibody for CA (Cell Biolabs, Cat No. 310810, 1:1000). HRP conjugated anti-Mouse IgG (H+L) (ThermoFisher Scientific, Cat No. 31430, 1:5000) and anti-Rabbit IgG (H+L) (Cat No. 31460, 1:5000) secondary antibodies were used in Western blotting. Chemiluminescent reagents (Pierce) were used to visualize the protein signals under the LAS-3000 system (Fujifilm).

RNA isolation from lentivirus or LVLPs and RT-qPCR analysis A miRNeasy Mini Kit (QIAGEN Cat No. 217004) was used to isolate RNA from concentrated lentivirus or LVLPs. The QuantiTect Reverse Transcription Kit (QIAGEN) was used to reverse-transcribe the RNA to cDNA. For sgRNA reverse transcription, half random primers provided in the kit and half sgRNA-specific primers (sgRNA-R2) were used for reverse transcription. Custom designed hydrolysis probes specific for SaCas9 and EGFP (ThermoFisher Scientific) were used in qPCR, together with TaqMan Universal PCR Master Mix (ThermoFisher Scientific). For HBB sgRNA1 and HBB sgRNA1^{Tetra-com} detection, sgRNA-F1 (SEQ ID NO: 30) and s0067RNA-R3 were used as primers in SybrGreen based RT-qPCR. PCR was run on an ABI 7500 instrument.

Removing membrane from LVLP core capsids Lentivirus-like particles were transiently treated with 0.5% Triton X-100 as described in Wiegers et al. J. Virol. 1998, 72:2846-2854. Briefly, LVLPs were centrifuged with a Sorvall T-890 rotor (2 h at 120 000 g) through step gradients containing a 1 ml layer of 10% sucrose in STE [100 mM NaCl, 50 mM Tris/HCl (pH 7.5), 1 mM EDTA] with or without 0.5% Triton X-100, and a cushion of 2 ml 20% sucrose in STE solution. Pelleted virus was directly lysed for Western blotting or RT-qPCR analysis.

Lentivirus and LVLP transduction Various amounts of concentrated lentivirus or LVLPs (equivalent to 10˜300 ng p24 protein) were added to 2.5×10⁴ cells grown in 24-well plates, with 8 μg/ml polybrene. Unconcentrated virus containing supernatant was diluted with fresh medium at a 1:1 ratio to transduce cells. The cells were incubated with the particle containing medium for 12-24 hours, after which normal medium was replaced.

Gene editing in human cells For gene editing with Cas9 expressed from AAV serotype 6, the SaCas9 expressing AAV6 and the HBB sgRNA1 expressing AAV6 were co-transduced into GFP reporter cells. For gene editing with LV or IDLV (packaged with packaging plasmids containing a D64V mutation in the integrase) expressing both SaCas9 and HBB sgRNA1, virus equivalent to 10-300 ng p24 was used to transduce 2.5×10⁴ cells in 24 well plates. For gene editing with SaCas9 mRNA LVLPs, various amounts of SaCas9 mRNA LVLPs (quantified by p24) were co-transduced into HEK293T cells or the GFP reporter cells with an IDLV expressing HBB sgRNA1. For transduction with Cas9/sgRNA RNP LVLPs, various amounts of Cas9/sgRNA RNP LVLPs were transduced into human cells. 48-72 hours after transduction, gene editing activity was analyzed by GFP-reporter assay or next-generation sequencing.

To examine gene editing in human lymphoblastoid cells immortalized by Epstein-Barr virus transformation, human lymphoblastoid cell lines with or without sickle cell mutations were purchased from Corrie Institute (GM16265, with Sickle cell mutation; ID00085, with mutation in IL2RG gene). The lymphoblasts were cultured in RPMI 1640 with 2 mmol/L L-glutamine and 15% fetal bovine serum at 37° C. under 5% carbone dioxide. For LVLP and IDLV transduction, 2×10⁵ cells were added to 0.5 ml RPMI growth medium. Then Cas9/sgRNA RNP LVLPs were added to the cells. Polybrene was added in the medium to a final concentration of 8 μg/ml. Fresh medium was replaced twenty hour hours after transduction. The cells were collected 72 hours after transduction for DNA analysis by next-generation sequencing.

Next-generation sequencing and data analysis The endogenous HBB target sequence and IL2RG target sequence, and the HBB and IL2RG target sequence in the integrated GFP-expression cassette were amplified for sequencing analysis. A nested PCR strategy was used to amplify the endogenous HBB target sequence to avoid amplifying the sequence from the viral vector template. First, primers HBB-1849F and HBB-5277R were used to amplify the 3.4 kb region from the HBB gene locus. These two primers cannot amplify sequences from the templates in the viral vectors. Then HBB-F1 and the HBB-F2 primers were used to amplify the target DNA for sequencing. To amplify the endogenous IL2RG target sequence, primers IL2RG-1029F and IL2RG-3301R were used to amplify the target region from the treated cells (unable to amplify sequences from the templates in the viral vectors), then primers IL2RG-F1 (SEQ ID NO: 90) and IL2RG-3301R were used to amplify the target DNA from the first PCR product for sequencing. To amplify the HBB target sequence from the integrated EGFP reporter for sequencing, Reporter-F and Reporter-R1 primers were used. The proofreading HotStart® ReadyMix from KAPA Biosystems (Wilmington, MA) was used for PCR. The purified PCR products were shipped to Genewiz Inc. (Morrisville, NC) to perform next generation sequencing (Amplicon EZ). Usually 50,000 reads/amplicon were obtained.

After removing the 3′ linker and 5′ barcode sequences, the resulting reads were submitted to the online Cas-Analyzer software for mutation analysis. When submitting the reference sequence, the primer sequences were excluded if they were at least 12 nt away from the sgRNA target. Otherwise primer sequences were included in the reference. Only those readings containing both the 5′ 12 nt and 3′ 12 nt of the reference sequence (primer sequences excluded) were included in Indel analysis. The total Indel rate was the difference between 1 and the percentage of readings without mutation. The top 8-10 most frequently observed readings were presented.

Monitoring the speed of GFP-positive cell emergence 2.5×10⁴ GFP-reporter cells seeded in 24-well plates were transduced with 50 ng p24 of Cas9/IL2RG sgRNA1^{Tetra-com} RNP LVLPs, or co-transduced with 100 ng p24 of Cas9 mRNA LVLPs and 100 ng p24 of IDLV expressing IL2RG sgRNA1. The cells were then incubated in the IncuCyte S3 system (Essen BioScience, Inc. Ann Arbor, Michigan) for timed GFP fluorescence scanning. Two wells from each treatment and 9 spots from each well were scanned. The scanning started right after transduction and the cells were scanned once every two hours for 48 hours. The GFP-positive rate of each image was calculated by dividing the GFP-positive area by the phase area (area occupied by cells) in that image.

Statistical analysis GraphPad Prism software was used for statistical analyses. T-tests were used to compare the averages of two groups. Analysis of Variance (ANOVA) was performed followed by Tukey post hoc tests to analyze data from more than two groups. Bonferroni post hoc tests were performed following ANOVA in cases of two factors. p<0.05 was regarded as statistically significant.

Replacing the sgRNA Tetraloop with aptamer best preserved the Cas9/sgRNA RNP activity. Packaging Cas9/sgRNA RNP into lentivirus-like particles was tested. The overall strategy was to incorporate ABP into lentivirus-like particles via fusing ABP with the lentiviral nucleocapsid protein (NC) as described above and add the corresponding aptamer into sgRNA, which complexes with Cas9 protein, and forms Cas9/sgRNA RNP during lentiviral capsid assembly. The Cas9/sgRNA RNP is packaged into the lentiviral capsids via the specific aptamer/ABP interaction (FIG. 14A, right diagram). After escaping from the endosomes, the Cas9/sgRNA RNP is released into the cytoplasm, following capsid uncoating, and the RNP complex then enters the nucleus to perform gene editing.

In order for this strategy to work, it is necessary to find a location in the sgRNA scaffold best tolerating aptamer insertion and preserving the nuclease activity after complexing with Cas9. The MS2 aptamer was used, since it mediated efficient Cas9 mRNA packaging. Three locations were tested: inserting MS2 into the Stem loop 2 (ST2), replacing the Tetraloop with MS2, and adding MS2 after the 3′ end of the sgRNA (FIG. 14B). When the plasmid DNAs co-expressing SaCas9 and the modified sgRNAs targeting HBB sgRNA1 were transfected into the GFP-reporter cells, Indels in HBB sgRNA1 target sequence may restore GFP expression. It was found that adding one MS2 at any of the three locations preserved gRNA activity in transfection experiments, although replacing the Tetraloop or the ST2 loop slightly decreased the percentage of GFP-positive reporter cells (FIG. 14C). The addition of two MS2 aptamers was also tested. One aptamer replaced the Tetraloop, and the other one was in the ST2 loop or at the 3′ end. In both cases, the GFP-positive percentages were consistently decreased, agreeing with observations that more than one copy of MS2 decrease RNA stability. Thus, one copy of aptamer was used in further experiments.

Whether these MS2-modified sgRNAs could be packaged and delivered by LVLPs was also tested. SaCas9 protein and MS2-modified HBB sgRNA1 were co-expressed during LVLP preparation, and the LVLPs were transduced into our GFP reporter cells. Flow cytometry analysis found that LVLPs containing HBB sgRNA1 with MS2 at the Tetraloop (HBB sgRNA1^{Tetra MS2}) gave the most GFP-positive cells, LVLPs containing HBB sgRNA1 with MS2 at the 3′ end (HBB sgRNA1^{3′MS2}) followed. In contrast with transfection experiments, where MS2 at Stem loop II (HBB sgRNA1^{ST2 MS2}) was active, HBB sgRNA1^{ST2 MS2} LVLPs had hardly any gene editing activity (FIG. 14D), suggesting that either HBB sgRNA1^{ST2 MS2} could not be packaged or could not survive the post-transduction process.

com/COM is an aptamer/ABP pair for sgRNA packaging Knowing that replacing the Tetraloop with aptamers performed the best, additional aptamers that could be used to replace the Tetraloop were investigated. Four aptamers, MS2 (23), PP7 (24), BoxB (25) and com (26), have been used in sgRNAs for target-binding purposes (22,27-29). The activities of HBB sgRNA1 with various aptamers (MS2, PP7, BoxB and com) replacing the Tetraloop (FIG. 15A) were tested. When plasmid DNA co-expressing SaCas9 and various aptamer-modified HBB sgRNA1 was transfected into GFP reporter cells, replacing Tetraloop with com aptamer generated the same rates of GFP-positive cells as the unmodified sgRNA, while replacing Tetraloop with the other three aptamers significantly reduced the GFP-positive rates (FIG. 15B).

Packaging of HBB sgRNA1 into LVLPs with different aptamers was tested. For this purpose, MCP (MS2 coat protein, binding to MS2), PCP (PP7 coat protein, binding to PP7), λ N22 peptide (binding to BoxB) and COM (binding to com) modified packaging plasmids, where ABPs were inserted after the second zinc finger domain of nucleocapsid protein (NC) as described above, were used to make LVLPs containing RNPs in which Cas9 complexed with various modified HBB sgRNA1. These LVLPs were transduced into GFP-reporter cells. Flow cytometry found that LVLPs generated by com/COM pair had the most GFP-positive cells, followed by those generated by MS2/MCP. PP7/PCP and BoxB/λ N22 generated LVLPs had the lowest activity (FIG. 15C). GFP-positive cells could only be observed when these LVLPs were used to transduce GFP-reporter cells but not HEK293T cells, excluding possible contamination of GFP-expressing DNA or virus (FIG. 16).

Since com-COM combination obtained the best results, whether COM-modification of the packaging plasmid impairs particle assembly was tested. A marginal 11% decrease in particle assembly efficiency compared with the unmodified packaging plasmid (FIG. 17) was observed. This slight decrease should not prevent production of sufficient particles. Through these experiments, the location (Tetraloop) and the aptamer/ABP pair (com/COM) for the most efficient packaging of Cas9 RNPs (or Cas9 mRNA and sgRNA) into lentiviral capsids was identified.

Cas9/sgRNA RNPs in the LVLPs accounted for the observed gene editing activity Although designed to package Cas9/sgRNA RNPs, considering previous observations that aptamer-free SaCas9 mRNA could be packaged for unknown mechanisms, the observed gene editing activity could be from the co-packaged SaCas9 mRNA and sgRNA. To determine whether Cas9/sgRNA RNP or SaCas9 mRNA/sgRNA contributed to the observed gene editing activity, the Cas9 expression cassette was separated from the HHB sgRNA1^{Tetra-com} expression cassette into two different plasmids. When the two plasmids were co-transfected into GFP reporter cells, they generated GFP-positive cells as efficiently as transfecting a single plasmid containing both expression cassettes (FIG. 18A). However, when HHB sgRNA1^{Tetra-com} was packaged into LVLPs, in the absence of Cas9 expression, these LVLPs had little gene editing activity when co-transduced into GFP-reporter cells with validated functional Cas9 mRNA LVLPs (FIG. 18B).

Cas9 protein is needed to protect the stability of sgRNA in cells. This was confirmed by expressing HBB sgRNA1^{Tetra-com} alone or co-expressing sgRNA with Cas9. Co-expressing Cas9 significantly increased the expression of HBB sgRNA1^{Tetra-com} (FIG. 18C). After DNA transfection, the sgRNA was constitutively expressed. Whereas after transduction, the sgRNAs had to complete the post-transduction intracellular traffic and there was no new sgRNA generation. Thus, it is expected that the protective effects of Cas9 protein on sgRNA would be more critical. These data suggest that sgRNA instability, especially sgRNA's inability to survive the post-transduction intracellular traffic, most likely explains why singly packaged HHB sgRNA1^{Tetra-com} LVLPs were inactive after co-transduction with Cas9 mRNA LVLPs.

These experiments show that, in addition to co-packaged Cas9 mRNA and sgRNA, the packaged Cas9/sgRNA RNPscan also be used for gene editing. To examine the presence of Cas9 protein in the HHB sgRNA1^{Tetra-com} LVLPs packaged with Cas9 expression, Western blotting analyses on the concentrated LVLPs (FIG. 18D) was performed. Viral proteins MA (p17) and CA (p24) were detected, as expected in size and amount, indicating that the processing of MA and CA was unaffected. Detection of p15, from which NC was processed, showed a band between 10 kd and 15 kd in GFP lentivirus (lane 1) and in LVLPs generated with the unmodified packaging plasmid (lane 3). In LVLPs generated by a NC-MCP modified packaging plasmid (lane 2), a strong band between 15˜20 kd was detected, which was slightly smaller than the expected 21.8 kd NC-MCP fusion protein. However, in LVLPs generated by NC-COM modified packaging plasmid (lanes 4˜6), a band slightly smaller than 15 kd was detected, which was slightly smaller than the expected 18.7 kd of the NC-COM fusion protein. Anti-p15 detected bands slightly smaller than expected in all samples (including GFP lentivirus), which could be caused by the SDS-PAGE system or by partial degradation of the p15 or p15-fusion proteins.

Surprisingly, Cas9 protein was detected in all types of LVLPs produced in cells with Cas9 expression, including samples without sgRNA (lane 3, observed after longer exposure), or without tetra-com aptamer in the sgRNA (lane 6). However, Cas9 was not detected in GFP-expressing lentivirus (FIG. 18D, lane 1). The detection of Cas9 proteins in the LVLPs suggested that Cas9 protein could contribute to the observed gene editing activities, but could not explain the lack of activity from LVLPs containing Cas9 protein and sgRNA without com-modification.

com-modification of sgRNA was necessary for efficiently packaging Cas9/sgRNA RNPs into the core capsid of LVLPs Cas9 protein was detected in LVLPs with HBB sgRNA1 without a com-modification (FIG. 18D, lane 6). These showed little gene editing activity in GFP-reporter assay (FIG. 18B). Therefore, the relative levels of Cas9 protein (normalized to capsid protein CA) in LVLPs with and without com aptamer were compared to determine if the amount of Cas9 protein per particle explains the difference in activity. It was found that the Cas9/HBB sgRNA1^{Tetra-com} LVLPs (com⁺) contained 2.9 fold Cas9 protein of Cas9/HBB sgRNA1 LVLPs (FIG. 18E, com⁻).

Since there was a less than 3-fold difference in Cas9 protein content, the compartmentalization of Cas9 protein was studied. Since the LVLPs were concentrated by tangential flow filtration, the LVLPs could have membrane structures, including exosomes. It was postulated that RNPs within the capsid core most likely preserve gene editing activity during the post-transduction intracellular traffic, thus the amount of detergent-resistant Cas9 protein matters. To detect Cas9 protein within the capsid core, the particles were transiently treated with 0.5% Triton X-100 to eliminate Cas9 proteins associated with membrane vesicles and capsid envelope. Triton X-100 treatment greatly decreased the amount of MA protein (detected by p17 antibody) associated with the capsid envelope, indicating that the treatment worked (FIG. 18F). The CA protein was reduced to variant degrees, which could reflect different core capsid stability of different types of particles. However, the Cas9 protein amount was greatly reduced in LVLPs with the unmodified sgRNA, but only slightly reduced in the LVLPs with Tetra-com modified sgRNA. Cas9/HBB sgRNA1^{Tetra-com} LVLPs had 6.8 fold detergent-resistant Cas9 protein compared to Cas9/HBB sgRNA1 LVLPs. These data show that Tetra-com modification of sgRNA facilitated the packaging of Cas9 protein in the detergent-resistant capsid core, and the amounts of core-protected Cas9/sgRNA RNPs correlated with gene editing activity.

The importance of com-aptamer on sgRNA packaging was examined by RT-qPCR analysis of sgRNA content in LVLPs. Although the PCR primers used produced amplicons of different sizes from HBB sgRNA1 and HBB sgRNA1^{Tetra-com}, due to the presence and absence of the 23 nt com-aptamer, the primers generated very similar standard curves when variant amounts of plasmid DNA were used as the templates for qPCR (FIG. 19), demonstrating similar amplification efficiency for the com-positive and com-negative sgRNA sequences. In LVLPs without Cas9 protein, the HHB sgRNA1^{Tetra-com} level was 2.5 times greater than in LVLPs with Cas9 protein. However, in LVLPs with Cas9 and com-negative HBB sgRNA1, the sgRNA level was less than 1/50 of that in LVLPs with Cas9 and HHB sgRNA1^{Tetra-com} (FIG. 18G). These data showed that the packaging of sgRNA was com-aptamer but not Cas9 protein-dependent. Cas9 protein slightly decreased the amount of com-modified sgRNA packaged, most likely due to the negative effects of Cas9/sgRNA association on com/COM interaction. Thus these data showed that the packaging of Cas9 protein into the capsid core is mediated by sgRNA^{Tetra-com} via com/COM interaction. Although Cas9 protein is not needed for packaging sgRNA into the LVLPs, it is needed to protect the sgRNAs during transduction. Except for experiments described in FIG. 18, where sgRNA with and without com-modification were used for comparison, com-modified sgRNA was used in all subsequent experiments. Hereafter, Cas9/sgRNA RNP was used to indicate Cas9/sgRNA^{Tetra-com} RNP for simplicity.

Cas9/sgRNA RNP LVLPs enabled efficient genome editing and improved discrimination of off-targets The gene editing activities of Cas9 RNP LVLPs described and the Cas9 mRNA LVLPs described above were compared. Cas9/HHB sgRNA1 RNP LVLPs showed comparable gene editing activities as compared to the SaCas9 mRNA LVLPs in GFP-reporter assays (FIG. 20A).

The gene editing activities of the RNP LVLPs were further examined by next generation sequencing (NGS) on another target, IL2RG, whose mutation causes the X-linked severe combined immunodeficiency, using IL2RG sgRNA1 described above. Cas9/IL2RG sgRNA1 RNP LVLPs were transduced into 2.5×10⁴ GFP reporter cells. 72 hours after transduction, the endogenous IL2RG region was amplified and subjected to NGS. 15, 45, and 100 ng p24 of these particles generated 4.1%, 8.1% and 21.1% Indels in the endogenous IL2RG gene (FIG. 20B). In the studies described above, 30 ng p24 of SaCas9 mRNA LVLPs and 60 ng of IL2RG sgRNA LV generated 13% Indels in IL2RG of HEK293T cells, thus these RNP LVLPs showed comparable or better gene editing activity. 200 ng p24 of IL2RG sgRNA1 RNP LVLPs were also transduced into 2×10⁵ B lymphoblastoids, and 18.3% Indels were detected (FIG. 21). This activity is also comparable to 100 ng p24 of SaCas9 mRNA LVLPs and 100 ng p24 of IL2RG sgRNA1 expressing IDLV, which generated 11% Indels in the same number of B lymphoblastoids.

These data showed that the RNP LVLPs were as active as Cas9 mRNA LVLPs in gene editing. Delivering Cas9 by RNP should offer better control of the amount of Cas9 protein delivered per cell and more transient Cas9 action. Both would help to reduce off-target rates. The predicted 9 potential HBB sgRNA1 off-targets all had very low Indels, preventing comparison of off-target rates between different delivery methods. However, the wild-type HBB sequence corresponding to the Sickle cell disease mutation has only 1 nucleotide mismatch with HBB sgRNA1 (FIG. 20C) and detectable off-target Indels could be generated by Cas9/HBB sgRNA1. Thus, the on-target Indel rates (in the Sickle cell disease mutation in GFP reporter cassette, shown at the bottom of FIG. 20C) and the off-target Indel rates (in the endogenous wild-type HBB sequence with 1 nucleotide mismatch to HBB sgRNA1, shown on the top of FIG. 20C) in GFP-reporter cells, when Cas9/HBB sgRNA1 was delivered by plasmid transfection, or by LV, IDLV and AAV, were studied. Results showed that Cas9/HBB sgRNA1 RNP LVLP had the highest ratio of on-target to off-target Indel rates, Cas9 mRNA/HBB sgRNA1 LVLPs had the second, and both were higher than those of LV, IDLV and AAV (FIG. 20D). Thus RNP LVLPs best distinguished on-target effects from off-target effects.

Cas9/sgRNA RNP LVLPs showed faster action than Cas9 mRNA LVLPs after transduction GFP reporter assays were used to compare the kinetics of GFP-positive cell emergence after Cas9/sgRNA RNP LVLP and Cas9 mRNA LVLP treatment. For this purpose, 2.5×10⁴ GFP reporter cells were transduced with 50 ng p24 of Cas9/IL2RG sgRNA1 RNP LVLPs, or co-transduced GFP reporter cells with 100 ng p24 of Cas9 mRNA LVLPs and 100 ng p24 of IDLVs expressing IL2RG sgRNA1. The emergence of GFP-positive cells was monitored every two hours. GFP-positive cells showed up at least 6 hours earlier in the RNP LVLP treated cells than in the mRNA LVLP treated cells (FIG. 20E). 34 hours after transduction, the GFP-positive area rates of the two treatments converged. The data showed that, although mRNA LVLPs can be used effectively, RNP LVLPs have faster actions than mRNA LVLPs and IDLVs. This is likely because the RNPs are available for function in the nucleus soon after escaping from the endosome, while the mRNA LVLPs and IDLVs need more time to express Cas9 protein and sgRNA. The difference in kinetics also indicates that the functional components in Cas9/sgRNA RNP LVLPs are different from those in the Cas9 mRNA LVLPs.

LVLPs mediated efficient homologous recombination in the presence of donor templates One of the applications of CRISPR/Cas9 is to facilitate homologous recombination in the presence of donor template. Integration-defective lentiviral vectors have been used to provide donor template. COM-modification of NC in the packaging plasmid slightly decreased the packaging of a GFP-expressing lentiviral vector (1.0±0.15, N=3 for unmodified packaging plasmid; 0.73±0.15, N=6 for NC-COM modified packaging plasmid; p>0.05), but almost eliminated GFP expression after transduction of these COM-modified GFP lentiviral vectors. In addition, packaging of the Cas9/sgRNA RNP decreased-mediated lentiviral genome packaging, although the difference did not reach statistical significance (0.53±0.16, N=3 with RNP; 0.93±0.21, N=3 without RNP; p>0.05). In view of these observations, donor template was delivered separately by an integration-defective lentiviral vector (IDLV).

Lentiviral vectors are widely used for ex vivo modification of cells. LVLPs were used to facilitate the insertion of a functional IL2RG cDNA downstream the endogenous IL2RG promoter. This cDNA insertion strategy can be used to treat SCID-X1 regardless of the nature of the disease causing mutations. The donor template contains the 1.5 kb 5′ homology arm, the 1.1 kb IL2RG cDNA, and the 3′ homology arm. The 3′ homology arm was designed so that after homologous recombination, the cDNA replaces similar length of the IL2RG genomic DNA. The IL2RG cDNA was codon optimized to maximize the difference with the original cDNA in order to reduce the chance of cDNA-mediated homologous recombination. In addition to the donor template, the lentiviral vector also contained two U6 promoters driving the expression of IL2RG sgRNA1 and sgRNA2 to facilitate the insertion of the cDNA (FIG. 22A.).

The donor template/sgRNAs were delivered to lymphoblast cells by IDLV, with or without Cas9/IL2RG sgRNA1 RNP LVLPs. 72 hours after transduction, genomic DNA was extracted and the target region was amplified by two rounds of PCR amplification (FIG. 22A). In the first PCRs, one primer was outside the donor template, and thus could not amplify from the template DNA without homologous recombination. The products of the first PCR were purified from the gel and used as the templates in the second PCRs to amplify the 5′ and 3′ junction DNA for sequencing. Three primers were used in each PCR, in addition to a common primer. One primer matched the inserted cDNA sequence to amplify the DNA with HR, and another primer matched the genomic DNA being replaced to amplify the DNA without HR. 100 ng p24 of LVLP and IDLV were transduced into 2.5×10⁴ HEK293T cells. At the 5′ junction, 46.2% sequences without HR (29.6% without Indels and the rest 16.6% with Indels) and 53.8% sequences with HR (FIG. 22B) were observed. In the readings with HR, Indel rate was similar to background since no target sequence was present in the donor templates. The data showed that the Cas9/sgRNA RNPs were active and greatly enhanced the targeted insertion of a 1.1 kb DNA. The observation of 57% readings with HR in the PCR products suggests that these LVLPs can be used together with IDLV to enhance HDR.

What is claimed is:

1. A lentiviral packaging plasmid comprising a eukaryotic promoter operably linked to a Gag nucleotide sequence, wherein the Gag nucleotide sequence comprises a nucleocapsid (NC) coding sequence and a matrix protein (MA) coding sequence, wherein one or both of the NC coding sequence or the MA coding sequence comprises at least one non-viral aptamer-binding protein (ABP) nucleotide sequence, and wherein the packaging plasmid does not encode a functional integrase protein.

2. The lentiviral packaging plasmid of claim 1, wherein the NC coding sequence comprises two functional zinc finger protein domains and functional native protease processing sequences.

3. The lentiviral packaging plasmid of claim 1 or 2, wherein the at least one non-viral ABP nucleotide sequence encodes MS2 coat protein, PP7 coat protein, lambda N peptide, or COM protein.

4. The lentiviral packaging plasmid of any one of claims 1-3, wherein the Gag nucleotide sequence comprises a first non-viral ABP nucleotide sequence and a second non-viral ABP nucleotide sequence in tandem.

5. The lentiviral packaging plasmid of claim 4, wherein the first non-viral ABP nucleotide sequence and the second non-viral ABP nucleotide sequence both encode the same ABP, wherein the ABP comprises MS2 coat protein, PP7 coat protein, lambda N peptide, or COM protein.

6. The lentiviral packaging plasmid of claim 4, wherein the first non-viral ABP nucleotide sequence and the second non-viral ABP nucleotide sequence encode different ABPs selected from the group consisting of MS2 coat protein, PP7 coat protein, lambda N peptide, and COM protein.

7. The lentiviral packaging plasmid of any one of claims 1-6, wherein the NC coding sequence comprises at least one first non-viral ABP nucleotide sequence and the MA coding sequence comprises at least one second non-viral ABP nucleotide sequence.

8. The lentiviral packaging plasmid of claim 7, wherein the at least one first non-viral ABP nucleotide sequence and the at least one second non-viral ABP nucleotide sequence both encode the same ABP, wherein the ABP comprises MS2 coat protein, PP7 coat protein, lambda N peptide, or COM protein.

9. The lentiviral packaging plasmid of claim 7 or 8, wherein the at least one first non-viral ABP nucleotide sequence and the at least one second non-viral ABP nucleotide sequence encode different ABPs selected from the group consisting of MS2 coat protein, PP7 coat protein, lambda N peptide, and COM protein.

10. The lentiviral packaging plasmid of any one of claims 1-9, further comprising a Rev nucleotide sequence and a Tat nucleotide sequence.

11. The lentiviral packaging plasmid of any one of claims 1-10, wherein the lentiviral packaging plasmid comprises an integrase coding sequence with an integrase-inactivating mutation therein.

12. The lentiviral packaging plasmid of claim 11, wherein the integrase-inactivating mutation is an aspartic acid to valine mutation at amino acid position 64 (D64V) of the integrase protein encoded by the integrase coding sequence.

13. The lentiviral packaging plasmid of any one of claims 1-12, wherein the lentiviral packaging plasmid comprises a deletion of all or a portion of an integrase coding sequence.

14. The lentiviral packaging plasmid of claim 1, wherein the lentiviral packaging plasmid comprises a deletion of all or a portion of a reverse transcriptase coding sequence.

15. The lentiviral packaging plasmid of any one of claims 1-14, wherein the eukaryotic promoter is a RNA polymerase II promoter.

16. A mammalian expression plasmid comprising an eukaryotic promoter operably linked to a Viral Protein R (VPR) coding sequence or a Negative Regulatory Factor (NEF) coding sequence, wherein the VPR coding sequence or the NEF coding sequence comprises at least one non-viral aptamer-binding protein (ABP) nucleotide sequence.

17. The mammalian expression plasmid of claim 16, wherein the at least one non-viral ABP nucleotide sequence encodes MS2 coat protein, PP7 coat protein, lambda N peptide, or COM protein.

18. The mammalian expression plasmid of claim 16 or claim 17, wherein the VPR coding sequence or the NEF coding sequence comprises a first non-viral ABP nucleotide sequence and a second non-viral ABP nucleotide sequence.

19. The mammalian expression plasmid of claim 18, wherein the first non-viral ABP nucleotide sequence and the second non-viral ABP nucleotide sequence are the same non-viral ABP nucleotide sequence.

20. The mammalian expression plasmid of claim 18, wherein the first non-viral ABP nucleotide sequence and the second non-viral ABP nucleotide sequence are different non-viral ABP nucleotide sequence.

21. The mammalian expression plasmid of claim 19, wherein the first non-viral ABP nucleotide sequence and the second non-viral ABP nucleotide sequence both encode an ABP selected from the group consisting of MS2 coat protein, PP7 coat protein, lambda N peptide, and COM protein.

22. The mammalian expression plasmid of claim 20, wherein the first non-viral ABP nucleotide sequence and the second non-viral ABP nucleotide sequence each encode a different ABP selected from the group consisting of MS2 coat protein, PP7 coat protein, lambda N peptide, and COM protein.

23. The lentiviral packaging plasmid of any one of claims 16-22, wherein the eukaryotic promoter is a RNA polymerase II promoter.

24. A mammalian expression plasmid comprising an eukaryotic promoter operably linked to a non-viral nucleic acid sequence, wherein the non-viral nucleic acid sequence comprises at least one aptamer coding sequence, and wherein the non-viral nucleic acid sequence comprises (i) one or both of a CRISPR-associated endonuclease coding sequence or a guide RNA (gRNA) coding sequence, and (ii) at least one aptamer coding sequence.

25. The mammalian expression plasmid of claim 24, wherein the CRISPR-associated endonuclease coding sequence encodes a Cas9 protein, a Cpf1 protein, or a derivative of either.

26. The mammalian expression plasmid of claim 24 or claim 25, wherein the gRNA coding sequence encodes an RNA molecule comprising a DNA targeting sequence and a constant region that interacts with the CRISPR-associated endonuclease.

27. The mammalian expression plasmid of any one of claims 24-26, wherein the gRNA coding sequence encodes a gRNA that comprises a transactivating crRNA (tracrRNA) sequence.

28. The mammalian expression plasmid of any one of claims 24-27, wherein the gRNA coding sequence encodes a gRNA that does not comprise a tracrRNA sequence.

29. The mammalian expression plasmid of any one of claims 24-28, wherein the non-viral nucleic acid sequence is a CRISPR-associated endonuclease coding sequence and the eukaryotic promoter operably linked thereto is a RNA polymerase II promoter.

30. The mammalian expression plasmid of any one of claims 24-28, wherein the non-viral nucleic acid sequence is a gRNA coding sequence and the eukaryotic promoter operably linked thereto is a RNA polymerase III promoter.

31. The mammalian expression plasmid of any one of claims 24-28, wherein the non-viral nucleic acid sequence comprises both a CRISPR-associated endonuclease coding sequence and a gRNA coding sequence, and wherein a RNA polymerase II promoter is operably linked to the CRISPR-associated endonuclease coding sequence and a RNA polymerase III promoter operably linked to the gRNA coding sequence.

32. The mammalian expression plasmid of any one of claims 24-31, wherein the at least one aptamer coding sequence encodes an aptamer sequence bound specifically by an ABP selected from the group consisting of MS2 coat protein, PP7 coat protein, lambda N RNA-binding domain, or COM protein.

33. The mammalian expression plasmid of any one of claims 24-32, wherein the non-viral nucleic acid sequence comprises two aptamer coding sequences.

34. The mammalian expression plasmid of any one of claims 24-33, wherein the at least one non-viral nucleic acid sequence comprises a CRISPR-associated endonuclease coding sequence comprising at least one first aptamer coding sequence and/or a gRNA coding sequence comprising at least one second aptamer coding sequence.

35. The mammalian expression plasmid of any one of claim 34, wherein the at least one non-viral nucleic acid sequence comprises a CRISPR-associated endonuclease coding sequence comprises at least one aptamer coding sequence.

36. The mammalian expression plasmid of claim 34 wherein the gRNA coding sequence comprises at least one aptamer coding sequence.

37. The mammalian expression plasmid of claim 36, wherein the at least one aptamer coding sequence is inserted into the tetraloop of the gRNA coding sequence.

38. The mammalian expression plasmid of claim 37, wherein the aptamer coding sequence binds to a COM protein.

39. The mammalian expression plasmid of claim 34, wherein the at least one first aptamer coding sequence and the at least one second coding aptamer sequence are the same aptamer coding sequence.

40. The mammalian expression plasmid of claim 34, wherein the at least one first aptamer coding sequence encodes an aptamer sequence bound specifically by a first ABP and the at least one second aptamer coding sequence encodes an aptamer sequence bound specifically by a second ABP, wherein the at least one first aptamer coding sequence and the at least one second aptamer coding sequence encode aptamer sequences bound by different first and second ABPs.

41. The mammalian expression plasmid of claim 40, wherein the at least one first aptamer coding sequence and the at least one second aptamer coding sequence encode an aptamer sequence bound specifically by an ABP selected from the group consisting of MS2 coat protein, PP7 coat protein, lambda N protein RNA binding domain, and COM protein.

42. The mammalian expression plasmid of claim 40, wherein the at least one first aptamer coding sequence and the at least one second aptamer coding sequence encode aptamer sequence that are each bound specifically by a different ABP selected from the group consisting of MS2 coat protein, PP7 coat protein, lambda N protein RNA binding domain, and COM protein.

43. The mammalian expression plasmid of any one of claims 24-42, wherein a polynucleotide sequence encoding a RNA-stabilizing sequence is positioned at the 3′ end of the non-viral nucleic acid sequence.

44. The mammalian expression plasmid of claim 43, wherein the polynucleotide sequence encoding a RNA-stabilizing sequence comprises a polynucleotide sequence encoding at least one 3′ UTR of human beta globin gene.

45. The mammalian expression plasmid of any one of claims 24-44, wherein the plasmid is a lentiviral transfer plasmid.

46. A lentiviral packaging system comprising:
a) a packaging plasmid comprising a eukaryotic promoter operably linked to a Gag nucleotide sequence, wherein the Gag nucleotide sequence comprises a nucleocapsid (NC) coding sequence and a matrix protein (MA) coding sequence, wherein one or both of the NC coding sequence or the MA coding sequence comprises at least one non-viral aptamer-binding protein (ABP) nucleotide sequence, and wherein the packaging plasmid does not encode a functional integrase protein;
b) at least one mammalian expression plasmid comprising a eukaryotic promoter operably linked to a non-viral nucleic acid sequence, wherein the non-viral nucleic acid sequence comprises a CRISPR-associated endonuclease coding sequence, a guide RNA (gRNA) coding sequence, or both a CRISPR-associated endonuclease coding sequence and a gRNA coding sequence; and
c) an envelope plasmid comprising an envelope glycoprotein coding sequence.

47. The lentiviral packaging system of claim 46, wherein the non-viral RNA sequence comprises at least one aptamer sequence.

48. The lentiviral packaging system of claim 46 or claim 47, wherein the packaging plasmid further comprises a Rev nucleotide sequence and a Tat nucleotide sequence.

49. The lentiviral packaging system of claim 46, further comprising a second packaging plasmid comprising a Rev nucleotide sequence.

50. The lentiviral packaging system of any one of claims 46-49, wherein the NC coding sequence comprises two functional zinc finger protein domains and functional native protease processing sequences.

51. The lentiviral packaging system of any one of claims 46-50, wherein the at least one non-viral ABP nucleotide sequence encodes MS2 coat protein, PP7 coat protein, lambda N peptide, or COM protein.

52. The lentiviral packaging system any one of claims 46-51, wherein in one or both of the NC coding sequence or the MA coding sequence comprises a first non-viral ABP nucleotide sequence and a second non-viral ABP nucleotide sequence immediately downstream of first non-viral ABP nucleotide sequence.

53. The lentiviral packaging system of claim 52, wherein the first non-viral ABP nucleotide sequence and the second non-viral ABP nucleotide sequence both encode the same ABP, wherein the ABP comprises MS2 coat protein, PP7 coat protein, lambda N peptide, or COM protein.

54. The lentiviral packaging system of claim 52, wherein the first non-viral ABP nucleotide sequence and the second non-viral ABP nucleotide sequence encode different ABPs selected from the group consisting of MS2 coat protein, PP7 coat protein, lambda N peptide, and COM protein.

55. The lentiviral packaging system of any one of claims 46-54, wherein the NC coding sequence comprises a first non-viral ABP nucleotide sequence and the MA coding sequence comprises at least one second non-viral ABP nucleotide sequence.

56. The lentiviral packaging system of claim 55, wherein the at least one first non-viral ABP nucleotide sequence and the at least one second non-viral ABP nucleotide sequence both encode the same ABP, wherein the ABP comprises MS2 coat protein, PP7 coat protein, lambda N peptide, or COM protein.

57. The lentiviral packaging system of claim 55, wherein the at least one first non-viral ABP nucleotide sequence and the at least one second non-viral ABP nucleotide sequence encode different ABPs selected from the group consisting of MS2 coat protein, PP7 coat protein, lambda N peptide, and COM protein.

58. The lentiviral packaging system of any one of claims 46-58, wherein the lentiviral packaging plasmid comprises an integrase coding sequence with an integrase-inactivating mutation.

59. The lentiviral packaging system of claim 58, wherein the integrase-inactivating mutation is an aspartic acid to valine mutation at amino acid position 64 (D64V) of the integrase protein encoded by the integrase coding sequence.

60. The lentiviral packaging system of any one of claims 46-59, wherein the lentiviral packaging plasmid comprises a deletion of all or a portion of an integrase coding sequence.

61. The lentiviral packaging system of any one of claims 46-60, wherein the lentiviral packaging plasmid comprises a deletion of all or a portion of a reverse transcriptase coding sequence.

62. The lentiviral packaging system of any one of claims 46-61, wherein the eukaryotic promoter operably linked to the Gag nucleotide sequence is a RNA polymerase II promoter.

63. The lentiviral packaging system of any one of claims 46-62, wherein the CRISPR-associated endonuclease coding sequence encodes a Cas9 protein, a Cpf1 protein, or a derivative of either.

64. The lentiviral packaging system of any one of claims 46-63, wherein the gRNA coding sequence encodes an RNA molecule comprising a DNA targeting sequence and a constant region that interacts with the CRISPR-associated endonuclease.

65. The lentiviral packaging system of any one of claims 46-64, wherein the gRNA encoded by the gRNA coding sequence comprises a transactivating crRNA (tracrRNA) sequence.

66. The lentiviral packaging system of any one of claims 46-65, wherein the gRNA encoded by the gRNA coding sequence does not comprise a tracrRNA sequence.

67. The lentiviral packaging system of any one of claims 46-66, wherein the non-viral nucleic acid sequence is a CRISPR-associated endonuclease coding sequence and the eukaryotic promoter operably linked thereto is a RNA polymerase II promoter.

68. The lentiviral packaging system of any one of claims 46-67, wherein the non-viral nucleic acid sequence is a gRNA coding sequence and the eukaryotic promoter operably linked thereto is a RNA polymerase III promoter.

69. The lentiviral packaging system of any one of claims 46-68, wherein the non-viral nucleic acid sequence comprises both a CRISPR-associated endonuclease coding sequence and a gRNA coding sequence, and wherein a RNA polymerase II promoter is operably linked to the CRISPR-associated endonuclease coding sequence and a RNA polymerase III promoter operably linked to the gRNA coding sequence.

70. The lentiviral packaging system of any one of claims 47-69, wherein the at least one aptamer sequence encodes an ABP target RNA binding sequence for an ABP selected from the group consisting of MS2 coat protein, PP7 coat protein, lambda N RNA-binding domain, or COM protein.

71. The lentiviral packaging system of any one of claims 47-70, wherein the non-viral nucleic acid sequence in the at least one mammalian expression plasmid comprises two aptamer sequences.

72. The lentiviral packaging system of any one of claims 47-71, wherein the at least one non-viral nucleic acid comprises a CRISPR-associated endonuclease coding sequence comprises at least one first aptamer sequence and/or a gRNA coding sequence comprising at least one second aptamer sequence.

73. The lentiviral packaging system of claim 47, wherein the CRISPR-associated endonuclease coding sequence comprises at least one aptamer coding sequence.

74. The lentiviral packaging system of claim 47, wherein the gRNA coding sequence comprises at least one aptamer coding sequence.

75. The lentiviral packaging system of claim 74, wherein the at least one aptamer coding sequence is inserted into the tetraloop of the gRNA coding sequence.

76. The lentiviral packaging system of claim 75, wherein the aptamer coding sequence binds to a COM protein.

77. The lentiviral packaging system of claim 72, wherein the at least one first aptamer sequence and the at least one second aptamer sequence are the same aptamer sequence.

78. The lentiviral packaging system of claim 72, wherein the at least one first aptamer sequence is an ABP target RNA binding sequence for a first ABP and the at least one second aptamer sequence is an ABP target RNA binding sequence for a second ABP, wherein the first and second aptamer sequences are ABP target RNA binding sequences for different first and second ABPs.

79. The lentiviral packaging system of claim 77, wherein the at least one first aptamer sequence and the at least one second aptamer sequence are an ABP target RNA binding sequence for an ABP selected from the group consisting of MS2 coat protein, PP7 coat protein, lambda N protein RNA binding domain, or COM protein.

80. The lentiviral packaging system of claim 78, wherein the at least one first aptamer sequence and the at least one second aptamer sequence are each a different ABP target RNA binding sequence for an ABP selected from the group consisting of MS2 coat protein, PP7 coat protein, lambda N protein RNA binding domain, or COM protein.

81. The lentiviral packaging system of any one of claims 46-80, wherein the the non-viral nucleic acid sequence comprises a RNA-stabilizing sequence positioned at the 3′ end thereof.

82. The lentiviral packaging system of claim 81, wherein the RNA-stabilizing sequence comprises at least one 3′ UTR of human beta globin gene.

83. The lentiviral packaging system of any one of claims 46-82, wherein the plasmid is a lentiviral transfer plasmid.

84. The lentiviral packaging system of any one of claims 46-83, wherein the at least one envelope plasmid comprises an envelope glycoprotein coding sequence that encodes an envelope glycoprotein.

85. The lentiviral packaging system of claim 84, wherein the envelope glycoprotein coding sequence encodes VSV-G.

86. The lentiviral packaging system of any one of claims 46-85, wherein the at least one ABP nucleotide sequence does not interfere with viral particle assembly when the packaging plasmid, the at least one mammalian expression plasmid, and the envelope plasmid are transfected into eukaryotic cells.

87. A lentiviral packaging system comprising:
a) a packaging plasmid, and wherein the packaging plasmid does not encode a functional integrase protein;
b) at least one mammalian expression plasmid comprising a eukaryotic promoter operably linked to a Viral Protein R (VPR) coding sequence or a Negative Regulatory Factor (NEF) coding sequence, wherein one or both of the VPR coding sequence or the NEF coding sequence comprises at least one non-viral aptamer-binding protein (ABP) nucleotide sequence;
c) at least one mammalian expression plasmid comprising a eukaryotic promoter operably linked to a non-viral nucleic acid sequence, wherein the non-viral nucleic acid sequence comprises a CRISPR-associated endonuclease coding sequence, a guide RNA (gRNA) coding sequence, or both a CRISPR-associated endonuclease coding sequence and a gRNA coding sequence; and
d) an envelope plasmid comprising an envelope glycoprotein coding sequence.

88. The lentiviral packaging system of claim 87, wherein the non-viral RNA sequence comprises at least one aptamer sequence.

89. The lentiviral packaging system of claim 87, wherein the CRISPR-associated endonuclease coding sequence comprises at least one aptamer coding sequence.

90. The lentiviral packaging system of claim 87, wherein the gRNA coding sequence comprises at least one aptamer coding sequence.

91. The lentiviral packaging system of claim 90, wherein the at least one aptamer coding sequence is inserted into the tetraloop of the gRNA coding sequence.

92. The lentiviral packaging system of claim 91, wherein the aptamer coding sequence binds to a COM protein.

93. The lentiviral packaging system of any one of claims 87-92, wherein the at least one non-viral ABP nucleotide sequence encodes MS2 coat protein, PP7 coat protein, lambda N peptide, or COM protein.

94. The lentiviral packaging system of any one of claims 87-93, wherein the eukaryotic promoter operably linked to the VPR coding sequence or the NEF coding sequence is a RNA polymerase II promoter.

95. The lentiviral packaging system of any one of claims 87-94, wherein the CRISPR-associated endonuclease coding sequence encodes a Cas9 protein, a Cpf1 protein, or a derivative of either.

96. The lentiviral packaging system of any one of claims 87-95, wherein the gRNA coding sequence encodes an RNA molecule comprising a DNA targeting sequence and a constant region that interacts with the CRISPR-associated endonuclease.

97. The lentiviral packaging system of any one of claims 87-96, wherein the non-viral nucleic acid sequence is a CRISPR-associated endonuclease coding sequence and the eukaryotic promoter operably linked thereto is a RNA polymerase II promoter.

98. The lentiviral packaging system of any one of claims 87-97, wherein the non-viral nucleic acid sequence is a gRNA coding sequence and the eukaryotic promoter operably linked thereto a RNA polymerase III promoter.

99. The lentiviral packaging system of any one of claims 88-98, wherein the at least one aptamer sequence encodes an ABP target RNA binding sequence for an ABP selected from the group consisting of MS2 coat protein, PP7 coat protein, lambda N RNA-binding domain, or COM protein.

100. The lentiviral packaging system of any one of claims 87-99, wherein the the non-viral nucleic acid sequence comprises a RNA-stabilizing sequence positioned at the 3′ end thereof

101. The lentiviral packaging system of any one of claims 87-100, wherein the at least one envelope plasmid comprises an envelope glycoprotein coding sequence that encodes an envelope glycoprotein.

102. A lentivirus-like particle comprising:
a) a fusion protein comprising a nucleocapsid (NC) protein or a matrix (MA) protein, wherein the NC protein or MA protein comprises at least one non-viral aptamer binding protein (ABP); and
b) at least one non-viral RNA molecule, wherein the non-viral RNA sequence comprises a CRISPR-associated endonuclease mRNA, a guide RNA (gRNA), or both a CRISPR-associated endonuclease mRNA and a gRNA;
wherein the lentivirus-like particle does not comprise a functional integrase protein.

103. The lentivirus-like particle of claim 102, wherein the at least one non-viral RNA molecule comprises at least one aptamer sequence.

104. The lentivirus-like particle of claim 103, wherein the at least one non-viral RNA molecule comprises two aptamer sequences.

105. The lentivirus-like particle of any one of claims 102-104, wherein the NC protein comprises two functional zinc finger protein domains.

106. The lentivirus-like particle of any one of claims 102-105, wherein the at least one non-viral ABP is MS2 coat protein, PP7 coat protein, lambda N protein RNA binding domain, or COM protein.

107. The lentivirus-like particle of any one of claims 102-106, wherein the fusion protein comprises a first non-viral ABP and a second non-viral ABP fused to a C′ terminal end of the first ABP.

108. The lentivirus-like particle of any one of claims 102-107, wherein the lentivirus-like particle comprises a NC protein comprising at least one first non-viral ABP and a MA protein comprising at least one second non-viral ABP.

109. The lentivirus-like particle of claim 107 or 108, wherein the first non-viral ABP and the second non-viral ABP are both MS2 coat protein, PP7 coat protein, lambda N protein RNA binding domain, or COM protein.

110. The lentivirus-like particle of claim 107 or 108, wherein the first non-viral ABP and the second non-viral aptamer ABP are different ABPs each selected from the group consisting of MS2 coat protein, PP7 coat protein, lambda N protein RNA binding domain, and COM protein.

111. The lentivirus-like particle of any one of claims 102-110, wherein the lentivirus-like particle comprises a non-functional integrase protein comprising an aspartic acid to valine mutation at amino acid position 64 (D64V).

112. The lentivirus-like particle of any one of claims 102-111, wherein the lentivirus-like particle does not comprise an integrase protein.

113. The lentivirus-like particle of any one of claims 102-112, wherein the lentivirus-like particle does not comprise a reverse transcriptase protein.

114. The lentivirus-like particle of any one of claims 102-113, wherein the non-viral RNA molecule comprises a CRISPR-associated endonuclease mRNA.

115. The lentivirus-like particle of any one of claims 102-114, wherein the non-viral RNA molecule comprises a CRISPR-associated endonuclease mRNA that encodes a Cas9 protein, a Cpf1 protein, or a derivative of either.

116. The lentivirus-like particle of any one of claims 102-115, wherein the non-viral RNA molecule comprises a gRNA.

117. The lentivirus-like particle of any one of claims 102-116, wherein the non-viral RNA molecule comprises a gRNA comprising a DNA targeting sequence and a constant region that interacts with the CRISPR-associated endonuclease.

118. The lentivirus-like particle of any one of claims 102-117, wherein the non-viral RNA molecule comprises a gRNA comprising a transactivating crRNA (tracrRNA) sequence.

119. The lentivirus-like particle of any one of claims 102-118, wherein the non-viral RNA molecule comprises a gRNA that does not comprise a tracrRNA sequence.

120. The lentivirus-like particle of any one of claims 103-119, wherein the at least one aptamer sequence comprises an ABP target binding sequence for an ABP selected from the group consisting of MS2 coat protein, PP7 coat protein, lambda N protein RNA binding domain, or COM protein.

121. The lentivirus-like particle of any one of claims 103-120, wherein the at least one non-viral RNA molecule comprises a CRISPR-associated endonuclease mRNA comprising at least one first aptamer sequence and a gRNA comprising at least one second aptamer sequence.

122. The lentivirus-like particle of claim 121, wherein the at least one first aptamer sequence and the at least one second aptamer are the same aptamer sequence.

123. The lentivirus-like particle of claim 121, wherein the at least one first aptamer sequence is an ABP target RNA binding sequence for a first ABP and the at least one second aptamer sequence is an ABP target RNA binding sequence for a second ABP, wherein the at least one first aptamer sequence and the at least one second aptamer sequence are ABP target RNA binding sequences for different ABPs.

124. The lentivirus-like particle of claim 122, wherein the at least one first aptamer sequence and the at least one second aptamer sequence are selected from the group consisting of MS2 coat protein, PP7 coat protein, lambda N peptide, and COM protein.

125. The lentivirus-like particle of claim 123, wherein the at least one first aptamer sequence and the at least one second aptamer sequence are each a different ABP selected from the group consisting of MS2 coat protein, PP7 coat protein, lambda N peptide, and COM protein.

126. The lentivirus-like particle of any one of claims 102-125, wherein one or more of the at least one non-viral RNA molecules comprises a RNA-stabilizing sequence positioned at the 3′ end.

127. The lentivirus-like particle of claim 126, wherein the RNA-stabilizing sequence comprises at least one 3′ untranslated region (UTR) of human beta globin gene.

128. The lentivirus-like particle of any one of claims 102-127, wherein the fusion protein does not interfere with lentivirus-like particle transduction of eukaryotic cells.

129. The lentivirus-like particle of any one of claims 103-128, wherein the at least one aptamer sequence of the at least one non-viral RNA molecule does not interfere with lentivirus-like particle transduction of eukaryotic cells.

130. The lentivirus-like particle of any one of claims 103-129, wherein expression of a CRISPR-associated endonuclease from the CRISPR-associated endonuclease mRNA comprising at least one aptamer sequence in eukaryotic cells following transduction with the lentivirus-like particle is equal to or greater than expression of the CRISPR-associated endonuclease from the CRISPR-associated endonuclease mRNA without the at least one aptamer sequence in eukaryotic cells following transduction with a lentivirus-like particle.

131. The lentivirus-like particle of any one of claims 103-130, wherein a CRISPR-associated endonuclease expressed from the CRISPR-associated endonuclease mRNA comprising the at least one aptamer sequence immediately downstream thereof in eukaryotic cells following transduction with the lentivirus-like particle is as functional as a CRISPR-associated endonuclease expressed from the CRISPR-associated endonuclease mRNA without the at least one aptamer sequence in eukaryotic cells following transduction with a lentivirus-like particle.

132. The lentivirus-like particle of any one of claims 103-131, wherein the gRNA comprising the at least one aptamer sequence in eukaryotic cells following transduction with the lentivirus-like particle is as functional as the gRNA without the at least one aptamer sequence in eukaryotic cells following transduction with a lentivirus-like particle.

133. A lentivirus-like particle comprising:
a) a fusion protein comprising a Viral Protein R (VPR) protein or a Negative Regulatory Factor (NEF) protein, wherein VPR protein or the NEF protein comprises at least one non-viral aptamer binding protein (ABP); and
b) at least one non-viral RNA molecule, wherein the non-viral RNA sequence comprises a CRISPR-associated endonuclease mRNA, a guide RNA (gRNA), or both a CRISPR-associated endonuclease mRNA and a gRNA;
wherein the lentivirus-like particle does not comprise a functional integrase protein.

134. The lentivirus-like particle of claim 133, wherein the at least one non-viral RNA molecule comprises at least one aptamer sequence.

135. The lentiviral-like particle of claim 133, wherein the CRISPR-associated endonuclease mRNA comprises at least one aptamer coding sequence.

136. The lentiviral-like particle of claim 133, wherein the gRNA comprises at least one aptamer coding sequence.

137. The lentiviral-like particle of claim 136, wherein the at least one aptamer coding sequence is inserted into the tetraloop of the gRNA coding sequence.

138. The lentiviral particle of claim 137, wherein the aptamer coding sequence binds to a COM protein.

139. The lentivirus-like particle of any one of claims 133-138, wherein the at least one non-viral ABP is MS2 coat protein, PP7 coat protein, lambda N protein RNA binding domain, or COM protein.

140. The lentivirus-like particle of any one of claims 133-138, wherein the non-viral RNA molecule comprises a CRISPR-associated endonuclease mRNA that encodes a Cas9 protein, a Cpf1 protein, or a derivative of either.

141. The lentivirus-like particle of any one of claims 133-140, wherein the non-viral RNA molecule comprises a gRNA.

142. The lentivirus-like particle of any one of claim 141, wherein the non-viral RNA molecule comprises a gRNA comprising a DNA targeting sequence and a constant region that interacts with the CRISPR-associated endonuclease.

143. The lentivirus-like particle of any one of claims 134-142, wherein the at least one aptamer sequence comprises an ABP target binding sequence for an ABP selected from the group consisting of MS2 coat protein, PP7 coat protein, lambda N protein RNA binding domain, or COM protein.

144. The lentivirus-like particle of any one of claims 134-143, wherein the at least one non-viral RNA molecule comprises a CRISPR-associated endonuclease mRNA comprising at least one first aptamer sequence and a gRNA comprising at least one second aptamer sequence.

145. The lentivirus-like particle of claim 144, wherein the at least one first aptamer sequence and the at least one second aptamer are the same aptamer sequence.

146. The lentivirus-like particle of claim 144, wherein the at least one first aptamer sequence and the at least one second aptamer are different aptamer sequences.

147. The lentivirus-like particle of any one of claims 134-146, wherein one or more of the at least one non-viral RNA molecules comprises a RNA-stabilizing sequence positioned at the 3′ end.

148. The lentivirus-like particle of claim 147, wherein the RNA-stabilizing sequence comprises at least one 3′ untranslated region (UTR) of human beta globin gene.

149. A lentivirus-like particle comprising:
a) a fusion protein comprising a Viral Protein R (VPR) protein or a Negative Regulatory Factor (NEF) protein, wherein VPR protein or the NEF protein comprises at least one non-viral aptamer binding protein (ABP); and
b) a ribonucleotide protein (RNP) complex comprising a CRISPR-associated endonuclease and a guide RNA;
wherein the lentivirus-like particle does not comprise a functional integrase protein.

150. A method of producing a lentiviral particle, the method comprising:
a) transfecting a plurality of eukaryotic cells with the packaging plasmid, the at least one mammalian expression plasmid, and the envelope plasmid of the system of any one of claims 46-101; and
b) culturing the transfected eukaryotic cells for sufficient time for lentiviral particles to be produced.

151. The method of claim 150, wherein the lentiviral particle comprises a CRISPR-associated endonuclease mRNA.

152. The method of claim 150, wherein the lentiviral particle comprises a RNP comprising a ribonucleotide protein (RNP) complex comprising a CRISPR-associated endonuclease and a guide RNA.

153. The method of any one of claims 150-152, wherein the plurality of eukaryotic cells are mammalian cells.

154. A method of producing a lentiviral particle, the method comprising:
a) transfecting a plurality of eukaryotic cells with the plasmids of the system of any one of claims 87-101; and
b) culturing the transfected eukaryotic cells for sufficient time for lentiviral particles to be produced.

155. The method of claim 154, wherein the plurality of eukaryotic cells are mammalian cells.

156. A method of modifying a genomic target sequence in a cell, the method comprising transducing a plurality of eukaryotic cells with a plurality of viral particles, wherein the plurality of viral particles comprise:
i) a lentivirus-like particle according to anyone one of claims 102-148, wherein which the non-viral RNA sequence comprises a CRISPR-associated endonuclease mRNA and a gRNA, or
ii) a lentivirus-like particle according to anyone one of claims 102-148, wherein which the non-viral RNA sequence comprises a CRISPR-associated endonuclease mRNA and a second viral particle comprising a gRNA or a gRNA coding sequence,
wherein a CRISPR-associated endonuclease is expressed from the CRISPR-associated endonuclease mRNA in cells transduced with the lentivirus-like particle,
wherein, if the second viral particle comprises a gRNA coding sequence, a gRNA is expressed from the gRNA coding sequence in cells transduced with the second viral particle, and
wherein the CRISPR-associated endonuclease and the gRNA form a complex that binds to the genomic target sequence in genomic DNA of the cell and the CRISPR-associated endonuclease cleaves the genomic DNA of the cell, thereby triggering cellular DNA repair mechanisms causing modification of the genomic target sequence.

157. The method of claim 156, wherein the second viral particle is a second lentivirus-like particle comprising a gRNA.

158. The method of claim 156 or 157, wherein the second viral particle is a lentivirus particle comprising a gRNA coding sequence.

159. The method of any one of claims 156-158, wherein the second viral particle is an adenovirus particle or an adeno-associated viral particle, and comprises a gRNA coding sequence operably linked to a eukaryotic promoter.

160. The method of any one of claims 156-159, wherein the second viral particle comprises a target template sequence.

161. The method of any one of claims 156-160, wherein the plurality of viral particles also comprise a third viral particle comprising a target template sequence, wherein the third viral particle is an adenovirus particle, an adeno-associated viral particle, or a lentivirus particle.

162. The method of claim 158 or 161, wherein one or both of the second viral particle or the third viral particle is an integration defective lentivirus particle.

163. The method of claim 160 or 161, wherein the target template sequence comprises nucleic acid sequences homologous to genomic DNA flanking the genomic target sequence.

164. The method of any one of claims 156-163, wherein the fusion protein of the lentivirus-like particle does not interfere with viral transduction of the plurality of eukaryotic cells.

165. The method of any one of claims 156-164, wherein expression of the CRISPR-associated endonuclease from the CRISPR-associated endonuclease mRNA comprising the at least one aptamer sequence in eukaryotic cells following transduction with the lentivirus-like particle is equal to or greater than expression of the CRISPR-associated endonuclease from the CRISPR-associated endonuclease mRNA without the at least one aptamer sequence in eukaryotic cells following transduction with a lentivirus-like particle.

166. The method of any one of claims 156-165, wherein the CRISPR-associated endonuclease expressed from the CRISPR-associated endonuclease mRNA comprising the at least one aptamer sequence in eukaryotic cells following transduction with the lentivirus-like particle is as functional as the CRISPR-associated endonuclease expressed from the CRISPR-associated endonuclease mRNA without the at least one aptamer sequence in eukaryotic cells following transduction with a lentivirus-like particle.

167. The method of any one of claims 156-166, wherein the gRNA comprising the at least one aptamer sequence in eukaryotic cells following transduction with the lentivirus-like particle is as functional as the gRNA without the at least one aptamer sequence in eukaryotic cells following transduction with the lentivirus-like particle.

168. A method of modifying a genomic target sequence in a cell, the method comprising transducing a plurality of eukaryotic cells with a plurality of viral particles, wherein the plurality of viral particles comprise: i) a lentivirus-like particle according claim 149, wherein the RNP binds to the genomic target sequence in genomic DNA of the cell and the CRISPR-associated endonuclease cleaves the genomic DNA of the cell, thereby triggering cellular DNA repair mechanisms causing modification of the genomic target sequence.

169. The method of any one of claims 156-168, wherein the plurality of eukaryotic cells are mammalian cells.

170. The method of any one of claims 156-169, wherein the plurality of eukaryotic cells are cells present in subject.

171. The method of any one of claims 156-170, wherein the subject is a human subject.

172. The method of claim 171, wherein the subject is injected with the plurality of viral particles.

173. The method of any one of claims 156-173, wherein the genomic target sequence is in a hemoglobin gene.

174. The method of any one of claims 156-173, wherein the genomic target sequence is in an oncogene.

175. A cell containing the plasmid of any one of claims 1-45.

176. A cell containing the lentiviral packaging system of any one of claims 46-101.

177. A cell containing the lentivirus-like particle of any one of claims 102-150.

178. A cell modified using the method of any one of claims 157-169.

179. A method for treating a disease in a subject comprising:
a) obtaining cells from the subject;
b) modifying the cells of the subject using the method of any one of claims 156-168; and
c) administering the modified cells to the subject.

180. The method of claim 179, wherein the disease is cancer.

181. The method of claim 180, wherein the disease is sickle cell anemia.

182. The method of any one of claims 176-181, wherein the cells are T cells.


SEQUENCE LISTING (219 sequences)

SEQ ID NO: 1 - DNA, 165 bp, synthetic construct
  misc_feature 1..165: note = synthetic construct
  source 1..165: mol_type = other DNA
atacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaat

SEQ ID NO: 2 - protein, 55 aa, synthetic construct
  REGION 1..55: note = synthetic construct
IQKGNFRNQRKTVKCFNCGKEGHIAKNCRAPRKKGCWKCGKEGHQMKDCTERQAN

SEQ ID NO: 3 - DNA, 396 bp, synthetic construct
  misc_feature 1..396: note = synthetic construct
  source 1..396: mol_type = other DNA
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattac

SEQ ID NO: 4 - protein, 131 aa, synthetic construct
  REGION 1..131: note = synthetic construct
GARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNY

SEQ ID NO: 5 - DNA, 291 bp, synthetic construct
  misc_feature 1..291: note = synthetic construct
  source 1..291: mol_type = other DNA
atggaacaagccccagaagaccagggaccgcagagggaaccatacaatgaatggacactagaacttttagaggaactcaagcgggaagcagtcagacactttcctagaccatggcttcatggcttaggacaacatatctatgaaacctatggagatacttggacgggggtggaagctataataagaattctgcaacgactactgtttgtccatttcagaattgggtgccagcatagccgaataggcattctaagacagagaagagcaagaaatggagccagtagatcctaa

SEQ ID NO: 6 - protein, 96 aa, synthetic construct
  REGION 1..96: note = synthetic construct
MEQAPEDQGPQREPYNEWTLELLEELKREAVRHFPRPWLHGLGQHIYETYGDTWTGVEAIIRILQRLLFVHFRIGCQHSRIGILRQRRARNGASRS

SEQ ID NO: 7 - DNA, 618 bp, synthetic construct
  misc_feature 1..618: note = synthetic construct
  source 1..618: mol_type = other DNA
atgggttgcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataagctggaagaggccaataaaggagagaacaccagcttgttacaccctgtgagcctgcatggaatggatgaccctggaagagaagtgttagagtggaggtttgacagccgcctagcatttcatcacgtggcccgagagctgcatccggagtacttcaagaactgc

SEQ ID NO: 8 - protein, 206 aa, synthetic construct
  REGION 1..206: note = synthetic construct
MGCKWSKSSVIGWPAVRERMRRAEPAADGVGAVSRDLEKHGAITSSNTAANNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKLEEANKGENTSLLHPVSLHGMDDPGREVLEWRFDSRLAFHHVARELHPEYFKNC

SEQ ID NO: 9 - DNA, 351 bp, synthetic construct
  misc_feature 1..351: note = synthetic construct
  source 1..351: mol_type = other DNA
atggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctac

SEQ ID NO: 10 - protein, 117 aa, synthetic construct
  REGION 1..117: note = synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 11 - DNA, 363 bp, synthetic construct
  misc_feature 1..363: note = synthetic construct
  source 1..363: mol_type = other DNA
tccaaaacaatagtcctctccgtaggggaggcaacacggactttgaccgaaatccagtcaaccgctgaccgacaaatctttgaagagaaagtagggcctcttgtgggccgactgcgcttgactgcaagcttgcgacaaaacggcgcaaagactgcctatagggtcaaccttaaactcgaccaagccgacgtggtcgatagcggtctccctaaggttcggtatacgcaggtctggagtcatgacgtaacaatcgtagcaaacagcacagaagcctcccgaaaaagcctctacgatctgacgaaatccttggtggctacgtcacaggtggaagacctcgttgtcaaccttgtacctctgggtcga

SEQ ID NO: 12 - protein, 121 aa, synthetic construct
  REGION 1..121: note = synthetic construct
SKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGR

SEQ ID NO: 13 - DNA, 66 bp, synthetic construct
  misc_feature 1..66: note = synthetic construct
  source 1..66: mol_type = other DNA
atggatgcacaaacacgccgccgcgaacgtcgcgcagagaaacaggctcaatggaaagcagcaaat

SEQ ID NO: 14 - protein, 22 aa, synthetic construct
  REGION 1..22: note = synthetic construct
MDAQTRRRERRAEKQAQWKAAN

SEQ ID NO: 15 - DNA, 193 bp, synthetic construct
  misc_feature 1..193: note = synthetic construct
  source 1..193: mol_type = other DNA
atgaaatcaattcgctgtaaaaactgcaacaaactgttatttaaggcggattcctttgatcacattgaaatcaggtgtccgcgttgcaaacgtcacatcataatgctgaatgcctgcgagcatcccacggagaaacattgtgggaaaagagaaaaaatcacgcattctgacgaaaccgtgcgttattgagtat

SEQ ID NO: 16 - protein, 62 aa, synthetic construct
  REGION 1..62: note = synthetic construct
MKSIRCKNCNKLLFKADSFDHIEIRCPRCKRHIIMLNACEHPTEKHCGKREKITHSDETVRY

SEQ ID NO: 17 - RNA, 19 nt, synthetic construct
  misc_feature 1..19: note = synthetic construct
  source 1..19: mol_type = other RNA
acatgaggatcacccatgt

SEQ ID NO: 18 - DNA, 19 bp, synthetic construct
  misc_feature 1..19: note = synthetic construct
  source 1..19: mol_type = other DNA
acatgaggatcacccatgt

SEQ ID NO: 19 - RNA, 25 nt, synthetic construct
  misc_feature 1..25: note = synthetic construct
  source 1..25: mol_type = other RNA
ggagcagacgatatggcgtcgctcc

SEQ ID NO: 20 - DNA, 25 bp, synthetic construct
  misc_feature 1..25: note = synthetic construct
  source 1..25: mol_type = other DNA
ggagcagacgatatggcgtcgctcc

SEQ ID NO: 21 - RNA, 19 nt, synthetic construct
  misc_feature 1..19: note = synthetic construct
  source 1..19: mol_type = other RNA
gggccctgaagaagggccc

SEQ ID NO: 22 - DNA, 19 bp, synthetic construct
  misc_feature 1..19: note = synthetic construct
  source 1..19: mol_type = other DNA
gggccctgaagaagggccc

SEQ ID NO: 23 - RNA, 19 nt, synthetic construct
  misc_feature 1..19: note = synthetic construct
  source 1..19: mol_type = other RNA
ctgaatgcctgcgagcatc

SEQ ID NO: 24 - DNA, 18 bp, synthetic construct
  misc_feature 1..18: note = synthetic construct
  source 1..18: mol_type = other DNA
ctgaatgcctgcgagcat

SEQ ID NO: 25 - DNA, 132 bp, synthetic construct
  misc_feature 1..132: note = synthetic construct
  source 1..132: mol_type = other DNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 26 - RNA, 132 nt, synthetic construct
  misc_feature 1..132: note = synthetic construct
  source 1..132: mol_type = other RNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 27 - DNA, 31 bp, synthetic construct
  misc_feature 1..31: note = synthetic construct
  source 1..31: mol_type = other DNA
ctcttctggtccccacagactcagagagaac

SEQ ID NO: 28 - DNA, 20 bp, synthetic construct
  misc_feature 1..20: note = synthetic construct
  source 1..20: mol_type = other DNA
aaaccgggaactacctgacc

SEQ ID NO: 29 - DNA, 20 bp, synthetic construct
  misc_feature 1..20: note = synthetic construct
  source 1..20: mol_type = other DNA
tcacgaccttgtttctgctg

SEQ ID NO: 30 - DNA, 21 bp, synthetic construct
  misc_feature 1..21: note = synthetic construct
  source 1..21: mol_type = other DNA
gagtaacggcagacttctcca

SEQ ID NO: 31 - DNA, 21 bp, synthetic construct
  misc_feature 1..21: note = synthetic construct
  source 1..21: mol_type = other DNA
cggcattttgccttgtttaag

SEQ ID NO: 32 - DNA, 20 bp, synthetic construct
  misc_feature 1..20: note = synthetic construct
  source 1..20: mol_type = other DNA
cagtgcttcagccgctaccc

SEQ ID NO: 33 - DNA, 20 bp, synthetic construct
  misc_feature 1..20: note = synthetic construct
  source 1..20: mol_type = other DNA
agctcgatgcggttcaccag

SEQ ID NO: 34 - DNA, 56 bp, synthetic construct
  misc_feature 1..56: note = synthetic construct
  source 1..56: mol_type = other DNA
tcgtcggcagcgtcagatgtgtataagagacagtgttcactagcaacctcaaacag

SEQ ID NO: 35 - DNA, 57 bp, synthetic construct
  misc_feature 1..57: note = synthetic construct
  source 1..57: mol_type = other DNA
tcgtcggcagcgtcagatgtgtataagagacagatgttcactagcaacctcaaacag

SEQ ID NO: 36 - DNA, 58 bp, synthetic construct
  misc_feature 1..58: note = synthetic construct
  source 1..58: mol_type = other DNA
tcgtcggcagcgtcagatgtgtataagagacaggatgttcactagcaacctcaaacag

SEQ ID NO: 37 - DNA, 59 bp, synthetic construct
  misc_feature 1..59: note = synthetic construct
  source 1..59: mol_type = other DNA
tcgtcggcagcgtcagatgtgtataagagacagcgatgttcactagcaacctcaaacag

SEQ ID NO: 38 - DNA, 60 bp, synthetic construct
  misc_feature 1..60: note = synthetic construct
  source 1..60: mol_type = other DNA
tcgtcggcagcgtcagatgtgtataagagacagtcgatgttcactagcaacctcaaacag

SEQ ID NO: 39 - DNA, 61 bp, synthetic construct
  misc_feature 1..61: note = synthetic construct
  source 1..61: mol_type = other DNA
tcgtcggcagcgtcagatgtgtataagagacagatcgatgttcactagcaacctcaaacag

SEQ ID NO: 40 - DNA, 58 bp, synthetic construct
  misc_feature 1..58: note = synthetic construct
  source 1..58: mol_type = other DNA
gtctcgtgggctcggagatgtgtataagagacagtccaataggcagagagagtcagtg

SEQ ID NO: 41 - DNA, 75 bp, synthetic construct
  misc_feature 1..75: note = synthetic construct
  source 1..75: mol_type = other DNA
ttacgcttaagaattctagaaaacatgaggatcacccatgtctgcaggtcgactctagaaaattcctagagctcg

SEQ ID NO: 42 - DNA, 75 bp, synthetic construct
  misc_feature 1..75: note = synthetic construct
  source 1..75: mol_type = other DNA
cgagctctaggaattttctagagtcgacctgcagacatgggtgatcctcatgttttctagaattcttaagcgtaa

SEQ ID NO: 43 - DNA, 101 bp, synthetic construct
  misc_feature 1..101: note = synthetic construct
  source 1..101: mol_type = other DNA
ttacgcttaagaattctagaaaacatgaggatcacccatgtctgcaggtcgactctagaaaacatgaggatcacccatgtctgcaaaattcctagagctcg

SEQ ID NO: 44 - DNA, 101 bp, synthetic construct
  misc_feature 1..101: note = synthetic construct
  source 1..101: mol_type = other DNA
cgagctctaggaattttgcagacatgggtgatcctcatgttttctagagtcgacctgcagacatgggtgatcctcatgttttctagaattcttaagcgtaa

SEQ ID NO: 45 - DNA, 101 bp, synthetic construct
  misc_feature 1..101: note = synthetic construct
  source 1..101: mol_type = other DNA
ttacgcttaagaattctagaaaacatgaggatcacccatgtctgcaggtcgactctagaaaacatgaggatcacccatgtctgcaaaattctagaaaacat

SEQ ID NO: 46 - DNA, 101 bp, synthetic construct
  misc_feature 1..101: note = synthetic construct
  source 1..101: mol_type = other DNA
atgttttctagaattttgcagacatgggtgatcctcatgttttctagagtcgacctgcagacatgggtgatcctcatgttttctagaattcttaagcgtaa

SEQ ID NO: 47 - DNA, 25 bp, synthetic construct
  misc_feature 1..25: note = synthetic construct
  source 1..25: mol_type = other DNA
caccgccctgtggggcaaggtgaac

SEQ ID NO: 48 - DNA, 25 bp, synthetic construct
  misc_feature 1..25: note = synthetic construct
  source 1..25: mol_type = other DNA
aaacgttcaccttgccccacagggc

SEQ ID NO: 49 - DNA, 26 bp, synthetic construct
  misc_feature 1..26: note = synthetic construct
  source 1..26: mol_type = other DNA
caccgagtaacggcagacttctccac

SEQ ID NO: 50 - DNA, 26 bp, synthetic construct
  misc_feature 1..26: note = synthetic construct
  source 1..26: mol_type = other DNA
aaacgtggagaagtctgccgttactc

SEQ ID NO: 51 - DNA, 57 bp, synthetic construct
  misc_feature 1..57: note = synthetic construct
  source 1..57: mol_type = other DNA
ttacgcttaagaattcggagcagacgatatggcgtcgctccgaattcctagagctcg

SEQ ID NO: 52 - DNA, 57 bp, synthetic construct
  misc_feature 1..57: note = synthetic construct
  source 1..57: mol_type = other DNA
cgagctctaggaattcggagcgacgccatatcgtctgctccgaattcttaagcgtaa

SEQ ID NO: 53 - DNA, 26 bp, synthetic construct
  misc_feature 1..26: note = synthetic construct
  source 1..26: mol_type = other DNA
caccgagtaacggcagacttctccac

SEQ ID NO: 54 - DNA, 26 bp, synthetic construct
  misc_feature 1..26: note = synthetic construct
  source 1..26: mol_type = other DNA
gaacgtggagaagtctgccgttactc

SEQ ID NO: 55 - DNA, 35 bp, synthetic construct
  misc_feature 1..35: note = synthetic construct
  source 1..35: mol_type = other DNA
ttgcaatgatctcgagggcctatttcccatgattc

SEQ ID NO: 56 - DNA, 36 bp, synthetic construct
  misc_feature 1..36: note = synthetic construct
  source 1..36: mol_type = other DNA
ctgcggccgctctagaaaaatctcgccaacaagttg

SEQ ID NO: 57 - DNA, 85 bp, synthetic construct
  misc_feature 1..85: note = synthetic construct
  source 1..85: mol_type = other DNA
catggtgcatctgacacctgtggagaagtctgccgttactgccctgtggggcaaggtgaacgtggatgaagttggtggtgaggcc

SEQ ID NO: 58 - DNA, 84 bp, synthetic construct
  misc_feature 1..84: note = synthetic construct
  source 1..84: mol_type = other DNA
cagggcctcaccaccaacttcatccacgttcaccttgccccacagggcagtaacggcagacttctccacaggtgtcagatgcac

SEQ ID NO: 59 - DNA, 38 bp, synthetic construct
  misc_feature 1..38: note = synthetic construct
  source 1..38: mol_type = other DNA
cttctgattttctagattgtgtaatcgtagtttcagag

SEQ ID NO: 60 - DNA, 39 bp, synthetic construct
  misc_feature 1..39: note = synthetic construct
  source 1..39: mol_type = other DNA
gcttgatatcgaattaaaaaatctcgccaacaagttgac

SEQ ID NO: 61 - DNA, 1041 bp, synthetic construct
  misc_feature 1..1041: note = synthetic construct
  source 1..1041: mol_type = other DNA
gctagccaccatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacggtggtggaggaggaatggcgtccaatttcacgcagttcgtcctggttgacaacggggggactggggacgttacggtcgctccgagcaactttgccaatggtattgcggagtggatttcttctaattcacggtcccaagcttacaaagtgacctgttccgtgcggcaaagttctgctcagaatagaaagtacactataaaggtcgaagtccctaagggggcctggcgatcatatctcaatatggagcttaccatcccaatatttgccactaattctgattgtgaattgattgtcaaagcaatgcaaggactcttgaaagacggaaacccaatccccagcgcaatcgcagccaactccggtatatacggaggtggtggaggaatggaacaagccccagaagaccagggaccgcagagggaaccatacaatgaatggacactagaacttttagaggaactcaagcgggaagcagtcagacactttcctagaccatggcttcatggcttaggacaacatatctatgaaacctatggagatacttggacgggggtggaagctataataagaattctgcaacgactactgtttgtccatttcagaattgggtgccagcatagccgaataggcattctaagacagagaagagcaagaaatggagccagtagatcctaagcggccgc

SEQ ID NO: 62 - protein, 340 aa, synthetic construct
  REGION 1..340: note = synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGGMEQAPEDQGPQREPYNEWTLELLEELKREAVRHFPRPWLHGLGQHIYETYGDTWTGVEAIIRILQRLLFVHFRIGCQHSRIGILRQRRARNGASRS

SEQ ID NO: 63 - DNA, 1371 bp, synthetic construct
  misc_feature 1..1371: note = synthetic construct
  source 1..1371: mol_type = other DNA
gctagccaccatgggttgcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataagctggaagaggccaataaaggagagaacaccagcttgttacaccctgtgagcctgcatggaatggatgaccctggaagagaagtgttagagtggaggtttgacagccgcctagcatttcatcacgtggcccgagagctgcatccggagtacttcaagaactgcggaggtggtggaggaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacggtggtggaggaggaatggcgtccaatttcacgcagttcgtcctggttgacaacggggggactggggacgttacggtcgctccgagcaactttgccaatggtattgcggagtggatttcttctaattcacggtcccaagcttacaaagtgacctgttccgtgcggcaaagttctgctcagaatagaaagtacactataaaggtcgaagtccctaagggggcctggcgatcatatctcaatatggagcttaccatcccaatatttgccactaattctgattgtgaattgattgtcaaagcaatgcaaggactcttgaaagacggaaacccaatccccagcgcaatcgcagccaactccggtatatactgagcggccgc

SEQ ID NO: 64 - protein, 451 aa, synthetic construct
  REGION 1..451: note = synthetic construct
TMGCKWSKSSVIGWPAVRERMRRAEPAADGVGAVSRDLEKHGAITSSNTAANNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKLEEANKGENTSLLHPVSLHGMDDPGREVLEWRFDSRLAFHHVARELHPEYFKNCGGGGGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 65 - DNA, 1234 bp, synthetic construct
  misc_feature 1..1234: note = synthetic construct
  source 1..1234: mol_type = other DNA
cctaggaaaaagggctgttggaaatgtggaaaggaaggacaccaaatgaaagattgttccggtggaggtggatccggtggaggttccatggcgtccaatttcacgcagttcgtcctggttgacaacggggggactggggacgttacggtcgctccgagcaactttgccaatggtattgcggagtggatttcttctaattcacggtcccaagcttacaaagtgacctgttccgtgcggcaaagttctgctcagaatagaaagtacactataaaggtcgaagtccctaagggggcctggcgatcatatctcaatatggagcttaccatcccaatatttgccactaattctgattgtgaattgattgtcaaagcaatgcaaggactcttgaaagacggaaacccaatccccagcgcaatcgcagccaactccggtatatactccggaggtggaggtggaactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagg

SEQ ID NO: 66 - protein, 226 aa, synthetic construct
  REGION 1..226: note = synthetic construct
PRKKGCWKCGKEGHQMKDCSGGGGSGGGSMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYSGGGGGTERQANFLGKIWPSHKGRPGNFLQSRPEPTAPPEESFRFGEETTTPSQKQEPIDKELYPLASLRSLFGSDPSSQ

SEQ ID NO: 67 - DNA, 1591 bp, synthetic construct
  misc_feature 1..1591: note = synthetic construct
  source 1..1591: mol_type = other DNA
cctaggaaaaagggctgttggaaatgtggaaaggaaggacaccaaatgaaagattgttccggtggaggtggatccatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacggatccggtggaggttccatggcgtccaatttcacgcagttcgtcctggttgacaacggggggactggggacgttacggtcgctccgagcaactttgccaatggtattgcggagtggatttcttctaattcacggtcccaagcttacaaagtgacctgttccgtgcggcaaagttctgctcagaatagaaagtacactataaaggtcgaagtccctaagggggcctggcgatcatatctcaatatggagcttaccatcccaatatttgccactaattctgattgtgaattgattgtcaaagcaatgcaaggactcttgaaagacggaaacccaatccccagcgcaatcgcagccaactccggtatatactccggaggtggaggtggaactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagg

SEQ ID NO: 68 - protein, 345 aa, synthetic construct
  REGION 1..345: note = synthetic construct
PRKKGCWKCGKEGHQMKDCSGGGGSMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSGGGSMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYSGGGGGTERQANFLGKIWPSHKGRPGNFLQSRPEPTAPPEESFRFGEETTTPSQKQEPIDKELYPLASLRSLFGSDPSSQ

SEQ ID NO: 69 - DNA, 1234 bp, synthetic construct
  misc_feature 1..1234: note = synthetic construct
  source 1..1234: mol_type = other DNA
cctaggaaaaagggctgttggaaatgtggaaaggaaggacaccaaatgaaagattgttccggtggaggtggatcctccaaaacaatagtcctctccgtaggggaggcaacacggactttgaccgaaatccagtcaaccgctgaccgacaaatctttgaagagaaagtagggcctcttgtgggccgactgcgcttgactgcaagcttgcgacaaaacggcgcaaagactgcctatagggtcaaccttaaactcgaccaagccgacgtggtcgatagcggtctccctaaggttcggtatacgcaggtctggagtcatgacgtaacaatcgtagcaaacagcacagaagcctcccgaaaaagcctctacgatctgacgaaatccttggtggctacgtcacaggtggaagacctcgttgtcaaccttgtacctctgggtcggtccggaggtggaggtggaactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagg

SEQ ID NO: 70 - protein, 226 aa, synthetic construct
  REGION 1..226: note = synthetic construct
PRKKGCWKCGKEGHQMKDCSGGGGSSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRSGGGGGTERQANFLGKIWPSHKGRPGNFLQSRPEPTAPPEESFRFGEETTTPSQKQEPIDKELYPLASLRSLFGSDPSSQ

SEQ ID NO: 71 - DNA, 1176 bp, synthetic construct
  misc_feature 1..1176: note = synthetic construct
  source 1..1176: mol_type = other DNA
cacgtgagatctgaattcgagatctgccgccgccatgggtgcgagagcgtcagtattaagcgggggagaattagatcgatgggaaaaaattcggttaaggccagggggaaagaaaaaatataaattaaaacatatagtatgggcaagcagggagctagaacgaggaggtggtggaggaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacggtggtggaggaggaatggcgtccaatttcacgcagttcgtcctggttgacaacggggggactggggacgttacggtcgctccgagcaactttgccaatggtattgcggagtggatttcttctaattcacggtcccaagcttacaaagtgacctgttccgtgcggcaaagttctgctcagaatagaaagtacactataaaggtcgaagtccctaagggggcctggcgatcatatctcaatatggagcttaccatcccaatatttgccactaattctgattgtgaattgattgtcaaagcaatgcaaggactcttgaaagacggaaacccaatccccagcgcaatcgcagccaactccggtatataccaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgc

SEQ ID NO: 72 - protein, 380 aa, synthetic construct
  REGION 1..380: note = synthetic construct
MGARASVLSGGELDRWEKIRLRPGGKKKYKLKHIVWASRELERGGGGGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYQVSQNYPIVQNIQGQMVHQAISPRTLNAWVKVVEEKAFSPEVIPMFSALSEGATPQDLNTMLNTVGGHQAAMQMLKETINEEAAEWDRVHPVH

SEQ ID NO: 73 - DNA, 861 bp, synthetic construct
  misc_feature 1..861: note = synthetic construct
  source 1..861: mol_type = other DNA
cacgtgagatctgaattcgagatctgccgccgccatgggtgcgagagcgtcagtattaagcgggggagaattagatcgatgggaaaaaattcggttaaggccagggggaaagaaaaaatataaattaaaacatatagtatgggcaagcagggagctagaacgaggaggtggtggaggaatggcttctaactttactcagttcgttctcgtcgactccaaaacaatagtcctctccgtaggggaggcaacacggactttgaccgaaatccagtcaaccgctgaccgacaaatctttgaagagaaagtagggcctcttgtgggccgactgcgcttgactgcaagcttgcgacaaaacggcgcaaagactgcctatagggtcaaccttaaactcgaccaagccgacgtggtcgatagcggtctccctaaggttcggtatacgcaggtctggagtcatgacgtaacaatcgtagcaaacagcacagaagcctcccgaaaaagcctctacgatctgacgaaatccttggtggctacgtcacaggtggaagacctcgttgtcaaccttgtacctctgggtcgaaaccaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgc

SEQ ID NO: 74 - protein, 275 aa, synthetic construct
  REGION 1..275: note = synthetic construct
MGARASVLSGGELDRWEKIRLRPGGKKKYKLKHIVWASRELERGGGGGMASNFTQFVLVDSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRNQVSQNYPIVQNIQGQMVHQAISPRTLNAWVKVVEEKAFSPEVIPMFSALSEGATPQDLNTMLNTVGGHQAAMQMLKETINEEAAEWDRVHPVH

SEQ ID NO: 75 - DNA, 1239 bp, synthetic construct
  misc_feature 1..1239: note = synthetic construct
  source 1..1239: mol_type = other DNA
cacgtgagatctgaattcgagatctgccgccgccatgggtgcgagagcgtcagtattaagcgggggagaattagatcgatgggaaaaaattcggttaaggccagggggaaagaaaaaatataaattaaaacatatagtatgggcaagcagggagctagaacgaggaggtggtggaggaatggcttctaactttactcagttcgttctcgtcgactccaaaacaatagtcctctccgtaggggaggcaacacggactttgaccgaaatccagtcaaccgctgaccgacaaatctttgaagagaaagtagggcctcttgtgggccgactgcgcttgactgcaagcttgcgacaaaacggcgcaaagactgcctatagggtcaaccttaaactcgaccaagccgacgtggtcgatagcggtctccctaaggttcggtatacgcaggtctggagtcatgacgtaacaatcgtagcaaacagcacagaagcctcccgaaaaagcctctacgatctgacgaaatccttggtggctacgtcacaggtggaagacctcgttgtcaaccttgtacctctgggtcgagcggatccgctcgcatcaaaaactattgtgctctccgtgggagaagccacccgcacgcttaccgaaattcaatcaacggcagacagacaaatctttgaggagaaagtaggtccgttggtgggtcggttgcgcttgaccgcaagcctccgccaaaacggagcgaaaaccgcataccgcgtaaacttgaagctggaccaagccgatgttgttgactccggcttgcccaaagtgcgatatactcaggtctggtctcatgatgtcacaatcgtcgctaattccactgaggctagtcgcaaaagtctgtatgacttgacaaagtccttggtagccacgtcacaggtggaagatttggtggtgaacctcgttccactgggaagaaaccaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgc

SEQ ID NO: 76 - protein, 401 aa, synthetic construct
  REGION 1..401: note = synthetic construct
MGARASVLSGGELDRWEKIRLRPGGKKKYKLKHIVWASRELERGGGGGMASNFTQFVLVDSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRADPLASKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRNQVSQNYPIVQNIQGQMVHQAISPRTLNAWVKVVEEKAFSPEVIPMFSALSEGATPQDLNTMLNTVGGHQAAMQMLKETINEEAAEWDRVHPVH

SEQ ID NO: 77 - DNA, 159 bp, synthetic construct
  misc_feature 1..159: note = synthetic construct
  source 1..159: mol_type = other DNA
ggtctcgcaccgagtaacggcagacttctccacgtttaagtactctggaaacagaatctacttaaacaaggcaaaatgccgtgtttatctcgtcaacttgttggcgagatggccaacatgaggatcacccatgtctgcagggcctttttttgcggccgc

SEQ ID NO: 78 - DNA, 154 bp, synthetic construct
  misc_feature 1..154: note = synthetic construct
  source 1..154: mol_type = other DNA
ggtctcgcaccgagtaacggcagacttctccacgtttaagtactctgggccaacatgaggatcacccatgtctgcagggcccagaatctacttaaacaaggcaaaatgccgtgtttatctcgtcaacttgttggcgagatttttttgcggccgc

SEQ ID NO: 79 - DNA, 154 bp, synthetic construct
  misc_feature 1..154: note = synthetic construct
  source 1..154: mol_type = other DNA
ggtctcgcaccgagtaacggcagacttctccacgtttaagtactctggaaacagaatctacttaaacaaggcaaaatgccgtgtttatctcgtcaaggccaacatgaggatcacccatgtctgcagggccttggcgagatttttttgcggccgc

SEQ ID NO: 80 - DNA, 184 bp, synthetic construct
  misc_feature 1..184: note = synthetic construct
  source 1..184: mol_type = other DNA
ggtctcgcaccgagtaacggcagacttctccacgtttaagtactctgggccaacatgaggatcacccatgtctgcagggcccagaatctacttaaacaaggcaaaatgccgtgtttatctcgtcaaggccaacatgaggatcacccatgtctgcagggccttggcgagatttttttgcggccgc

SEQ ID NO: 81 - DNA, 394 bp, synthetic construct
  misc_feature 1..394: note = synthetic construct
  source 1..394: mol_type = other DNA
ggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagtaacggcagacttctccacgttctagtactctggaaacagaatctactagaacaaggcaaaatgccgtgtttatctcgtcaacttgttggcgagatggagcagacgatatggcgtcgctcctttttttgcggccgc

SEQ ID NO: 82 - DNA, 3376 bp, synthetic construct
  misc_feature 1..3376: note = synthetic construct
  source 1..3376: mol_type = other DNA
gcggccgcttgtgtaatcgtagtttcagagtgttagagctgaaaggaagaagtaggagaaacatgcaaagtaaaagtataacactttccttactaaaccgacatgggtttccaggtaggggcaggattcaggatgactgacagggcccttagggaacactgagaccctacgctgacctcataaatgcttgctacctttgctgttttaattacatcttttaatagcaggaagcagaactctgcacttcaaaagtttttcctcacctgaggagttaatttagtacaaggggaaaaagtacagggggatgggagaaaggcgatcacgttgggaagctatagagaaagaagagtaaattttagtaaaggaggtttaaacaaacaaaatataaagagaaataggaacttgaatcaaggaaatgattttaaaacgcagtattcttagtggactagaggaaaaaaataatctgagccaagtagaagaccttttcccctcctacccctactttctaagtcacagaggctttttgttcccccagacactcttgcagattagtccaggcagaaacagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtatatatacacacatatatatatatattttttcttttcttaccagaaggttttaatccaaataaggagaagatatgcttagaaccgaggtagagttttcatccattctgtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactcctgaggagaaaagcgctgtgacagctctctggggaaaagtcaatgtcgacgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatctcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccctgtggggcaaggtgaacgttttagtactctggaaacagaatctactaaaacaaggcaaaatgccgtgtttatctcgtcaacttgttggcgagatttttctagagcggccgc

SEQ ID NO: 83 - DNA, 275 bp, synthetic construct
  misc_feature 1..275: note = synthetic construct
  source 1..275: mol_type = other DNA
ggatcctaagctcgctttcttgctgtccaatttctattaaaggttcctttgttccctaagtccaactactaaactgggggatattatgaagggccttgagcatctggattctgcctaataaaaaacatttattttcattgctagctcgctttcttgctgtccaatttctattaaaggttcctttgttccctaagtccaactactaaactgggggatattatgaagggccttgagcatctggattctgcctaataaaaaacatttattttcattgc

SEQ ID NO: 84 - DNA, 530 bp, synthetic construct
  misc_feature 1..530: note = synthetic construct
  source 1..530: mol_type = other DNA
cggccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagtaacggcagacttctccacgttctagtactctggaaacagaatctactagaacaaggcaaaatgccgtgtttatctcgtcaacttgttggcgagatatcggagcagacgatatggcgtcgctccagcgctcgctttcttgctgtccaatttctattaaaggttcctttgttccctaagtccaactactaaactgggggatattatgaagggccttgagcatctggattctgcctaataaaaaacatttattttcattgctttttttgcggccg

SEQ ID NO: 85 - DNA, 313 bp, synthetic construct
  misc_feature 1..313: note = synthetic construct
  source 1..313: mol_type = other DNA
gatatcggagcagacgatatggcgtcgctccagcgctcgctttcttgctgtccaatttctattaaaggttcctttgttccctaagtccaactactaaactgggggatattatgaagggccttgagcatctggattctgcctaataaaaaacatttattttcattgcgctcgctttcttgctgtccaatttctattaaaggttcctttgttccctaagtccaactactaaactgggggatattatgaagggccttgagcatctggattctgcctaataaaaaacatttattttcattgctttttttgcggccgc

SEQ ID NO: 86 - DNA, 60 bp, synthetic construct
  misc_feature 1..60: note = synthetic construct
  source 1..60: mol_type = other DNA
ctggctaactaccggctcttctggtccccacagactcagagagaaccggtgccaccatgg

SEQ ID NO: 87 - DNA, 60 bp, synthetic construct
  misc_feature 1..60: note = synthetic construct
  source 1..60: mol_type = other DNA
ccatggtggcaccggttctctctgagtctgtggggaccagaagagccggtagttagccag

SEQ ID NO: 88 - DNA, 73 bp, synthetic construct
  misc_feature 1..73: note = synthetic construct
  source 1..73: mol_type = other DNA
aaaaaagaaaaagctttagaaaacatgaggatcacccatgtctgcaggtcgactctagaattcctagagctcg

SEQ ID NO: 89 - DNA, 73 bp, synthetic construct
  misc_feature 1..73: note = synthetic construct
  source 1..73: mol_type = other DNA
cgagctctaggaattctagagtcgacctgcagacatgggtgatcctcatgttttctaaagctttttctttttt

SEQ ID NO: 90 - DNA, 24 bp, synthetic construct
  misc_feature 1..24: note = synthetic construct
  source 1..24: mol_type = other DNA
accggcgcttgctcttcattccct

SEQ ID NO: 91 - DNA, 24 bp, synthetic construct
  misc_feature 1..24: note = synthetic construct
  source 1..24: mol_type = other DNA
aaacagggaatgaagagcaagcgc

SEQ ID NO: 92 - DNA, 67 bp, synthetic construct
  misc_feature 1..67: note = synthetic construct
  source 1..67: mol_type = other DNA
acttgttggcgagatatctagaaaacatgaggatcacccatgtctgcagagcgctcgctttcttgct

SEQ ID NO: 93 - DNA, 67 bp, synthetic construct
  misc_feature 1..67: note = synthetic construct
  source 1..67: mol_type = other DNA
agcaagaaagcgagcgctctgcagacatgggtgatcctcatgttttctagatatctcgccaacaagt

SEQ ID NO: 94 - DNA, 27 bp, synthetic construct
  misc_feature 1..27: note = synthetic construct
  source 1..27: mol_type = other DNA
actcctgtggagaagtctgccgttact

SEQ ID NO: 95 - DNA, 119 bp, synthetic construct
  misc_feature 1..119: note = synthetic construct
  source 1..119: mol_type = other DNA
gaacccaggttcctgacacagacagactacacccagggaatgaagagcaagcgccatactcctgtggagaagtctgccgttactgccctgtggggcaaggtgaacgtggattggctagc

SEQ ID NO: 96 - DNA, 24 bp, synthetic construct
  misc_feature 1..24: note = synthetic construct
  source 1..24: mol_type = other DNA
cgatcacgttgggaagctatagag

SEQ ID NO: 97 - DNA, 23 bp, synthetic construct
  misc_feature 1..23: note = synthetic construct
  source 1..23: mol_type = other DNA
aacatcctgaggaagaatgggac

SEQ ID NO: 98 - DNA, 54 bp, synthetic construct
  misc_feature 1..54: note = synthetic construct
  source 1..54: mol_type = other DNA
tcgtcggcagcgtcagatgtgtataagagacagttccatttcaggtgtcgtgag

SEQ ID NO: 99 - DNA, 55 bp, synthetic construct
  misc_feature 1..55: note = synthetic construct
  source 1..55: mol_type = other DNA
tcgtcggcagcgtcagatgtgtataagagacagattccatttcaggtgtcgtgag

SEQ ID NO: 100 - DNA, 56 bp, synthetic construct
  misc_feature 1..56: note = synthetic construct
  source 1..56: mol_type = other DNA
tcgtcggcagcgtcagatgtgtataagagacaggattccatttcaggtgtcgtgag

SEQ ID NO: 101 - DNA, 57 bp, synthetic construct
  misc_feature 1..57: note = synthetic construct
  source 1..57: mol_type = other DNA
tcgtcggcagcgtcagatgtgtataagagacagcgattccatttcaggtgtcgtgag

SEQ ID NO: 102 - DNA, 55 bp, synthetic construct
  misc_feature 1..55: note = synthetic construct
  source 1..55: mol_type = other DNA
gtctcgtgggctcggagatgtgtataagagacagtgaacttcagggtcagcttgc

SEQ ID NO: 103 - DNA, 61 bp, synthetic construct
  misc_feature 1..61: note = synthetic construct
  source 1..61: mol_type = other DNA
gaagagcaagcgccatactcctgtggagaagtctgccgttactgccctgtggggcaaggtg

SEQ ID NO: 104 - DNA, 49 bp, synthetic construct
  misc_feature 1..49: note = synthetic construct
  source 1..49: mol_type = other DNA
gaagagcaagcgccatactcctgccgttactgccctgtggggcaaggtg

SEQ ID NO: 105 - DNA, 60 bp, synthetic construct
  misc_feature 1..60: note = synthetic construct
  source 1..60: mol_type = other DNA
gaagagcaagcgccatactcctgtgagaagtctgccgttactgccctgtggggcaaggtg

SEQ ID NO: 106 - DNA, 58 bp, synthetic construct
  misc_feature 1..58: note = synthetic construct
  source 1..58: mol_type = other DNA
gaagagcaagcgccatactcctgtgaagtctgccgttactgccctgtggggcaaggtg

SEQ ID NO: 107 - DNA, 53 bp, synthetic construct
  misc_feature 1..53: note = synthetic construct
  source 1..53: mol_type = other DNA
gaagagcaagcgccatactcctgtctgccgttactgccctgtggggcaaggtg

SEQ ID NO: 108 - DNA, 58 bp, synthetic construct
  misc_feature 1..58: note = synthetic construct
  source 1..58: mol_type = other DNA
gaagagcaagcgccatactcctgagaagtctgccgttactgccctgtggggcaaggtg

SEQ ID NO: 109 - DNA, 59 bp, synthetic construct
  misc_feature 1..59: note = synthetic construct
  source 1..59: mol_type = other DNA
gaagagcaagcgccatactcctggagaagtctgccgttactgccctgtggggcaaggtg

SEQ ID NO: 110 - DNA, 35 bp, synthetic construct
  misc_feature 1..35: note = synthetic construct
  source 1..35: mol_type = other DNA
gaagagcaagcgccatactcctgtggggcaaggtg

SEQ ID NO: 111 - DNA, 55 bp, synthetic construct
  misc_feature 1..55: note = synthetic construct
  source 1..55: mol_type = other DNA
gaagagcaagcgccatactcctgtgtctgccgttactgccctgtggggcaaggtg

SEQ ID NO: 112 - DNA, 38 bp, synthetic construct
  misc_feature 1..38: note = synthetic construct
  source 1..38: mol_type = other DNA
gaagagcaagcgccgttactgccctgtggggcaaggtg

SEQ ID NO: 113 - DNA, 4004 bp, synthetic construct
  misc_feature 1..4004: note = synthetic construct
  source 1..4004: mol_type = other DNA
gcggccgctctcgaactcctcaagcaatccacctgccttggcctcccaaagtgctgggattgcaggcgtgagtcactgcacccagccgagagaataaatttctgttggtttaagccactcagtttggggataacttatggcagccctagcaaactaatacatactaaagatacatactaaatactaagctgggccatatagtccagttttcctgagactcccaggcaagtgctgtttttctttgcttaatatcctacaccactttctgtctggtaaaattacactcattctttaagatgccactgaaatagcacctcttcagcacagccttcactaaactatccccctctccatcttggtaaatttagttacttcctcttctgtgctcacatactttgtagtatctctacatttatgctataggacttgttacactatgttgtattacttgtttatgtcttccccacttttctgtgagtgtctagaaatatgaggatgtcttgttggtctatttccagaacataagcacagtgcctggcacatattaaaaacgtaataaatgtttgctgaataaatagtttctgtaagtggcttctccaatcacctctgtgttttcggggaaggtaaaactggcaacaggatgaagaatggattagagagcagagggcctttagaaagggaggccagttgatggagtctagatagaatcatgactagagctaatgaaagactgatttagcagagtggctgtggtaatggaaaggaggaaaccgttgggagaaacaccacagaagcagagtgggttatattctctgggtgagagagggggagaaattgaagctgattctgaggtttcaagtctgggtgactgagagggtgacgataccattgactgaggtggggaaggcaggaagagaagcagagttgggggaagatgggaagcttgaagctagtattgttgttcctccatttctagaatatttttgtattataagtcacacttcctcgccagtctcaacagggacccagctcaggcagcagctaagggtgggtattctggtttggattagatcagaggaaagacagctgtatatgtgcccacaggagccaagacggtattttccatcctcccaaaacagtagagctttgacagagatttaagggtgaccaagtcaaggaagaggcatggcatagaacggtgatgtcgggggtgggggttcagaacttccattatagaaggtaatgatttagaggagaaggtggttgagaatggtgctagtggtagtgaacagatccttcccaggatctaggtgggctgaggatttttgagtctgtgacactattgtatatccagctttagtttctgtttaccaccttacagcagcacctaatctcctagaggacttagcccgtgtcacacagcacatatttgccacaccctctgtaaagccctggtttataaggttctttccaccggaagctatgacagaggaaacgtgtgggtggggaggggtagtgggtgagggacccaggttcctgccaccatgctcaaaccttccctgccttttaccagcttgctgtttctccagctccctctcctcggcgtcggactcaatacaactatcctgacacctaacggaaacgaggatacaaccgccgacttttttctcacaaccatgcctacagatagcttgtccgtgagcaccctccctctgcctgaagtgcagtgcttcgtctttaacgtggaatatatgaactgtacctggaattcctccagcgaacctcagccaacaaatctgacactccactactggtataaaaatagcgacaacgacaaggtgcagaaatgttcccattacttgtttagcgaggagattaccagcggatgccagctccagaagaaagaaattcatctgtatcagaccttcgtggtgcagctgcaggatcctagagagcctagaaggcaggctacccagatgttgaagctccagaacctcgtcattccttgggcccctgaaaatctgaccttgcataagctctccgagagccagctggagctcaattggaataacaggtttctgaatcattgcctggaacatctcgtccaatatagaaccgattgggatcattcctggaccgagcagagcgtcgactaccggcacaaatttagcctgccaagcgtcgacggacagaagagatataccttcagagtgagatccagattcaatcctctgtgcggctccgcacagcactggtccgagtggtcccatcctattcattggggatccaacaccagcaaggaaaacccatttctgttcgctctggaggctgtcgtgattagcgtgggaagcatgggcctgatcatttccttgctgtgcgtttacttttggctggagagaaccatgcctaggatccctacactcaaaaatctggaagacttggtgacagagtatcatggaaatttcagcgcttggtccggagtcagcaaaggcctcgccgaaagcctccagcctgattatagcgagaggctgtgtctggtgagcgaaatccctcctaagggcggagctctgggcgaaggaccaggcgctagcccttgtaatcagcactccccttattgggctcctccttgctatacattgaaaccagagacataagggaacccaggagacaggccacacagatgctaaaactgcagaatctgggtaatttggaaagaaagggtcaagagaccagggatactgtgggacattggagtctacagagtagtgttcttttatcataagggtacatgggcagaaaagaggaggtaggggatcatgatgggaagggaggaggtattaggggcactaccttcaggatcctgacttgtctaggccaggggaatgaccacatatgcacacatatctccagtgatcccctgggctccagagaacctaacacttcacaaactgagtgaatcccagctagaactgaactggaacaacagattcttgaaccactgtttggagcacttggtgcagtaccggactgactgggaccacagctggactgtgagtgactagggacgtgaatgtagcagctaaggccaagaaagtagggctaaaggattcaaccagacagatagaaggacctaatatcaagctcctgttctctgcctcccagcttctctgctcaccccctaccctccctcctccaactcctttcctcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtggcctgtctcctgggttcccgttttagtactctggaaacagaatctactaaaacaaggcaaaatgccgtgtttatctcgtcaacttgttggcgagattttttgttttagagctagaaatagcaagttaaaataaggctagtccgtttttagcgcgtgcgccaattctgcagacaaatgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggacacagacagactacacccagttttagtactctggaaacagaatctactaaaacaaggcaaaatgccgtgtttatctcgtcaacttgttggcgagatttttggtaccggtgcggccgc

SEQ ID NO: 114 - DNA, 3372 bp, synthetic construct
  misc_feature 1..3372: note = synthetic construct
  source 1..3372: mol_type = other DNA
gcggccgcttgtgtaatcgtagtttcagagtgttagagctgaaaggaagaagtaggagaaacatgcaaagtaaaagtataacactttccttactaaaccgacatgggtttccaggtaggggcaggattcaggatgactgacagggcccttagggaacactgagaccctacgctgacctcataaatgcttgctacctttgctgttttaattacatcttttaatagcaggaagcagaactctgcacttcaaaagtttttcctcacctgaggagttaatttagtacaaggggaaaaagtacagggggatgggagaaaggcgatcacgttgggaagctatagagaaagaagagtaaattttagtaaaggaggtttaaacaaacaaaatataaagagaaataggaacttgaatcaaggaaatgattttaaaacgcagtattcttagtggactagaggaaaaaaataatctgagccaagtagaagaccttttcccctcctacccctactttctaagtcacagaggctttttgttcccccagacactcttgcagattagtccaggcagaaacagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtatatatacacacatatatatatatattttttcttttcttaccagaaggttttaatccaaataaggagaagatatgcttagaaccgaggtagagttttcatccattctgtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactcctgaggagaaaagcgctgtgacagctctctggggaaaagtcaatgtcgacgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatctcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagtaacggcagacttctccacgttttagtactctggaaacagaatctactaaaacaaggcaaaatgccgtgtttatctcgtcaacttgttggcgagatttttgcggccgc

SEQ ID NO: 115 - DNA, 22 bp, synthetic construct
  misc_feature 1..22: note = synthetic construct
  source 1..22: mol_type = other DNA
gggcagagccatctattgctta

SEQ ID NO: 116 - DNA, 25 bp, synthetic construct
  misc_feature 1..25: note = synthetic construct
  source 1..25: mol_type = other DNA
tgggaaaatagaccaataggcagag

SEQ ID NO: 117 - DNA, 20 bp, synthetic construct
  misc_feature 1..20: note = synthetic construct
  source 1..20: mol_type = other DNA
cgccaacaagttgacgagat

SEQ ID NO: 118 - DNA, 23 bp, synthetic construct
  misc_feature 1..23: note = synthetic construct
  source 1..23: mol_type = other DNA
gataaacacggcattttgccttg

SEQ ID NO: 119 - DNA, 55 bp, synthetic construct
  misc_feature 1..55: note = synthetic construct
  source 1..55: mol_type = other DNA
gtctcgtgggctcggagatgtgtataagagacagtggctgaggtgggagaatcac

SEQ ID NO: 120 - DNA, 54 bp, synthetic construct
  misc_feature 1..54: note = synthetic construct
  source 1..54: mol_type = other DNA
tcgtcggcagcgtcagatgtgtataagagacagtatttctcctctgcactgccc

SEQ ID NO: 121 - DNA, 55 bp, synthetic construct
  misc_feature 1..55: note = synthetic construct
  source 1..55: mol_type = other DNA
gtctcgtgggctcggagatgtgtataagagacagtggagcctcggcctagatttc

SEQ ID NO: 122 - DNA, 54 bp, synthetic construct
  misc_feature 1..54: note = synthetic construct
  source 1..54: mol_type = other DNA
tcgtcggcagcgtcagatgtgtataagagacagttgatcccttccaccaatgtc

SEQ ID NO: 123 - DNA, 55 bp, synthetic construct
  misc_feature 1..55: note = synthetic construct
  source 1..55: mol_type = other DNA
gtctcgtgggctcggagatgtgtataagagacagtcacggctaggaatagcaagg

SEQ ID NO: 124 - DNA, 58 bp, synthetic construct
  misc_feature 1..58: note = synthetic construct
  source 1..58: mol_type = other DNA
tcgtcggcagcgtcagatgtgtataagagacagtaaaggaaattccatcagactaacg

SEQ ID NO: 125 - DNA, 55 bp, synthetic construct
  misc_feature 1..55: note = synthetic construct
  source 1..55: mol_type = other DNA
gtctcgtgggctcggagatgtgtataagagacagtctttgaaaatgccgtccatc

SEQ ID NO: 126 - DNA, 56 bp, synthetic construct
  misc_feature 1..56: note = synthetic construct
  source 1..56: mol_type = other DNA
tcgtcggcagcgtcagatgtgtataagagacagtaagacaaaagcagaaggtaagc

SEQ ID NO: 127 - DNA, 58 bp, synthetic construct
  misc_feature 1..58: note = synthetic construct
  source 1..58: mol_type = other DNA
gtctcgtgggctcggagatgtgtataagagacagtaagttgaagataggaccccactc

SEQ ID NO: 128 - DNA, 55 bp, synthetic construct
  misc_feature 1..55: note = synthetic construct
  source 1..55: mol_type = other DNA
gtctcgtgggctcggagatgtgtataagagacagtggcaacaagagcaaaactcc

SEQ ID NO: 129 - DNA, 55 bp, synthetic construct
  misc_feature 1..55: note = synthetic construct
  source 1..55: mol_type = other DNA
tcgtcggcagcgtcagatgtgtataagagacagtccagaaagggaaaacttgcac

SEQ ID NO: 130 - DNA, 55 bp, synthetic construct
  misc_feature 1..55: note = synthetic construct
  source 1..55: mol_type = other DNA
gtctcgtgggctcggagatgtgtataagagacagtccactgacccaatcttttcc

SEQ ID NO: 131 - DNA, 54 bp, synthetic construct
  misc_feature 1..54: note = synthetic construct
  source 1..54: mol_type = other DNA
tcgtcggcagcgtcagatgtgtataagagacagtctgctctttgcctgttggag

SEQ ID NO: 132 - DNA, 55 bp, synthetic construct
  misc_feature 1..55: note = synthetic construct
  source 1..55: mol_type = other DNA
gtctcgtgggctcggagatgtgtataagagacagtgctaaagctggaaggctgtg

SEQ ID NO: 133 - DNA, 20 bp, synthetic construct
  misc_feature 1..20: note = synthetic construct
  source 1..20: mol_type = other DNA
atgccctctgtagtgggttg

SEQ ID NO: 134 - DNA, 20 bp, synthetic construct
  misc_feature 1..20: note = synthetic construct
  source 1..20: mol_type = other DNA
ggcagctgcaggaataagag

SEQ ID NO: 135 - DNA, 55 bp, synthetic construct
  misc_feature 1..55: note = synthetic construct
  source 1..55: mol_type = other DNA
tcgtcggcagcgtcagatgtgtataagagacagtgaagctatgacagaggaaacg

SEQ ID NO: 136 - DNA, 40 bp, synthetic construct
  misc_feature 1..40: note = Synthetic construct
  source 1..40: mol_type = other DNA
agcgccatactcctgtggagaagtctgccgttactgccct

SEQ ID NO: 137 - DNA, 40 bp, synthetic construct; identical to SEQ ID NO: 136
  misc_feature 1..40: note = Synthetic construct
  source 1..40: mol_type = other DNA

SEQ ID NO: 138 - DNA, 27 bp, synthetic construct
  misc_feature 1..27: note = Synthetic construct
  source 1..27: mol_type = other DNA
agcgccatactcctgccgttactgcct

SEQ ID NO: 139 - DNA, 39 bp, synthetic construct
  misc_feature 1..39: note = Synthetic construct
  source 1..39: mol_type = other DNA
agcgccatactcctgtgagaagtctgccgttactgccct

SEQ ID NO: 140 - DNA, 38 bp, synthetic construct
  misc_feature 1..38: note = Synthetic construct
  source 1..38: mol_type = other DNA
agccgccatactcctgtgaagtctgccgttactgccct

SEQ ID NO: 141 - DNA, 32 bp, synthetic construct
  misc_feature 1..32: note = Synthetic construct
  source 1..32: mol_type = other DNA
agcgccatactcctgtctgccgttactgccct

SEQ ID NO: 142 - DNA, 37 bp, synthetic construct
  misc_feature 1..37: note = Synthetic construct
  source 1..37: mol_type = other DNA
agcgccatactcctgagaagtctgccgttactgccct

SEQ ID NO: 143 - DNA, 38 bp, synthetic construct
  misc_feature 1..38: note = Synthetic construct
  source 1..38: mol_type = other DNA
agcgccatactcctggagaagtctgccgttactgccct

SEQ ID NO: 144 - DNA, 14 bp, synthetic construct
  misc_feature 1..14: note = Synthetic construct
  source 1..14: mol_type = other DNA
agcgccatactcct

SEQ ID NO: 145 - DNA, 34 bp, synthetic construct
  misc_feature 1..34: note = Synthetic construct
  source 1..34: mol_type = other DNA
agcgccatactcctgtgtctgccgttactgccct

SEQ ID NO: 146 - DNA, 17 bp, synthetic construct
  misc_feature 1..17: note = Synthetic construct
  source 1..17: mol_type = other DNA
agcgccgttactgccct

SEQ ID NO: 147 - DNA, 33 bp, synthetic construct
  misc_feature 1..33: note = Synthetic construct
  source 1..33: mol_type = other DNA
cgccatactcctgtggagaagtctgccgttact

SEQ ID NO: 148 - DNA, 39 bp, synthetic construct
  misc_feature 1..39: note = Synthetic construct
  source 1..39: mol_type = other DNA
ttcctgacacagacagactacacccagggaatgaagagc

SEQ ID NO: 149 - DNA, 38 bp, synthetic construct
  misc_feature 1..38: note = Synthetic construct
  source 1..38: mol_type = other DNA
ttcctgacacagacagactacaccagggaatgaagagc

SEQ ID NO: 150 - DNA, 35 bp, synthetic construct
  misc_feature 1..35: note = Synthetic construct
  source 1..35: mol_type = other DNA
ttcctgacacagacagactacagggaatgaagagc

SEQ ID NO: 151 - DNA, 37 bp, synthetic construct
  misc_feature 1..37: note = Synthetic construct
  source 1..37: mol_type = other DNA
ttcctgacacagacagactacacagggaatgaagagc

SEQ ID NO: 152 - DNA, 28 bp, synthetic construct
  misc_feature 1..28: note = Synthetic construct
  source 1..28: mol_type = other DNA
ttcctgacacagacagggaatgaagagc

SEQ ID NO: 153 - DNA, 36 bp, synthetic construct
  misc_feature 1..36: note = Synthetic construct
  source 1..36: mol_type = other DNA
ttcctgacacagacagactaccagggaatgaagagc

SEQ ID NO: 154 - DNA, 30 bp, synthetic construct
  misc_feature 1..30: note = Synthetic construct
  source 1..30: mol_type = other DNA
ttcctgacacagacagactacacgaagagc

SEQ ID NO: 155 - DNA, 32 bp, synthetic construct
  misc_feature 1..32: note = Synthetic construct
  source 1..32: mol_type = other DNA
ttcctgacacagacagacagggaatgaagagc

SEQ ID NO: 156 - DNA, 39 bp, synthetic construct; identical to SEQ ID NO: 148
  misc_feature 1..39: note = Synthetic construct
  source 1..39: mol_type = other DNA

SEQ ID NO: 157 - DNA, 38 bp, synthetic construct; identical to SEQ ID NO: 149
  misc_feature 1..38: note = Synthetic construct
  source 1..38: mol_type = other DNA

SEQ ID NO: 158 - DNA, 35 bp, synthetic construct; identical to SEQ ID NO: 150
  misc_feature 1..35: note = Synthetic construct
  source 1..35: mol_type = other DNA

SEQ ID NO: 159 - DNA, 37 bp, synthetic construct; identical to SEQ ID NO: 151
  misc_feature 1..37: note = Synthetic construct
  source 1..37: mol_type = other DNA

SEQ ID NO: 160 - DNA, 28 bp, synthetic construct
  misc_feature 1..28: note = Synthetic construct
  source 1..28: mol_type = other DNA
ttcctgacacagacagggaatgaagagc

SEQ ID NO: 161 - DNA, 36 bp, synthetic construct; identical to SEQ ID NO: 153
  misc_feature 1..36: note = Synthetic construct
  source 1..36: mol_type = other DNA

SEQ ID NO: 162 - DNA, 30 bp, synthetic construct; identical to SEQ ID NO: 154
  misc_feature 1..30: note = Synthetic construct
  source 1..30: mol_type = other DNA

SEQ ID NO: 163 - DNA, 32 bp, synthetic construct; identical to SEQ ID NO: 155
  misc_feature 1..32: note = Synthetic construct
  source 1..32: mol_type = other DNA

SEQ ID NO: 164 - RNA, 21 nt, synthetic construct
  misc_feature 1..21: note = Synthetic construct
  source 1..21: mol_type = other RNA
cacctcttcagacggcaatga

SEQ ID NO: 165 - DNA, 40 bp, synthetic construct
  misc_feature 1..40: note = Synthetic construct
  source 1..40: mol_type = other DNA
atggtgcacctgactcctgaggagaagtctgccgttactg

SEQ ID NO: 166 - DNA, 37 bp, synthetic construct
  misc_feature 1..37: note = Synthetic construct
  source 1..37: mol_type = other DNA
atggtgcacctgactcctgagaagtctgccgttactg

SEQ ID NO: 167 - DNA, 39 bp, synthetic construct
  misc_feature 1..39: note = Synthetic construct
  source 1..39: mol_type = other DNA
atggtgcacctgactcctgagagaagtctgccgttactg

SEQ ID NO: 168 - DNA, 35 bp, synthetic construct
  misc_feature 1..35: note = Synthetic construct
  source 1..35: mol_type = other DNA
atggtgcacctgactcctgaagtctgccgttactg

SEQ ID NO: 169 - DNA, 38 bp, synthetic construct
  misc_feature 1..38: note = Synthetic construct
  source 1..38: mol_type = other DNA
atggtgcacctgactcctggagaagtctgccgttactg

SEQ ID NO: 170 - DNA, 34 bp, synthetic construct
  misc_feature 1..34: note = Synthetic construct
  source 1..34: mol_type = other DNA
atggtgcacctgactcctgagtctgccgttactg

SEQ ID NO: 171 - DNA, 28 bp, synthetic construct
  misc_feature 1..28: note = Synthetic construct
  source 1..28: mol_type = other DNA
atggtgcacctgactcctgccgttactg

SEQ ID NO: 172 - DNA, 30 bp, synthetic construct
  misc_feature 1..30: note = Synthetic construct
  source 1..30: mol_type = other DNA
atggtgcacctgagaagtctgccgttactg

SEQ ID NO: 173 - DNA, 35 bp, synthetic construct
  misc_feature 1..35: note = Synthetic construct
  source 1..35: mol_type = other DNA
atggtgcacctgactcgagaagtctgccgttactg

SEQ ID NO: 174 - DNA, 35 bp, synthetic construct
  misc_feature 1..35: note = Synthetic construct
  source 1..35: mol_type = other DNA
atggtgcacctgactcctgaggtctgccgttactg

SEQ ID NO: 175 - DNA, 32 bp, synthetic construct
  misc_feature 1..32: note = Synthetic construct
  source 1..32: mol_type = other DNA
atggtgcacctgactcctgagtgccgttactg

SEQ ID NO: 176 - DNA, 28 bp, synthetic construct
  misc_feature 1..28: note = Synthetic construct
  source 1..28: mol_type = other DNA
agtcacaggcagacttctccacggggag

SEQ ID NO: 177 - RNA, 21 nt, synthetic construct
  misc_feature 1..21: note = Synthetic construct
  source 1..21: mol_type = other RNA
agtaacggcagacttctccac

SEQ ID NO: 178 - DNA, 62 bp, synthetic construct
  misc_feature 1..62: note = Synthetic construct
  source 1..62: mol_type = other DNA
gacccaggttcctgacacagacagactacacccagggaatgaagagcaagcgccatgttgaa

SEQ ID NO: 179 - DNA, 61 bp, synthetic construct
  misc_feature 1..61: note = Synthetic construct
  source 1..61: mol_type = other DNA
gacccaggttcctgacacagacagactacaccagggaatgaagagcaagcgccatgttgaa

SEQ ID NO: 180 - DNA, 58 bp, synthetic construct
  misc_feature 1..58: note = Synthetic construct
  source 1..58: mol_type = other DNA
gacccaggttcctgacacagacagactacagggaatgaagagcaagcgccatgttgaa

SEQ ID NO: 181 - DNA, 60 bp, synthetic construct
  misc_feature 1..60: note = Synthetic construct
  source 1..60: mol_type = other DNA
gacccaggttcctgacacagacagactacacagggaatgaagagcaagcgccatgttgaa

SEQ ID NO: 182 - DNA, 60 bp, synthetic construct
  misc_feature 1..60: note = Synthetic construct
  source 1..60: mol_type = other DNA
gacccaggttcctgacacagacagactacccagggaatgaagagcaagcgccatgttgaa

SEQ ID NO: 183 - DNA, 60 bp, synthetic construct; identical to SEQ ID NO: 182
  misc_feature 1..60: note = Synthetic construct
  source 1..60: mol_type = other DNA

SEQ ID NO: 184 - DNA, 51 bp, synthetic construct
  misc_feature 1..51: note = Synthetic construct
  source 1..51: mol_type = other DNA
gacccaggttcctgacacagacagggaatgaagagcaagcgccatgttgaa

SEQ ID NO: 185 - DNA, 56 bp, synthetic construct
  misc_feature 1..56: note = Synthetic construct
  source 1..56: mol_type = other DNA
gacccaggttcctgacacagacagactacacaatgaagagcaagcgccatgttgaa

SEQ ID NO: 186 - DNA, 53 bp, synthetic construct
  misc_feature 1..53: note = Synthetic construct
  source 1..53: mol_type = other DNA
gacccaggttcctgacacagacagactacacgaagagcaagcgccatgttgaa

SEQ ID NO: 187 - DNA, 34 bp, synthetic construct
  misc_feature 1..34: note = Synthetic construct
  source 1..34: mol_type = other DNA
gacccagggaatgaagagcaagcgccatgttgaa

SEQ ID NO: 188 - DNA, 54 bp, synthetic construct
  misc_feature 1..54: note = Synthetic construct
  source 1..54: mol_type = other DNA
gacccaggttcctgacacaggaaccagggaatgaagagcaagcgccatgttgaa

SEQ ID NO: 189 - DNA, 29 bp, synthetic construct
  misc_feature 1..29: note = Synthetic construct
  source 1..29: mol_type = other DNA
actcctgaggagaagtctgccgttactgc

SEQ ID NO: 190 - RNA, 21 nt, synthetic construct
  misc_feature 1..21: note = synthetic construct
  source 1..21: mol_type = other RNA
cacctcttcagacggcaatga

SEQ ID NO: 191 - DNA, 29 bp, synthetic construct
  misc_feature 1..29: note = Synthetic construct
  source 1..29: mol_type = other DNA
actcctgtggagaagtctgccgttactgc

SEQ ID NO: 192 - DNA, 80 bp, synthetic construct
  misc_feature 1..80: note = Synthetic construct
  source 1..80: mol_type = other DNA
tgggtgagggacccaggttcctgacacagacagactacacccagggaatgaagagcaagcgccatgttgaagccatcatt

SEQ ID NO: 193 - DNA, 79 bp, synthetic construct
  misc_feature 1..79: note = Synthetic construct
  source 1..79: mol_type = other DNA
tgggtgagggacccaggttcctgacacagacagactacaccagggaatgaagagcaagcgccatgttgaagccatcatt

SEQ ID NO: 194 - DNA, 76 bp, synthetic construct
  misc_feature 1..76: note = Synthetic construct
  source 1..76: mol_type = other DNA
tgggtgagggacccaggttcctgacacagacagactacagggaatgaagagcaagcgccatgttgaagccatcatt

SEQ ID NO: 195 - DNA, 69 bp, synthetic construct
  misc_feature 1..69: note = Synthetic construct
  source 1..69: mol_type = other DNA
tgggtgagggacccaggttcctgacacagacagggaatgaagagcaagcgccatgttgaagccatcatt

SEQ ID NO: 196 - DNA, 77 bp, synthetic construct
  misc_feature 1..77: note = Synthetic construct
  source 1..77: mol_type = other DNA
tgggtgagggacccaggttcctgacacagacagactaccagggaatgaagagcaagcgccatgttgaagccatcatt

SEQ ID NO: 197 - DNA, 78 bp, synthetic construct
  misc_feature 1..78: note = Synthetic construct
  source 1..78: mol_type = other DNA
tgggtgagggacccaggttcctgacacagacagactacacagggaatgaagagcaagcgccatgttgaagccatcatt

SEQ ID NO: 198 - DNA, 62 bp, synthetic construct
  misc_feature 1..62: note = Synthetic construct
  source 1..62: mol_type = other DNA
tgggtgagggacccaggttcctgacacagacagactacaagcgccatgttgaagccatcatt

SEQ ID NO: 199 - DNA, 83 bp, synthetic construct
  misc_feature 1..83: note = Synthetic construct
  source 1..83: mol_type = other DNA
tgggtgagggacccaggttcctgacacagacagactactacagacagggaatgaagagcaagcgccatgttgaagccatcatt

SEQ ID NO: 200 - DNA, 73 bp, synthetic construct
  misc_feature 1..73: note = Synthetic construct
  source 1..73: mol_type = other DNA
tgggtgagggacccaggttcctgacacagacagacagggaatgaagagcaagcgccatgttgaagccatcatt

SEQ ID NO: 201 - DNA, 91 bp, synthetic construct
  misc_feature 1..91: note = Synthetic construct
  source 1..91: mol_type = other DNA
tgggtgagggacccaggttcctgacacagacagactacacagggaatgaagacagggaatgaagagcaagcgccatgttgaagccatcatt

SEQ ID NO: 202 - DNA, 61 bp, synthetic construct
  misc_feature 1..61: note = Synthetic construct
  source 1..61: mol_type = other DNA
acccaggttcctgacacagacagactacacccagggaatgaagagcaagcgccatgttgaa

SEQ ID NO: 203 - DNA, 60 bp, synthetic construct
  misc_feature 1..60: note = Synthetic construct
  source 1..60: mol_type = other DNA
acccaggttcctgacacagacagactacaccagggaatgaagagcaagcgccatgttgaa

SEQ ID NO: 204 - DNA, 57 bp, synthetic construct
  misc_feature 1..57: note = Synthetic construct
  source 1..57: mol_type = other DNA
acccaggttcctgacacagacagactacagggaatgaagagcaagcgccatgttgaa

SEQ ID NO: 205 - DNA, 59 bp, synthetic construct
  misc_feature 1..59: note = Synthetic construct
  source 1..59: mol_type = other DNA
acccaggttcctgacacagacagactacacagggaatgaagagcaagcgccatgttgaa

SEQ ID NO: 206 - DNA, 50 bp, synthetic construct
  misc_feature 1..50: note = Synthetic construct
  source 1..50: mol_type = other DNA
acccaggttcctgacacagacagggaatgaagagcaagcgccatgttgaa

SEQ ID NO: 207 - DNA, 58 bp, synthetic construct
  misc_feature 1..58: note = Synthetic construct
  source 1..58: mol_type = other DNA
acccaggttcctgacacagacagactaccagggaatgaagagcaagcgccatgttgaa

SEQ ID NO: 208 - DNA, 52 bp, synthetic construct
  misc_feature 1..52: note = Synthetic construct
  source 1..52: mol_type = other DNA
acccaggttcctgacacagacagactacacgaagagcaagcgccatgttgaa

SEQ ID NO: 209 - DNA, 59 bp, synthetic construct
  misc_feature 1..59: note = Synthetic construct
  source 1..59: mol_type = other DNA
acccaggttcctgacacagacagactacccagggaatgaagagcaagcgccatgttgaa

SEQ ID NO: 210 - DNA, 61 bp, synthetic construct
  misc_feature 1..61: note = Synthetic construct
  source 1..61: mol_type = other DNA
acccaggttcctgccaccatgctcaaaccttccctgccttttaccagcttgctgtttctcc

SEQ ID NO: 211 - RNA, 19 nt, synthetic construct
  misc_feature 1..19: note = Synthetic construct
  source 1..19: mol_type = other RNA
acatgaggatcacccatgt

SEQ ID NO: 212 - RNA, 93 nt, synthetic construct
  misc_feature 1..93: note = synthetic construct
  misc_feature 1..8: note = n is a, c, g, or u
  source 1..93: mol_type = other RNA
nnnnnnnngtttaagtactctggaaacagaatctacttaaacaaaggcaaaatgccgtgtttatctcgtcaacttgtttttttagagcggttg

SEQ ID NO: 213 - RNA, 34 nt, synthetic construct
  misc_feature 1..34: note = synthetic construct
  source 1..34: mol_type = other RNA
ggccaacatgaggatcacccatgtctgcagggcc

SEQ ID NO: 214 - RNA, 34 nt, synthetic construct; identical to SEQ ID NO: 213
  misc_feature 1..34: note = Synthetic construct
  source 1..34: mol_type = other RNA

SEQ ID NO: 215 - RNA, 91 nt, synthetic construct
  misc_feature 1..91: note = Synthetic construct
  misc_feature 1..8: note = n is a, c, g, or u
  source 1..91: mol_type = other RNA
nnnnnnnngtttaagtactctggaaacagaatctacttaaacaaggcaaaatgccgtgtttatctcgtcaacttgttggcgagattttttt

SEQ ID NO: 216 - RNA, 34 nt, synthetic construct; identical to SEQ ID NO: 213
  misc_feature 1..34: note = synthetic construct
  source 1..34: mol_type = other RNA

SEQ ID NO: 217 - RNA, 23 nt, synthetic construct
  misc_feature 1..23: note = synthetic construct
  source 1..23: mol_type = other RNA
ctgaatgcctgcgagcatcccac

SEQ ID NO: 218 - RNA, 25 nt, synthetic construct
  misc_feature 1..25: note = synthetic construct
  source 1..25: mol_type = other RNA
ggagcagacgatatggcgtcgctcc

SEQ ID NO: 219 - RNA, 19 nt, synthetic construct
  misc_feature 1..19: note = Synthetic construct
  source 1..19: mol_type = other RNA
gggccctgaagaagggccc